(19) United States
(12) Patent Application Publication
(10) Pub. No.: US 2015/0225369 A1
(43) Pub. Date: Aug. 13, 2015

(54) DDR2 INHIBITORS FOR THE TREATMENT OF OSTEOARTHRITIS

(71) Applicant: Merck Patent GmbH, Darmstadt (DE)

(72) Inventors: Margarita Wucherer-Plietker, Messel (DE); Daniela Werkmann, Frankfurt am Main (DE); Anne Gigout, Darmstadt (DE); Daniel Kuhn, Rossdorf (DE); Edgar Sawatzky, Darmstadt (DE)

(73) Assignee: Merck Patent GmbH, Darmstadt (DE)

(21) Appl. No.: 14/424,498

(22) PCT Filed: Jul. 29, 2013
(86) PCT No.: PCT/EP2013/002236

§ 371 Date: Feb. 27, 2015

(30) Foreign Application Priority Data
Aug. 29, 2012 (EP) 12006134.6

(51) Int. Cl. C07D 401/12; A61K 31/4725; A61K 31/4709; C07D 213/68; A61K 31/4409; C07D 213/81; A61K 31/44; C07D 417/12; A61K 31/4439; A61K 31/5377; A61K 31/496; C07D 491/048; A61K 31/4355; C07D 239/47; C07D 471/04; A61K 31/444

(52) U.S. Cl. CPC C07D 401/12; A61K 31/444; A61K 31/4725; A61K 31/4709; C07D 213/68; A61K 31/4409; C07D 213/81; A61K 31/44; C07D 417/12; A61K 31/4439; A61K 31/5377; A61K 31/496; C07D 491/048; A61K 31/4355; C07D 239/47; C07D 471/04

(57) ABSTRACT

The present invention relates to compounds of the formula I and in particular medicaments comprising at least one compound of the formula I for use in the treatment and/or prophylaxis of physiological and/or pathophysiological states in the triggering of which DDR2 is involved, in particular for use in the treatment and/or prophylaxis of osteoarthritis, hepatocirrhosis, traumatic cartilage injuries, pain, allodynia or hyperalgesia.

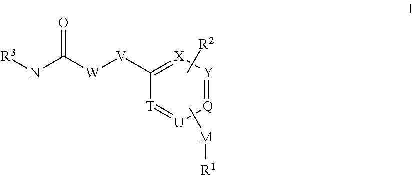

The present invention relates to compounds of the formula I and in particular medicaments comprising at least one compound of the formula I for use in the treatment and/or prophylaxis of physiological and/or pathophysiological states in the triggering of which DDR2 is involved, in particular for use in the treatment and/or prophylaxis of osteoarthritis, hepatocirrhosis, traumatic cartilage injuries, pain, allodynia or hyperalgesia.

### BACKGROUND OF THE INVENTION

Osteoarthritis (OA) is one of the most disabling diseases in developed countries. The prevalence of OA is estimated to one in ten men and one in five women aged over 60 years worldwide. As such, the disease accounts for considerable health care expenditure and therefore represents a significant socio-economic burden. To date, no disease modifying treatment is available. Current treatment is therefore entirely symptomatic up to the point when total joint replacement may be indicated.

In spite of this significant importance for the health system, the causes of OA remain unclear to date and effective preventative measures furthermore remain a distant aim. A reduction in the joint gap (caused by destruction of the joint cartilage), together with changes in the subchondral bone and osteophyte formation, are the radiological characteristics of the disease. For the patient, however, pain (load-dependent and nocturnal rest pain) with subsequent function impairments are to the fore. It is also these which force the patient into social isolation with corresponding secondary diseases.

The term osteoarthritis according to an unofficial definition denotes “joint wear” which exceeds the usual extent for the age. The causes are regarded as being excessive load (for example increased body weight), connatal or traumatic causes, such as malposition of the joint, or also bone deformations due to bone diseases, such as osteoporosis. Osteoarthritis can likewise arise as a consequence of another disease, for example joint inflammation (arthritis) (secondary osteoarthritis), or accompany overload-induced effusion (secondary inflammation reaction) (activated osteoarthritis). The Anglo-American specialist literature differentiates between osteoarthritis (OA), in which the destruction of the joint surfaces can probably be attributed principally to the effects of load, and arthritis (rheumatoid arthritis, RA), in which joint degeneration due to an inflammatory component is to the fore.

In principle, osteoarthritis is also differentiated according to its cause. Arthrosis alcaptonurica is based on increased deposition of homogentisic acid in joints in the case of previously existing alcaptonuria. In the case of haemophilic arthrosis, regular intra-articular bleeding occurs in the case of haemophilia (haemophilic joint). Arthrosis urica is caused by the mechanical influence of urate crystals (uric acid) on the healthy cartilage (Pschyrembel W. et al.: Klinisches Wörterbuch, Verlag Walter de Gruyter & Co, 253rd Edition, 1977).

The classical cause of osteoarthritis is dysplasia of joints. Using the example of the hip, it becomes clear that the zone with the greatest mechanical stress in the case of a physiological hip position represents a significantly larger area than in the case of a dysplastic hip. However, the stresses caused by the forces acting on the joint are substantially independent of the joint shape. They are essentially distributed over the main stress zone(s). A greater pressure will thus arise in the case of a relatively small zone than in the case of a larger one. The biomechanical pressure on the joint cartilage is thus greater in the case of a dysplastic hip than in the case of a physiological hip position. This rule is generally regarded as the cause of the increased occurrence of arthrotic changes in supporting joints which differ from the ideal anatomical shape.

If the consequences of an injury are responsible for premature wear, the term post-traumatic arthrosis is used. Further causes of secondary arthrosis or osteoarthritis that are being discussed are mechanical, inflammatory, metabolic, chemical (quinolones), trophic, hormonal, neurological and genetic reasons. In most cases, however, the diagnosis given is idiopathic arthrosis, by which the doctor means an apparent absence of a causal disease (H. I. Roach and S. Tilley, Bone and Osteoarthritis, F. Bronner and M. C. Farach-Carson (Editors), Verlag Springer, Volume 4, 2007).

Medicinal causes of osteoarthritis can be, for example, antibiotics of the gyrase inhibitor type (fluoroquinolones, such as ciprofloxacin, levofloxacin). These medicaments result in complexing of magnesium ions in poorly vascularised tissues (hyaline joint cartilage, tendon tissue), which has the consequence that irreversible damage occurs to connective tissue. This damage is generally more pronounced in the growth phase in children and juveniles. Tendinopathies and arthropathies are known side effects of this class of medicaments. In adults, these antibiotics result in accelerated physiological degradation of the hyaline joint cartilage according to information from independent pharmacologists and rheumatologists (Menschik M. et al., Antimicrob. Agents Chemother. 41, pp. 2562-2565, 1997; Egerbacher M. et al., Arch. Toxicol. 73, pp. 557-563, 2000; Chang H. et al., Scand. J. Infect. Dis. 28, pp. 641-643, 1996; Chaslerie A. et al., Therapie 47, p. 80, 1992). Extended treatment with phenprocoumone can also favour arthrosis by decreasing bone density in the case of stresses of the joint internal structure.

Besides age, known risk factors for osteoarthrosis are mechanical overload, (micro)traumas, joint destabilisation caused by loss of the securing mechanisms, and genetic factors. However, neither the occurrence nor possible interventions have been fully explained (H. I. Roach and S. Tilley, Bone and Osteoarthritis, F. Bronner and M. C. Farach-Carson (Editors), Verlag Springer, Volume 4, 2007).

In a joint affected by osteoarthritis, the content of nitrogen monoxide is increased in some cases. A similar situation has been observed due to high mechanical irritation of cartilage tissue (Das P. et al., Journal of Orthopaedic Research 15, pp. 87-93, 1997; Farrell A. J. et al., Annals of the Rheumatic Diseases 51, pp. 1219-1222, 1992; Fermor B. et al., Journal of Orthopaedic Research 19, pp. 729-737, 2001), whereas moderate mechanical stimulation tends to have a positive effect. The action of mechanical forces is thus causally involved in the progress of osteoarthritis (Liu X. et al., Biorheology 43, pp. 183-190, 2006).

In principle, osteoarthritis therapy follows two aims: firstly freedom from pain under normal load and secondly the prevention of mechanical restrictions or changes in a joint. These aims cannot be achieved in the long term by pain treatment as a purely symptomatic therapy approach, since this cannot halt the progress of the disease. If the latter is to be achieved, the cartilage destruction must be stopped. Since the joint cartilage in adult patients cannot regenerate, the elimination of pathogenetic factors, such as joint dysplasia or malpositions, which result in increased point pressure on the joint cartilage, is in addition enormously important.

Finally, it is attempted to prevent or stop the degeneration processes in the cartilage tissue with the aid of medicaments.

An essential factor for the functioning state and thus the resistance of the joint cartilage to stress is the extracellular matrix, which primarily consists of collagens, proteoglycans and water. The enzymes involved in degradation of the extracellular matrix include, in particular the metalloproteases, aggrecanases and cathepsin enzymes.

The discoidin domain receptors (DDRs) DDR2 (discoidin domain receptor family member 2, also known as CCK-2, tyro-10 or TKT) and DDR1 (discoidin domain receptor family member 1; also known as MCK-10, DDR, NEP, cak, trkE, RTK6 or ptk3) are members of a receptor tyrosine kinase subfamily, which are activated by collagens.

These proteins are characterized by an extracellular discoidin domain, a domain first identified in the slime mold Dictyostelium discoideum that functions in cell aggregation, and a large cytoplasmic juxtamembrane region. Each protein also contains two immunoglobulin domains. Sequence comparisons show that non-mammalian orthologs of DDRs exist: three closely related genes in Caenorhabditis and one in the sponge Geodia cydonium.

Various types of collagen have been identified as ligands of the two mammalian discoidin domain receptor tyrosine kinases, DDR1 and DDR2. The interaction with collagen both inhibits fibrillogenesis of collagen and regulates expression of matrix-metalloproteases (MMP), enzymes that cleave native fibrillar collagen (Vogel W., FASEB, 13, S77, 1999; Xu et al, J. Biol. Chem. 280:548-55, 2005; Mihai et al., J. Mol. Biol. 361:864-76, 2006). Collagen directly interacts with the extracellular domains and evokes tyrosine phosphorylation of DDRs in a time and concentration dependent manner. DDRs are structurally different from other receptor tyrosine kinases by a discoidin domain and unlike most other receptor tyrosine kinases they are not fully activated within minutes. The binding of collagen to DDRs results in a delayed but sustained tyrosine kinase activation. The maximal activation occurs several hours after collagen stimulation. DDR2 has a much longer juxta-membrane region with supposed autoinhibitory function. DDR2 is only activated by fibrillar collagens (I-III).

Both receptors, DDR1 and DDR2, display several potential tyrosine phosphorylation sites that are able to relay the activation signal by interacting with cytoplasmic effector proteins (Vogel W., FASEB, 13: 577-582, 1999). DDR2 requires srk kinase to be maximally phosphorylated and to activate the matrix metalloproteinase-2 promoter.

The normal function of DDR2 is largely unknown. DDR2 is known to regulate fibroblast and chondrocyte proliferation and migration through the extracellular matrix in association with transcriptional activation of matrix metalloproteinase-2 (Labrador et al., EMBO Reports 2, 5: 446-452, 2001). DDR2 is induced in hepatic stellate cells in response to collagen during liver injury and overexpression of DDR2 enhanced hepatic stellate cell proliferation, activated expression of MMP-2, and enhanced cellular invasion through Matrigel (Olaso et al., J. Clin. Invest., 108: 1369-1378, 2001). DDR2 activation and adhesion in response to collagen may require Wnt and G-protein signaling (Dejmek et al., Int. J. Cancer 103: 344-351, 2003). The lack of DDR2 expression results in dwarfism in mice, probably due to decreased proliferation of cartilage cells during bone growth (Labrador et al., EMBO Reports 2, 5: 446-452, 2001).

It has been reported that DDR1 is over-expressed in numerous human tumors including breast, ovarian, esophageal and brain cancers and in metastatic cancer cells (Barker et al., Oncogene 11: 569-575, 1995; Laval et al., Cell Growth Diff. 5: 1173-1183, 1994; Nemoto et al., Pathobiol. 65: 165-203, 1997; Weiner et al., Pediatr. Neurosurg. 25: 64-72, 1996; Weiner et al., Neurosurgery 47: 1400-1409, 2000; Heinzelmann et al., 10: 4427-4436, 2004). DDR1 and DDR2 have mutually exclusive expression in ovarian and lung tumors, with transcripts for DDR1 in highly invasive tumor cells and transcripts for DDR2 detected in the surrounding stromal cells (Alves et al., Oncogene 10: 609-618, 1995; Barker et al., Oncogene 11: 569-575, 1995). Furthermore, DDR2 expression is associated with invasive mammary carcinomas (Evitmova et al., 2003, Tumor Biol. 24:189-98). Thus the identification of DDR2 as a marker of cancer stem cells suggests that targeting these receptors may prove therapeutically effective in treating human cancers.

An increase in DDR2 expression has been reported to cause an increase in the expression of matrix metailoproteinase-13 (MMP-13) in mice, a protein that remodels the extracellular matrix by degrading major matrix components. These mice exhibited age-related osteoarthritis-like changes in various joints (Li Y et al., J. Biol. Chem. 2005, 280: 548-555). Activation of DDR2 by collagen was also shown to result in the up-regulation of matrix metalloproteinase-1 (MMP-1) expression.

Thus, DDR2 seems to be directly involved in pathophysiological events in osteoarthritis by regulating cell adhesion, proliferation and extracellular matrix remodeling (repress matrix protein production & increased matrix break down).

The scientific rationale for the use of DDR2 inhibitors for the treatment of osteoarthritis follows the line of evidence starting with chondrocytes, osteoarthritis chondrocytes, cartilage animal explants, animal osteoarthritis models and human osteoarthritis cartilage regarding mRNA and protein expression. The protein expression in humans correlates to the cartilage damage and expression of osteoarthritis markers.

Following steps occur during osteoarthritis pathogenesis: The earliest event is a cartilage injury (cartilage impact) or, in senescence, the loss of growth factor sensitivity of articular chondrocytes. This results in an increased expression or activity of HTRA1 by chondrocytes resulting in a break-down of the pericellular collagen VI rich matrix shielding the DDR2 receptor on the chondrocytes surface. If this shield is lost collagen II fibres or fragments become close to the DDR2 receptor and activate this pathway which results in the release of cytokines and degradative proteases (e.g. MMP13, ADAMTS5) and consequently in cartilage degradation. Thus, The DDR2 receptor is regarded as a key receptor in cartilage injury and osteoarthritis.

Besides cancer and osteoarthritis DDR2 seems to be involved in various other human diseases, in particular atherosclerosis, hepatocirrhosis, inflammation, arthritis, and tissue fibrosis.

The WO2005092896 discloses furopyrimidine compounds as DDR inhibitors for hepatocirrhosis, rheumatism and cancer.

Compounds similar to the compounds of the present invention are disclosed in WO2004037789 and WO2006042599 being described as Tie-2 and cRaf inhibitors and in WO2011017142 being described as Aurora and RON kinase inhibitors, all in particular used for the treatment of cancer.

The invention was based on the object of finding novel compounds having valuable properties, in particular those which can be used for the preparation of medicaments.

The object of the present invention was, in particular, to find novel active compounds and particularly preferably novel DDR2 inhibitors which can be employed for the prevention and treatment of osteoarthritis and have, in particular, high selectivity for DDR2. In addition, the aim was to find novel DDR2 inhibitors which are sufficiently stable, at least on local or intra-articular administration.

### SUMMARY OF THE INVENTION

Surprisingly, it has been found that the compounds of formula I according to the invention inhibit DDR2 highly effectively, which plays a crucial role in the development of osteoarthritis. The data show that not only cellular potency can be achieved but also inhibition of pro-MMP13 is observed, which is a biomarker for the initiation and progression of osteoarthritis. It was surprising to find that the compounds of the present invention bearing phenyl or hetero-aromatic rings in the R³ position are strong and selective inhibitors of DDR2 and thus few side effects can be expected. Additionally, it was shown that the potentially genotoxic anilinic moiety can be replaced by amino hetero-aromatic rings. In addition, the compounds according to the invention have adequately good stability in synovial fluid, meaning that they are suitable for intra-articular administration and thus for the treatment of osteoarthritis.

The invention relates to compounds of the formula I,

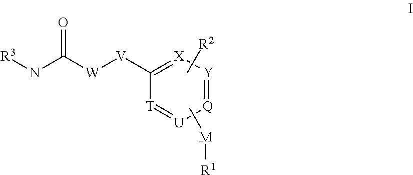

in which
W is O, N, CH₂, CH₂CH₂, CH₂CHOH or —(CH₂)O—,
X, Y, Q, U, T are independently from one another C or N, with the proviso that one or more of X, Y, Q, U and T are carbon atoms and that M is bonded to a carbon atom,
V is a single bond or —CR⁴R⁵—,
M is O or —CR⁴R⁵—,
R¹ is mono- or bicyclic heteroaryl, heterocyclyl or aryl containing 3 to 14 carbon atoms and 1 or 4 heteroatoms, independently selected from N, O and S, which is unsubstituted or mono-, di- or trisubstituted by R⁶,
R² is H, A, CN, OH, OA or Hal,
R³ is mono- or bicyclic heteroaryl, heterocyclyl or aryl containing 3 to 14 carbon atoms and 1 or 4 heteroatoms, independently selected from N, O and S, which is unsubstituted or mono-, di- or trisubstituted by R⁷,
R⁴, R⁵ are independently from one another selected from the group consisting of H and A,
R², R⁶ and R⁷ are independently from one another selected from the group consisting of H, A, Hal, CH₂Hal, CH(Hal)₂, C(Hal)₃, NO₂, (CH₂)ₙCN, (CH₂)ₙNR⁸R⁹, (CH₂)ₙO(CH₂)ₖNR⁸R⁹, (CH₂)ₙNR⁸(CH₂)ₖNR⁸R⁹, (CH₂)ₙO(CH₂)ₖOR¹⁸, (CH₂)ₙNR⁸(CH₂)ₖOR⁹, (CH₂)ₙCOOR¹⁰, (CH₂)ₙCOR¹⁰, (CH₂)ₙCONR⁸R⁹, C(O)NHA, C(O)NHANH₂(CH₂)ₙNR⁸COR¹⁰, (CH₂)ₙNR⁸CONR⁸R⁹, (CH₂)ₙNR⁸SO₂A, (CH₂)ₙSO₂NR⁸R⁹, (CH₂)ₙS(O)ᵤR¹⁰, (CH₂)ₙOC(O)R¹⁰, (CH₂)ₙCOR¹⁰, (CH₂)ₙSR⁸, CH═N—OA, CH₂CH═N—OA, (CH₂)ₙNHOA, (CH₂)ₙCH═N—R⁸, (CH₂)ₙOC(O)NR⁸R⁹, (CH₂)ₙNR⁸COOR¹⁰, (CH₂)ₙN(R⁸)CH₂CH₂OR¹⁰, (CH₂)ₙN(R⁸)CH₂CH₂OCF₃, (CH₂)ₙN(R⁸)C(R¹⁰)HCOOR⁹, (CH₂)ₙN(R⁸)C(R¹⁰)HCOR⁹, (CH₂)ₙN(R⁸)CH₂CH₂N(R⁹)CH₂COOR⁸, (CH₂)ₙN(R⁸)CH₂CH₂NR⁸R⁹, CH═CHCOOR¹⁰, CH═CHCH₂NR⁸R⁹, CH═CHCH₂NR⁸R⁹, CH═CHCH₂OR¹⁰, (CH₂)ₙN(COOR¹⁰)COOR¹¹, (CH₂)ₙN(CONH₂)COOR¹⁰, (CH₂)ₙN(CONH₂)CONH₂, (CH₂)ₙN(CH₂COOR¹⁰)COOR¹¹, (CH₂)ₙN(CH₂CONH₂)COOR¹⁰, (CH₂)ₙN(CH₂CONH₂)CONH₂, (CH₂)ₙCHR¹⁰COR¹¹, (CH₂)ₙCHR¹⁰COOR¹¹, (CH₂)ₙCHR¹⁰CH₂OR¹¹, (CH₂)ₙOCN and (CH₂)ₙNCO,
R⁸, R⁹ are independently from one another selected from the group consisting of H, A, (CH₂)ₘAr¹ and (CH₂)ₘHet, or in NR⁸R¹⁹R⁸ and R⁹ form, together with the N-atom they are bound to, a 5-, 6- or 7-membered heterocyclus which optionally contains 1 or 2 additional hetero atoms, selected from N, O and S,
R¹⁰, R¹¹ are independently from one another selected from the group consisting of H, Hal, A, (CH₂)ₘAr² and (CH₂)ₘHet,
A is selected from the group consisting of alkyl, alkenyl and cycloalkyl,
Ar¹, Ar² are independently from one another aromatic hydrocarbon residues comprising 5 to 12 and preferably 5 to 10 carbon atoms which are optionally substituted by one or more substituents, selected from a group consisting of A, Hal, NO₂, CN, OR¹², NR¹²R¹³, COOR¹², CONR¹²R¹³, NR¹²COR¹³, NR¹²CONR¹²R¹³, NR¹²SO₂A, COR¹², SO₂R¹²R¹³, S(O)ᵤA and OOCR¹²,
Het is a saturated, unsaturated or aromatic mono- or bicyclic heterocyclic residue containing 3 to 14 carbon atoms and 1 or 4 heteroatoms, independently selected from N, O and S, which is optionally substituted by one or more substituents, selected from a group consisting of A, Hal, NO₂, CN, OR¹², NR¹²R¹³, COOR¹², CONR¹²R¹³, NR¹²COR¹³, NR¹²CONR¹²R¹³, NR¹²SO₂A, COR¹², SO₂R¹²R¹², S(O)ᵤA and OOCR¹²,
R¹², R¹³ are independently from one another selected from the group consisting of H, A, and (CH₂)ₘAr³,
Ar³ is a 5- or 6-membered aromatic hydrocarbon which is optionally substituted by one or more substituents selected from a group consisting of methyl, ethyl, propyl, 2-propyl, tert.-butyl, Hal, CN, OH, NH₂ and CF₃,
k, u, n and m are independently from one another 0, 1, 2, 3, 4, or 5,
Hal is independently selected from one another from the group consisting of F, Cl, Br and I,
   and physiologically acceptable salts, derivatives, solvates, prodrugs and stereoisomers thereof, including mixtures thereof in all ratios.

The invention preferably relates to all above-mentioned compounds of the formula I in which
R¹ is

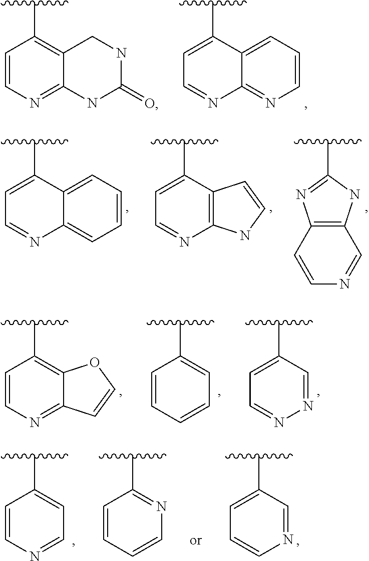

which is unsubstituted or monosubstituted by R⁶,
R³ is

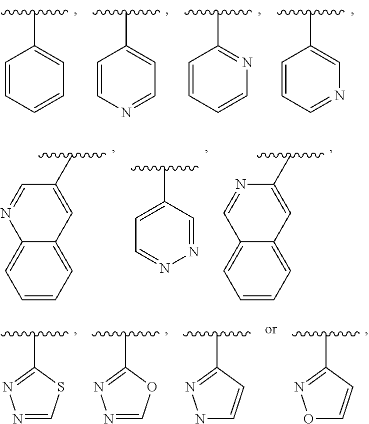

which is unsubstituted or mono-, di- or trisubstituted by R⁷, and
R⁶ and R⁷ independently from one another have the meanings as disclosed above and physiologically acceptable salts, derivatives, solvates, prodrugs and stereoisomers thereof, including mixtures thereof in all ratios.

The invention preferably relates to all above-mentioned compounds of the formula I in which
V is —CR⁴R⁵—,
R¹ is

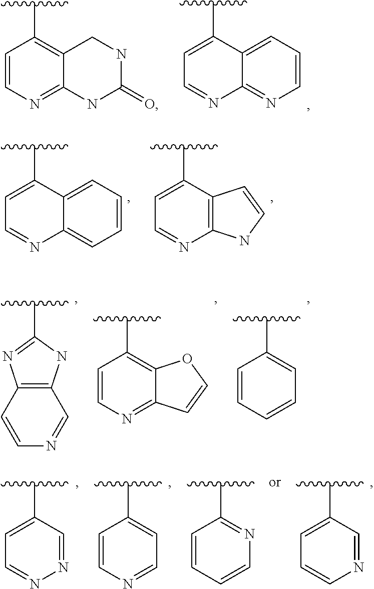

which is unsubstituted or monosubstituted by R⁶,
R³ is

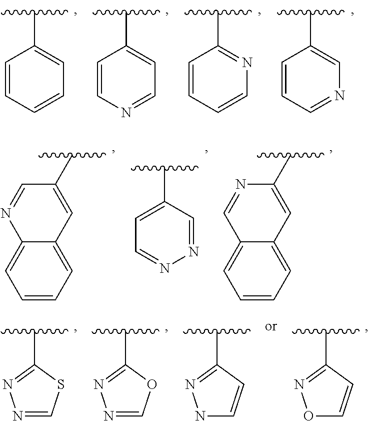

which is unsubstituted or mono-, di- or trisubstituted by R⁷, and
R⁶ and R⁷ independently from one another have the meanings as disclosed above and physiologically acceptable salts, derivatives, solvates, prodrugs and stereoisomers thereof, including mixtures thereof in all ratios.

The invention preferably relates to all above-mentioned compounds of the formula I in which
V is a single bond
R¹ is

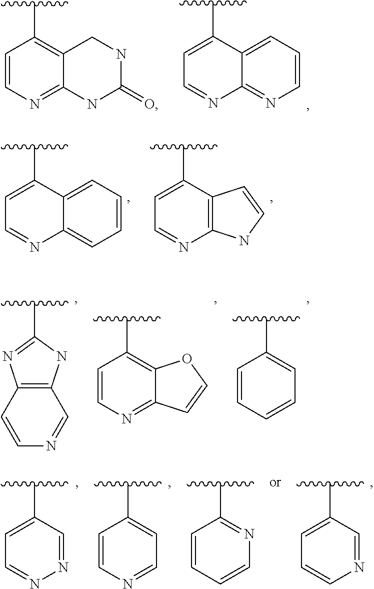

which is unsubstituted or monosubstituted by R⁶,
R³ is

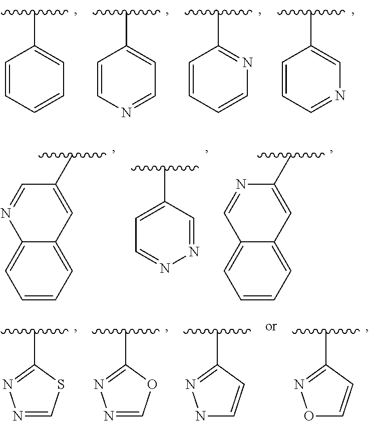

which is unsubstituted or mono-, di- or trisubstituted by R⁷, and
R⁶ and R⁷ independently from one another have the meanings as disclosed above and physiologically acceptable salts, derivatives, solvates, prodrugs and stereoisomers thereof, including mixtures thereof in all ratios.

Another preferred embodiment of the present invention preferably are compounds of the formula I in which
W is N,
X, Y, Q, U, T are independently from one another C or N, with the proviso that one or more of X, Y, Q, U and T are carbon atoms and that M is bonded to a carbon atom,
V is a single bond or —CR⁴R⁵—,
M is O,
R¹ is

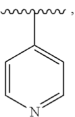 [SMILES: *c1ccncc1]

which is unsubstituted or monosubstituted by R⁶,
R³ is

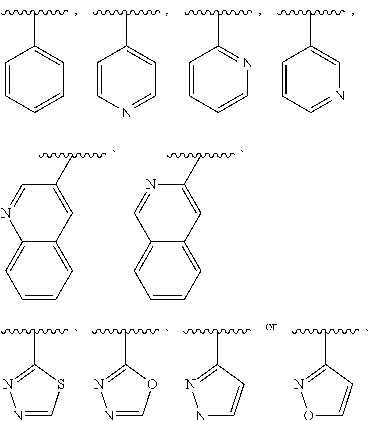

which is unsubstituted or mono-, di- or trisubstituted by R⁷,
R⁴, R⁵ are independently from one another selected from the group consisting of H, alkyl and cycloalkyl, and
R², R⁶ and R⁷ independently from one another have the meanings as disclosed above
   and physiologically acceptable salts, derivatives, solvates, prodrugs and stereoisomers thereof, including mixtures thereof in all ratios.

A particularly preferred embodiment of the present invention are compounds of the formula I in which
W is N,
X, Y, Q, U, T are C,
V is —CR⁴R⁵—,
M is O,
R¹ is

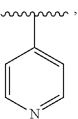 [SMILES: *c1ccncc1]

which is unsubstituted or monosubstituted by R⁶,
R² is H, alkyl with 1 to 5 C-atoms, CN, OH, OA or Hal,
R³ is

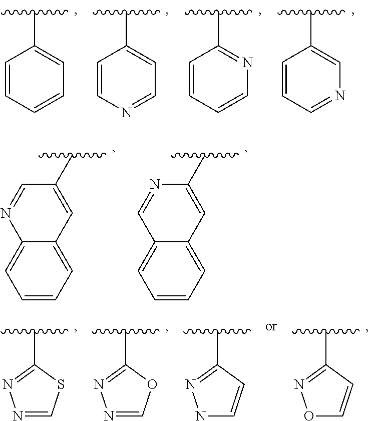

which is unsubstituted or mono-, di- or trisubstituted by R⁷,
R⁴, R⁵ are independently from one another selected from the group consisting of H, alkyl and cycloalkyl,
R⁶ is H, alkyl, C(O)NHA or C(O)NHANH₂,
R⁷ is H, alkyl, cycloalkyl, Hal, CF₃, ═O, CN, SA, C(O)A, COOH, CONH₂, CONHA, CONA₂, CONHANHA, (CH₂)ₙOH, (CH₂)ₙOA, OCH₂C(O)OA, O(CH₂)ₙNH₂, O(CH₂)ₙNHA, O(CH₂)ₙNA₂, O(CH₂)ₙNASO₂A, AOH, OAOH, OAC(O)NH₂, O(CH₂)ₙheterocyclyl, heterocyclyl, SO₂CF₃ or OANAC(O)OA and
n is 0-3
   and physiologically acceptable salts, derivatives, solvates, prodrugs and stereoisomers thereof, including mixtures thereof in all ratios.

A particularly preferred embodiment of the present invention are compounds of the formula I in which
W is N,
X, Y, Q, U, T are C,
V is a single bond,
M is O,
R¹ is

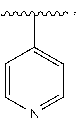 [SMILES: *c1ccncc1]

which is unsubstituted or monosubstituted by R⁶,
R² is H, alkyl with 1 to 5 C-atoms, CN, OH, OA or Hal,
R³ is

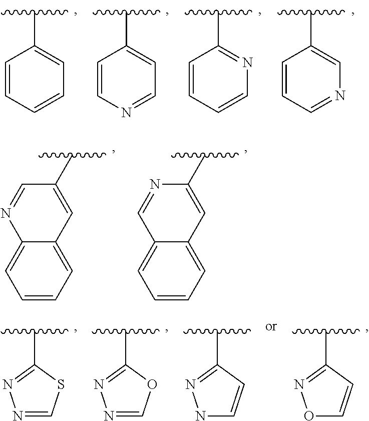

which is unsubstituted or mono-, di- or trisubstituted by R⁷,
R⁴, R⁵ are independently from one another selected from the group consisting of H, alkyl and cycloalkyl,
R⁶ is H, alkyl, C(O)NHA or C(O)NHANH₂,
R⁷ is H, alkyl, cycloalkyl, Hal, CF₃, ═O, CN, SA, C(O)A, COOH, CONH₂, CONHA, CONA₂, CONHANHA, (CH₂)ₙOH, (CH₂)ₙOA, OCH₂C(O)OA, O(CH₂)ₙNH₂, O(CH₂)ₙNHA, O(CH₂)ₙNA₂, O(CH₂)ₙNASO₂A, AOH, OAOH, OAC(O)NH₂, O(CH₂)ₙheterocyclyl, heterocyclyl, SO₂CF₃ or OANAC(O)OA and
n is 0-3
   and physiologically acceptable salts, derivatives, solvates, prodrugs and stereoisomers thereof, including mixtures thereof in all ratios.

Very particular preference is given to the following compounds of the formula I selected from the group consisting of
a) 4-{4-[3-(3,5-Dichloro-pyridin-4-yl)-ureidomethyl]-phenoxy}-pyridine-2-carboxylic acid methylamide
b) 4-{4-[3-(2,6-Dichloro-pyridin-4-yl)-ureidomethyl]-phenoxy}-pyridine-2-carboxylic acid methylamide
c) 4-{4-[(3-Pyridin-2-yl-ureido)-methyl]-phenoxy}-pyridine-2-carboxylic acid methylamide
d) 4-{4-[3-(2-Methoxy-pyridin-3-yl)-ureidomethyl]-phenoxy}-pyridine-2-carboxylic acid methylamide
e) 4-{4-[(3-Pyridin-3-yl-ureido)-methyl]-phenoxy}-pyridine-2-carboxylic acid methylamide
f) 4-{4-[3-(2-Chloro-pyridin-4-yl)-ureidomethyl]-phenoxy}-pyridine-2-carboxylic acid methylamide
g) 4-{4-[3-(3-Chloro-5-trifluoromethyl-pyridin-2-yl)-ureidomethyl]-phenoxy}-pyridine-2-carboxylic acid methylamide
h) 4-{4-[3-(2,5-Dichloro-pyridin-3-yl)-ureidomethyl]-phenoxy}-pyridine-2-carboxylic acid methylamide
i) 4-{4-[(3-Isoquinolin-3-yl-ureido)-methyl]-phenoxy}-pyridine-2-carboxylic acid methylamide
j) 4-{4-[(3-Quinolin-3-yl-ureido)-methyl]-phenoxy}-pyridine-2-carboxylic acid methylamide
k) 4-{4-[3-(2-Methoxy-quinolin-3-yl)-ureidomethyl]-phenoxy}-pyridine-2-carboxylic acid methylamide
l) 4-{4-[3-(5-Methyl-pyridin-2-yl)-ureidomethyl]-phenoxy}-pyridine-2-carboxylic acid methylamide
m) 4-{2-Methyl-4-[3-(5-methyl-pyridin-2-yl)-ureidomethyl]-phenoxy}-pyridine-2-carboxylic acid methylamide
n) 4-{4-[3-(4-Methyl-pyridin-2-yl)-ureidomethyl]-phenoxy}-pyridine-2-carboxylic acid methylamide
o) 4-{2-Methyl-4-[3-(4-methyl-pyridin-2-yl)-ureidomethyl]-phenoxy}-pyridine-2-carboxylic acid methylamide
p) 4-{4-[3-(2-Chloro-pyridin-3-yl)-ureidomethyl]-phenoxy}-pyridine-2-carboxylic acid methylamide
q) 4-{4-[3-(6-Methoxy-pyridin-3-yl)-ureidomethyl]-phenoxy}-pyridine-2-carboxylic acid methylamide
r) 1-(2-Methoxy-5-methyl-pyridin-3-yl)-3-[4-(pyridin-4-yloxy)-benzyl]-urea
s) 4-{4-[3-(2-Methoxy-5-methyl-pyridin-3-yl)-ureidomethyl]-phenoxy}-pyridine-2-carboxylic acid methylamide
t) 4-{4-[3-(2-Methoxy-5-methyl-pyridin-3-yl)-ureidomethyl]-2-methyl-phenoxy}-pyridine-2-carboxylic acid methylamide
u) 4-{4-[3-(5-Chloro-2-methoxy-pyridin-3-yl)-ureidomethyl]-phenoxy}-pyridine-2-carboxylic acid methylamide
v) 4-{4-[3-(2-Chloro-5-methyl-pyridin-3-yl)-ureidomethyl]-phenoxy}-pyridine-2-carboxylic acid methylamide
w) 4-{4-[3-(5-Chloro-2-methoxy-pyridin-3-yl)-ureidomethyl]-2-methyl-phenoxy}-pyridine-2-carboxylic acid methylamide
x) 4-{4-[3-(2-Chloro-5-trifluoromethyl-pyridin-3-yl)-ureidomethyl]-phenoxy}-pyridine-2-carboxylic acid methylamide
y) 4-{4-[3-(2-Chloro-5-methyl-pyridin-3-yl)-ureidomethyl]-2-methyl-phenoxy}-pyridine-2-carboxylic acid methylamide
z) 4-{4-[3-(2-Chloro-5-trifluoromethyl-pyridin-3-yl)-ureidomethyl]-2-methyl-phenoxy}-pyridine-2-carboxylic acid methylamide
aa) 1-(5-Chloro-2-methoxy-pyridin-3-yl)-3-[4-(2-methyl-pyridin-4-yloxy)-benzyl]-urea
bb) 1-(5-Chloro-2-methoxy-pyridin-3-yl)-3-[3-methyl-4-(2-methyl-pyridin-4-yloxy)-benzyl]-urea
cc) 1-(2-Chloro-5-trifluoromethyl-pyridin-3-yl)-3-[4-(2-methyl-pyridin-4-yloxy)-benzyl]-urea
dd) 1-(2-Chloro-5-trifluoromethyl-pyridin-3-yl)-3-[3-methyl-4-(2-methyl-pyridin-4-yloxy)-benzyl]-urea
ee) 1-(2-Methoxy-5-methyl-pyridin-3-yl)-3-[3-methyl-4-(2-methyl-pyridin-4-yloxy)-benzyl]-urea
ff) 1-[3-Methyl-4-(2-methyl-pyridin-4-yloxy)-benzyl]-3-quinolin-3-yl-urea
gg) 1-(2-Methoxy-quinolin-3-yl)-3-[3-methyl-4-(2-methyl-pyridin-4-yloxy)-benzyl]-urea
hh) 1-lsoquinolin-3-yl-3-[3-methyl-4-(2-methyl-pyridin-4-yloxy)-benzyl]-urea
   and physiologically acceptable salts, derivatives, solvates, prodrugs and stereoisomers thereof, including mixtures thereof in all ratios.

Especially preferred are also compounds of the formula I selected from the group consisting of

| No. | Compound (chemical name) |
|---|---|
| 1 | 1-(3-Chloro-phenyl)-3-[4-(pyridin-4-yloxy)-benzyl]-urea |
| 2 | 1-[4-(Pyridin-4-yloxy)-benzyl]-3-(2,4,5-trichloro-phenyl)-urea |
| 3 | 4-{4-[3-(3,4-Dichloro-phenyl)-ureidomethyl]-phenoxy}- |
| | pyridine-2-carboxylic acid methylamide |
| 4 | 1-[4-(Pyridin-4-yloxy)-benzyl]-3-(3-trifluoromethyl-phenyl)- |
| | urea |
| 5 | 1-(2,4-Dichloro-phenyl)-3-[4-(pyridin-4-yloxy)-benzyl]-urea |
| 6 | 1-[4-(Pyridin-4-yloxy)-benzyl]-3-m-tolyl-urea |
| 7 | 1-(3-Acetyl-phenyl)-3-[4-(pyridin-4-yloxy)-benzyl]-urea |
| 8 | 4-{4-[3-(2,4-Dichloro-phenyl)-ureidomethyl]-phenoxy}- |
| | pyridine-2-carboxylic acid methylamide |
| 9 | 1-(4-Bromo-phenyl)-3-[4-(pyridin-4-yloxy)-benzyl]-urea |
| 10 | 1-(2,5-Dichloro-phenyl)-3-[4-(pyridin-4-yloxy)-benzyl]-urea |
| 11 | 1-(4-Fluoro-phenyl)-3-[4-(pyridin-4-yloxy)-benzyl]-urea |
| 12 | 4-{4-[3-(4-Fluoro-phenyl)-ureidomethyl]-phenoxy}-pyridine- |
| | 2-carboxylic acid methylamide |
| 13 | 1-(2,3-Dichloro-phenyl)-3-[4-(pyridin-4-yloxy)-benzyl]-urea |
| 14 | 4-{4-[3-(2-Methoxy-phenyl)-ureidomethyl]-phenoxy}- |
| | pyridine-2-carboxylic acid methylamide |
| 15 | 1-(2,5-Dimethoxy-phenyl)-3-[4-(pyridin-4-yloxy)-benzyl]- |
| | urea |
| 16 | 1-(4-Chloro-phenyl)-3-[4-(pyridin-4-yloxy)-benzyl]-urea |
| 17 | 1-(4-Methoxy-phenyl)-3-[4-(pyridin-4-yloxy)-benzyl]-urea |
| 18 | 1-[4-(Pyridin-4-yloxy)-benzyl]-3-p-tolyl-urea |
| 19 | 4-{4-[3-(5-Chloro-2-methoxy-phenyl)-ureidomethyl]- |
| | phenoxy}-pyridine-2-carboxylic acid methylamide |
| 20 | 4-{4-[3-(2,5-Dimethoxy-phenyl)-ureidomethyl]-phenoxy}- |
| | pyridine-2-carboxylic acid methylamide |
| 21 | 1-[4-(Pyridin-4-yloxy)-benzyl]-3-(4-trifluoromethyl-phenyl)- |
| | urea |
| 22 | 1-(3,5-Bis-trifluoromethyl-phenyl)-3-[4-(pyridin-4-yloxy)- |
| | benzyl]-urea |
| 23 | 4-{4-[3-(2,4,5-Trichloro-phenyl)-ureidomethyl]-phenoxy}- |
| | pyridine-2-carboxylic acid methylamide |
| 24 | 1-(2,3-Dimethyl-phenyl)-3-[4-(pyridin-4-yloxy)-benzyl]-urea |
| 25 | 1-(2,5-Dimethyl-phenyl)-3-[4-(pyridin-4-yloxy)-benzyl]-urea |
| 26 | 1-[4-(Pyridin-4-yloxy)-benzyl]-3-(2-trifluoromethyl-phenyl)- |
| | urea |
| 27 | 1-(3-Chloro-4-methyl-phenyl)-3-[4-(pyridin-4-yloxy)-benzyl]- |
| | urea |
| 28 | 1-(2-Ethyl-phenyl)-3-[4-(pyridin-4-yloxy)-benzyl]-urea |
| 29 | 1-[4-(Pyridin-4-yloxy)-benzyl]-3-o-tolyl-urea |
| 30 | 1-(2,4-Dimethyl-phenyl)-3-[4-(pyridin-4-yloxy)-benzyl]-urea |
| 31 | 4-{4-[3-(3,5-Dichloro-phenyl)-ureidomethyl]-phenoxy}- |
| | pyridine-2-carboxylic acid methylamide |
| 32 | 1-(5-Chloro-2-methoxy-phenyl)-3-[4-(pyridin-4-yloxy)- |
| | benzyl]-urea |
| 33 | 1-(2-Chloro-phenyl)-3-[4-(pyridin-4-yloxy)-benzyl]-urea |
| 34 | 1-(3-Methylsulfanyl-phenyl)-3-[4-(pyridin-4-yloxy)-benzyl]- |
| | urea |
| 35 | 1-(4-Bromo-2-chloro-phenyl)-3-[4-(pyridin-4-yloxy)-benzyl]- |
| | urea |
| 36 | 1-(2-Methoxy-phenyl)-3-[4-(pyridin-4-yloxy)-benzyl]-urea |
| 37 | 1-(2-Chloro-4-trifluoromethyl-phenyl)-3-[4-(pyridin-4-yloxy)- |
| | benzyl]-urea |
| 38 | 1-(3,4-Dichloro-phenyl)-3-[4-(pyridin-4-yloxy)-benzyl]-urea |
| 39 | 4-{4-[3-(3-Chloro-4-methoxy-phenyl)-ureidomethyl]- |
| | phenoxy}-pyridine-2-carboxylic acid methylamide |
| 40 | 1-(4-Chloro-2-trifluoromethyl-phenyl)-3-[4-(pyridin-4-yloxy)- |
| | benzyl]-urea |
| 41 | 1-(4-Ethoxy-phenyl)-3-[4-(pyridin-4-yloxy)-benzyl]-urea |
| 42 | 4-{4-[3-(5-Chloro-2-methyl-phenyl)-ureidomethyl]-phenoxy}- |
| | pyridine-2-carboxylic acid methylamide |
| 43 | 1-(2-Chloro-5-trifluoromethyl-phenyl)-3-[4-(pyridin-4-yloxy)- |
| | benzyl]-urea |
| 44 | 1-(3,5-Dimethyl-phenyl)-3-[4-(pyridin-4-yloxy)-benzyl]-urea |
| 45 | 1-(3-Methoxy-phenyl)-3-[4-(pyridin-4-yloxy)-benzyl]-urea |
| 46 | 1-(4-Fluoro-3-trifluoromethyl-phenyl)-3-[4-(pyridin-4-yloxy)- |
| | benzyl]-urea |
| 47 | 1-(4-Acetyl-phenyl)-3-[4-(pyridin-4-yloxy)-benzyl]-urea |
| 48 | 1-(2-Bromo-phenyl)-3-[4-(pyridin-4-yloxy)-benzyl]-urea |
| 49 | 1-(4-Isopropyl-phenyl)-3-[4-(pyridin-4-yloxy)-benzyl]-urea |
| 50 | 1-(5-Chloro-2-methyl-phenyl)-3-[4-(pyridin-4-yloxy)-benzyl]- |
| | urea |
| 51 | 1-(4-Methylsulfanyl-phenyl)-3-[4-(pyridin-4-yloxy)-benzyl]- |
| | urea |
| 52 | 1-(4-Ethyl-phenyl)-3-[4-(pyridin-4-yloxy)-benzyl]-urea |
| 53 | 1-(3-Bromo-phenyl)-3-[4-(pyridin-4-yloxy)-benzyl]-urea |
| 54 | 1-(4-Chloro-2-methyl-phenyl)-3-[4-(pyridin-4-yloxy)-benzyl]- |
| | urea |
| 55 | 1-[4-(Pyridin-4-yloxy)-benzyl]-3-(4-trifluoromethoxy-phenyl)- |
| | urea |
| 56 | 1-(4-tert-Butyl-phenyl)-3-[4-(pyridin-4-yloxy)-benzyl]-urea |
| 57 | 1-(3,5-Dichloro-phenyl)-3-[4-(pyridin-4-yloxy)-benzyl]-urea |
| 58 | 1-(4-Bromo-3-methyl-phenyl)-3-[4-(pyridin-4-yloxy)-benzyl]- |
| | urea |
| 59 | 1-(3,4-Dimethyl-phenyl)-3-[4-(pyridin-4-yloxy)-benzyl]-urea |
| 60 | 1-(3-Chloro-4-methoxy-phenyl)-3-[4-(pyridin-4-yloxy)- |
| | benzyl]-urea |
| 61 | 1-(3-Ethyl-phenyl)-3-[4-(pyridin-4-yloxy)-benzyl]-urea |
| 62 | 1-(2-Methoxy-5-trifluoromethyl-phenyl)-3-[4-(pyridin-4- |
| | yloxy)-benzyl]-urea |
| 63 | 4-{4-[3-(2-Methoxy-5-trifluoromethyl-phenyl)-ureidomethyl]- |
| | phenoxy}-pyridine-2-carboxylic acid methylamide |
| 64 | 1-(4-Bromo-3-trifluoromethyl-phenyl)-3-[4-(pyridin-4-yloxy)- |
| | benzyl]-urea |
| 65 | 1-[4-(Pyridin-4-yloxy)-benzyl]-3-(3-trifluoromethoxy-phenyl)- |
| | urea |
| 66 | 4-{4-[3-(5-Chloro-2-methoxy-4-methyl-phenyl)- |
| | ureidomethyl]-phenoxy}-pyridine-2-carboxylic acid |
| | methylamide |
| 67 | 4-{4-[3-(4-Chloro-2-methoxy-5-trifluoromethyl-phenyl)- |
| | ureidomethyl]-phenoxy}-pyridine-2-carboxylic acid |
| | methylamide |
| 68 | 1-(4-Chloro-3-trifluoromethyl-phenyl)-3-{1-[4-(pyridin-4- |
| | yloxy)-phenyl]-cyclopropyl}-urea |
| 69 | 4-(4-{1-[3-(2-Methoxy-5-trifluoromethyl-phenyl)-ureido]- |
| | ethyl}-phenoxy)-pyridine-2-carboxylic acid methylamide |
| 70 | 4-(4-{1-[3-(2,4,5-Trichloro-phenyl)-ureido]-ethyl}-phenoxy)- |
| | pyridine-2-carboxylic acid methylamide |
| 71 | 4-(4-{1-[3-(3,4-Dichloro-phenyl)-ureido]-ethyl}-phenoxy)- |
| | pyridine-2-carboxylic acid methylamide |
| 72 | 4-(4-{1-[3-(5-Chloro-2-methoxy-phenyl)-ureido]-ethyl}- |
| | phenoxy)-pyridine-2-carboxylic acid methylamide |
| 73 | 4-(4-{1-[3-(3-Chloro-4-methyl-phenyl)-ureido]-ethyl}- |
| | phenoxy)-pyridine-2-carboxylic acid methylamide |
| 74 | 4-{4-[3-(4-Chloro-3-methyl-phenyl)-ureidomethyl]-phenoxy}- |
| | pyridine-2-carboxylic acid methylamide |
| 75 | 4-{4-[3-(2-Methoxy-5-methyl-phenyl)-ureidomethyl]- |
| | phenoxy}-pyridine-2-carboxylic acid methylamide |
| 76 | 4-{4-[(3-Benzo[1,2,5]thiadiazol-5-yl-ureido)-methyl]- |
| | phenoxy}-pyridine-2-carboxylic acid methylamide |
| 77 | 4-(4-{3-[2-(2-Dimethylamino-ethoxy)-5-trifluoromethyl- |
| | phenyl]-ureidomethyl}-phenoxy)-pyridine-2-carboxylic acid |
| | methylamide |
| 78 | 4-{4-[3-(4-Chloro-2-methoxy-5-methyl-phenyl)- |
| | ureidomethyl]-phenoxy}-pyridine-2-carboxylic acid |
| | methylamide |
| 79 | 4-{4-[3-(4-Chloro-2-methoxy-phenyl)-ureidomethyl]- |
| | phenoxy}-pyridine-2-carboxylic acid methylamide |
| 80 | 4-{4-[3-(3,4,5-Trimethoxy-phenyl)-ureidomethyl]-phenoxy}- |
| | pyridine-2-carboxylic acid methylamide |
| 81 | 4-{4-[3-(2,5-Dimethoxy-4-nitro-phenyl)-ureidomethyl]- |
| | phenoxy}-pyridine-2-carboxylic acid methylamide |
| 82 | 3-Methoxy-4-{3-[4-(2-methylcarbamoyl-pyridin-4-yloxy)- |
| | benzyl]-ureido}-benzoic acid methyl ester |
| 83 | 4-{4-[3-(4-Chloro-2,5-dimethoxy-phenyl)-ureidomethyl]- |
| | phenoxy}-pyridine-2-carboxylic acid methylamide |
| 84 | 4-{4-[3-(3,5-Dichloro-pyridin-4-yl)-ureidomethyl]-phenoxy}- |
| | pyridine-2-carboxylic acid methylamide |
| 85 | 4-{4-[(3-Pyridin-2-yl-ureido)-methyl]-phenoxy}-pyridine-2- |
| | carboxylic acid methylamide |
| 86 | 4-(4-{3-[2-(3-Dimethylamino-propoxy)-phenyl]- |
| | ureidomethyl}-phenoxy)-pyridine-2-carboxylic acid |
| | methylamide |
| 87 | 4-{4-[3-(2-Methoxy-pyridin-3-yl)-ureidomethyl]-phenoxy}- |
| | pyridine-2-carboxylic acid methylamide |
| 88 | 4-{4-[(3-Pyridin-3-yl-ureido)-methyl]-phenoxy}-pyridine-2- |
| | carboxylic acid methylamide |
| 89 | 4-{4-[3-(2-Chloro-pyridin-4-yl)-ureidomethyl]-phenoxy}- |
| | pyridine-2-carboxylic acid methylamide |
| 90 | 4-{4-[(3-Pyridin-4-yl-ureido)-methyl]-phenoxy}-pyridine-2- |
| | carboxylic acid methylamide |
| 91 | 4-{4-[3-(3-Chloro-5-trifluoromethyl-pyridin-2-yl)- |
| | ureidomethyl]-phenoxy}-pyridine-2-carboxylic acid |
| | methylamide |
| 92 | (2-{3-[4-(2-Methylcarbamoyl-pyridin-4-yloxy)-benzyl]- |
| | ureido}-4-trifluoromethyl-phenoxy)-acetic acid methyl ester |
| 93 | 4-{4-[3-(2,5-Dichloro-pyridin-3-yl)-ureidomethyl]-phenoxy}- |
| | pyridine-2-carboxylic acid methylamide |
| 94 | (2-{3-[4-(2-Methylcarbamoyl-pyridin-4-yloxy)-benzyl]- |
| | ureido}-4-trifluoromethyl-phenoxy)-acetic acid |
| 95 | 4-(4-{3-[4-Chloro-2-(2-dimethylamino-ethoxy)-5- |
| | trifluoromethyl-phenyl]-ureidomethyl}-phenoxy)-pyridine-2- |
| | carboxylic acid methylamide |
| 96 | 4-{4-[(3-Isoquinolin-3-yl-ureido)-methyl]-phenoxy}-pyridine- |
| | 2-carboxylic acid methylamide |
| 97 | 4-{4-[(3-Quinolin-3-yl-ureido)-methyl]-phenoxy}-pyridine-2- |
| | carboxylic acid methylamide |
| 98 | (5-Chloro-2-{3-[4-(2-methylcarbamoyl-pyridin-4-yloxy)- |
| | benzyl]-ureido}-4-trifluoromethyl-phenoxy)-acetic acid |
| 99 | 4-{4-[3-(4-Chloro-3-trifluoromethyl-phenyl)-ureidomethyl]- |
| | phenoxy}-pyridine-2-carboxylic acid |
| 100 | 4-{4-[3-(5-Chloro-2-methoxy-phenyl)-ureidomethyl]-2- |
| | methyl-phenoxy}-pyridine-2-carboxylic acid methylamide |
| 101 | 4-{4-[3-(2-Methoxy-quinolin-3-yl)-ureidomethyl]-phenoxy}- |
| | pyridine-2-carboxylic acid methylamide |
| 102 | 4-{4-[3-(2-Methoxy-5-trifluoromethyl-phenyl)-ureidomethyl]- |
| | 2-methyl-phenoxy}-pyridine-2-carboxylic acid methylamide |
| 103 | 4-{4-[3-(2,4-Dichloro-6-methoxy-3-methyl-phenyl)- |
| | ureidomethyl]-phenoxy}-pyridine-2-carboxylic acid |
| | methylamide |
| 104 | 4-{4-[3-(5-Chloro-2-methoxy-phenyl)-ureidomethyl]-3- |
| | methyl-phenoxy}-pyridine-2-carboxylic acid methylamide |
| 105 | 4-{4-[3-(2-Methoxy-5-trifluoromethyl-phenyl)-ureidomethyl]- |
| | 3-methyl-phenoxy}-pyridine-2-carboxylic acid methylamide |
| 106 | 4-(4-{3-[2-(2-Pyrrolidin-1-yl-ethoxy)-5-trifluoromethyl- |
| | phenyl]-ureidomethyl}-phenoxy)-pyridine-2-carboxylic acid |
| | methylamide |
| 107 | (5-Chloro-2-{3-[4-(2-methylcarbamoyl-pyridin-4-yloxy)- |
| | benzyl]-ureido}-4-trifluoromethyl-phenoxy)-acetic acid tert- |
| | butyl ester |
| 108 | 4-(4-{3-[2-(2-Diethylamino-ethoxy)-5-trifluoromethyl-phenyl]- |
| | ureidomethyl}-phenoxy)-pyridine-2-carboxylic acid |
| | methylamide |
| 109 | 4-(4-{3-[2-(2-Morpholin-4-yl-ethoxy)-5-trifluoromethyl- |
| | phenyl]-ureidomethyl}-phenoxy)-pyridine-2-carboxylic acid |
| | methylamide |
| 110 | 4-(4-{3-[4-Chloro-5-methyl-2-(2-morpholin-4-yl-ethoxy)- |
| | phenyl]-ureidomethyl}-phenoxy)-pyridine-2-carboxylic acid |
| | methylamide |
| 111 | 4-(4-{3-[2-(2-Methylamino-ethoxy)-5-trifluoromethyl-phenyl]- |
| | ureidomethyl}-phenoxy)-pyridine-2-carboxylic acid |
| | methylamide |
| 112 | 4-(4-{3-[2-(2-Piperazin-1-yl-ethoxy)-5-trifluoromethyl- |
| | phenyl]-ureidomethyl}-phenoxy)-pyridine-2-carboxylic acid |
| | methylamide |
| 113 | (5-Chloro-2-{3-[4-(2-methylcarbamoyl-pyridin-4-yloxy)- |
| | benzyl]-ureido}-4-trifluoromethyl-phenoxy)-acetic acid |
| | methyl ester |
| 114 | (5-Chloro-2-{3-[4-(2-methylcarbamoyl-pyridin-4-yloxy)- |
| | benzyl]-ureido}-4-trifluoromethyl-phenoxy)-acetic acid |
| | isopropyl ester |
| 115 | 4-(4-{3-[2-(Piperidin-4-yloxy)-5-trifluoromethyl-phenyl]- |
| | ureidomethyl}-phenoxy)-pyridine-2-carboxylic acid |
| | methylamide |
| 116 | 4-(4-{3-[4-Chloro-5-methyl-2-(2-pyrrolidin-1-yl-ethoxy)- |
| | phenyl]-ureidomethyl}-phenoxy)-pyridine-2-carboxylic acid |
| | methylamide |
| 117 | 4-(4-{3-[4-Chloro-2-(2-diethylamino-ethoxy)-5-methyl- |
| | phenyl]-ureidomethyl}-phenoxy)-pyridine-2-carboxylic acid |
| | methylamide |
| 118 | 4-(4-{3-[4-Chloro-2-(2-dimethylamino-ethoxy)-5-methyl- |
| | phenyl]-ureidomethyl}-phenoxy)-pyridine-2-carboxylic acid |
| | methylamide |
| 119 | 4-{4-[3-(4-Chloro-2-methoxy-5-methyl-phenyl)- |
| | ureidomethyl]-2-methyl-phenoxy}-pyridine-2-carboxylic acid |
| | methylamide |
| 120 | 4-(4-{3-[4-Chloro-5-methyl-2-(2-piperazin-1-yl-ethoxy)- |
| | phenyl]-ureidomethyl}-phenoxy)-pyridine-2-carboxylic acid |
| | methylamide |
| 121 | 4-(4-{3-[2-(2-Amino-ethoxy)-5-trifluoromethyl-phenyl]- |
| | ureidomethyl}-phenoxy)-pyridine-2-carboxylic acid |
| | methylamide |
| 122 | 4-{4-[3-(2-Piperazin-1-yl-5-trifluoromethyl-phenyl)- |
| | ureidomethyl]-phenoxy}-pyridine-2-carboxylic acid |
| | methylamide |
| 123 | 4-{4-[3-(4-Chloro-2-methoxy-5-methyl-phenyl)- |
| | ureidomethyl]-phenoxy}-pyridine-2-carboxylic acid (2- |
| | amino-ethyl)-amide |
| 124 | 4-(4-{3-[4-Chloro-5-methyl-2-(piperidin-4-yloxy)-phenyl]- |
| | ureidomethyl}-phenoxy)-pyridine-2-carboxylic acid |
| | methylamide |
| 125 | 4-(4-{3-[4-Chloro-5-methyl-2-(2-methylamino-ethoxy)- |
| | phenyl]-ureidomethyl}-phenoxy)-pyridine-2-carboxylic acid |
| | methylamide |
| 126 | 4-{4-[3-(4-Chloro-2-methoxy-5-methyl-phenyl)- |
| | ureidomethyl]-phenoxy}-pyridine-2-carboxylic acid (6- |
| | amino-hexyl)-amide |
| 127 | 4-(4-{3-[4-Chloro-2-(2-methylamino-ethoxy)-5- |
| | trifluoromethyl-phenyl]-ureidomethyl}-phenoxy)-pyridine-2- |
| | carboxylic acid methylamide |
| 128 | 4-(4-{3-[4-Chloro-2-(2-piperazin-1-yl-ethoxy)-5- |
| | trifluoromethyl-phenyl]-ureidomethyl}-phenoxy)-pyridine-2- |
| | carboxylic acid methylamide |
| 129 | 4-(4-{3-[4-Chloro-2-(2-pyrrolidin-1-yl-ethoxy)-5- |
| | trifluoromethyl-phenyl]-ureidomethyl}-phenoxy)-pyridine-2- |
| | carboxylic acid methylamide |
| 130 | 4-(4-{3-[4-Chloro-2-(piperidin-4-yloxy)-5-trifluoromethyl- |
| | phenyl]-ureidomethyl}-phenoxy)-pyridine-2-carboxylic acid |
| | methylamide |
| 131 | 4-(4-{3-[4-Chloro-2-(2-morpholin-4-yl-ethoxy)-5- |
| | trifluoromethyl-phenyl]-ureidomethyl}-phenoxy)-pyridine-2- |
| | carboxylic acid methylamide |
| 132 | 4-(4-{3-[4-Chloro-2-(2-diethylamino-ethoxy)-5- |
| | trifluoromethyl-phenyl]-ureidomethyl}-phenoxy)-pyridine-2- |
| | carboxylic acid methylamide |
| 133 | 4-(4-{3-[4-Chloro-5-methyl-2-(2-morpholin-4-yl-ethoxy)- |
| | phenyl]-ureidomethyl}-3-methyl-phenoxy)-pyridine-2- |
| | carboxylic acid methylamide |
| 134 | 4-(4-{3-[4-Chloro-5-methyl-2-(2-pyrrolidin-1-yl-ethoxy)- |
| | phenyl]-ureidomethyl}-3-methyl-phenoxy)-pyridine-2- |
| | carboxylic acid methylamide |
| 135 | 4-{4-[3-(3-Methanesulfonyl-phenyl)-ureidomethyl]-phenoxy}- |
| | pyridine-2-carboxylic acid methylamide |
| 136 | 4-(4-{3-[4-Chloro-2-(2-diethylamino-ethoxy)-5-methyl- |
| | phenyl]-ureidomethyl}-2-methyl-phenoxy)-pyridine-2- |
| | carboxylic acid methylamide |
| 137 | 4-(4-{3-[4-Chloro-5-methyl-2-(2-pyrrolidin-1-yl-ethoxy)- |
| | phenyl]-ureidomethyl}-2-methyl-phenoxy)-pyridine-2- |
| | carboxylic acid methylamide |
| 138 | 4-(4-{3-[4-Chloro-5-methyl-2-(2-morpholin-4-yl-ethoxy)- |
| | phenyl]-ureidomethyl}-2-methyl-phenoxy)-pyridine-2- |
| | carboxylic acid methylamide |
| 139 | 4-(4-{3-[4-Chloro-2-(2-dimethylamino-ethoxy)-5-methyl- |
| | phenyl]-ureidomethyl}-2-methyl-phenoxy)-pyridine-2- |
| | carboxylic acid methylamide |
| 140 | 4-(4-{3-[2-(2-Amino-ethoxy)-4-chloro-5-trifluoromethyl- |
| | phenyl]-ureidomethyl}-phenoxy)-pyridine-2-carboxylic acid |
| | methylamide |
| 141 | 4-(4-{3-[4-Chloro-5-methyl-2-(2-methylamino-ethoxy)- |
| | phenyl]-ureidomethyl}-2-methyl-phenoxy)-pyridine-2- |
| | carboxylic acid methylamide |
| 142 | 4-(4-{3-[4-Chloro-5-methyl-2-(piperidin-4-yloxy)-phenyl]- |
| | ureidomethyl}-2-methyl-phenoxy)-pyridine-2-carboxylic acid |
| | methylamide |
| 143 | 4-(4-{3-[4-Chloro-5-methyl-2-(2-piperazin-1-yl-ethoxy)- |
| | phenyl]-ureidomethyl}-2-methyl-phenoxy)-pyridine-2- |
| | carboxylic acid methylamide |
| 144 | 4-(4-{3-[2-(2-Amino-ethoxy)-4-chloro-5-methyl-phenyl]- |
| | ureidomethyl}-2-methyl-phenoxy)-pyridine-2-carboxylic acid |
| | methylamide |
| 145 | 4-(4-{3-[2-(2-Amino-ethoxy)-4-chloro-5-methyl-phenyl]- |
| | ureidomethyl}-phenoxy)-pyridine-2-carboxylic acid |
| | methylamide |
| 146 | 4-(2-Methyl-4-{3-[2-(2-methylamino-ethoxy)-5- |
| | trifluoromethyl-phenyl]-ureidomethyl}-phenoxy)-pyridine-2- |
| | carboxylic acid methylamide |
| 147 | 4-(4-{3-[4-Chloro-2-(2-methylamino-ethoxy)-5- |
| | trifluoromethyl-phenyl]-ureidomethyl}-2-methyl-phenoxy)- |
| | pyridine-2-carboxylic acid methylamide |
| 148 | 4-{4-[3-(4-Chloro-3-trifluoromethyl-phenyl)-ureidomethyl]- |
| | phenoxy}-pyridine-2-carboxylic acid (2-amino-ethyl)-amide |
| 149 | 4-{4-[3-(4-Chloro-3-trifluoromethyl-phenyl)-ureidomethyl]- |
| | phenoxy}-pyridine-2-carboxylic acid (2-methylamino-ethyl)- |
| | amide |
| 150 | 4-(4-{3-[2-(2-Dimethylamino-ethoxy)-5-trifluoromethyl- |
| | phenyl]-ureidomethyl}-2-methyl-phenoxy)-pyridine-2- |
| | carboxylic acid methylamide |
| 151 | 4-{4-[3-(5-Chloro-2-methoxy-4-methyl-phenyl)- |
| | ureidomethyl]-2-methyl-phenoxy}-pyridine-2-carboxylic acid |
| | methylamide |
| 152 | 4-(4-{3-[4-Chloro-2-(2-dimethylamino-ethoxy)-5- |
| | trifluoromethyl-phenyl]-ureidomethyl}-2-methyl-phenoxy)- |
| | pyridine-2-carboxylic acid methylamide |
| 153 | 4-(4-{3-[4-Chloro-2-(3-dimethylamino-propoxy)-5-methyl- |
| | phenyl]-ureidomethyl}-phenoxy)-pyridine-2-carboxylic acid |
| | methylamide |
| 154 | 4-{4-[3-(3-Methanesulfonyl-phenyl)-ureidomethyl]-2-methyl- |
| | phenoxy}-pyridine-2-carboxylic acid methylamide |
| 155 | 4-(4-{3-[2-(2-Amino-ethoxy)-5-trifluoromethyl-phenyl]- |
| | ureidomethyl}-2-methyl-phenoxy)-pyridine-2-carboxylic acid |
| | methylamide |
| 156 | 4-(4-{3-[2-(Pyrrolidin-2-ylmethoxy)-5-trifluoromethyl-phenyl]- |
| | ureidomethyl}-phenoxy)-pyridine-2-carboxylic acid |
| | methylamide |
| 157 | 4-{4-[3-(3-Sulfamoyl-phenyl)-ureidomethyl]-phenoxy}- |
| | pyridine-2-carboxylic acid methylamide |
| 158 | 4-{4-[3-(3-Isopropylsulfamoyl-phenyl)-ureidomethyl]- |
| | phenoxy}-pyridine-2-carboxylic acid methylamide |
| 159 | 4-(4-{3-[5-Methyl-2-(piperidin-4-yloxy)-phenyl]- |
| | ureidomethyl}-phenoxy)-pyridine-2-carboxylic acid |
| | methylamide |
| 160 | 4-(4-{3-[2-(2-Amino-2-methyl-propoxy)-5-trifluoromethyl- |
| | phenyl]-ureidomethyl}-phenoxy)-pyridine-2-carboxylic acid |
| | methylamide |
| 161 | 4-(4-{3-[4-Chloro-2-(4-dimethylamino-butoxy)-5-methyl- |
| | phenyl]-ureidomethyl}-phenoxy)-pyridine-2-carboxylic acid |
| | methylamide |
| 162 | 4-[4-(3-{4-Chloro-2-[(2-dimethylamino-ethyl)-methyl-amino]- |
| | 5-methyl-phenyl}-ureidomethyl)-phenoxy]-pyridine-2- |
| | carboxylic acid methylamide |
| 163 | 4-(4-{3-[4-Chloro-2-(3-dimethylamino-propoxy)-5-methyl- |
| | phenyl]-ureidomethyl}-2-methyl-phenoxy)-pyridine-2- |
| | carboxylic acid methylamide |
| 164 | 4-[4-(3-{4-Chloro-2-[(2-dimethylamino-ethyl)-methyl-amino]- |
| | 5-methyl-phenyl}-ureidomethyl)-2-methyl-phenoxy]-pyridine- |
| | 2-carboxylic acid methylamide |
| 165 | 4-(4-{3-[4-Chloro-2-(4-dimethylamino-butoxy)-5-methyl- |
| | phenyl]-ureidomethyl}-2-methyl-phenoxy)-pyridine-2- |
| | carboxylic acid methylamide |
| 166 | 4-(4-{3-[2-(2-Methoxy-ethoxy)-5-trifluoromethyl-phenyl]- |
| | ureidomethyl}-phenoxy)-pyridine-2-carboxylic acid |
| | methylamide |
| 167 | 4-{4-[3-(3-Methanesulfonylamino-phenyl)-ureidomethyl]-2- |
| | methyl-phenoxy}-pyridine-2-carboxylic acid methylamide |
| 168 | 4-(4-{3-[2-(2-Methoxy-ethoxy)-5-trifluoromethyl-phenyl]- |
| | ureidomethyl}-2-methyl-phenoxy)-pyridine-2-carboxylic acid |
| | methylamide |
| 169 | 4-(4-{3-[2-(2-Methylamino-ethoxy)-phenyl]-ureidomethyl}- |
| | phenoxy)-pyridine-2-carboxylic acid methylamide |
| 170 | 4-(4-{3-[5-Methyl-2-(2-methylamino-ethoxy)-phenyl]- |
| | ureidomethyl}-phenoxy)-pyridine-2-carboxylic acid |
| | methylamide |
| 171 | 4-(2-Methyl-4-{3-[5-methyl-2-(piperidin-4-yloxy)-phenyl]- |
| | ureidomethyl}-phenoxy)-pyridine-2-carboxylic acid |
| | methylamide |
| 172 | 4-(4-{3-[2-(2-Isopropylamino-ethoxy)-5-trifluoromethyl- |
| | phenyl]-ureidomethyl}-phenoxy)-pyridine-2-carboxylic acid |
| | methylamide |
| 173 | 4-(4-{3-[5-Chloro-4-methyl-2-(2-pyrrolidin-1-yl-ethoxy)- |
| | phenyl]-ureidomethyl}-phenoxy)-pyridine-2-carboxylic acid |
| | methylamide |
| 174 | 4-(4-{3-[5-Chloro-2-(2-dimethylamino-ethoxy)-4-methyl- |
| | phenyl]-ureidomethyl}-phenoxy)-pyridine-2-carboxylic acid |
| | methylamide |
| 175 | 4-(4-{3-[2-(2-Amino-ethyl)-5-trifluoromethyl-phenyl]- |
| | ureidomethyl}-phenoxy)-pyridine-2-carboxylic acid |
| | methylamide |
| 176 | 4-(4-{3-[2-(2-Amino-ethoxy)-5-chloro-4-methyl-phenyl]- |
| | ureidomethyl}-phenoxy)-pyridine-2-carboxylic acid |
| | methylamide |
| 177 | 4-(4-{3-[5-Chloro-4-methyl-2-(2-methylamino-ethoxy)- |
| | phenyl]-ureidomethyl}-phenoxy)-pyridine-2-carboxylic acid |
| | methylamide |
| 178 | 4-(4-{3-[5-Chloro-4-methyl-2-(piperidin-4-yloxy)-phenyl]- |
| | ureidomethyl}-phenoxy)-pyridine-2-carboxylic acid |
| | methylamide |
| 179 | 4-(4-{3-[5-Chloro-4-methyl-2-(2-piperazin-1-yl-ethoxy)- |
| | phenyl]-ureidomethyl}-phenoxy)-pyridine-2-carboxylic acid |
| | methylamide |
| 180 | 4-(4-{3-[2-(2-Methanesulfonylamino-ethoxy)-5- |
| | trifluoromethyl-phenyl]-ureidomethyl}-phenoxy)-pyridine-2- |
| | carboxylic acid methylamide |
| 181 | 4-{4-[(3-Phenyl-ureido)-methyl]-phenoxy}-pyridine-2- |
| | carboxylic acid methylamide |
| 182 | 4-(4-{3-[5-Chloro-4-methyl-2-(pyrrolidin-2-ylmethoxy)- |
| | phenyl]-ureidomethyl}-phenoxy)-pyridine-2-carboxylic acid |
| | methylamide |
| 183 | 4-(4-{3-[5-Chloro-2-(2-isopropylamino-ethoxy)-4-methyl- |
| | phenyl]-ureidomethyl}-phenoxy)-pyridine-2-carboxylic acid |
| | methylamide |
| 184 | 4-{2-Methyl-4-[3-(2-piperazin-1-yl-5-trifluoromethyl-phenyl)- |
| | ureidomethyl]-phenoxy}-pyridine-2-carboxylic acid |
| | methylamide |
| 185 | 4-(4-{3-[2-(2-Amino-2-methyl-propoxy)-5-chloro-4-methyl- |
| | phenyl]-ureidomethyl}-phenoxy)-pyridine-2-carboxylic acid |
| | methylamide |
| 186 | 4-{4-[3-(2-Acetylamino-4-chloro-5-methyl-phenyl)- |
| | ureidomethyl]-2-methyl-phenoxy}-pyridine-2-carboxylic acid |
| | methylamide |
| 187 | 4-[4-(3-{4-Chloro-5-methyl-2-[2-(2,2,2-trifluoro-acetylamino)- |
| | ethoxy]-phenyl}-ureidomethyl)-phenoxy]-pyridine-2- |
| | carboxylic acid methylamide |
| 188 | 4-(4-{3-[2-(2-Methylamino-ethoxy)-5- |
| | trifluoromethanesulfonyl-phenyl]-ureidomethyl}-phenoxy)- |
| | pyridine-2-carboxylic acid methylamide |
| 189 | 4-(2-Methyl-4-{3-[2-(2-methylamino-ethoxy)-5- |
| | trifluoromethanesulfonyl-phenyl]-ureidomethyl}-phenoxy)- |
| | pyridine-2-carboxylic acid methylamide |
| 190 | 4-{4-[3-(2-Carbamoylmethoxy-5-trifluoromethyl-phenyl)- |
| | ureidomethyl]-phenoxy}-pyridine-2-carboxylic acid |
| | methylamide |
| 191 | 4-(4-{3-[2-(3-Amino-propoxy)-4-chloro-5-trifluoromethyl- |
| | phenyl]-ureidomethyl}-phenoxy)-pyridine-2-carboxylic acid |
| | methylamide |
| 192 | 4-{4-[3-(2-Piperazin-1-ylmethyl-5-trifluoromethyl-phenyl)- |
| | ureidomethyl]-phenoxy}-pyridine-2-carboxylic acid |
| | methylamide |
| 193 | 4-(4-{3-[4-Chloro-2-(2-methanesulfonylamino-ethoxy)-5- |
| | methyl-phenyl]-ureidomethyl}-phenoxy)-pyridine-2- |
| | carboxylic acid methylamide |
| 194 | 4-(4-{3-[4-Chloro-2-(2-methanesulfonylamino-ethoxy)-5- |
| | trifluoromethyl-phenyl]-ureidomethyl}-phenoxy)-pyridine-2- |
| | carboxylic acid methylamide |
| 195 | 4-(4-{3-[2-(2-Hydroxy-ethyl)-phenyl]-ureidomethyl}-2- |
| | methyl-phenoxy)-pyridine-2-carboxylic acid methylamide |
| 196 | 4-{4-[3-(2-Hydroxymethyl-4-methyl-phenyl)-ureidomethyl]-2- |
| | methyl-phenoxy}-pyridine-2-carboxylic acid methylamide |
| 197 | 4-(4-{3-[2-(2-Hydroxy-ethoxy)-5-trifluoromethyl-phenyl]- |
| | ureidomethyl}-phenoxy)-pyridine-2-carboxylic acid |
| | methylamide |
| 198 | 4-{4-[3-(2-Hydroxymethyl-phenyl)-ureidomethyl]-2-methyl- |
| | phenoxy}-pyridine-2-carboxylic acid methylamide |
| 199 | 4-(4-{3-[2-(1 -Carbamoyl-1-methyl-ethoxy)-5-trifluoromethyl- |
| | phenyl]-ureidomethyl}-phenoxy)-pyridine-2-carboxylic acid |
| | methylamide |
| 200 | 4-{2-Methyl-4-[3-(2-methylcarbamoylmethyl-5- |
| | trifluoromethyl-phenyl)-ureidomethyl]-phenoxy}-pyridine-2- |
| | carboxylic acid methylamide |
| 201 | 4-{4-[3-(2-[1,2,4]Triazol-1-yl-5-trifluoromethyl-phenyl)- |
| | ureidomethyl]-phenoxy}-pyridine-2-carboxylic acid |
| | methylamide |
| 202 | 4-{2-Methyl-4-[3-(2-[1,2,4]triazol-1-yl-5-trifluoromethyl- |
| | phenyl)-ureidomethyl]-phenoxy}-pyridine-2-carboxylic acid |
| | methylamide |
| 203 | 4-{2-Methyl-4-[3-(2-[1,2,3]triazol-1-yl-5-trifluoromethyl- |
| | phenyl)-ureidomethyl]-phenoxy}-pyridine-2-carboxylic acid |
| | methylamide |
| 204 | 4-{4-[3-(2-Hydroxy-5-trifluoromethyl-phenyl)-ureidomethyl]- |
| | 2-methyl-phenoxy}-pyridine-2-carboxylic acid methylamide |
| 205 | 2-Oxo-6-trifluoromethyl-2,3-dihydro-indole-1-carboxylic acid |
| | 4-(2-methylcarbamoyl-pyridin-4-yloxy)-benzylamide |
| 206 | 4-{4-[3-(2-[1,2,3]Triazol-1-yl-5-trifluoromethyl-phenyl)- |
| | ureidomethyl]-phenoxy}-pyridine-2-carboxylic acid |
| | methylamide |
| 207 | 4-{4-[3-(2-Carbamoylmethyl-5-trifluoromethyl-phenyl)- |
| | ureidomethyl]-2-methyl-phenoxy}-pyridine-2-carboxylic acid |
| | methylamide |
| 208 | 4-(4-{3-[2-(2-Oxo-piperazin-1-ylmethyl)-5-trifluoromethyl- |
| | phenyl]-ureidomethyl}-phenoxy)-pyridine-2-carboxylic acid |
| | methylamide |
| 209 | 4-{4-[3-(2-Carbamoylmethyl-5-trifluoromethyl-phenyl)- |
| | ureidomethyl]-phenoxy}-pyridine-2-carboxylic acid |
| | methylamide |
| 210 | 4-{4-[3-(5-Methyl-pyridin-2-yl)-ureidomethyl]-phenoxy}- |
| | pyridine-2-carboxylic acid methylamide |
| 211 | 4-{2-Methyl-4-[3-(5-methyl-pyridin-2-yl)-ureidomethyl]- |
| | phenoxy}-pyridine-2-carboxylic acid methylamide |
| 212 | 4-{4-[3-(4-Methyl-pyridin-2-yl)-ureidomethyl]-phenoxy}- |
| | pyridine-2-carboxylic acid methylamide |
| 213 | 4-{2-Methyl-4-[3-(4-methyl-pyridin-2-yl)-ureidomethyl]- |
| | phenoxy}-pyridine-2-carboxylic acid methylamide |
| 214 | 4-(4-{3-[2-(Acetylamino-methyl)-5-trifluoromethyl-phenyl]- |
| | ureidomethyl}-2-methyl-phenoxy)-pyridine-2-carboxylic acid |
| | methylamide |
| 215 | 4-(2-Methyl-4-{3-[5-methyl-2-(2-methylamino-ethoxy)- |
| | phenyl]-ureidomethyl}-phenoxy)-pyridine-2-carboxylic acid |
| | methylamide |
| 216 | 4-{4-[3-(5-Chloro-2-methoxy-4-methyl-phenyl)- |
| | ureidomethyl]-2-fluoro-phenoxy}-pyridine-2-carboxylic acid |
| | methylamide |
| 217 | 4-{2-Fluoro-4-[3-(2-methoxy-5-trifluoromethyl-phenyl)- |
| | ureidomethyl]-phenoxy}-pyridine-2-carboxylic acid |
| | methylamide |
| 218 | 4-{4-[3-(4-Chloro-2-methoxy-5-methyl-phenyl)- |
| | ureidomethyl]-2-fluoro-phenoxy}-pyridine-2-carboxylic acid |
| | methylamide |
| 219 | 4-{4-[3-(4-Chloro-5-methyl-2-pyrrol-1-yl-phenyl)- |
| | ureidomethyl]-phenoxy}-pyridine-2-carboxylic acid |
| | methylamide |
| 220 | (2-{3-[3-Methyl-4-(2-methylcarbamoyl-pyridin-4-yloxy)- |
| | benzyl]-ureido}-4-trifluoromethyl-phenyl)-acetic acid |
| 221 | 4-{4-[3-(2-Aminomethyl-5-trifluoromethyl-phenyl)- |
| | ureidomethyl]-2-methyl-phenoxy}-pyridine-2-carboxylic acid |
| | methylamide |
| 222 | 4-{4-[3-(5-Trifluoromethyl-[1,3,4]thiadiazol-2-yl)- |
| | ureidomethyl]-phenoxy}-pyridine-2-carboxylic acid |
| | methylamide |
| 223 | 4-{4-[3-(5-tert-Butyl-2H-pyrazol-3-yl)-ureidomethyl]- |
| | phenoxy}-pyridine-2-carboxylic acid methylamide |
| 224 | 4-{4-[3-(5-tert-Butyl-isoxazol-3-yl)-ureidomethyl]-phenoxy}- |
| | pyridine-2-carboxylic acid methylamide |
| 225 | 4-(4-{3-[3-Chloro-4-(3-oxo-morpholin-4-yl)-phenyl]- |
| | ureidomethyl}-phenoxy)-pyridine-2-carboxylic acid |
| | methylamide |
| 226 | 4-{4-[3-(2-Chloro-pyridin-3-yl)-ureidomethyl]-phenoxy}- |
| | pyridine-2-carboxylic acid methylamide |
| 227 | 4-{4-[3-(6-Methoxy-pyridin-3-yl)-ureidomethyl]-phenoxy}- |
| | pyridine-2-carboxylic acid methylamide |
| 228 | 4-{4-[3-(3-Dimethylamino-phenyl)-ureidomethyl]-phenoxy}- |
| | pyridine-2-carboxylic acid methylamide |
| 229 | 4-{4-[3-(2-Ethoxy-5-trifluoromethyl-phenyl)-ureidomethyl]-2- |
| | methyl-phenoxy}-pyridine-2-carboxylic acid methylamide |
| 230 | 4-{4-[3-(2-Ethoxy-5-trifluoromethyl-phenyl)-ureidomethyl]- |
| | phenoxy}-pyridine-2-carboxylic acid methylamide |
| 231 | 4-{4-[3-(4-Chloro-2-methoxy-5-trifluoromethyl-phenyl)- |
| | ureidomethyl]-2-fluoro-phenoxy}-pyridine-2-carboxylic acid |
| | methylamide |
| 232 | 4-{4-[3-(5-Chloro-2-methoxy-phenyl)-ureidomethyl]-2-fluoro- |
| | phenoxy}-pyridine-2-carboxylic acid methylamide |
| 233 | 4-{2-Fluoro-4-[3-(3-trifluoromethyl-phenyl)-ureidomethyl]- |
| | phenoxy}-pyridine-2-carboxylic acid methylamide |
| 234 | 4-{4-[3-(3-Chloro-4-methyl-phenyl)-ureidomethyl]-2-fluoro- |
| | phenoxy}-pyridine-2-carboxylic acid methylamide |
| 235 | 4-{2-Fluoro-4-[3-(2-[1,2,4]triazol-1-yl-5-trifluoromethyl- |
| | phenyl)-ureidomethyl]-phenoxy}-pyridine-2-carboxylic acid |
| | methylamide |
| 236 | 4-{4-[3-(5-Methyl-isoxazol-3-yl)-ureidomethyl]-phenoxy}- |
| | pyridine-2-carboxylic acid methylamide |
| 237 | 4-(4-{3-[2-(2-Acetylamino-ethoxy)-4-chloro-5-methyl- |
| | phenyl]-ureidomethyl}-phenoxy)-pyridine-2-carboxylic acid |
| | methylamide |
| 238 | 4-(4-{3-[2-(2-Acetylamino-ethoxy)-4-chloro-5-trifluoromethyl- |
| | phenyl]-ureidomethyl}-phenoxy)-pyridine-2-carboxylic acid |
| | methylamide |
| 239 | 4-(4-{3-[2-(2-Acetylamino-ethoxy)-5-trifluoromethyl-phenyl]- |
| | ureidomethyl}-phenoxy)-pyridine-2-carboxylic acid |
| | methylamide |
| 240 | 4-{4-[3-(2-Imidazol-1-yl-5-trifluoromethyl-phenyl)- |
| | ureidomethyl]-2-methyl-phenoxy}-pyridine-2-carboxylic acid |
| | methylamide |
| 241 | 4-{4-[3-(4-Acetylamino-3-trifluoromethyl-phenyl)- |
| | ureidomethyl]-phenoxy}-pyridine-2-carboxylic acid |
| | methylamide |
| 242 | 4-[4-(3-{4-Chloro-2-[2-(1,3-dioxo-1,3-dihydro-isoindol-2-yl)- |
| | ethoxy]-5-trifluoromethyl-phenyl}-ureidomethyl)-phenoxy]- |
| | pyridine-2-carboxylic acid methylamide |
| 243 | 4-{4-[3-(2-Imidazol-1-yl-5-trifluoromethyl-phenyl)- |
| | ureidomethyl]-phenoxy}-pyridine-2-carboxylic acid |
| | methylamide |
| 244 | [2-(5-Chloro-4-methyl-2-{3-[4-(2-methylcarbamoyl-pyridin-4- |
| | yloxy)-benzyl]-ureido}-phenoxy)-ethyl]-methyl-carbamic acid |
| | tert-butyl ester |
| 245 | 1-Phenyl-3-[4-(pyridin-4-yloxy)-benzyl]-urea |
| 246 | 1-[4-Chloro-2-(2-dimethylamino-ethoxy)-5-methyl-phenyl]-3- |
| | [3-methyl-4-(pyridin-4-yloxy)-benzyl]-urea |
| 247 | N-[4-(4-{3-[4-Chloro-2-(2-dimethylamino-ethoxy)-5-methyl- |
| | phenyl]-ureidomethyl}-2-methyl-phenoxy)-pyridin-2-yl]- |
| | acetamide |
| 248 | 1-(4-Chloro-2-methoxy-5-methyl-phenyl)-3-[3-methyl-4- |
| | (pyridin-4-yloxy)-benzyl]-urea |
| 249 | 1-[4-(2-Methyl-furo[3,2-b]pyridin-7-yloxy)-benzyl]-3-phenyl- |
| | urea |
| 250 | 1-[4-Chloro-2-(2-dimethylamino-ethoxy)-5-methyl-phenyl]-3- |
| | [3-methyl-4-(pyrimidin-4-yloxy)-benzyl]-urea |
| 251 | 1-[4-(6-Amino-pyrimidin-4-yloxy)-3-methyl-benzyl]-3-[4- |
| | chloro-2-(2-dimethylamino-ethoxy)-5-methyl-phenyl]-urea |
| 252 | 1-(4-Chloro-2-methoxy-5-methyl-phenyl)-3-[3-methyl-4-(2- |
| | methyl-furo[3,2-b]pyridin-7-yloxy)-benzyl]-urea |
| 253 | 1-[4-Chloro-2-(2-dimethylamino-ethoxy)-5-methyl-phenyl]-3- |
| | [3-methyl-4-(2-methyl-furo[3,2-b]pyridin-7-yloxy)-benzyl]- |
| | urea |
| 254 | 1-[4-(2-Amino-pyridin-4-yloxy)-3-methyl-benzyl]-3-[4-chloro- |
| | 2-(2-dimethylamino-ethoxy)-5-methyl-phenyl]-urea |
| 255 | 4-[4-[[4-chloro-3-(trifluoromethyl)-phenyl]carbamoylamino] |
| | phenoxy]-N-methyl-pyridine-2-carboxamide |

and physiologically acceptable salts, derivatives, solvates, prodrugs and stereoisomers thereof, including mixtures thereof in all ratios.

Another preferred embodiment of the present invention preferably are compounds of the formula I in which
W is N,
X, Y, Q, U, T are independently from one another C or N, with the proviso that one or more of X, Y, Q, U and T are carbon atoms and that M is bonded to a carbon atom,
V is a single bond or —CR⁴R⁵—,
M is O,
R¹ is

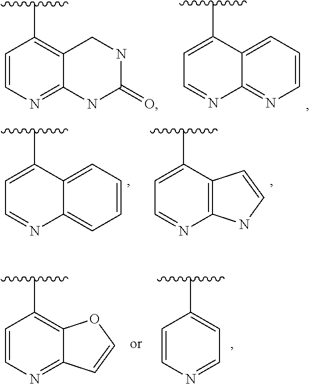

which is unsubstituted or monosubstituted by R⁶,
R³ is

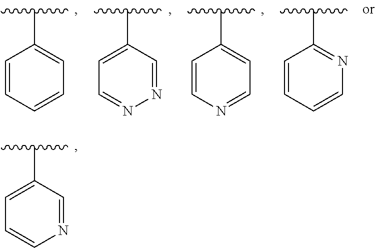

which is unsubstituted or mono-, di- or trisubstituted by R⁷, and
R², R⁶ and R⁷ independently from one another have the meanings as disclosed above
   and physiologically acceptable salts, derivatives, solvates, prodrugs and stereoisomers thereof, including mixtures thereof in all ratios.
   and physiologically acceptable salts, derivatives, solvates, prodrugs and stereoisomers thereof, including mixtures thereof in all ratios.

A particularly preferred embodiment of the present invention are compounds of the formula I in which
W is N,
X, Y, Q, U, T are C,
V is a single bond or —CR⁴R⁵—,
M is O,
R¹ is

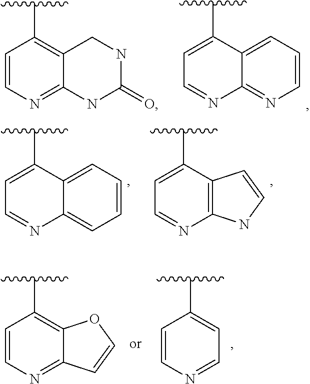

which is unsubstituted or mono-, di- or trisubstituted by R⁶,
R² is H, A, CN, OH, OA or Hal,
R³ is

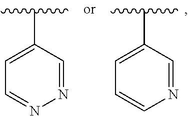 [SMILES: *c1ccnnc1 ; *c1cccnc1]

which is unsubstituted or mono-, di- or trisubstituted by R⁷,
R⁴, R⁵ are H,
R⁶ is H, A, ═O, CN, CF₃, Hal, COOH, C(O)NH₂, C(O)NHA, C(O)NA₂, (CH₂)ₙOH, (CH₂)ₙOA, (CH₂)ₙaryl, (CH₂)ₙ heteroaryl or (CH₂)ₙheterocyclyl,
R⁷ is H, A, ═O, CN, CH₂)ₙOH, (CH₂)ₙOA, CF₃, Hal, COOH, (CH₂)ₙaryl, (CH₂)ₙ heteroaryl or (CH₂)ₙheterocyclyl,
A is alkyl, and
n is 0-3
   and physiologically acceptable salts, derivatives, solvates, prodrugs and stereoisomers thereof, including mixtures thereof in all ratios.

A particularly preferred embodiment of the present invention are compounds of the formula I in which
W is N,
X, Y, Q, U, T are C,
V is a single bond or —CR⁴R⁵—,
M is O,
R¹ is

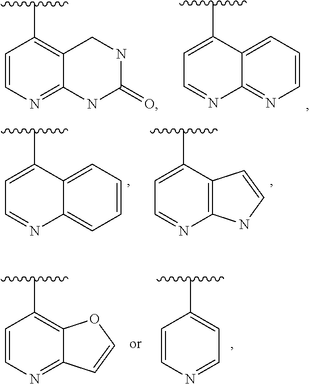

which is unsubstituted or mono-, di- or trisubstituted by R⁶,
R² is H, A, CN, OH, OA or Hal,
R³ is

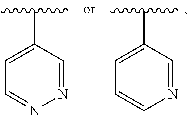 [SMILES: *c1ccnnc1 ; *c1cccnc1]

which is unsubstituted or mono-, di- or trisubstituted by R⁷,
R⁴, R⁵ are H,
R⁶ is H, alkyl, cycloalkyl, ═O, CF₃, CN, (CH₂)ₙOH, (CH₂)ₙOA, Hal, COOH, C(O)NH₂or C(O)NHA,
R⁷ is H, ═O, A, CN, (CH₂)ₙOH, (CH₂)ₙOA, (CH₂)ₙaryl, Hal or CF₃,
A is alkyl, and
n is 0-3
   and physiologically acceptable salts, derivatives, solvates, prodrugs and stereoisomers thereof, including mixtures thereof in all ratios.

Particularly preferred are compounds of the formula I in which
W is N,
X, Y, Q, U, T are C,
V —CR⁴R⁵—,
M is O,
R¹ and R⁶ together are

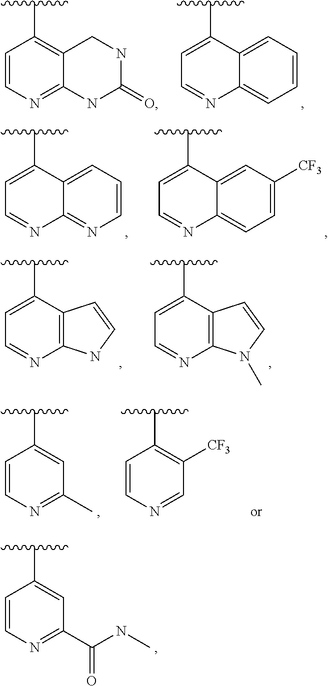

R² is H or alkyl with 1 to 5 C-atoms,
R³ and R⁷ together are

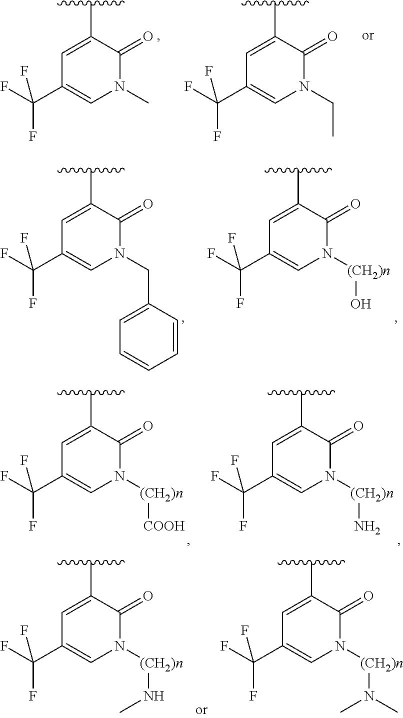

R⁴, R⁵ are H and
n 0-3
   and physiologically acceptable salts, derivatives, solvates, prodrugs and stereoisomers thereof, including mixtures thereof in all ratios.

Particularly preferred are compounds of the formula I in which
W is N,
X, Y, Q, U, T are C,
V a single bond,
M is O,
R¹ and R⁶ together are

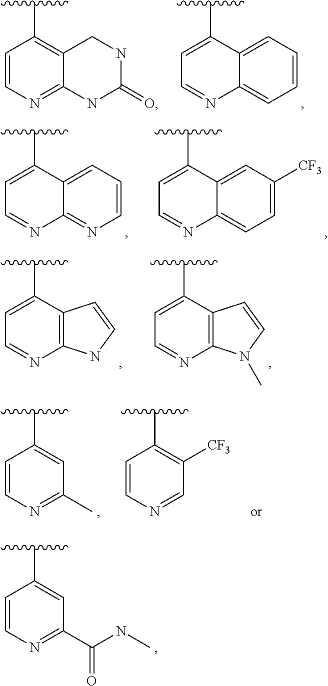

R² is H or alkyl with 1 to 5 C-atoms,
R³ and R⁷ together are

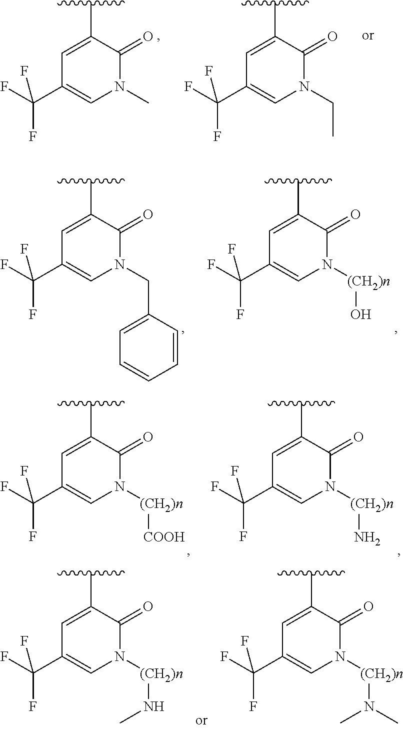

R⁴, R⁵ are H and
n 0-3
   and physiologically acceptable salts, derivatives, solvates, prodrugs and stereoisomers thereof, including mixtures thereof in all ratios.

Very particular preference is given to the following compounds of the formula I selected from the group consisting of
a) 1-(1-Methyl-2-oxo-5-trifluoromethyl-1,2-dihydro-pyridin-3-yl)-3-[4-(1H-pyrrolo[2,3-b]pyridin-4-yloxy)-benzyl]-urea
b) 1-(1-Methyl-2-oxo-5-trifluoromethyl-1,2-dihydro-pyridin-3-yl)-3-[4-(6-trifluoromethyl-quinolin-4-yloxy)-benzyl]-urea
c) 1-(1-Methyl-2-oxo-5-trifluoromethyl-1,2-dihydro-pyridin-3-yl)-3-[4-(1H-pyrrolo[2,3-b]pyridin-4-yloxy)-benzyl]-urea
d) 1-(1-Methyl-2-oxo-5-trifluoromethyl-1,2-dihydro-pyridin-3-yl)-3-[4-([1,8]naphthyridin-4-yloxy)-benzyl]-urea
e) 1-(1-Methyl-2-oxo-5-trifluoromethyl-1,2-dihydro-pyridin-3-yl)-3-[4-(2-oxo-1,2,3,4-tetrahydro-pyrido[2,3-d]pyrimidin-5-yloxy)-benzyl]-urea
f) 1-[3-Methyl-4-(2-methyl-pyridin-4-yloxy)-benzyl]-3-(1-methyl-2-oxo-5-trifluoromethyl-1,2-dihydro-pyridin-3-yl)-urea
g) 4-{2-Methyl-4-[3-(1-methyl-2-oxo-5-trifluoromethyl-1,2-dihydro-pyridin-3-yl)-ureidomethyl]-phenoxy}-pyridine-2-carboxylic acid methylamide
h) 4-{4-[3-(1-Methyl-2-oxo-5-trifluoromethyl-1,2-dihydro-pyridin-3-yl)-ureidomethyl]-phenoxy}-pyridine-2-carboxylic acid methylamide
i) 1-(1-Methyl-2-oxo-5-trifluoromethyl-1,2-dihydro-pyridin-3-yl)-3-[4-(quinolin-4-yloxy)-benzyl]-urea
j) 1-[3-Methyl-4-(2-oxo-1,2,3,4-tetrahydro-pyrido[2,3-d]pyrimidin-5-yloxy)-benzyl]-3-(1-methyl-2-oxo-5-trifluoromethyl-1,2-dihydro-pyridin-3-yl)-urea
k) 4-{4-[3-(1-Ethyl-2-oxo-5-trifluoromethyl-1,2-dihydro-pyridin-3-yl)-ureidomethyl]-phenoxy}-pyridine-2-carboxylic acid methylamide
l) 4-{4-[3-(1-Benzyl-2-oxo-5-trifluoromethyl-1,2-dihydro-pyridin-3-yl)-ureidomethyl]-phenoxy}-pyridine-2-carboxylic acid methylamide
m) 1-(1-Methyl-2-oxo-5-trifluoromethyl-1,2-dihydro-pyridin-3-yl)-3-[4-(3-trifluoromethyl-pyridin-4-yloxy)-benzyl]-urea
n) 4-{4-[3-(1-Hydroxymethyl-2-oxo-5-trifluoromethyl-1,2-dihydro-pyridin-3-yl)-ureidomethyl]-phenoxy}-pyridine-2-carboxylic acid methylamide
o) (3-{3-[4-(2-Methylcarbamoyl-pyridin-4-yloxy)-benzyl]-ureido}-2-oxo-5-trifluoro-methyl-2H-pyridin-1-yl)-acetic acid
p) 4-{4-[3-(1-Aminomethyl-2-oxo-5-trifluoromethyl-1,2-dihydro-pyridin-3-yl)-ureidomethyl]-phenoxy}-pyridine-2-carboxylic acid methylamide
q) 4-{4-[3-(1-Methylaminomethyl-2-oxo-5-trifluoromethyl-1,2-dihydro-pyridin-3-yl)-ureidomethyl]-phenoxy}-pyridine-2-carboxylic acid methylamide
r) 4-{4-[3-(1-Dimethylaminomethyl-2-oxo-5-trifluoromethyl-1,2-dihydro-pyridin-3-yl)-ureidomethyl]-phenoxy}-pyridine-2-carboxylic acid methylamide
   and physiologically acceptable salts, derivatives, solvates, prodrugs and stereoisomers thereof, including mixtures thereof in all ratios.

Another preferred embodiment of the present invention preferably are compounds of the formula I in which
W is O,
X, Y, Q, U, T are independently from one another C or N, with the proviso that one or more of X, Y, Q, U and T are carbon atoms and that M is bonded to a carbon atom,
V a single bond or —CR⁴R⁵—,
M is O,
R¹ is

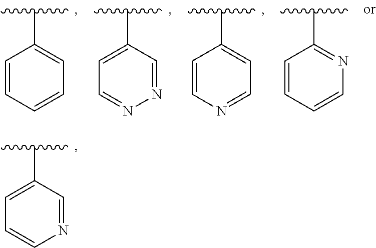

which is unsubstituted or mono-, di- or trisubstituted by R⁶,
R³ is

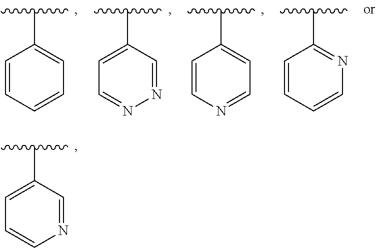

which is unsubstituted or mono-, di- or trisubstituted by R⁷,
R⁴, R⁵ are independently from one another selected from the group consisting of H, alkyl and cycloalkyl, and
R², R⁶ and R⁷ independently from one another have the meanings as disclosed above
   and physiologically acceptable salts, derivatives, solvates, prodrugs and stereoisomers thereof, including mixtures thereof in all ratios.

Another preferred embodiment of the present invention preferably are compounds of the formula I in which
W is O,
X, Y, Q, U, T are C,
V a single bond or —CR⁴R⁵—,
M is O,
R¹ is

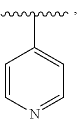 [SMILES: *c1ccncc1]

which is unsubstituted or mono-, di- or trisubstituted by R⁶,
R³ is

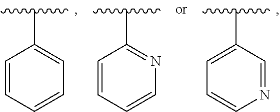

which is unsubstituted or mono-, di- or trisubstituted by R⁷,
R⁴, R⁵ are independently from one another selected from the group consisting of H, alkyl and cycloalkyl, and
R², R⁶ and R⁷ independently from one another have the meanings as disclosed above
   and physiologically acceptable salts, derivatives, solvates, prodrugs and stereoisomers thereof, including mixtures thereof in all ratios.

Particularly preferred are compounds of the formula I in which
W is O,
X, Y, Q, U, T are C,
V —CR⁴R⁵—,
M is O,
R¹ and R⁶ together are

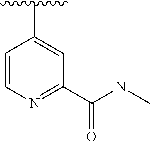 [SMILES: *c1ccnc(C(=O)NC)c1]

R² is H or alkyl with 1 to 5 C-atoms,
R³ is

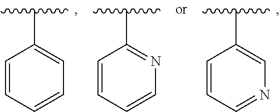

which is unsubstituted or mono-, di- or trisubstituted by R⁷,
R⁴, R⁵ are H,
R⁷ alkyl with 1-5 C-atoms, CN, OH, OA, Hal or CF₃
   and physiologically acceptable salts, derivatives, solvates, prodrugs and stereoisomers thereof, including mixtures thereof in all ratios.

Particularly preferred are compounds of the formula I in which
W is O,
X, Y, Q, U, T are C,
V a single bond,
M is O,
R¹ and R⁶ together are

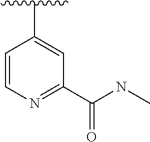 [SMILES: *c1ccnc(C(=O)NC)c1]

R² is H or alkyl with 1 to 5 C-atoms,
R³ is

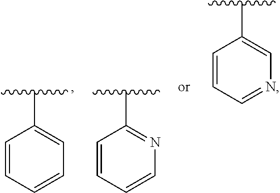

which is unsubstituted or mono-, di- or trisubstituted by R⁷,
R⁴, R⁵ are H,
R⁷ alkyl with 1-5 C-atoms, CN, OH, OA, Hal or CF₃
   and physiologically acceptable salts, derivatives, solvates, prodrugs and stereoisomers thereof, including mixtures thereof in all ratios.

Very particular preference is given to the following compounds of the formula I selected from the group consisting of
a) (2-Hydroxy-5-trifluoromethyl-pyridin-3-yl)-carbamic acid 3-methyl-4-(2-methylcarbamoyl-pyridin-4-yloxy)-benzyl ester
b) (2-Hydroxy-5-methyl-pyridin-3-yl)-carbamic acid 4-(2-methylcarbamoyl-pyridin-4-yloxy)-benzyl ester
c) (4-Trifluoromethyl-pyridin-2-yl)-carbamic acid 4-(2-methylcarbamoyl-pyridin-4-yloxy)-benzyl ester
d) (4-Trifluoromethyl-pyridin-2-yl)-carbamic acid 3-methyl-4-(2-methylcarbamoyl-pyridin-4-yloxy)-benzyl ester
e) (2-Hydroxy-5-trifluoromethyl-pyridin-3-yl)-carbamic acid 4-(2-methylcarbamoyl-pyridin-4-yloxy)-benzyl ester
f) (4-Chloro-3-trifluoromethyl-phenyl)-carbamic acid 4-(2-methylcarbamoyl-pyridin-4-yloxy)-benzyl ester
   and physiologically acceptable salts, derivatives, solvates, prodrugs and stereoisomers thereof, including mixtures thereof in all ratios.

Another preferred embodiment of the present invention preferably are compounds of the formula I in which
W N,
X, Y, Q, U, T are independently from one another C or N, with the proviso that one or more of X, Y, Q, U and T are carbon atoms and that M is bonded to a carbon atom,
V is a single bond,
M is O
R¹ is

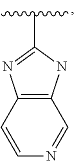 [SMILES: *c1nc2ccncc2[nH]1]

which is unsubstituted or mono-, di- or trisubstituted by R⁶,
R³ is

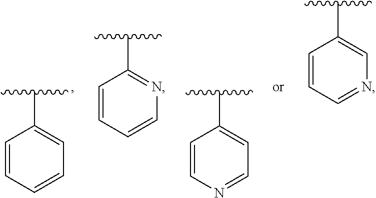

which is unsubstituted or mono-, di- or trisubstituted by R⁷,
R⁴, R⁵ are independently from one another selected from the group consisting of H, alkyl and cycloalkyl, and
R², R⁶ and R⁷ independently from one another have the meanings as disclosed above
   and physiologically acceptable salts, derivatives, solvates, prodrugs and stereoisomers thereof, including mixtures thereof in all ratios.

Another preferred embodiment of the present invention preferably are compounds of the formula I in which
W N,
X, Y, Q, U, T are C,
V is a single bond,
M is —CR⁴R⁵—,
R¹ is

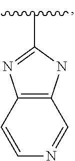 [SMILES: *c1nc2ccncc2[nH]1]

which is unsubstituted or mono-, di- or trisubstituted by R⁶,
R² is H, A, CN, OH, OA or Hal,

R³ is

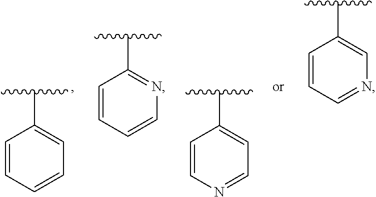

which is unsubstituted or mono-, di- or trisubstituted by R⁷,
R⁴, R⁵ are independently from one another selected from the group consisting of H, alkyl and cycloalkyl, and
R⁶, R⁷ are independently from one another selected from the group consisting of H, alkyl with 1-5 C-atoms, ═O, CN, Hal, CF₃, OH, OA, COOH, C(O)NH₂ and C(O)NHA,
   and physiologically acceptable salts, derivatives, solvates, prodrugs and stereoisomers thereof, including mixtures thereof in all ratios.

Particularly preferred are compounds of the formula I in which
W N,
X, Y, Q, U, T are C,
V is a single bond,
M is —CR⁴R⁵—,
R¹ and R⁶ together are

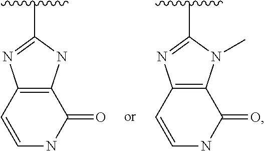 [SMILES: *c1nc2cc[nH]c(=O)c2[nH]1 ; "or" ; *c1nc2cc[nH]c(=O)c2n1C]

R² is H,
R³ is

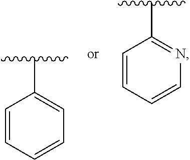 [SMILES: *c1ccccc1 ; "or" ; *c1ccccn1]

which is unsubstituted or mono-, di- or trisubstituted by R⁷,
R⁴, R⁵ are H,
R⁷ is H, alkyl with 1-5 C-atoms, ═O, CF₃, OH, OA or Hal,
   and physiologically acceptable salts, derivatives, solvates, prodrugs and stereoisomers thereof, including mixtures thereof in all ratios.

Very particular preference is given to the following compounds of the formula I selected from the group consisting of
a) 1-[4-(4-Oxo-4,5-dihydro-3H-imidazo[4,5-c]pyridin-2-ylmethyl)-phenyl]-3-(3-trifluoromethyl-phenyl)-urea
b) 1-[4-(3-Methyl-4-oxo-4,5-dihydro-3H-imidazo[4,5-c]pyridin-2-ylmethyl)-phenyl]-3-(1-methyl-2-oxo-5-trifluoromethyl-1,2-dihydro-pyridin-3-yl)-urea
c) 1-(2-Methoxy-5-trifluoromethyl-phenyl)-3-[4-(3-methyl-4-oxo-4,5-dihydro-3H-imidazo[4,5-c]pyridin-2-ylmethyl)-phenyl]-urea
d) 1-(1-Methyl-2-oxo-5-trifluoromethyl-1,2-dihydro-pyridin-3-yl)-3-[4-(4-oxo-4,5-dihydro-3H-imidazo[4,5-c]pyridin-2-ylmethyl)-phenyl]-urea
e) 1-(5-Methyl-pyridin-3-yl)-3-[4-(4-oxo-4,5-dihydro-3H-imidazo[4,5-c]pyridin-2-ylmethyl)-phenyl]-urea
   and physiologically acceptable salts, derivatives, solvates, prodrugs and stereoisomers thereof, including mixtures thereof in all ratios.

Another preferred embodiment of the present invention preferably are compounds of the formula I in which
W is CH₂, CH₂CH₂, CH₂CHOH or —(CH₂)O—,
X, Y, Q, U, T are C,
V is a single bond,
M is O,
R¹ is

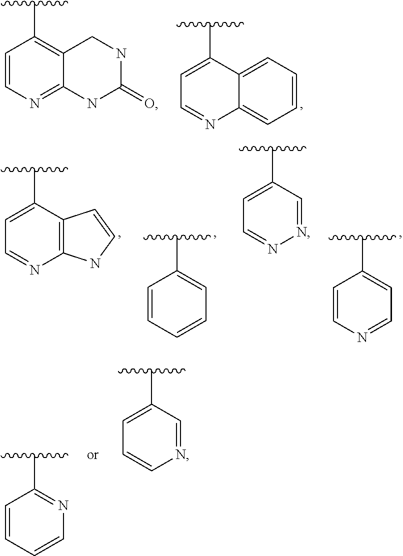

which is unsubstituted or mono-, di- or trisubstituted by R⁶,
R³ is

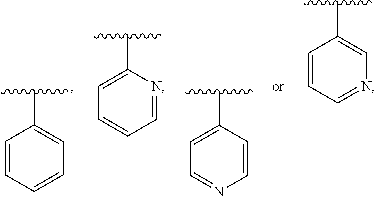

which is unsubstituted or mono-, di- or trisubstituted by R⁷,
R², R⁶ and R⁷ independently from one another have the meanings as disclosed above
   and physiologically acceptable salts, derivatives, solvates, prodrugs and stereoisomers thereof, including mixtures thereof in all ratios.

Another preferred embodiment of the present invention preferably are compounds of the formula I in which
W is CH₂, CH₂CH₂, CH₂CHOH or —(CH₂)O—,
X, Y, Q, U, T are C,
V is a single bond,
M is O,
R¹ is

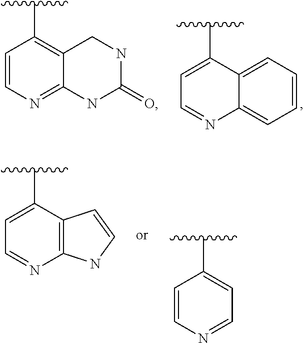

which is unsubstituted or mono-, di- or trisubstituted by R⁶,
R² is H or alkyl with 1-5 C-atoms,
R³ is

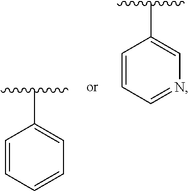 [SMILES: *c1ccccc1 ; *c1cccnc1]

which is unsubstituted or mono-, di- or trisubstituted by R⁷,
R⁶ is H, alkyl, cycloalkyl, ═O, CF₃, CN, (CH₂)ₙOH, (CH₂)ₙOA, Hal, COOH, C(O)NH₂ or C(O)NHA,
R⁷ is H, ═O, A, CN, (CH₂)ₙOH, (CH₂)ₙOA, Hal or CF₃,
A is alkyl, and
n is 0-3
   and physiologically acceptable salts, derivatives, solvates, prodrugs and stereoisomers thereof, including mixtures thereof in all ratios.

Another preferred embodiment of the present invention preferably are compounds of the formula I in which
W is CH₂, CH₂CH₂, CH₂CHOH or —(CH₂)O—,
X, Y, Q, U, T are C,
V is a single bond,
M is O,
R¹ and R⁶ together are

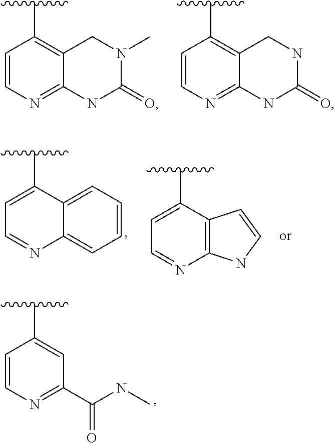

R² is H or alkyl with 1-5 C-atoms, and
R³ and R⁷ together are

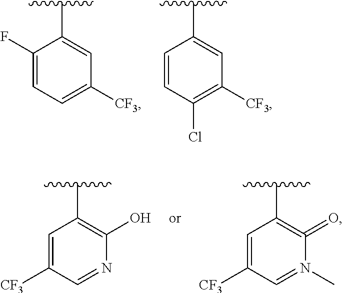

and physiologically acceptable salts, derivatives, solvates, prodrugs and stereoisomers thereof, including mixtures thereof in all ratios.

Very particular preference is given to the following compounds of the formula I selected from the group consisting of
a) 4-{4-[(4-Chloro-3-trifluoromethyl-phenylcarbamoyl)-methyl]-2-methyl-phenoxy}-pyridine-2-carboxylic acid methylamide
b) 4-{4-[2-(4-Chloro-3-trifluoromethyl-phenylcarbamoyl)-1-hydroxy-ethyl]-2-methyl-phenoxy}-pyridine-2-carboxylic acid methylamide
c) 4-{4-[2-(4-Chloro-3-trifluoromethyl-phenylcarbamoyl)-ethyl]-2-methyl-phenoxy}-pyridine-2-carboxylic acid methylamide
d) 4-{4-[(1-Methyl-2-oxo-5-trifluoromethyl-1,2-dihydro-pyridin-3-ylcarbamoyl)-methoxy]-phenoxy}-pyridine-2-carboxylic acid methylamide
e) N-(1-Methyl-2-oxo-5-trifluoromethyl-1,2-dihydro-pyridin-3-yl)-2-[4-(2-oxo-1,2,3,4-tetrahydro-pyrido[2,3-d]pyrimidin-5-yloxy)-phenoxy]-acetamide
f) N-(2-Fluoro-5-trifluoromethyl-phenyl)-2-[4-(2-oxo-1,2,3,4-tetrahydro-pyrido[2,3-d]pyrimidin-5-yloxy)-phenoxy]-acetamide
g) N-(1-Methyl-2-oxo-5-trifluoromethyl-1,2-dihydro-pyridin-3-yl)-2-[4-(quinolin-4-yloxy)-phenoxy]-acetamide
h) 2-[4-(3a,7a-Dihydro-1H-pyrrolo[2,3-b]pyridin-4-yloxy)-phenoxy]-N-(1-methyl-2-oxo-5-trifluoromethyl-1,2-dihydro-pyridin-3-yl)-acetamide
i) 4-{4-[(2-Hydroxy-5-trifluoromethyl-pyridin-3-ylcarbamoyl)-methyl]-2-methyl-phenoxy}-pyridine-2-carboxylic acid methylamide
j) 4-{2-Methyl-4-[(1-methyl-2-oxo-5-trifluoromethyl-1,2-dihydro-pyridin-3-ylcarbamoyl)-methyl]-phenoxy}-pyridine-2-carboxylic acid methylamide
k) 2-[3-Methyl-4-(3-methyl-2-oxo-1,2,3,4-tetrahydro-pyrido[2,3-d]pyrimidin-5-yloxy)-phenyl]-N-(1-methyl-2-oxo-5-trifluoromethyl-1,2-dihydro-pyridin-3-yl)-acetamide
l) N-(2-Fluoro-5-trifluoromethyl-phenyl)-2-[3-methyl-4-(3-methyl-2-oxo-1,2,3,4-tetrahydro-pyrido[2,3-d]pyrimidin-5-yloxy)-phenyl]-acetamide
   and physiologically acceptable salts, derivatives, solvates, prodrugs and stereoisomers thereof, including mixtures thereof in all ratios.

If the above-mentioned amino acids can occur in a plurality of enantiomeric forms, all these forms and also mixtures thereof (for example DL forms) are included above and below.

Furthermore, the abbreviations have the following meanings:

Boc tert-butoxycarbonyl
CBZ benzyloxycarbonyl
DNP 2,4-dinitrophenyl
FMOC 9-fluorenylmethoxycarbonyl
imi-DNP 2,4-dinitrophenyl in the 1-position of the imidazole ring
OMe methyl ester
POA phenoxyacetyl
DCCI dicyclohexylcarbodiimide
HOBt 1-hydroxybenzotriazole

Hal denotes fluorine, chlorine, bromine or iodine, in particular fluorine or chlorine.

A is an unbranched (linear), branched or cyclic hydrocarbon chain and has 1, 2, 3, 4, 5, 6, 7, 8, 9 or 10 C atoms. A preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, linear or branched heptyl, octyl, nonyl or decyl.

Cyclic alkyl or cycloalkyl preferably denotes (if A is cyclic it denotes) cyclopropyl, cyclobutyl, cyclopentyl, cyclohexyl or cycloheptyl.

Additionally, A denotes also alkenyl such as ethenyl, propylenyl, butenyl and the like.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Especially preferred is C₁-C₅alkyl. A C₁-C₅alkyl radical is for example a methyl, ethyl, propyl, isopropyl, butyl, isobutyl, tert-butyl or pentyl.

“Aryl”, Ar” or “aromatic hydrocarbon residue” means a mono- or polycyclic aromatic ring system containing carbon ring atoms. The preferred aryls are monocyclic or bicyclic 6-10 membered aromatic ring systems. Examples of “aryl” groups include, but are not limited to Phenyl, 2-naphthyl, 1-naphthyl, biphenyl, indanyl as well as substituted derivatives thereof. The most preferred aryl is phenyl.

“Heterocycle” and “heterocyclyl” refer to saturated or unsaturated non-aromatic rings or ring systems containing at least one heteroatom selected from O. S and N. further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, and the like.

“Heteroaryl” means an aromatic or partially aromatic heterocycle that contains at least one ring heteroatom selected from O. S and N. Heteroaryls thus includes heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoxazolyl, isoindolyl, dihydrobenzothienyl, indolizinyl, cinnolinyl, phthalazinyl, quinazolinyl, naphthyridinyl, carbazolyl, benzdioxinyl, benzodioxolyl, quinoxalinyl, purinyl, furazanyl, thiophenyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

All physiologically acceptable salts, derivatives, solvates and stereoisomers of these compounds, including mixtures thereof in all ratios, are also in accordance with the invention.

The invention also relates to the optically active forms (stereoisomers), the enantiomers, the racemates, the diastereomers and hydrates and solvates of these compounds.

Compounds of the formula I according to the invention may be chiral owing to their molecular structure and may accordingly occur in various enantiomeric forms. They may therefore be in racemic or optically active form. Since the pharmaceutical efficacy of the racemates or stereoisomers of the compounds according to the invention may differ, it may be desirable to use the enantiomers. In these cases, the end product, but also even the intermediates, may be separated into enantiomeric compounds by chemical or physical measures known to the person skilled in the art or already employed as such in the synthesis.

Pharmaceutically or physiologically acceptable derivatives are taken to mean, for example, salts of the compounds according to the invention and also so-called prodrug compounds. Prodrug compounds are taken to mean compounds of the formula I which have been modified with, for example, alkyl or acyl groups (see also amino- and hydroxyl-protecting groups below), sugars or oligopeptides and which are rapidly cleaved or liberated in the organism to form the effective compounds according to the invention. These also include biodegradable polymer derivatives of the compounds according to the invention, as described, for example, in Int. J. Pharm. 115 (1995), 61-67.

Suitable acid-addition salts are inorganic or organic salts of all physiologically or pharmacologically acceptable acids, for example halides, in particular hydrochlorides or hydrobromides, lactates, sulfates, citrates, tartrates, maleates, fumarates, oxalates, acetates, phosphates, methylsulfonates or p-toluenesulfonates.

Solvates of the compounds of the formula I are taken to mean adductions of inert solvent molecules onto the compounds of the formula I which form owing to their mutual attractive force. Solvates are, for example, hydrates, such as monohydrates or dihydrates, or alcoholates, i.e. addition compounds with alcohols, such as, for example, with methanol or ethanol.

The invention also relates to mixtures of the compounds of the formula I according to the invention, for example mixtures of two diastereomers, for example in the ratio 1:1, 1:2, 1:3, 1:4, 1:5, 1:10, 1:100 or 1:1000. They are particularly preferably mixtures of two stereoisomeric compounds.

Another embodiment of the present invention is a process for the preparation of the compounds of the formula I, characterized in that the compounds are prepared by stepwise reactions of building blocks (see example 2).

It is possible to carry out the reactions stepwise in each case and to modify the sequence of the linking reactions of the building blocks with adaptation of the protecting-group concept.

The starting materials or starting compounds are generally known. If they are novel, they can be prepared by methods known per se.

If desired, the starting materials can also be formed in situ by not isolating them from the reaction mixture, but instead immediately converting them further into the compounds of the formula I.

The compounds of the formula I are preferably obtained by liberating them from their functional derivatives by solvolysis, in particular by hydrolysis, or by hydrogenolysis. Preferred starting materials for the solvolysis or hydrogenolysis are those which contain correspondingly protected amino, carboxyl and/or hydroxyl groups instead of one or more free amino, carboxyl and/or hydroxyl groups, preferably those which carry an amino-protecting group instead of an H atom which is connected to an N atom. Preference is furthermore given to starting materials which carry a hydroxyl-protecting group instead of the H atom of a hydroxyl group. Preference is also given to starting materials which carry a protected carboxyl group instead of a free carboxyl group. It is also possible for a plurality of identical or different protected amino, carboxyl and/or hydroxyl groups to be present in the molecule of the starting material. If the protecting groups present are different from one another, they can in many cases be cleaved off selectively.

The functional derivatives of the compounds of the formula I to be used as starting materials can be prepared by known methods of amino-acid and peptide synthesis, as described, for example, in the said standard works and patent applications.

The compounds of the formula I are liberated from their functional derivatives, depending on the protecting group used, for example, with the aid of strong acids, advantageously using trifluoroacetic acid or perchloric acid, but also using other strong inorganic acids, such as hydrochloric acid or sulfuric acid, strong organic acids, such as trichloroacetic acid, or sulfonic acids, such as benzoyl- or p-toluenesulfonic acid. The presence of an additional inert solvent and/or a catalyst is possible, but is not always necessary.

Depending on the respective synthetic route, the starting materials can optionally be reacted in the presence of an inert solvent.

Suitable inert solvents are, for example, heptane, hexane, petroleum ether, DMSO, benzene, toluene, xylene, trichloroethylene, 1,2-dichloroethane, carbon tetrachloride, chloroform or dichloromethane; alcohols, such as methanol, ethanol, isopropanol, n-propanol, n-butanol or tert-butanol; ethers, such as diethyl ether, diisopropyl ether (preferably for substitution on the indole nitrogen), tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide, N-methylpyrrolidone (NMP) or dimethylformamide (DMF); nitriles, such as acetonitrile; esters, such as ethyl acetate, carboxylic acids or acid anhydrides, such as, for example, acetic acid or acetic anhydride, nitro compounds, such as nitromethane or nitrobenzene, optionally also mixtures of the said solvents with one another or mixtures with water.

The amount of solvent is not crucial; 10 g to 500 g of solvent can preferably be added per g of the compound of the formula I to be reacted.

It may be advantageous to add an acid-binding agent, for example an alkali or alkaline-earth metal hydroxide, carbonate or bicarbonate or other alkali or alkaline-earth metal salts of weak acids, preferably a potassium, sodium or calcium salt, or to add an organic base, such as, for example, triethylamine, dimethylamine, pyridine or quinoline, or an excess of the amine component.

The resultant compounds according to the invention can be separated from the corresponding solution in which they are prepared (for example by centrifugation and washing) and can be stored in another composition after separation, or they can remain directly in the preparation solution. The resultant compounds according to the invention can also be taken up in desired solvents for the particular use.

Suitable reaction temperatures are temperatures from 0 to 40° C., preferably 5 to 25° C.

The reaction duration depends on the reaction conditions selected. In general, the reaction duration is 0.5 hour to 10 days, preferably 1 to 24 hours.

On use of a microwave, the reaction time can be reduced to values of 1 to 60 minutes.

The compounds of the formula I and also the starting materials for their preparation are, in addition, prepared by known methods, as described in the literature (for example in standard works, such as Houben-Weyl, Methoden der organischen Chemie [Methods of Organic Chemistry], Georg-Thieme-Verlag, Stuttgart), for example under reaction conditions which are known and suitable for the said reactions. Use can also be made here of variants known per se, which are not described here in greater detail.

Conventional work-up steps, such as, for example, addition of water to the reaction mixture and extraction, enable the compounds to be obtained after removal of the solvent. It may be advantageous, for further purification of the product, to follow this with a distillation or crystallisation or to carry out a chromatographic purification.

Another embodiment of the present invention is a process for the preparation of the compounds of the formula I, characterized in that
a) the base of a compound of the formula I is converted into one of its salts by treatment with an acid, or
b) an acid of a compound of the formula I is converted into one of its salts by treatment with a base.

An acid of the formula I can be converted into the associated addition salt using a base, for example by reaction of equivalent amounts of the acid and base in an inert solvent, such as ethanol, and subsequent evaporation. Suitable bases for this reaction are, in particular, those which give physiologically acceptable salts. Thus, the acid of the formula I can be converted into the corresponding metal salt, in particular alkali or alkaline-earth metal salt, using a base (for example sodium hydroxide, potassium hydroxide, sodium carbonate or potassium carbonate) or into the corresponding ammonium salt. Organic bases which give physiologically acceptable salts, such as, for example, ethanolamine, are also suitable for this reaction.

On the other hand, a base of the formula I can be converted into the associated acid-addition salt using an acid, for example by reaction of equivalent amounts of the base and acid in an inert solvent, such as ethanol, with subsequent evaporation. Suitable acids for this reaction are, in particular, those which give physiologically acceptable salts. Thus, it is possible to use inorganic acids, for example sulfuric acid, nitric acid, hydrohalic acids, such as hydrochloric acid or hydrobromic acid, phosphoric acids, such as orthophosphoric acid, sulfamic acid, furthermore organic acids, in particular aliphatic, alicyclic, araliphatic, aromatic or heterocyclic, mono- or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxysulfonic acid, benzenesulfonic acid, p-toluenesulfonic acid, naphthalenemono- and -disulfonic acids or laurylsulfuric acid. Salts with physiologically unacceptable acids, for example picrates, can be used for the isolation and/or purification of the compounds of the formula I.

It has been found that the compounds of the formula I are well tolerated and have valuable pharmacological properties, since they selectively inhibit DDR2.

The invention therefore furthermore relates to the use of compounds according to the invention for the preparation of a medicament for the treatment and/or prophylaxis of diseases which are caused, promoted and/or propagated by DDR2 and/or by DDR2-promoted signal transduction.

The invention thus also relates, in particular, to a medicament comprising at least one compound according to the invention and/or one of its physiologically acceptable salts, derivatives, solvates and stereoisomers, including mixtures thereof in all ratios, for use in the treatment and/or prophylaxis of physiological and/or pathophysiological states.

Particular preference is given, in particular, to physiological and/or pathophysiological states which are connected to DDR2.

Physiological and/or pathophysiological states are taken to mean physiological and/or pathophysiological states which are medically relevant, such as, for example, diseases or illnesses and medical disorders, complaints, symptoms or complications and the like, in particular diseases.

The invention furthermore relates to a medicament comprising at least one compound according to the invention and/or one of its physiologically acceptable salts, derivatives, solvates, prodrugs and stereoisomers, including mixtures thereof in all ratios, for use in the treatment and/or prophylaxis of physiological and/or pathophysiological states selected from the group consisting of osteoarthritis, hepatocirrhosis, traumatic cartilage injuries, pain, allodynia or hyperalgesia.

An especially preferred embodiment of the present invention is a medicament comprising at least one compound according to the invention and/or one of its physiologically acceptable salts, derivatives, solvates, prodrugs and stereoisomers, including mixtures thereof in all ratios, for use in the treatment and/or prophylaxis of physiological and/or pathophysiological states selected from the group consisting of osteoarthritis and pain.

The invention furthermore relates to a medicament comprising at least one compound according to the invention and/or one of its physiologically acceptable salts, derivatives, solvates, prodrugs and stereoisomers, including mixtures thereof in all ratios, for use in the treatment and/or prophylaxis of physiological and/or pathophysiological states selected from the group consisting of Alzheimer's disease, Huntington's disease, mucolipidosis, contact dermatitis, late-onset hypersensitivity reaction, inflammation, endometriosis, scarring, rickets, skin diseases, such as, for example, psoriasis, immunological diseases, autoimmune diseases and immunodeficiency diseases.

Pain is a complex sensory perception which, as an acute event, has the character of a warning and control signal, but as chronic pain has lost this and in this case (as chronic pain syndrome) should be regarded and treated today as an independent syndrome. Hyperalgesia is the term used in medicine for excessive sensitivity to pain and reaction to a stimulus which is usually painful. Stimuli which can trigger pain are, for example, pressure, heat, cold or inflammation. Hyperalgesia is a form of hyperaesthesia, the generic term for excessive sensitivity to a stimulus. Allodynia is the term used in medicine for the sensation of pain which is triggered by stimuli which do not usually cause pain.

It is intended that the medicaments disclosed above include a corresponding use of the compounds according to the invention for the preparation of a medicament for the treatment and/or prophylaxis of the above physiological and/or pathophysiological states.

It is additionally intended that the medicaments disclosed above include a corresponding method for the treatment and/or prophylaxis of the above physiological and/or pathophysiological states in which at least one compound according to the invention is administered to a patient in need of such a treatment.

The compounds according to the invention preferably exhibit an advantageous biological activity which can easily be demonstrated in enzyme assays and animal experiments, as described in the examples. In such enzyme-based assays, the compounds according to the invention preferably exhibit and cause an inhibiting effect, which is usually documented by IC₅₀ values in a suitable range, preferably in the micromolar range and more preferably in the nanomolar range.

The compounds according to the invention can be administered to humans or animals, in particular mammals, such as apes, dogs, cats, rats or mice, and can be used in the therapeutic treatment of the human or animal body and in the combating of the above-mentioned diseases. They can furthermore be used as diagnostic agents or as reagents.

Furthermore, compounds according to the invention can be used for the isolation and investigation of the activity or expression of DDR2. In addition, they are particularly suitable for use in diagnostic methods for diseases in connection with disturbed DDR2 activity. The invention therefore furthermore relates to the use of the compounds according to the invention for the isolation and investigation of the activity or expression of DDR2 or as binders and inhibitors of DDR2.

For diagnostic purposes, the compounds according to the invention can, for example, be radioactively labelled. Examples of radioactive labels are ³H, ¹⁴C, ²³¹I and ¹²⁵I. A preferred labelling method is the iodogen method (Fraker et al., 1978). In addition, the compounds according to the invention can be labelled by enzymes, fluorophores and chemophores. Examples of enzymes are alkaline phosphatase, β-galactosidase and glucose oxidase, an example of a fluorophore is fluorescein, an example of a chemophore is luminol, and automated detection systems, for example for fluorescent colorations, are described, for example, in U.S. Pat. No. 4,125,828 and U.S. Pat. No. 4,207,554.

The compounds of the formula I can be used for the preparation of pharmaceutical compositions, in particular by non-chemical methods. In this case, they are brought into a suitable dosage form together with at least one solid, liquid and/or semi-liquid excipient or adjuvant and optionally in combination with one or more further active ingredient(s).

The invention therefore furthermore relates to pharmaceutical compositions comprising at least one compound of the formula I and/or physiologically acceptable salts, derivatives, solvates, prodrugs and stereoisomers thereof, including mixtures thereof in all ratios. In particular, the invention also relates to pharmaceutical compositions which comprise further excipients and/or adjuvants, and also to pharmaceutical compositions which comprise at least one further medicament active ingredient.

In particular, the invention also relates to a process for the preparation of a pharmaceutical composition, characterised in that a compound of the formula I and/or one of its physiologically acceptable salts, derivatives, solvates, prodrugs and stereoisomers, including mixtures thereof in all ratios, is brought into a suitable dosage form together with a solid, liquid or semi-liquid excipient or adjuvant and optionally with a further medicament active ingredient.

The pharmaceutical compositions according to the invention can be used as medicaments in human or veterinary medicine. The patient or host can belong to any mammal species, for example a primate species, particularly humans; rodents, including mice, rats and hamsters; rabbits; horses, cattle, dogs, cats, etc. Animal models are of interest for experimental investigations, where they provide a model for the treatment of a human disease.

Suitable carrier substances are organic or inorganic substances which are suitable for enteral (for example oral), parenteral or topical administration and do not react with the novel compounds, for example water, vegetable oils (such as sunflower oil or cod-liver oil), benzyl alcohols, polyethylene glycols, gelatine, carbohydrates, such as lactose or starch, magnesium stearate, talc, lanolin or Vaseline. Owing to his expert knowledge, the person skilled in the art is familiar with which adjuvants are suitable for the desired medicament formulation. Besides solvents, for example water, physiological saline solution or alcohols, such as, for example, ethanol, propanol or glycerol, sugar solutions, such as glucose or mannitol solutions, or a mixture of the said solvents, gel formers, tablet assistants and other active-ingredient carriers, it is also possible to use, for example, lubricants, stabilisers and/or wetting agents, emulsifiers, salts for influencing the osmotic pressure, antioxidants, dispersants, antifoams, buffer substances, flavours and/or aromas or flavour correctants, preservatives, solubilisers or dyes. If desired, compositions or medicaments according to the invention may comprise one or more further active ingredients, for example one or more vitamins.

The terms “pharmaceutical formulation” and “pharmaceutical composition” are used as synonyms for the purposes of the present invention.

As used here, “pharmaceutically tolerated” relates to medicaments, precipitation reagents, excipients, adjuvants, stabilisers, solvents and other agents which facilitate the administration of the pharmaceutical compositions obtained therefrom to a mammal without undesired physiological side effects, such as, for example, nausea, dizziness, digestion problems or the like.

In pharmaceutical compositions for parenteral administration, there is a requirement for isotonicity, euhydration and tolerability and safety of the formulation (low toxicity), of the adjuvants employed and of the primary packaging. Surprisingly, the compounds according to the invention preferably have the advantage that direct use is possible and further purification steps for the removal of toxicologically unacceptable agents, such as, for example, high concentrations of organic solvents or other toxicologically unacceptable adjuvants, are thus unnecessary before use of the compounds according to the invention in pharmaceutical formulations.

The invention particularly preferably also relates to pharmaceutical compositions comprising at least one compound according to the invention in precipitated non-crystalline, precipitated crystalline or in dissolved or suspended form, and optionally excipients and/or adjuvants and/or further pharmaceutical active ingredients.

The solid compounds according to the invention preferably enable the preparation of highly concentrated formulations without unfavourable, undesired aggregation of the compounds according to the invention occurring. Thus, ready-to-use solutions having a high active-ingredient content can be prepared with the aid of compounds according to the invention with aqueous solvents or in aqueous media.

The compounds and/or physiologically acceptable salts and solvates thereof can also be lyophilised and the resultant lyophilisates used, for example, for the preparation of injection preparations.

Aqueous compositions can be prepared by dissolving or suspending compounds according to the invention in an aqueous solution and optionally adding adjuvants. To this end, defined volumes of stock solutions comprising the said further adjuvants in defined concentration are advantageously added to a solution or suspension having a defined concentration of compounds according to the invention, and the mixture is optionally diluted with water to the pre-calculated concentration. Alternatively, the adjuvants can be added in solid form. The amounts of stock solutions and/or water which are necessary in each case can subsequently be added to the aqueous solution or suspension obtained. Compounds according to the invention can also advantageously be dissolved or suspended directly in a solution comprising all further adjuvants.

The solutions or suspensions comprising compounds according to the invention and having a pH of 4 to 10, preferably having a pH of 5 to 9, and an osmolality of 250 to 350 mOsmol/kg can advantageously be prepared. The pharmaceutical composition can thus be administered directly substantially without pain intravenously, intra-arterially, intra-articularly, subcutaneously or percutaneously. In addition, the preparation may also be added to infusion solutions, such as, for example, glucose solution, isotonic saline solution or Ringer's solution, which may also contain further active ingredients, thus also enabling relatively large amounts of active ingredient to be administered.

Pharmaceutical compositions according to the invention may also comprise mixtures of a plurality of compounds according to the invention.

The compositions according to the invention are physiologically well tolerated, easy to prepare, can be dispensed precisely and are preferably stable with respect to assay, decomposition products and aggregates throughout storage and transport and during multiple freezing and thawing processes. They can preferably be stored in a stable manner over a period of at least three months to two years at refrigerator temperature (2-8° C.) and at room temperature (23-27° C.) and 60% relative atmospheric humidity (R.H.).

For example, the compounds according to the invention can be stored in a stable manner by drying and when necessary converted into a ready-to-use pharmaceutical composition by dissolution or suspension. Possible drying methods are, for example, without being restricted to these examples, nitrogen-gas drying, vacuum-oven drying, lyophilisation, washing with organic solvents and subsequent air drying, liquid-bed drying, fluidised-bed drying, spray drying, roller drying, layer drying, air drying at room temperature and further methods.

The term “effective amount” denotes the amount of a medicament or of a pharmaceutical active ingredient which causes in a tissue, system, animal or human a biological or medical response which is sought or desired, for example, by a researcher or physician.

In addition, the term “therapeutically effective amount” denotes an amount which, compared with a corresponding subject who has not received this amount, has the following consequence: improved treatment, healing, prevention or elimination of a disease, syndrome, disease state, complaint, disorder or prevention of side effects or also a reduction in the progress of a disease, complaint or disorder. The term “therapeutically effective amount” also encompasses the amounts which are effective for increasing normal physiological function.

On use of compositions or medicaments according to the invention, the compounds according to the invention and/or physiologically acceptable salts and solvates thereof are generally used analogously to known, commercially available compositions or preparations, preferably in dosages of between 0.1 and 500 mg, in particular 5 and 300 mg, per use unit. The daily dose is preferably between 0.001 and 250 mg/kg, in particular 0.01 and 100 mg/kg, of body weight. The composition can be administered one or more times per day, for example two, three or four times per day. However, the individual dose for a patient depends on a large number of individual factors, such as, for example, on the efficacy of the particular compound used, on the age, body weight, general state of health, sex, nutrition, on the time and method of administration, on the excretion rate, on the combination with other medicaments and on the severity and duration of the particular disease.

A measure of the uptake of a medicament active ingredient in an organism is its bioavailability. If the medicament active ingredient is delivered to the organism intravenously in the form of an injection solution, its absolute bioavailability, i.e. the proportion of the pharmaceutical which reaches the systemic blood, i.e. the major circulation, in unchanged form, is 100%. In the case of oral administration of a therapeutic active ingredient, the active ingredient is generally in the form of a solid in the formulation and must therefore first be dissolved in order that it is able to overcome the entry barriers, for example the gastrointestinal tract, the oral mucous membrane, nasal membranes or the skin, in particular the stratum corneum, or can be absorbed by the body. Data on the pharmacokinetics, i.e. on the bioavailability, can be obtained analogously to the method of J. Shaffer et al., J. Pharm. Sciences, 88 (1999), 313-318.

Furthermore, medicaments of this type can be prepared by means of one of the processes generally known in the pharmaceutical art.

Medicaments can be adapted for administration via any desired suitable route, for example by the oral (including buccal or sublingual), rectal, pulmonary, nasal, topical (including buccal, sublingual or transdermal), vaginal or parenteral (including subcutaneous, intramuscular, intravenous, intradermal and in particular intra-articular) routes. Medicaments of this type can be prepared by means of all processes known in the pharmaceutical art by, for example, combining the active ingredient with the excipient(s) or adjuvant(s).

Parenteral administration is preferably suitable for administration of the medicaments according to the invention. In the case of parenteral administration, intra-articular administration is particularly preferred.

The invention thus preferably also relates to the use of a pharmaceutical composition according to the invention for intra-articular administration in the treatment and/or prophylaxis of physiological and/or pathophysiological states selected from the group consisting of osteoarthritis, traumatic cartilage injuries, pain, allodynia or hyperalgesia.

Intra-articular administration has the advantage that the compound according to the invention can be administered directly into the synovial fluid in the vicinity of the joint cartilage and is also able to diffuse from there into the cartilage tissue. Pharmaceutical compositions according to the invention can thus also be injected directly into the joint gap and thus develop their action directly at the site of action as intended. The compounds according to the invention are also suitable for the preparation of medicaments to be administered parenterally having slow, sustained and/or controlled release of active ingredient. They are thus also suitable for the preparation of delayed-release formulations, which are advantageous for the patient since administration is only necessary at relatively large time intervals.

The medicaments adapted to parenteral administration include aqueous and non-aqueous sterile injection solutions comprising antioxidants, buffers, bacteriostatics and solutes, by means of which the formulation is rendered isotonic with the blood or synovial fluid of the recipient to be treated; as well as aqueous and non-aqueous sterile suspensions, which can comprise suspension media and thickeners. The formulations can be delivered in single-dose or multi-dose containers, for example sealed ampoules and vials, and stored in the freeze-dried (lyophilised) state, so that only the addition of the sterile carrier liquid, for example water for injection purposes, immediately before use is necessary. Injection solutions and suspensions prepared in accordance with the formulation can be prepared from sterile powders, granules and tablets.

The compounds according to the invention can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds according to the invention can also be coupled to soluble polymers as targeted medicament excipients. Such polymers can encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds according to the invention can furthermore be coupled to a class of biodegradable polymers which are suitable for achieving slow release of a medicament, for example polylactic acid, poly-epsilon-caprolactone, polyhydroxybutyric acid, polyorthoesters, polyacetals, polydihydroxypyrans, polycyanoacrylates, polylactic-co-glycolic acid, polymers, such as conjugates between dextran and methacrylates, polyphosphoesters, various polysaccharides and polyamines and poly-ε-caprolactone, albumin, chitosan, collagen or modified gelatine and cross-linked or amphipathic block copolymers of hydrogels.

Suitable for enteral administration (oral or rectal) are, in particular, tablets, dragees, capsules, syrups, juices, drops or suppositories, and suitable for topical use are ointments, creams, pastes, lotions, gels, sprays, foams, aerosols, solutions (for example solutions in alcohols, such as ethanol or isopropanol, acetonitrile, DMF, dimethylacetamide, 1,2-propanediol or mixtures thereof with one another and/or with water) or powders. Also particularly suitable for topical uses are liposomal compositions.

In the case of formulation to give an ointment, the active ingredient can be employed either with a paraffinic or a water-miscible cream base. Alternatively, the active ingredient can be formulated to a cream with an oil-in-water cream base or a water-in-oil base.

Medicaments adapted to transdermal administration can be delivered as independent plasters for extended, close contact with the epidermis of the recipient. Thus, for example, the active ingredient can be supplied from the plaster by means of iontophoresis, as described in general terms in Pharmaceutical Research, 3(6), 318 (1986).

It goes without saying that, besides the constituents particularly mentioned above, the medicaments according to the invention may also comprise other agents usual in the art with respect to the particular type of pharmaceutical formulation.

The invention also relates to a set (kit) consisting of separate packs of
a) an effective amount of a compound of the formula I and/or physiologically acceptable salts, derivatives, solvates, prodrugs and stereoisomers thereof, including mixtures thereof in all ratios, and
b) an effective amount of a further medicament active ingredient.

The set comprises suitable containers, such as boxes or cartons, individual bottles, bags or ampoules. The set may, for example, comprise separate ampoules each containing an effective amount of a compound of the formula I and/or pharmaceutically acceptable derivatives, solvates, prodrugs and stereoisomers thereof, including mixtures thereof in all ratios, and an effective amount of a further medicament active ingredient in dissolved or lyophilised form.

Furthermore, the medicaments according to the invention can be used in order to provide additive or synergistic effects in certain known therapies and/or can be used in order to restore the efficacy of certain existing therapies.

Besides the compounds according to the invention, the pharmaceutical compositions according to the invention may also comprise further medicament active ingredients, for example for use in the treatment of osteoarthritis other DDR2 inhibitors, cathepsin D inhibitors, ADAMTS5 inhibitors, NSAIDS, Cox-2 inhibitors, glucocorticoids, hyaluronic acid, azathioprine, methotrexate, anti-CAM antibodies, such as, for example, anti-ICAM-1 antibody, FGF-18. For the treatment of the other diseases mentioned, the pharmaceutical compositions according to the invention may also, besides the compounds according to the invention, comprise further medicament active ingredients which are known to the person skilled in the art in the treatment thereof.

Even without further comments, it is assumed that a person skilled in the art will be able to use the above description in the broadest scope. The preferred embodiments should therefore merely be regarded as descriptive disclosure which is absolutely not limiting in any way.

The following examples are thus intended to explain the invention without limiting it. Unless indicated otherwise, percent data denote percent by weight. All temperatures are indicated in degrees Celsius. “Conventional work-up”: water is added if necessary, the pH is adjusted, if necessary, to values between 2 and 10, depending on the constitution of the end product, the mixture is extracted with ethyl acetate or dichloromethane, the phases are separated, the organic phase is dried over sodium sulfate, filtered and evaporated, and the product is purified by chromatography on silica gel and/or by crystallisation.

Rf values on silica gel; mass spectrometry: EI (electron impact ionisation): M⁺, FAB (fast atom bombardment): (M+H)⁺, THF (tetrahydrofuran), NMP (N-methylpyrrolidone), DMSO (dimethyl sulfoxide), EA (ethyl acetate), MeOH (methanol), TLC (thin-layer chromatography).

The following substances have been synthesised and characterised. However, the preparation and characterisation of the substances can also be carried out by other methods by the person skilled in the art.

### EXAMPLE 1

### Illustrative Compounds of the Formula I

TABLE 1a

| | | | | IC50 | IC50 | stability | |
|---|---|---|---|---|---|---|---|
| | | Compound | IC50 | [p- | [pro- | in | ESI MS |
| | Compound | (chemical | [DDR2] | DDR2] | MMP13] | synovial | Rt/ |
| No. | (structure) | name) | M | M | M | fluid | M + H |
| 1 | 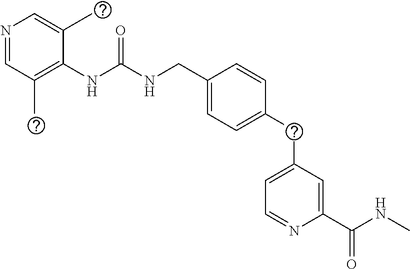 [SMILES: CNC(=O)c1cc(Cc2ccc(CNC(=O)Nc3c(C)cncc3C)cc2)ccn1] | 4-{4-[3-(3,5- Dichloro- pyridin-4-yl)- ureidomethyl]- phenoxy}- pyridine-2- carboxylic acid methylamide | 8.10E−07 | | | | |
| 2 | 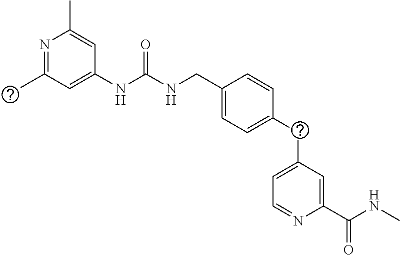 | 4-{4-[3-(2,6- Dichloro- pyridin-4-yl)- ureidomethyl]- phenoxy}- pyridine-2- carboxylic acid methylamide | 1.50E−06 | | | | |
| 3 | 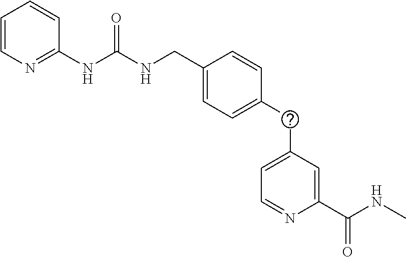 [SMILES: CNC(=O)c1cc(Cc2ccc(CNC(=O)Nc3ccccn3)cc2)ccn1] | 4-{4-[(3- Pyridin-2-yl- ureido)- methyl]- phenoxy}- pyridine-2- carboxylic acid methylamide | 3.80E−06 | | | | |
| 4 | 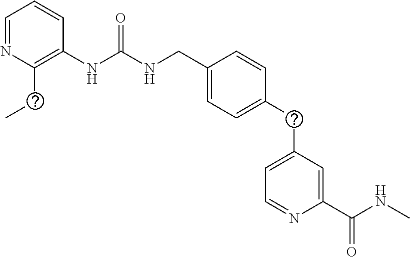 [SMILES: CCc1ncccc1NC(=O)NCc1ccc(Cc2ccnc(C(=O)NC)c2)cc1] | 4-{4-[3-(2- Methoxy- pyridin-3-yl)- ureidomethyl]- phenoxy}- pyridine-2- carboxylic acid methylamide | 1.60E−06 | | | | |
| 5 | 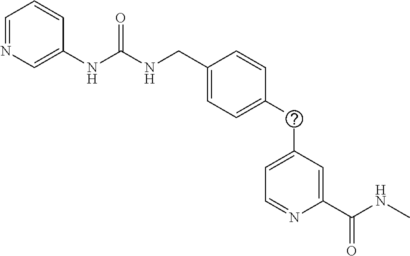 [SMILES: CNC(=O)c1cc(Cc2ccc(CNC(=O)Nc3cccnc3)cc2)ccn1] | 4-{4-[(3- Pyridin-3-yl- ureido)- methyl]- phenoxy}- pyridine-2- carboxylic acid methylamide | 1.50E−05 | | | | |
| 6 | 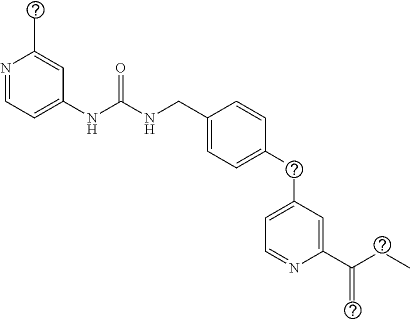 [SMILES: C=C(CC)c1cc(Cc2ccc(CNC(=O)Nc3ccnc(C)c3)cc2)ccn1] | 4-{4-[3-(2- Chloro- pyridin-4-yl)- ureidomethyl]- phenoxy}- pyridine-2- carboxylic acid methylamide | 1.00E−06 | | | | |
| 7 | 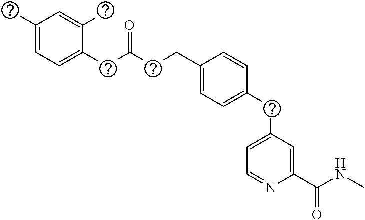 | 4-{4-[3-(3- Chloro-5- trifluoromethyl- pyridin-2-yl)- ureidomethyl]- phenoxy}- pyridine-2- carboxylic acid methylamide | 6.60E−06 | | | | |
| 8 | 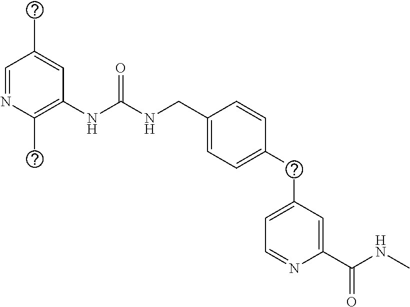 [SMILES: CNC(=O)c1cc(Cc2ccc(CNC(=O)Nc3cc(C)cnc3C)cc2)ccn1] | 4-{4-[3-(2,5- Dichloro- pyridin-3-yl)- ureidomethyl]- phenoxy}- pyridine-2- carboxylic acid methylamide | 1.30E−07 | | 2.78E−07 | | 2.246/ 447 |
| 9 | 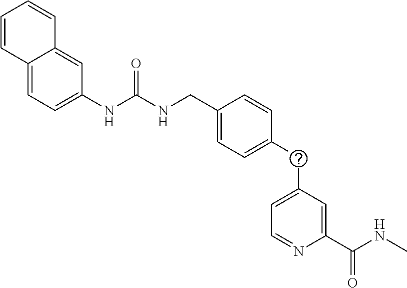 [SMILES: CNC(=O)c1cc(Cc2ccc(CNC(=O)Nc3ccc4ccccc4c3)cc2)ccn1] | 4-{4-[(3- lsoquinolin-3- yl-ureido)- methyl]- phenoxy}- pyridine-2- carboxylic acid methylamide | 3.70E−08 | | 3.67E−07 | | |
| 10 | 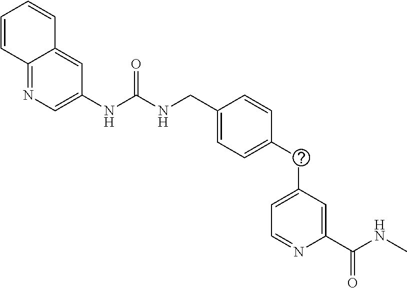 [SMILES: CNC(=O)c1cc(Cc2ccc(CNC(=O)Nc3cnc4ccccc4c3)cc2)ccn1] | 4-{4-[(3- Quinolin-3-yl- ureido)- methyl]- phenoxy}- pyridine-2- carboxylic acid methylamide | 2.90E−07 | | | | |
| 11 | 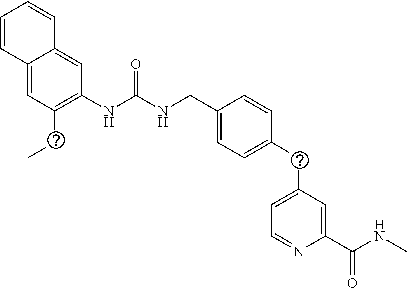 [SMILES: CCc1cc2ccccc2cc1NC(=O)NCc1ccc(Cc2ccnc(C(=O)NC)c2)cc1] | 4-{4-[3-(2- Methoxy- quinolin-3-yl)- ureidomethyl]- phenoxy}- pyridine-2- carboxylic acid methylamide | 1.90E−07 | | | | |
| 12 | 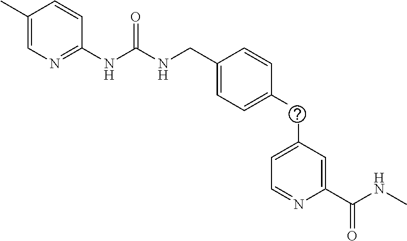 | 4-{4-[3-(5- Methyl- pyridin-2-yl)- ureidomethyl]- phenoxy}- pyridine-2- carboxylic acid methylamide | 2.00E−06 | | | | |
| 13 | 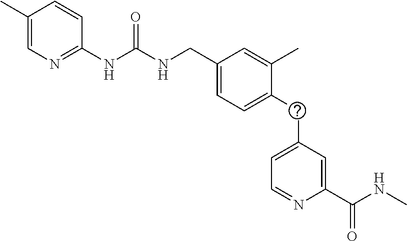 | 4-{2-Methyl-4- [3-(5-methyl- pyridin-2-yl)- ureidomethyl]- phenoxy}- pyridine-2- carboxylic acid methylamide | 7.80E−07 | | | | |
| 14 | 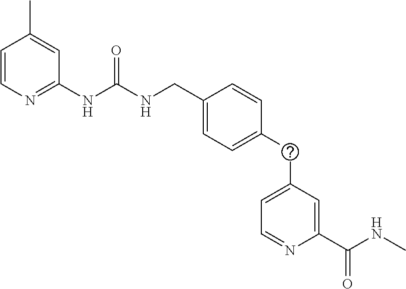 [SMILES: CNC(=O)c1cc(Cc2ccc(CNC(=O)Nc3cc(C)ccn3)cc2)ccn1] | 4-{4-[3-(4- Methyl- pyridin-2-yl)- ureidomethyl]- phenoxy}- pyridine-2- carboxylic acid methylamide | 5.70E−07 | | | | |
| 15 | 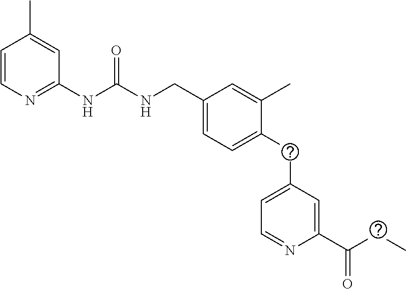 [SMILES: CCC(=O)c1cc(Cc2ccc(CNC(=O)Nc3cc(C)ccn3)cc2C)ccn1] | 4-{2-Methyl-4- [3-(4-methyl- pyridin-2-yl)- ureidomethyl]- phenoxy}- pyridine-2- carboxylic acid methylamide | 4.00E−07 | | | | |
| 16 | 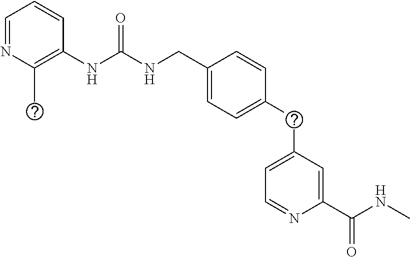 [SMILES: CNC(=O)c1cc(Cc2ccc(CNC(=O)Nc3cccnc3C)cc2)ccn1] | 4-{4-[3-(2- Chloro- pyridin-3-yl)- ureidomethyl]- phenoxy}- pyridine-2- carboxylic acid methylamide | 1.10E−05 | | | | |
| 17 | 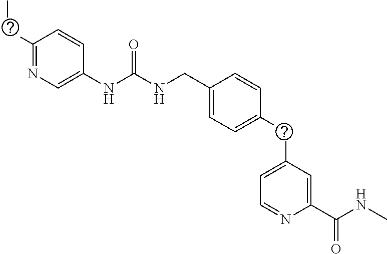 [SMILES: CCc1ccc(NC(=O)NCc2ccc(Cc3ccnc(C(=O)NC)c3)cc2)cn1] | 4-{4-[3-(6- Methoxy- pyridin-3-yl)- ureidomethyl]- phenoxy}- pyridine-2- carboxylic acid methylamide | 5.60E−06 | | | | |
| 18 | 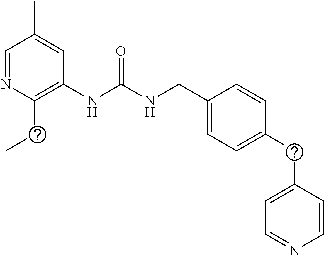 [SMILES: CCc1ncc(C)cc1NC(=O)NCc1ccc(Cc2ccncc2)cc1] | 1-(2-Methoxy- 5-methyl- pyridin-3-yl)-3- [4-(pyridin-4- yloxy)-benzyl]- urea | 1.00E−07 | 3.00E−08 | 7.33E−07 | | 1.451 min/ 365 |
| 19 | 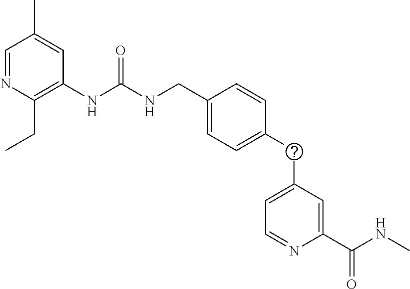 [SMILES: CCc1ncc(C)cc1NC(=O)NCc1ccc(Cc2ccnc(C(=O)NC)c2)cc1] | 4-{4-[3-(2- Methoxy-5- methyl- pyridin-3-yl)- ureidomethyl]- phenoxy}- pyridine-2- carboxylic acid methylamide | 6.30E−08 | 2.40E−08 | 1.55E−07 | stable | 2.049/ 422 |
| 20 | 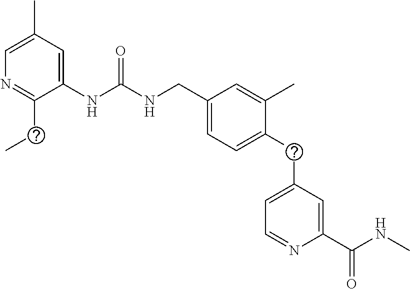 [SMILES: CCc1ncc(C)cc1NC(=O)NCc1ccc(Cc2ccnc(C(=O)NC)c2)c(C)c1] | 4-{4-[3-(2- Methoxy-5- methyl- pyridin-3-yl)- ureidomethyl]- 2-methyl- phenoxy}- pyridine-2- carboxylic acid methylamide | 1.10E−07 | 4.10E−08 | | | 2.142/ 436 |
| 21 | 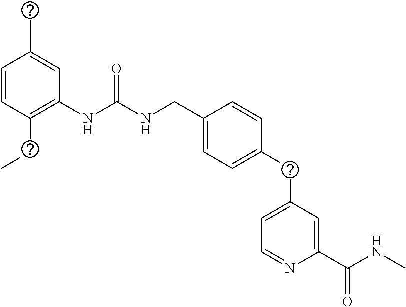 [SMILES: CCc1ccc(C)cc1NC(=O)NCc1ccc(Cc2ccnc(C(=O)NC)c2)cc1] | 4-{4-[3-(5- Chloro-2- methoxy- pyridin-3-yl)- ureidomethyl]- phenoxy}- pyridine-2- carboxylic acid methylamide | 4.40E−08 | 1.40E−08 | 6.90E−08 | stable | 2.260/ 442 |
| 22 | 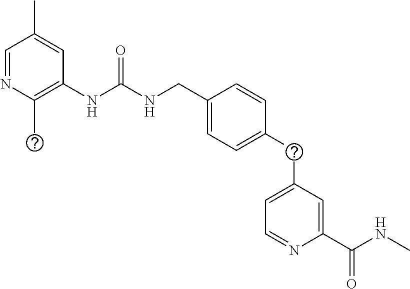 [SMILES: CNC(=O)c1cc(Cc2ccc(CNC(=O)Nc3cc(C)cnc3C)cc2)ccn1] | 4-{4-[3-(2- Chloro-5- methyl- pyridin-3-yl)- ureidomethyl]- phenoxy}- pyridine-2- carboxylic acid methylamide | 7.10E−07 | | | | 2.033/ 426 |
| 23 | 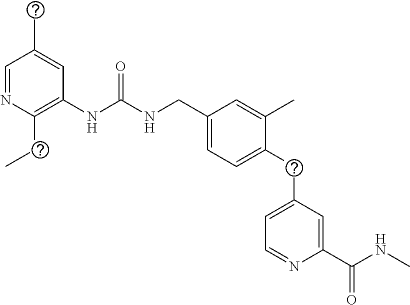 [SMILES: CCc1ncc(C)cc1NC(=O)NCc1ccc(Cc2ccnc(C(=O)NC)c2)c(C)c1] | 4-{4-[3-(5- Chloro-2- methoxy- pyridin-3-yl)- ureidomethyl]- 2-methyl- phenoxy}- pyridine-2- carboxylic acid methylamide | 3.70E−08 | 6.80E−09 | 4.54E−08 | stable | 2.370/ 456 |
| 24 | 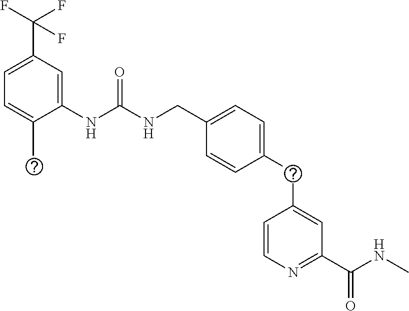 [SMILES: CNC(=O)c1cc(Cc2ccc(CNC(=O)Nc3cc(C(F)(F)F)ccc3C)cc2)ccn1] | 4-{4-[3-(2- Chloro-5- trifluoromethyl- pyridin-3-yl)- ureidomethyl]- phenoxy}- pyridine-2- carboxylic acid methylamide | 1.30E−07 | 5.80E−08 | | | 2.336/ 480 |
| 25 | 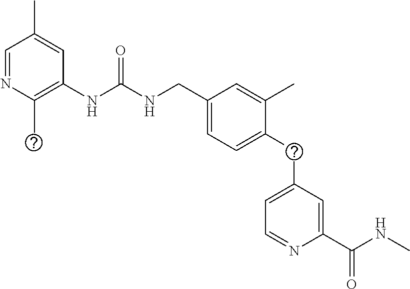 [SMILES: CNC(=O)c1cc(Cc2ccc(CNC(=O)Nc3cc(C)cnc3C)cc2C)ccn1] | 4-{4-[3-(2- Chloro-5- methyl- pyridin-3-yl)- ureidomethyl]- 2-methyl- phenoxy}- pyridine-2- carboxylic acid methylamide | 4.10E−07 | 8.70E−08 | | | 2.126/ 440 |
| 26 | 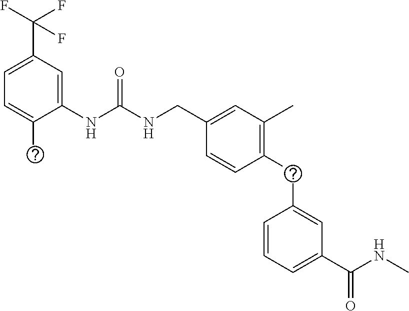 [SMILES: CNC(=O)c1cccc(Cc2ccc(CNC(=O)Nc3cc(C(F)(F)F)ccc3C)cc2C)c1] | 4-{4-[3-(2- Chloro-5- trifluoromethyl- pyridin-3-yl)- ureidomethyl]- 2-methyl- phenoxy}- pyridine-2- carboxylic acid methylamide | 5.20E−08 | 4.30E−08 | 4.24E−08 | | 2.421/ 494 |
| 27 | 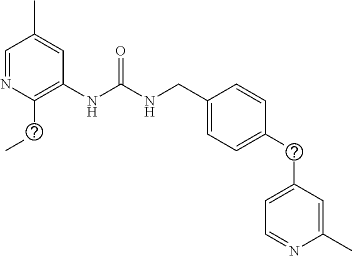 [SMILES: CCc1ncc(C)cc1NC(=O)NCc1ccc(Cc2ccnc(C)c2)cc1] | 1-(5-Chloro-2- methoxy- pyridin-3-yl)-3- [4-(2-methyl- pyridin-4- yloxy)-benzyl]- urea | 1.90E−07 | 4.20E−08 | | | 1.629/ 399 |
| 28 | 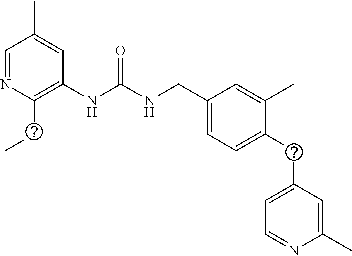 [SMILES: CCc1ncc(C)cc1NC(=O)NCc1ccc(Cc2ccnc(C)c2)c(C)c1] | 1-(5-Chloro-2- methoxy- pyridin-3-yl)-3- [3-methyl-4- (2-methyl- pyridin-4- yloxy)-benzyl]- urea | 1.10E−07 | 1.70E−08 | 3.48E−07 | | 1.673/ 413 |
| 29 | 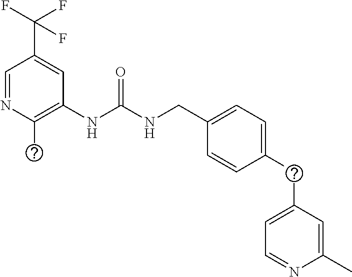 [SMILES: Cc1cc(Cc2ccc(CNC(=O)Nc3cc(C(F)(F)F)cnc3C)cc2)ccn1] | 1-(2-Chloro-5- trifluoromethyl- pyridin-3-yl)- 3-[4-(2- methyl- pyridin-4- yloxy)-benzyl]- urea | 3.50E−07 | 9.40E−08 | | | 1.695/ 437 |
| 30 | 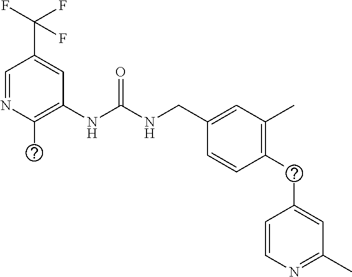 [SMILES: Cc1cc(Cc2ccc(CNC(=O)Nc3cc(C(F)(F)F)cnc3C)cc2C)ccn1] | 1-(2-Chloro-5- trifluoromethyl- pyridin-3-yl)- 3-[3-methyl-4- (2-methyl- pyridin-4- yloxy)-benzyl]- urea | 1.00E−07 | 6.00E−08 | 9.60E−07 | | 1.756/ 451 |
| 31 | 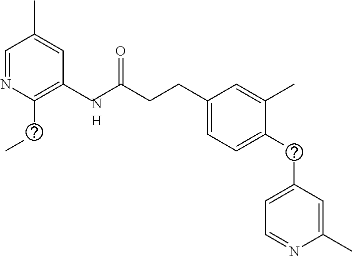 [SMILES: CCc1ncc(C)cc1CC(=O)CCc1ccc(Cc2ccnc(C)c2)c(C)c1] | 1-(2-Methoxy- 5-methyl- pyridin-3-yl)-3- [3-methyl-4- (2-methyl- pyridin-4- yloxy)-benzyl]- urea | 2.30E−07 | | | | 1.529/ 393 |
| 32 | 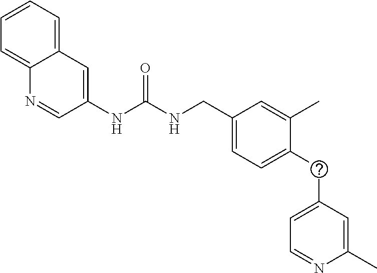 [SMILES: Cc1cc(Cc2ccc(CNC(=O)Nc3cnc4ccccc4c3)cc2C)ccn1] | 1-[3-Methyl-4- (2-methyl- pyridin-4- yloxy)-benzyl]- 3-quinolin-3- yl-urea | 7.10E−06 | | | | 1.390/ 399 |
| 33 | 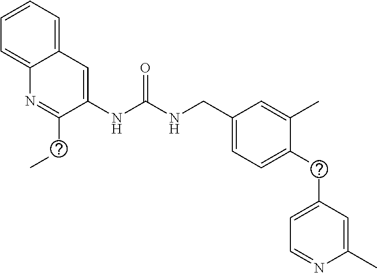 [SMILES: CCc1nc2ccccc2cc1NC(=O)NCc1ccc(Cc2ccnc(C)c2)c(C)c1] | 1-(2-Methoxy- quinolin-3-yl)- 3-[3-methyl-4- (2-methyl- pyridin-4- yloxy)-benzyl]- urea | 1.70E−06 | | | | 1.758/ 429 |
| 34 | 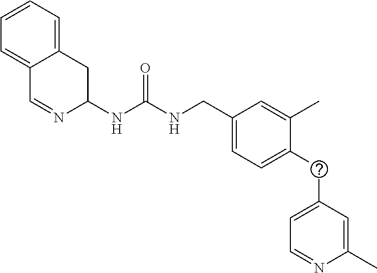 [SMILES: Cc1cc(Cc2ccc(CNC(=O)NC3Cc4ccccc4C=N3)cc2C)ccn1] | 1-Isoquinolin- 3-yl-3-[3- methyl-4-(2- methyl- pyridin-4- yloxy)-benzyl]- urea | 1.10E−05 | | | | 1.644/ 399 |

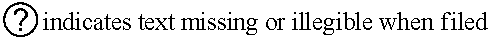

TABLE 1b
NMR data of the compounds of table 1a

| No. of | |
|---|---|
| compound | |
| of table 1a | 1HNMR |
| 1 | 1H NMR (500 MHz, DMSO) ppm = 9.59 (s, 1H), 8.74 (q, J = 4.8, 1H), 8.51 (d, |
| | J = 5.5, 1H), 7.52 (s, 2H), 7.46-7.41 (m, 2H), 7.36 (d, J = 2.6, 1H), 7.27 (t, J = 6.0, |
| | 1H), 7.23-7.18 (m, 2H), 7.16 (dd, J = 5.6, 2.6, 1H), 4.36 (d, J = 5.9, 2H), 2.78 (d, |
| | J = 4.8, 3H). |
| 2 | 1H NMR (500 MHz, DMSO) ppm = 9.58 (s, 1H), 8.78-8.71 (m, 1H), 8.51 (d, |
| | J = 5.5, 1H), 7.52 (s, 2H), 7.44 (d, J = 8.0, 2H), 7.36 (d, J = 2.6, 1H), 7.27 (t, J = 6.0, |
| | 1H), 7.23-7.19 (m, 2H), 7.19-7.14 (m, 1H), 4.36 (d, J = 5.9, 2H), 2.78 (d, |
| | J = 4.8, 3H). |
| 3 | 1H NMR (500 MHz, DMSO) ppm = 9.29 (s, 1H), 8.77-8.71 (m, 1H), 8.66 (t, |
| | J = 6.0, 1H), 8.50 (d, J = 5.6, 1H), 8.21-8.17 (m, 1H), 7.72-7.64 (m, 1H), |
| | 7.48-7.42 (m, 2H), 7.41-7.35 (m, 2H), 7.23-7.17 (m, 2H), 7.17-7.13 (m, 1H), |
| | 6.96-6.90 (m, 1H), 4.46 (d, J = 5.9, 2H), 2.78 (d, J = 4.8, 3H). |
| 4 | 1H NMR (500 MHz, DMSO) ppm = 8.78-8.71 (m, 1H), 8.53-8.49 (m, 1H), |
| | 8.37 (dd, J = 7.7, 1.7, 1H), 8.17 (s, 1H), 7.72-7.67 (m, 1H), 7.48-7.36 (m, 4H), |
| | 7.24-7.18 (m, 2H), 7.18-7.13 (m, 1H), 6.93-6.87 (m, 1H), 4.36 (d, J = 5.7, |
| | 2H), 3.93 (s, 3H), 2.78 (d, J = 4.9, 3H). |
| 5 | 1H NMR (500 MHz, DMSO) ppm = 8.81 (s, 1H), 8.74 (dd, J = 9.7, 4.8, 1H), 8.56 |
| | (d, J = 2.6, 1H), 8.51 (d, J = 5.5, 1H), 8.14-8.09 (m, 1H), 7.94-7.88 (m, 1H), |
| | 7.48-7.41 (m, 2H), 7.37 (d, J = 2.6, 1H), 7.29-7.23 (m, 1H), 7.23-7.18 (m, |
| | 2H), 7.18-7.13 (m, 1H), 6.87 (t, 1H), 4.36 (d, J = 5.9, 2H), 2.78 (d, J = 4.8, 3H). |
| 6 | 1H NMR (500 MHz, DMSO) ppm = 9.37 (s, 1H), 8.77-8.71 (m, 1H), 8.51 (d, |
| | J = 5.6, 1H), 8.12 (d, J = 5.7, 1H), 7.65-7.61 (m, 1H), 7.44 (d, J = 8.0, 2H), 7.36 |
| | (d, J = 2.6, 1H), 7.30-7.25 (m, 1H), 7.23-7.18 (m, 2H), 7.18-7.14 (m, 1H), |
| | 7.11 (t, J = 6.0, 1H), 4.36 (d, J = 5.9, 2H), 2.78 (d, J = 4.9, 3H). |
| 7 | 1H NMR (500 MHz, DMSO) ppm = 9.27 (t, 1H), 8.85 (s, 1H), 8.78-8.70 (m, |
| | 1H), 8.64-8.59 (m, 1H), 8.51 (d, J = 5.6, 1H), 8.39 (d, J = 2.2, 1H), 7.51-7.45 |
| | (m, 2H), 7.37 (d, J = 2.6, 1H), 7.24-7.18 (m, 2H), 7.18-7.14 (m, 1H), 4.52 (d, |
| | J = 6.0, 2H), 2.78 (d, J = 4.8, 3H). |
| 8 | 1H NMR (400 MHz, DMSO) ppm = 8.78-8.72 (m, 1H), 8.70 (d, J = 2.5, 1H), |
| | 8.52 (d, J = 5.6, 1H), 8.45 (s, 1H), 8.06 (d, J = 2.4, 1H), 7.79-7.72 (m, 1H), |
| | 7.50-7.44 (m, 2H), 7.38 (d, J = 2.6, 1H), 7.25-7.20 (m, 2H), 7.17 (dd, J = 5.6, 2.6, 1H), |
| | 4.39 (d, J = 5.7, 2H), 2.79 (d, J = 4.8, 3H). |
| 9 | 1H NMR (500 MHz, DMSO) ppm = 9.17 (s, 1H), 9.05 (s, 1H), 8.78-8.70 (m, |
| | 1H), 8.51 (d, J = 5.6, 1H), 8.06 (s, 1H), 7.98 (d, J = 8.2, 1H), 7.79 (d, J = 8.4, 1H), |
| | 7.68-7.59 (m, 2H), 7.50-7.45 (m, 2H), 7.45-7.40 (m, 1H), 7.38 (d, J = 2.6, |
| | 1H), 7.24-7.19 (m, 2H), 7.17-7.14 (m, 1H), 4.44 (d, J = 5.8, 2H), 2.78 (d, |
| | J = 4.9, 3H). |
| 10 | 1H NMR (500 MHz, DMSO) ppm = 9.11 (s, 1H), 8.80 (d, J = 2.6, 1H), 8.77-8.71 |
| | (m, 1H), 8.51 (d, J = 5.6, 1H), 8.48 (d, J = 2.5, 1H), 7.93-7.81 (m, 2H), |
| | 7.60-7.51 (m, 2H), 7.51-7.45 (m, 2H), 7.38 (d, J = 2.6, 1H), 7.25-7.19 (m, 2H), 7.17 |
| | (dd, J = 5.5, 2.6, 1H), 7.01-6.94 (m, 1H), 4.41 (d, J = 5.9, 2H), 2.78 (d, J = 4.9, |
| | 3H). |
| 11 | 1H NMR (500 MHz, DMSO) ppm = 8.77 (s, 1H), 8.76-8.70 (m, 1H), 8.51 (d, |
| | J = 5.6, 1H), 8.47 (s, 1H), 7.78-7.72 (m, 1H), 7.69 (d, J = 8.2, 1H), 7.56 (t, 1H), |
| | 7.52-7.44 (m, 3H), 7.41-7.33 (m, 2H), 7.26-7.19 (m, 2H), 7.19-7.13 (m, |
| | 1H), 4.40 (d, J = 5.7, 2H), 4.10 (s, 3H), 2.78 (d, J = 4.8, 3H). |
| 12 | not present |
| 13 | not present |
| 14 | not present |
| 15 | not present |
| 16 | 1H NMR (500 MHz, DMSO) ppm = 8.75 (d, J = 4.9, 1H), 8.59-8.48 (m, 2H), |
| | 8.27 (s, 1H), 7.98 (dd, J = 4.6, 1.8, 1H), 7.63 (t, 1H), 7.49-7.43 (m, 2H), |
| | 7.39-7.32 (m, 2H), 7.25-7.20 (m, 2H), 7.19-7.13 (m, 1H), 4.38 (d, J = 5.7, 2H), 2.78 |
| | (d, J = 4.8, 3H). |
| 17 | 1H NMR (500 MHz, DMSO) ppm = 8.77-8.72 (m, 1H), 8.53-8.49 (m, 2H), |
| | 8.15 (d, J = 2.7, 1H), 7.81-7.76 (m, 1H), 7.46-7.41 (m, 2H), 7.37 (d, J = 2.6, |
| | 1H), 7.23-7.18 (m, 2H), 7.16 (dd, J = 5.6, 2.6, 1H), 6.75-6.70 (m, 2H), 4.34 (d, |
| | J = 5.9, 2H), 3.79 (s, 3H), 2.78 (d, J = 4.8, 3H). |
| 18 | 1H NMR (400 MHz, DMSO) ppm = 8.79-8.72 (m, 2H), 8.24 (d, J = 2.1, 1H), |
| | 8.13 (s, 1H), 7.53-7.43 (m, 4H), 7.42-7.36 (m, 2H), 7.32-7.27 (m, 2H), 4.37 |
| | (d, J = 5.7, 2H), 3.90 (s, 3H), 2.18 (s, 3H). |
| 19 | 1H NMR (500 MHz, DMSO) ppm = 8.78-8.71 (m, 1H), 8.51 (d, J = 5.6, 1H), |
| | 8.25 (d, J = 2.1, 1H), 8.10 (s, 1H), 7.53-7.49 (m, 1H), 7.46-7.39 (m, 3H), 7.38 |
| | (d, J = 2.6, 1H), 7.23-7.18 (m, 2H), 7.16 (dd, J = 5.6, 2.6, 1H), 4.35 (d, J = 5.5, |
| | 2H), 3.90 (s, 3H), 2.78 (d, J = 4.8, 3H), 2.18 (s, 3H). |
| 20 | 1H NMR (500 MHz, DMSO) ppm = 8.79-8.72 (m, 1H), 8.50 (d, J = 5.6, 1H), |
| | 8.25 (d, J = 2.1, 1H), 8.09 (s, 1H), 7.52-7.49 (m, 1H), 7.39 (t, 1H), 7.34-7.32 |
| | (m, 1H), 7.30 (d, J = 2.6, 1H), 7.28-7.24 (m, 1H), 7.15-7.09 (m, 2H), 4.32 (d, |
| | J = 5.3, 2H), 3.90 (s, 3H), 2.78 (d, J = 4.8, 3H), 2.18 (s, 3H), 2.10 (s, 3H). |
| 21 | 1H NMR (400 MHz, DMSO) ppm = 8.79-8.70 (m, 1H), 8.51 (d, J = 5.6, 1H), |
| | 8.47 (d, J = 2.5, 1H), 8.39 (s, 1H), 7.73 (d, J = 2.4, 1H), 7.51 (t, J = 5.8, 1H), |
| | 7.47-7.41 (m, 2H), 7.38 (d, J = 2.6, 1H), 7.24-7.18 (m, 2H), 7.16 (dd, J = 5.6, 2.6, 1H), |
| | 4.37 (d, J = 5.7, 2H), 3.94 (s, 3H), 2.79 (d, J = 4.8, 3H). |
| 22 | 1H NMR (500 MHz, DMSO) ppm = 8.79-8.70 (m, 1H), 8.51 (d, J = 5.6, 1H), |
| | 8.41 (d, J = 2.1, 1H), 8.19 (s, 1H), 7.85-7.80 (m, 1H), 7.62 (t, J = 5.8, 1H), |
| | 7.49-7.43 (m, 2H), 7.38 (d, J = 2.6, 1H), 7.25-7.19 (m, 2H), 7.19-7.14 (m, 1H), 4.38 |
| | (d, J = 5.7, 2H), 2.78 (d, J = 4.8, 3H), 2.26 (s, 3H). |
| 23 | 1H NMR (500 MHz, DMSO) ppm = 8.80-8.71 (m, 1H), 8.50 (d, J = 5.6, 1H), |
| | 8.47 (d, J = 2.4, 1H), 8.39 (s, 1H), 7.73 (d, J = 2.4, 1H), 7.50 (t, J = 5.8, 1H), 7.34 |
| | (d, J = 2.1, 1H), 7.29 (d, J = 2.6, 1H), 7.26 (dd, J = 8.3, 2.2, 1H), 7.17-7.08 (m, |
| | 2H), 4.34 (d, J = 5.6, 2H), 3.94 (s, 3H), 2.78 (d, J = 4.8, 3H), 2.10 (s, 3H). |
| 24 | 1H NMR (500 MHz, DMSO) ppm = 8.96 (d, J = 2.2, 1H), 8.77-8.72 (m, 1H), |
| | 8.63 (s, 1H), 8.51 (d, J = 5.6, 1H), 8.39 (d, J = 2.2, 1H), 7.80 (t, J = 5.8, 1H), |
| | 7.49-7.45 (m, 2H), 7.38 (d, J = 2.6, 1H), 7.24-7.20 (m, 2H), 7.17 (dd, J = 5.6, 2.6, 1H), |
| | 4.40 (d, J = 5.7, 2H), 2.78 (d, J = 4.8. 3H). |
| 25 | 1H NMR (500 MHz, DMSO) ppm = 8.78-8.72 (m, 1H), 8.51 (d, J = 5.6, 1H), |
| | 8.41 (d, J = 2.1, 1H), 8.18 (s, 1H), 7.84-7.80 (m, 1H), 7.61 (t, J = 5.7, 1H), 7.35 |
| | (d, J = 2.1, 1H), 7.31-7.26 (m, 2H), 7.17-7.10 (m, 2H), 4.35 (d, J = 5.6, 2H), |
| | 2.78 (d, J = 4.8, 3H), 2.27 (s, 3H), 2.10 (s, 3H). |
| 26 | 1H NMR (400 MHz, DMSO) ppm = 8.98 (d, J = 2.3, 1H), 8.78-8.71 (m, 1H), |
| | 8.63 (s, 1H), 8.52 (d, J = 5.6, 1H), 8.40 (dd, J = 2.3, 1.0, 1H), 7.79 (t, J = 5.7, 1H), |
| | 7.38 (d, J = 2.1, 1H), 7.33-7.27 (m, 2H), 7.18-7.10 (m, 2H), 4.39 (d, J = 5.6, |
| | 2H), 2.79 (d, J = 4.8, 3H), 2.12 (s, 3H). |
| 27 | 1H NMR (500 MHz, DMSO) ppm = 8.63 (d, J = 6.8, 1H), 8.46 (d, J = 2.5, 1H), |
| | 8.43 (s, 1H), 7.73 (d, J = 2.5, 1H), 7.58 (t, 1H), 7.50-7.45 (m, 2H), 7.30-7.25 |
| | (m, 3H), 7.25-7.22 (m, 1H), 4.38 (d, J = 5.8, 2H), 3.94 (s, 3H), 2.61 (s, 3H). |
| 28 | 1H NMR (500 MHz, DMSO) ppm = 8.64 (d, J = 6.8, 1H), 8.46 (d, J = 2.5, 1H), |
| | 8.42 (s, 1H), 7.73 (d, J = 2.5, 1H), 7.59-7.52 (m, 1H), 7.37 (d, J = 2.1, 1H), |
| | 7.33-7.28 (m, 1H), 7.28-7.15 (m, 3H), 4.35 (d, J = 5.8, 2H , 3.94 (s, 3H), 2.62 (s, |
| | 3H), 2.12 (s, 3H). |
| 29 | 1H NMR (500 MHz, DMSO) ppm = 8.97 (d, J = 2.2, 1H), 8.63 (s, 1H), 8.43-8.36 |
| | (m, 1H), 8.31 (d, J = 5.7, 1H), 7.78 (t, 1H), 7.47-7.39 (m, 2H), 7.19-7.10 (m, 2H), |
| | 6.76 (d, J = 2.4, 1H), 6.73-6.67 (m, 1H), 4.38 (d, J = 5.7, 2H), 2.40 (s, 3H) |
| | 1.91 (s, 0H), 1.23 (s, 0H). |
| 30 | 1H NMR (500 MHz, DMSO) ppm = 8.97 (d, J = 2.2, 1H), 8.62 (s, 1H), 8.42-8.37 |
| | (m, 1H), 8.29 (d, J = 5.7, 1H), 7.76 (t, 1H), 7.35-7.30 (m, 1H), 7.28-7.23 (m, |
| | 1H), 7.07 (d, J = 8.2, 1H), 6.67 (d, J = 2.4, 1H), 6.63-6.58 (m, 1H), 4.36 (d, |
| | J = 5.6, 2H), 2.39 (s, 3H), 2.11 (s, 3H). |
| 31 | 1H NMR (500 MHz, DMSO) ppm = 8.65 (d, J = 6.8, 1H), 8.24 (d, J = 2.1, 1H), |
| | 8.13 (s, 1H), 7.54-7.49 (m, 1H), 7.44 (t, 1H), 7.36 (d, J = 2.1, 1H), 7.33-7.27 |
| | (m, 1H), 7.26 (d, J = 2.7, 1H), 7.24-7.17 (m, 2H), 4.34 (d, J = 5.7, 2H), 3.90 (s, |
| | 3H), 2.62 (s, 3H), 2.18 (s, 3H), 2.12 (s, 3H). |
| 32 | 1H NMR (500 MHz, DMSO) ppm = 9.32 (s, 1H), 8.91 (d, J = 2.5, 1H), 8.62-8.59 |
| | (m, 1H), 8.55-8.52 (m, 1H), 7.97-7.87 (m, 2H), 7.65-7.61 (m, 1H), |
| | 7.61-7.55 (m, 1H), 7.41-7.38 (m, 1H), 7.36-7.31 (m, 1H), 7.25 (d, J = 2.6, 1H), |
| | 7.22-7.16 (m, 2H), 7.14 (t, J = 6.1, 1H), 4.39 (d, J = 5.8, 2H), 2.61 (s, 3H), 2.11 (s, |
| | 3H). |
| 33 | 1H NMR (500 MHz, DMSO) ppm = 8.77 (s, 1H), 8.61 (d, J = 6.7, 1H), 8.48 (s, |
| | 1H), 7.75-7.67 (m, 2H), 7.57 (t, 1H), 7.51-7.46 (m, 1H), 7.41-7.31 (m, 3H), |
| | 7.23-7.16 (m, 3H), 4.39 (d, J = 5.8, 2H), 4.10 (s, 3H), 2.60 (s, 3H), 2.12 (s, 3H). |
| 34 | 1H NMR (500 MHz, DMSO) ppm = 9.25 (s, 1H), 9.05 (s, 1H), 8.64 (d, J = 6.8, |
| | 1H), 8.04 (s, 1H), 8.02-7.96 (m, 1H), 7.78 (d, J = 8.3, 1H), 7.71-7.60 (m, 2H), |
| | 7.49-7.38 (m, 2H), 7.38-7.30 (m, 1H), 7.30-7.16 (m, 3H), 4.43 (d, J = 5.8, |
| | 2H), 2.62 (s, 3H), 2.12 (s, 3H). |

TABLE 2

| | | | | IC50 | |
|---|---|---|---|---|---|
| | | | IC50 | [p- | |
| | Compound | Compound | [DDR2] | DDR2] | |
| No. | (structure) | (chemical name) | nM | nM | Mass |
| 1 | 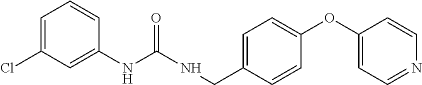 [SMILES: O=C(NCc1ccc(Oc2ccncc2)cc1)Nc1cccc(Cl)c1] | 1-(3-Chloro-phenyl)-3-[4- (pyridin-4-yloxy)-benzyl]- urea | 110 | | 353.81 |
| 2 | 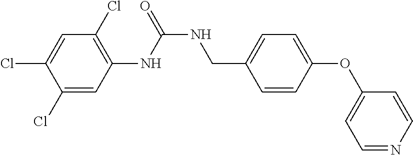 [SMILES: O=C(NCc1ccc(Oc2ccncc2)cc1)Nc1cc(Cl)c(Cl)cc1Cl] | 1-[4-(Pyridin-4-yloxy)- benzyl]-3-(2,4,5-trichloro- phenyl)-urea | 1400 | | 422.70 |
| 3 | 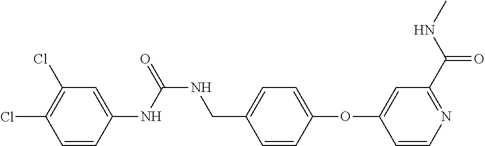 [SMILES: CNC(=O)c1cc(Oc2ccc(CNC(=O)Nc3ccc(Cl)c(Cl)c3)cc2)ccn1] | 4-{4-[3-(3,4-Dichloro- phenyl)-ureidomethyl]- phenoxy}-pyridine-2- carboxylic acid methylamide | | 195 | 445.31 |
| 4 | 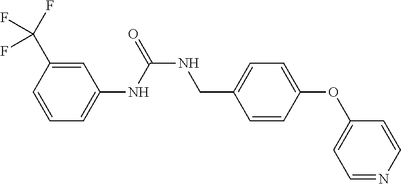 [SMILES: O=C(NCc1ccc(Oc2ccncc2)cc1)Nc1cccc(C(F)(F)F)c1] | 1-[4-(Pyridin-4-yloxy)- benzyl]-3-(3- trifluoromethyl-phenyl)- urea | 36 | | 387.36 |
| 5 | 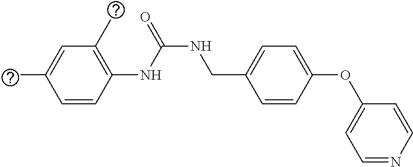 [SMILES: Cc1ccc(NC(=O)NCc2ccc(Oc3ccncc3)cc2)c(C)c1] | 1-(2,4-Dichloro-phenyl)-3- [4-(pyridin-4-yloxy)- benzyl]-urea | 1400 | | 388.25 |
| 6 | 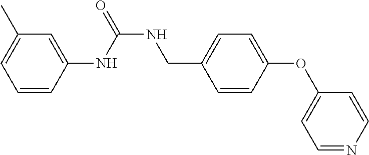 [SMILES: Cc1cccc(NC(=O)NCc2ccc(Oc3ccncc3)cc2)c1] | 1-[4-(Pyridin-4-yloxy)- benzyl]-3-m-tolyl-urea | 1000 | | 333.39 |
| 7 | 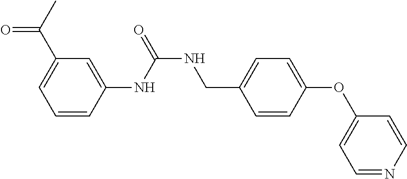 [SMILES: CC(=O)c1cccc(NC(=O)NCc2ccc(Oc3ccncc3)cc2)c1] | 1-(3-Acetyl-phenyl)-3-[4- (pyridin-4-yloxy)-benzyl]- urea | 10000 | | 361.40 |
| 8 | 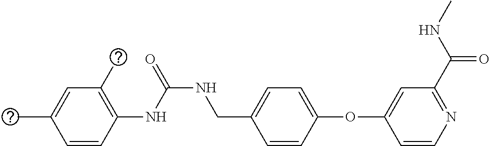 [SMILES: CNC(=O)c1cc(Oc2ccc(CNC(=O)Nc3ccc(C)cc3C)cc2)ccn1] | 4-{4-[3-(2,4-Dichloro- phenyl)-ureidomethyl]- phenoxy}-pyridine-2- carboxylic acid methylamide | | 255 | 445.31 |
| 9 | 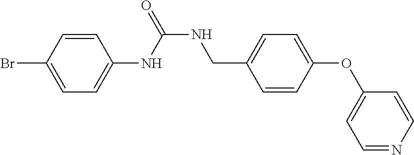 [SMILES: O=C(NCc1ccc(Oc2ccncc2)cc1)Nc1ccc(Br)cc1] | 1-(4-Bromo-phenyl)-3-[4- (pyridin-4-yloxy)-benzyl]- urea | 3800 | | 398.26 |
| 10 | 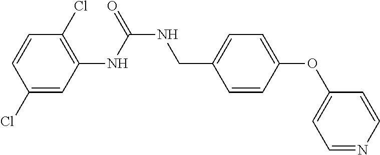 [SMILES: O=C(NCc1ccc(Oc2ccncc2)cc1)Nc1cc(Cl)ccc1Cl] | 1-(2,5-Dichloro-phenyl)-3- [4-(pyridin-4-yloxy)- benzyl]-urea | 270 | | 388.25 |
| 11 | 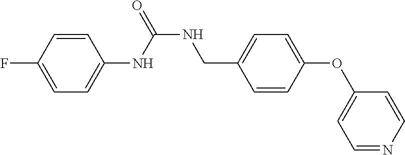 [SMILES: O=C(NCc1ccc(Oc2ccncc2)cc1)Nc1ccc(F)cc1] | 1-(4-Fluoro-phenyl)-3-[4- (pyridin-4-yloxy)-benzyl]- urea | 3400 | | 337.35 |
| 12 | 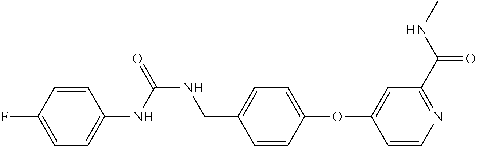 [SMILES: CNC(=O)c1cc(Oc2ccc(CNC(=O)Nc3ccc(F)cc3)cc2)ccn1] | 4-{4-[3-(4-Fluoro-phenyl)- ureidomethyl]-phenoxy}- pyridine-2-carboxylic acid methylamide | | 775 | 394.41 |
| 13 | 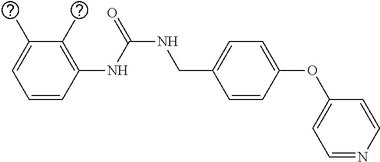 [SMILES: Cc1cccc(NC(=O)NCc2ccc(Oc3ccncc3)cc2)c1C] | 1-(2,3-Dichloro-phenyl)-3- [4-(pyridin-4-yloxy)- benzyl]-urea | 100000 | | 388.25 |
| 14 | 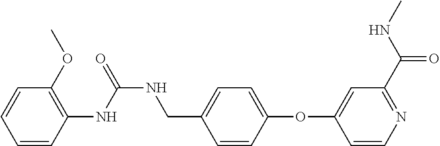 [SMILES: CNC(=O)c1cc(Oc2ccc(CNC(=O)Nc3ccccc3OC)cc2)ccn1] | 4-{4-[3-(2-Methoxy- phenyl)-ureidomethyl]- phenoxy}-pyridine-2- carboxylic acid methylamide | 1900 | | 406.44 |
| 15 | 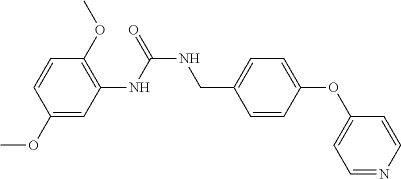 [SMILES: COc1ccc(OC)c(NC(=O)NCc2ccc(Oc3ccncc3)cc2)c1] | 1-(2,5-Dimethoxy-phenyl)- 3-[4-(pyridin-4-yloxy)- benzyl]-urea | 440 | | 379.42 |
| 16 | 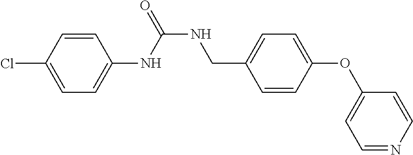 [SMILES: O=C(NCc1ccc(Oc2ccncc2)cc1)Nc1ccc(Cl)cc1] | 1-(4-Chloro-phenyl)-3-[4- (pyridin-4-yloxy)-benzyl]- urea | 5900 | | 353.81 |
| 17 | 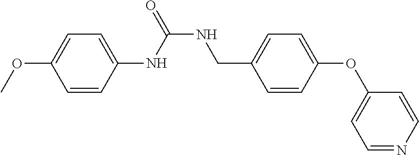 [SMILES: COc1ccc(NC(=O)NCc2ccc(Oc3ccncc3)cc2)cc1] | 1-(4-Methoxy-phenyl)-3-[4- (pyridin-4-yloxy)-benzyl]- urea | 13000 | | 349.39 |
| 18 | 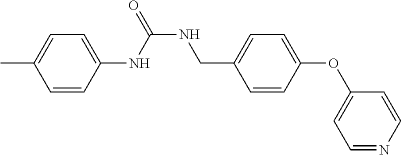 [SMILES: Cc1ccc(NC(=O)NCc2ccc(Oc3ccncc3)cc2)cc1] | 1-[4-(Pyridin-4-yloxy)- benzyl]-3-p-tolyl-urea | 4100 | | 333.39 |
| 19 | 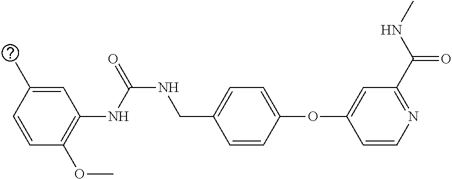 [SMILES: CNC(=O)c1cc(Oc2ccc(CNC(=O)Nc3cc(C)ccc3OC)cc2)ccn1] | 4-{4-[3-(5-Chloro-2- methoxy-phenyl)- ureidomethyl]-phenoxy}- pyridine-2-carboxylic acid methylamide | 30 | 14 | 440.89 |
| 20 | 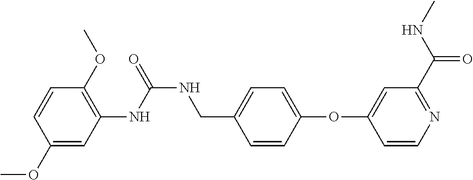 [SMILES: CNC(=O)c1cc(Oc2ccc(CNC(=O)Nc3cc(OC)ccc3OC)cc2)ccn1] | 4-{4-[3-(2,5-Dimethoxy- phenyl)-ureidomethyl]- phenoxy}-pyridine-2- carboxylic acid methylamide | 98 | | 436.47 |
| 21 | 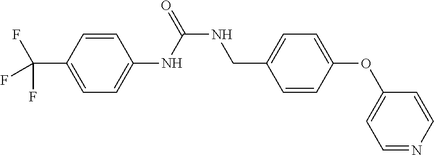 [SMILES: O=C(NCc1ccc(Oc2ccncc2)cc1)Nc1ccc(C(F)(F)F)cc1] | 1-[4-(Pyridin-4-yloxy)- benzyl]-3-(4- trifluoromethyl-phenyl)- urea | 2000 | | 387.36 |
| 22 | 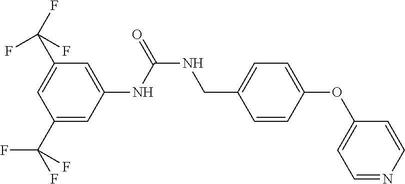 [SMILES: O=C(NCc1ccc(Oc2ccncc2)cc1)Nc1cc(C(F)(F)F)cc(C(F)(F)F)c1] | 1-(3,5-Bis-trifluoromethyl- phenyl)-3-[4-(pyridin-4- yloxy)-benzyl]-urea | 560 | | 455.36 |
| 23 | 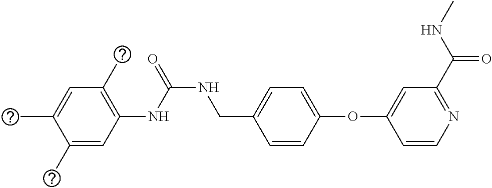 [SMILES: CNC(=O)c1cc(Oc2ccc(CNC(=O)Nc3cc(C)c(C)cc3C)cc2)ccn1] | 4-{4-[3-(2,4,5-Trichloro- phenyl)-ureidomethyl]- phenoxy}-pyridine-2- carboxylic acid methylamide | | 100 | 479.75 |
| 24 | 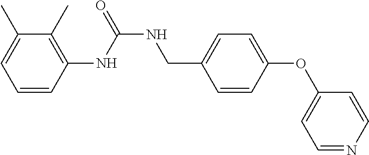 [SMILES: Cc1cccc(NC(=O)NCc2ccc(Oc3ccncc3)cc2)c1C] | 1-(2,3-Dimethyl-phenyl)-3- [4-(pyridin-4-yloxy)- benzyl]-urea | 100000 | | 347.42 |
| 25 | 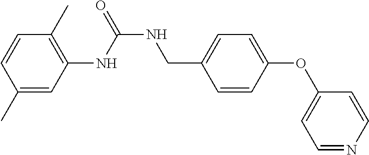 [SMILES: Cc1ccc(C)c(NC(=O)NCc2ccc(Oc3ccncc3)cc2)c1] | 1-(2,5-Dimethyl-phenyl)-3- [4-(pyridin-4-yloxy)- benzyl]-urea | 13000 | | 347.42 |
| 26 | 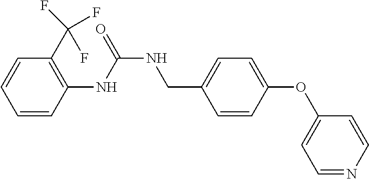 [SMILES: O=C(NCc1ccc(Oc2ccncc2)cc1)Nc1ccccc1C(F)(F)F] | 1-[4-(Pyridin-4-yloxy)- benzyl]-3-(2- trifluoromethyl-phenyl)- urea | 20000 | | 387.36 |
| 27 | 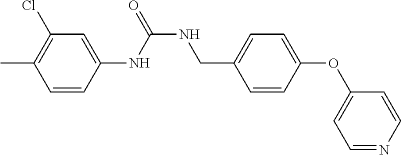 [SMILES: Cc1ccc(NC(=O)NCc2ccc(Oc3ccncc3)cc2)cc1Cl] | 1-(3-Chloro-4-methyl- phenyl)-3-[4-(pyridin-4- yloxy)-benzyl]-urea | 740 | | 367.84 |
| 28 | 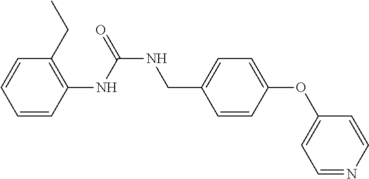 [SMILES: CCc1ccccc1NC(=O)NCc1ccc(Oc2ccncc2)cc1] | 1-(2-Ethyl-phenyl)-3-[4- (pyridin-4-yloxy)-benzyl]- urea | 100000 | | 347.42 |
| 29 | 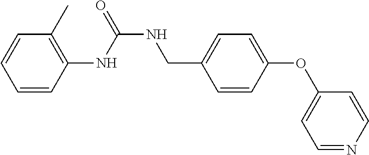 [SMILES: Cc1ccccc1NC(=O)NCc1ccc(Oc2ccncc2)cc1] | 1-[4-(Pyridin-4-yloxy)- benzyl]-3-o-tolyl-urea | 100000 | | 333.39 |
| 30 | 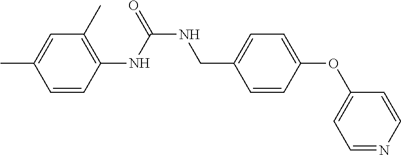 [SMILES: Cc1ccc(NC(=O)NCc2ccc(Oc3ccncc3)cc2)c(C)c1] | 1-(2,4-Dimethyl-phenyl)-3- [4-(pyridin-4-yloxy)- benzyl]-urea | 28000 | | 347.42 |
| 31 | 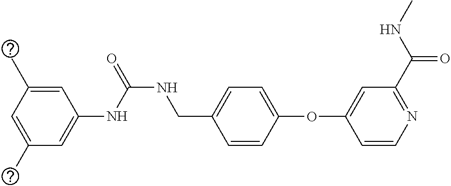 [SMILES: CNC(=O)c1cc(Oc2ccc(CNC(=O)Nc3cc(C)cc(C)c3)cc2)ccn1] | 4-{4-[3-(3,5-Dichloro- phenyl)-ureidomethyl]- phenoxy}-pyridine-2- carboxylic acid methylamide | | 175 | 445.31 |
| 32 | 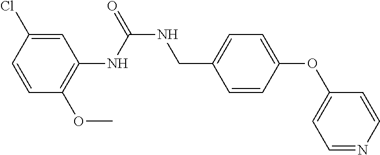 [SMILES: COc1ccc(Cl)cc1NC(=O)NCc1ccc(Oc2ccncc2)cc1] | 1-(5-Chloro-2-methoxy- phenyl)-3-[4-(pyridin-4- yloxy)-benzyl]-urea | 87 | | 383.83 |
| 33 | 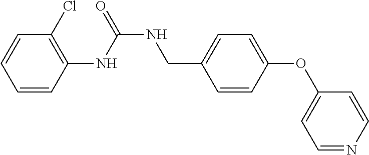 [SMILES: O=C(NCc1ccc(Oc2ccncc2)cc1)Nc1ccccc1Cl] | 1-(2-Chloro-phenyl)-3-[4- (pyridin-4-yloxy)-benzyl]- urea | 100000 | | 353.81 |
| 34 | 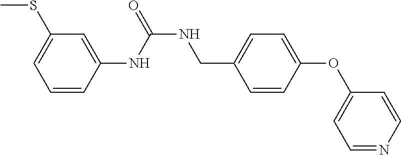 [SMILES: CSc1cccc(NC(=O)NCc2ccc(Oc3ccncc3)cc2)c1] | 1-(3-Methylsulfanyl- phenyl)-3-[4-(pyridin-4- yloxy)-benzyl]-urea | 5500 | | 365.45 |
| 35 | 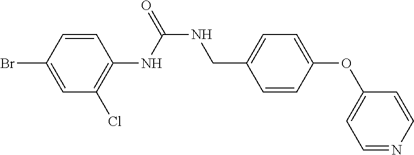 [SMILES: O=C(NCc1ccc(Oc2ccncc2)cc1)Nc1ccc(Br)cc1Cl] | 1-(4-Bromo-2-chloro- phenyl)-3-[4-(pyridin-4- yloxy)-benzyl]-urea | 9500 | | 432.71 |
| 36 | 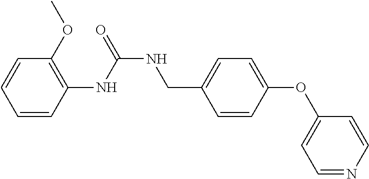 [SMILES: COc1ccccc1NC(=O)NCc1ccc(Oc2ccncc2)cc1] | 1-(2-Methoxy-phenyl)-3-[4- (pyridin-4-yloxy)-benzyl]- urea | 1700 | | 349.39 |
| 37 | 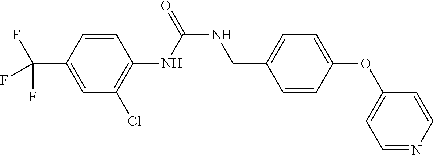 [SMILES: O=C(NCc1ccc(Oc2ccncc2)cc1)Nc1ccc(C(F)(F)F)cc1Cl] | 1-(2-Chloro-4- trifluoromethyl-phenyl)-3- [4-(pyridin-4-yloxy)- benzyl]-urea | 2800 | | 421.81 |
| 38 | 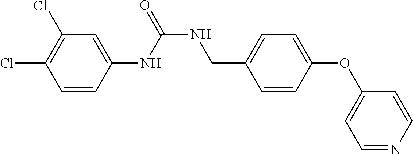 [SMILES: O=C(NCc1ccc(Oc2ccncc2)cc1)Nc1ccc(Cl)c(Cl)c1] | 1-(3,4-Dichloro-phenyl)-3- [4-(pyridin-4-yloxy)- benzyl]-urea | 2100 | | 388.25 |
| 39 | 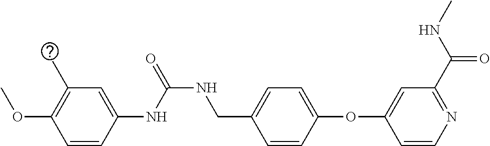 [SMILES: CNC(=O)c1cc(Oc2ccc(CNC(=O)Nc3ccc(OC)c(C)c3)cc2)ccn1] | 4-{4-[3-(3-Chloro-4- methoxy-phenyl)- ureidomethyl]-phenoxy}- pyridine-2-carboxylic acid methylamide | | 64 | 440.89 |
| 40 | 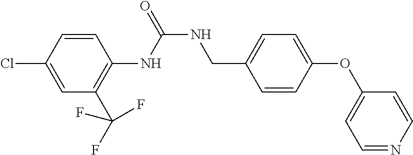 [SMILES: O=C(NCc1ccc(Oc2ccncc2)cc1)Nc1ccc(Cl)cc1C(F)(F)F] | 1-(4-Chloro-2- trifluoromethyl-phenyl)-3- [4-(pyridin-4-yloxy)- benzyl]-urea | 9900 | | 421.81 |
| 41 | 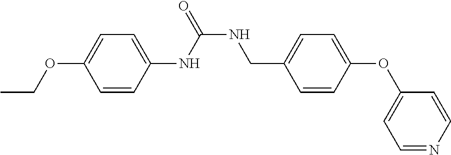 [SMILES: CCOc1ccc(NC(=O)NCc2ccc(Oc3ccncc3)cc2)cc1] | 1-(4-Ethoxy-phenyl)-3-[4- (pyridin-4-yloxy)-benzyl]- urea | 100000 | | 363.42 |
| 42 | 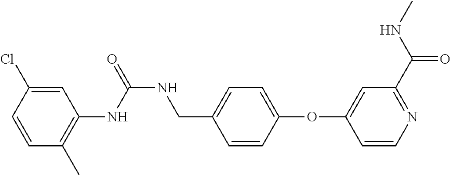 [SMILES: CNC(=O)c1cc(Oc2ccc(CNC(=O)Nc3cc(Cl)ccc3C)cc2)ccn1] | 4-{4-[3-(5-Chloro-2- methyl-phenyl)- ureidomethyl]- phenoxy}-pyridine-2- carboxylic acid methylamide | | 230 | 424.89 |
| 43 | 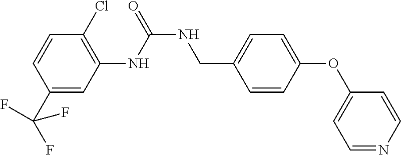 [SMILES: O=C(NCc1ccc(Oc2ccncc2)cc1)Nc1cc(C(F)(F)F)ccc1Cl] | 1-(2-Chloro-5- trifluoromethyl-phenyl)-3- [4-(pyridin-4-yloxy)- benzyl]-urea | 42 | | 421.81 |
| 44 | 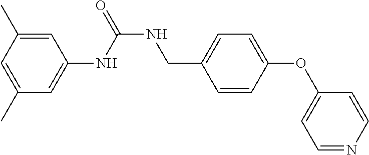 [SMILES: Cc1cc(C)cc(NC(=O)NCc2ccc(Oc3ccncc3)cc2)c1] | 1-(3,5-Dimethyl-phenyl)-3- [4-(pyridin-4-yloxy)- benzyl]-urea | 1800 | | 347.42 |
| 45 | 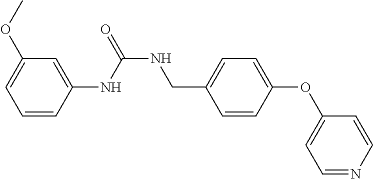 [SMILES: COc1cccc(NC(=O)NCc2ccc(Oc3ccncc3)cc2)c1] | 1-(3-Methoxy-phenyl)-3-[4- (pyridin-4-yloxy)-benzyl]- urea | 4000 | | 349.39 |
| 46 | 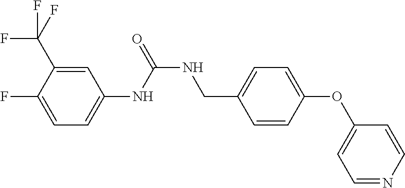 [SMILES: O=C(NCc1ccc(Oc2ccncc2)cc1)Nc1ccc(F)c(C(F)(F)F)c1] | 1-(4-Fluoro-3- trifluoromethyl-phenyl)-3- [4-(pyridin-4-yloxy)- benzyl]-urea | 100 | | 405.35 |
| 47 | 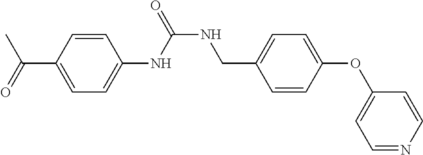 [SMILES: CC(=O)c1ccc(NC(=O)NCc2ccc(Oc3ccncc3)cc2)cc1] | 1-(4-Acetyl-phenyl)-3-[4- (pyridin-4-yloxy)-benzyl]- urea | 8800 | | 361.40 |
| 48 | 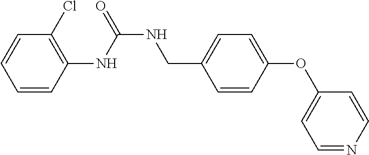 [SMILES: O=C(NCc1ccc(Oc2ccncc2)cc1)Nc1ccccc1Cl] | 1-(2-Bromo-phenyl)-3-[4- (pyridin-4-yloxy)-benzyl]- urea | 100000 | | 398.26 |
| 49 | 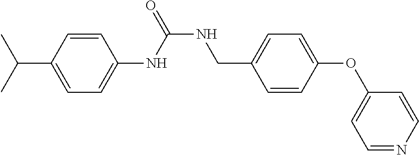 [SMILES: CC(C)c1ccc(NC(=O)NCc2ccc(Oc3ccncc3)cc2)cc1] | 1-(4-lsopropyl-phenyl)-3- [4-(pyridin-4-yloxy)- benzyl]-urea | 9600 | | 361.44 |
| 50 | 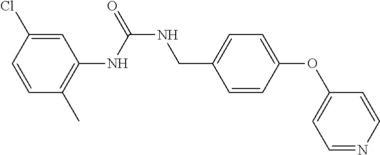 [SMILES: Cc1ccc(Cl)cc1NC(=O)NCc1ccc(Oc2ccncc2)cc1] | 1-(5-Chloro-2-methyl- phenyl)-3-[4-(pyridin-4- yloxy)-benzyl]-urea | 1700 | | 367.84 |
| 51 | 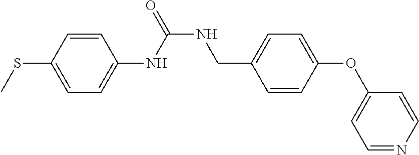 [SMILES: CSc1ccc(NC(=O)NCc2ccc(Oc3ccncc3)cc2)cc1] | 1-(4-Methylsulfanyl- phenyl)-3-[4-(pyridin-4- yloxy)-benzyl]-urea | 10000 | | 365.45 |
| 52 | 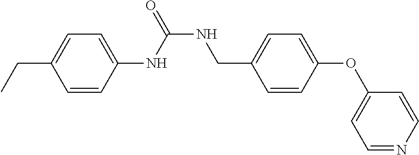 [SMILES: CCc1ccc(NC(=O)NCc2ccc(Oc3ccncc3)cc2)cc1] | 1-(4-Ethyl-phenyl)-3-[4- (pyridin-4-yloxy)-benzyl]- urea | 9900 | | 347.42 |
| 53 | 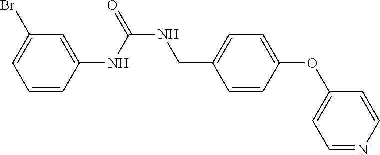 [SMILES: O=C(NCc1ccc(Oc2ccncc2)cc1)Nc1cccc(Br)c1] | 1-(3-Bromo-phenyl)-3-[4- (pyridin-4-yloxy)-benzyl]- urea | 56 | | 398.26 |
| 54 | 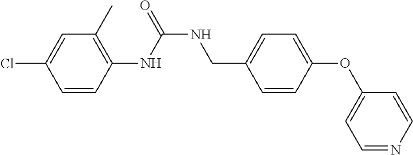 [SMILES: Cc1cc(Cl)ccc1NC(=O)NCc1ccc(Oc2ccncc2)cc1] | 1-(4-Chloro-2-methyl- phenyl)-3-[4-(pyridin-4- yloxy)-benzyl]-urea | 100000 | | 367.84 |
| 55 | 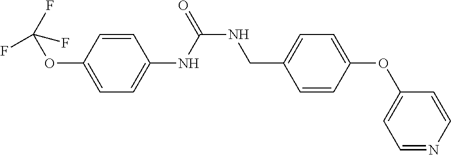 [SMILES: O=C(NCc1ccc(Oc2ccncc2)cc1)Nc1ccc(OC(F)(F)F)cc1] | 1-[4-(Pyridin-4-yloxy)- benzyl]-3-(4- trifluoromethoxy-phenyl)- urea | 7500 | | 403.36 |
| 56 | 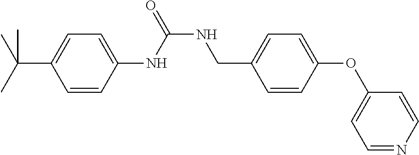 [SMILES: CC(C)(C)c1ccc(NC(=O)NCc2ccc(Oc3ccncc3)cc2)cc1] | 1-(4-tert-Butyl-phenyl)-3- [4-(pyridin-4-yloxy)- benzyl]-urea | 16000 | | 375.47 |
| 57 | 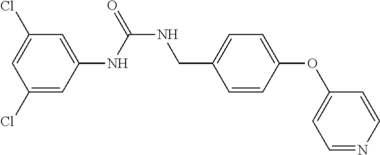 [SMILES: O=C(NCc1ccc(Oc2ccncc2)cc1)Nc1cc(Cl)cc(Cl)c1] | 1-(3,5-Dichloro-phenyl)-3- [4-(pyridin-4-yloxy)- benzyl]-urea | 1200 | | 388.25 |
| 58 | 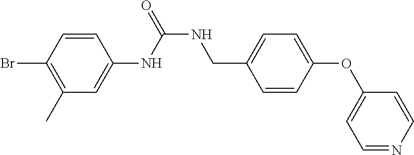 [SMILES: Cc1cc(NC(=O)NCc2ccc(Oc3ccncc3)cc2)ccc1Br] | 1-(4-Bromo-3-methyl- phenyl)-3-[4-(pyridin-4- yloxy)-benzyl]-urea | 4700 | | 412.29 |
| 59 | 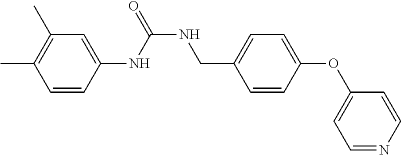 [SMILES: Cc1ccc(NC(=O)NCc2ccc(Oc3ccncc3)cc2)cc1C] | 1-(3,4-Dimethyl-phenyl)-3- [4-(pyridin-4-yloxy)- benzyl]-urea | 2100 | | 347.42 |
| 60 | 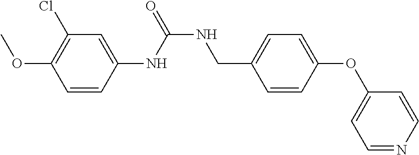 [SMILES: COc1ccc(NC(=O)NCc2ccc(Oc3ccncc3)cc2)cc1Cl] | 1-(3-Chloro-4-methoxy- phenyl)-3-[4-(pyridin-4- yloxy)-benzyl]-urea | 2800 | | 383.83 |
| 61 | 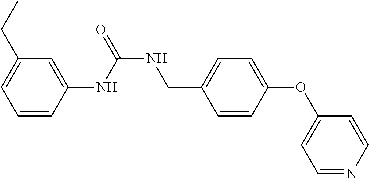 [SMILES: CCc1cccc(NC(=O)NCc2ccc(Oc3ccncc3)cc2)c1] | 1-(3-Ethyl-phenyl)-3-[4- (pyridin-4-yloxy)-benzyl]- urea | 490 | | 347.42 |
| 62 | 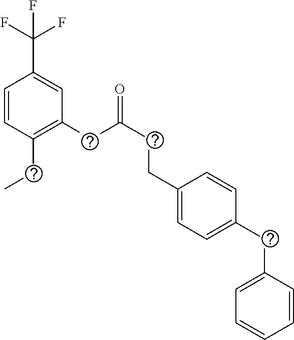 [SMILES: CCc1ccc(C(F)(F)F)cc1CC(=O)CCc1ccc(Cc2ccccc2)cc1] | 1-(2-Methoxy-5- trifluoromethyl-phenyl)-3- [4-(pyridin-4-yloxy)- benzyl]-urea | 2 | | 417.39 |
| 63 | 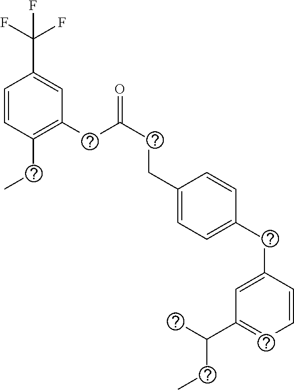 [SMILES: CCc1ccc(C(F)(F)F)cc1CC(=O)CCc1ccc(Cc2cccc(C(C)CC)c2)cc1] | 4-{4-[3-(2-Methoxy-5- trifluoromethyl-phenyl)- ureidomethyl]-phenoxy}- pyridine-2-carboxylic acid methylamide | 61 | | 474.44 |
| 64 | 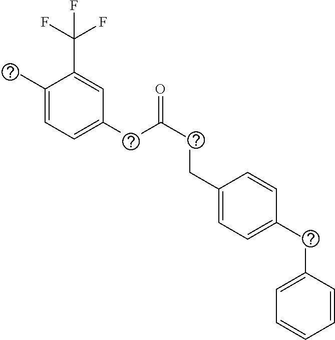 [SMILES: Cc1ccc(CC(=O)CCc2ccc(Cc3ccccc3)cc2)cc1C(F)(F)F] | 1-(4-Bromo-3- trifluoromethyl-phenyl)-3- [4-(pyridin-4-yloxy)- benzyl]-urea | 1700 | | 466.26 |
| 65 | 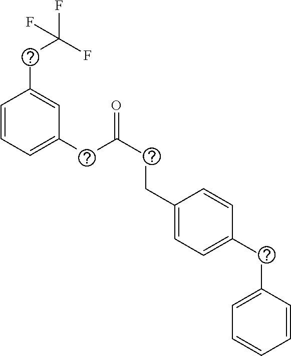 [SMILES: O=C(CCc1ccc(Cc2ccccc2)cc1)Cc1cccc(CC(F)(F)F)c1] | 1-[4-(Pyridin-4-yloxy)- benzyl]-3-(3- trifluoromethoxy-phenyl)- urea | 35 | | 403.36 |
| 66 | 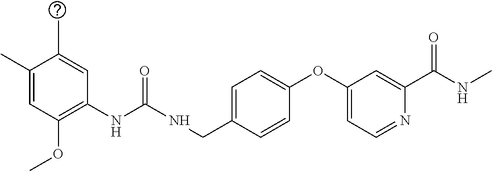 | 4-{4-[3-(5-Chloro-2- methoxy-4-methyl-phenyl)- ureidomethyl]-phenoxy}- pyridine-2-carboxylic acid methylamide | 55 | 9 | 454.91 |
| 67 | 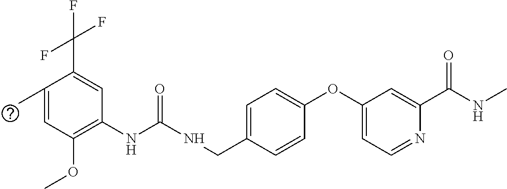 | 4-{4-[3-(4-Chloro-2- methoxy-5-trifluoromethyl- phenyl)-ureidomethyl]- phenoxy}-pyridine-2- carboxylic acid methylamide | 175 | | 508.88 |
| 68 | 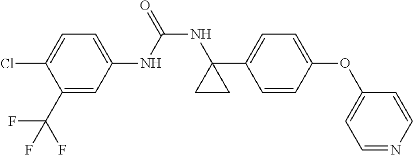 [SMILES: O=C(Nc1ccc(Cl)c(C(F)(F)F)c1)NC1(c2ccc(Oc3ccncc3)cc2)CC1] | 1-(4-Chloro-3- trifluoromethyl-phenyl)-3- {1-[4-(pyridin-4-yloxy)- phenyl]-cyclopropyl}-urea | 4600 | | 447.84 |
| 69 | 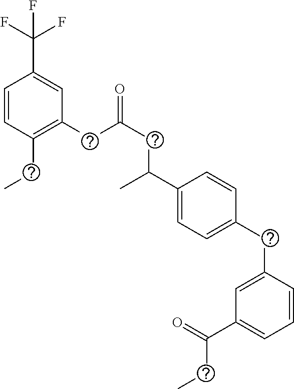 [SMILES: CCC(=O)c1cccc(Cc2ccc(C(C)CC(=O)Cc3cc(C(F)(F)F)ccc3CC)cc2)c1] | 4-(4-{1-[3-(2-Methoxy-5- trifluoromethyl-phenyl)- ureido]-ethyl}-phenoxy)- pyridine-2-carboxylic acid methylamide | 150 | | 488.47 |
| 70 | 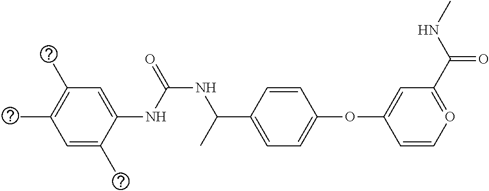 | 4-(4-{1-[3-(2,4,5-Trichloro- phenyl)-ureido]-ethyl}- phenoxy)-pyridine-2- carboxylic acid methylamide | | 735 | 493.78 |
| 71 | 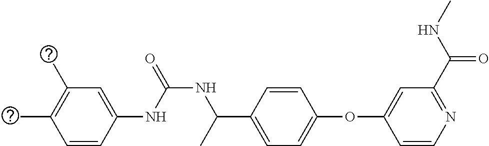 [SMILES: CNC(=O)c1cc(Oc2ccc(C(C)NC(=O)Nc3ccc(C)c(C)c3)cc2)ccn1] | 4-(4-{1-[3-(3,4-Dichloro- phenyl)-ureido]-ethyl}- phenoxy)-pyridine-2- carboxylic acid methylamide | | 1800 | 459.33 |
| 72 | 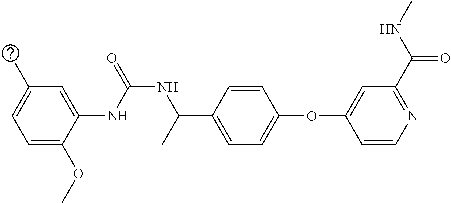 [SMILES: CNC(=O)c1cc(Oc2ccc(C(C)NC(=O)Nc3cc(C)ccc3OC)cc2)ccn1] | 4-(4-{1-[3-(5-Chloro-2- methoxy-phenyl)-ureido]- ethyl}-phenoxy)-pyridine-2- carboxylic acid methylamide | 340 | 74 | 454.91 |
| 73 | 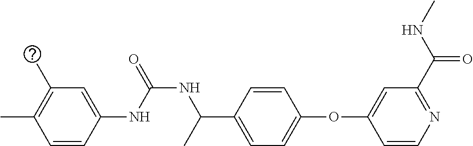 [SMILES: CNC(=O)c1cc(Oc2ccc(C(C)NC(=O)Nc3ccc(C)c(C)c3)cc2)ccn1] | 4-(4-{1-[3-(3-Chloro-4- methyl-phenyl)-ureido]- ethyl}-phenoxy)-pyridine-2- carboxylic acid methylamide | | 1750 | 438.91 |
| 74 | 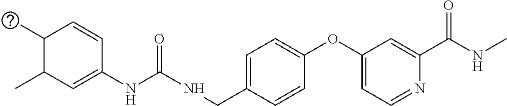 | 4-{4-[3-(4-Chloro-3- methyl-phenyl)- ureidomethyl]- phenoxy}-pyridine-2- carboxylic acid methylamide | | 175 | 424.89 |
| 75 | 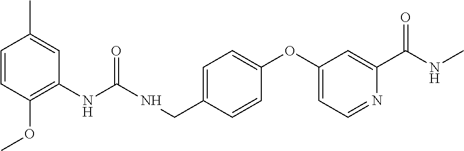 [SMILES: CNC(=O)c1cc(Oc2ccc(CNC(=O)Nc3cc(C)ccc3OC)cc2)ccn1] | 4-{4-[3-(2-Methoxy-5- methyl-phenyl)- ureidomethyl]-phenoxy}- pyridine-2-carboxylic acid methylamide | 69 | | 420.47 |
| 76 | 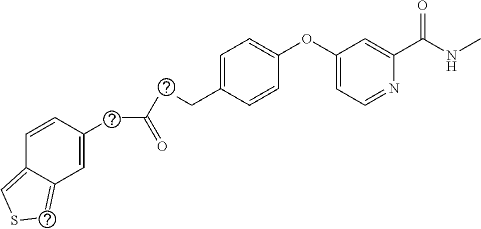 [SMILES: CNC(=O)c1cc(Oc2ccc(CCC(=O)Cc3ccc4cscc4c3)cc2)ccn1] | 4-{4-[(3- Benzo[1,2,5]thiadiazol-5- yl-ureido)-methyl]- phenoxy]-pyridine-2- carboxylic acid methylamide | | 670 | 434.48 |
| 77 | 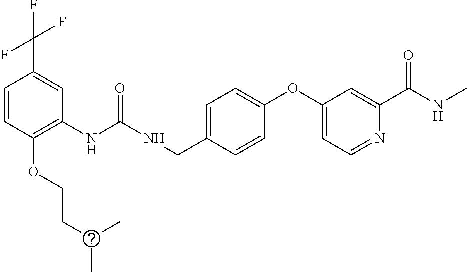 [SMILES: CNC(=O)c1cc(Oc2ccc(CNC(=O)Nc3cc(C(F)(F)F)ccc3OCCC(C)C)cc2)ccn1] | 4-(4-{3-[2-(2- Dimethylamino-ethoxy)-5- trifluoromethyl-phenyl]- ureidomethyl}-phenoxy)- pyridine-2-carboxylic acid methylamide | 78 | | 531.53 |
| 78 | 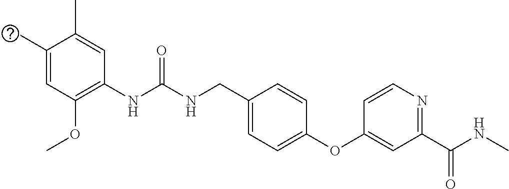 | 4-{4-[3-(4-Chloro-2- methoxy-5-methyl-phenyl)- ureidomethyl]-phenoxy}- pyridine-2-carboxylic acid methylamide | 100 | 24 | 454.91 |
| 79 | 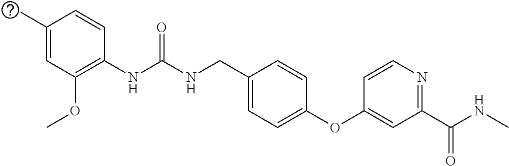 | 4-{4-[3-(4-Chloro-2- methoxy-phenyl)- ureidomethyl]-phenoxy}- pyridine-2-carboxylic acid methylamide | 410 | | 440.89 |
| 80 | 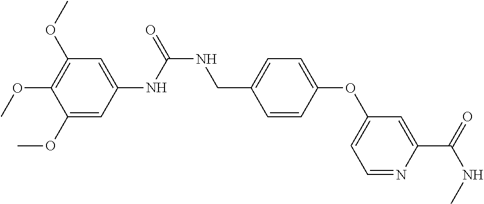 [SMILES: CNC(=O)c1cc(Oc2ccc(CNC(=O)Nc3cc(OC)c(OC)c(OC)c3)cc2)ccn1] | 4-{4-[3-(3,4,5-Trimethoxy- phenyl)-ureidomethyl]- phenoxy}-pyridine-2- carboxylic acid methylamide | | 750 | 466.49 |
| 81 | 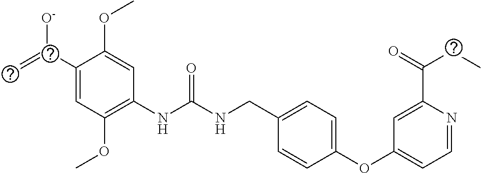 [SMILES: C=C([O-])c1cc(OC)c(NC(=O)NCc2ccc(Oc3ccnc(C(=O)CC)c3)cc2)cc1OC] | 4-{4-[3-(2,5-Dimethoxy-4- nitro-phenyl)- ureidomethyl]-phenoxy}- pyridine-2-carboxylic acid methylamide | 230 | | 482.47 |
| 82 | 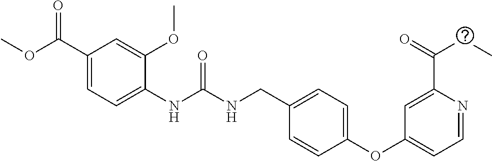 [SMILES: CCC(=O)c1cc(Oc2ccc(CNC(=O)Nc3ccc(C(=O)OC)cc3OC)cc2)ccn1] | 3-Methoxy-4-{3-[4-(2- methylcarbamoyl-pyridin- 4-yloxy)-benzyl]-ureido}- benzoic acid methyl ester | 2900 | | 464.48 |
| 83 | 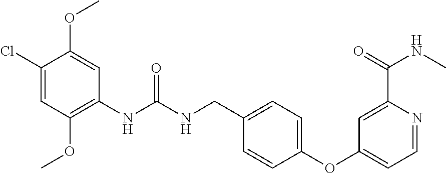 [SMILES: CNC(=O)c1cc(Oc2ccc(CNC(=O)Nc3cc(OC)c(Cl)cc3OC)cc2)ccn1] | 4-{4-[3-(4-Chloro-2,5- dimethoxy-phenyl)- ureidomethyl]-phenoxy}- pyridine-2-carboxylic acid methylamide | 760 | 140 | 470.91 |
| 84 | 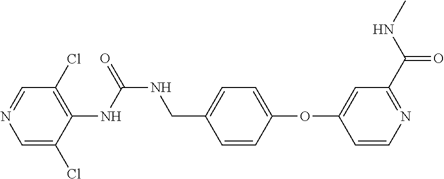 [SMILES: CNC(=O)c1cc(Oc2ccc(CNC(=O)Nc3c(Cl)cncc3Cl)cc2)ccn1] | 4-{4-[3-(3,5-Dichloro- pyridin-4-yl)-ureidomethyl]- phenoxy}-pyridine-2- carboxylic acid methylamide | 810 | 745 | 446.29 |
| 85 | 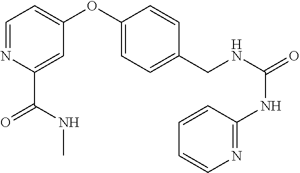 [SMILES: CNC(=O)c1cc(Oc2ccc(CNC(=O)Nc3ccccn3)cc2)ccn1] | 4-{4-[(3-Pyridin-2-yl- ureido)-methyl]-phenoxy}- pyridine-2-carboxylic acid methylamide | 3800 | | 377.40 |
| 86 | 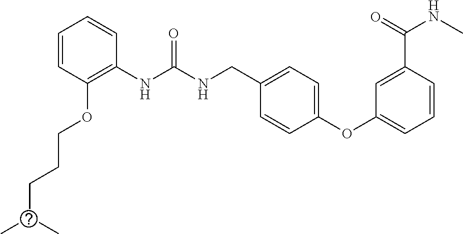 [SMILES: CNC(=O)c1cccc(Oc2ccc(CNC(=O)Nc3ccccc3OCCCC(C)C)cc2)c1] | 4-(4-{3-[2-(3- Dimethylamino-propoxy)- phenyl]-ureidomethyl}- phenoxy)-pyridine-2- carboxylic acid methylamide | 1200 | | 477.56 |
| 87 | 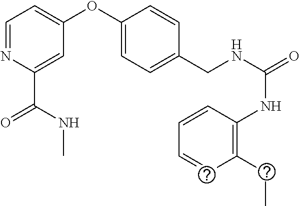 [SMILES: CCc1ccccc1NC(=O)NCc1ccc(Oc2ccnc(C(=O)NC)c2)cc1] | 4-{4-[3-(2-Methoxy-pyridin- 3-yl)-ureidomethyl]- phenoxy}-pyridine-2- carboxylic acid methylamide | 1600 | | 407.43 |
| 88 | 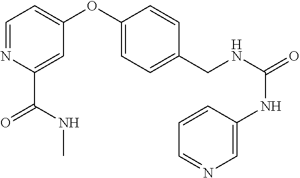 [SMILES: CNC(=O)c1cc(Oc2ccc(CNC(=O)Nc3cccnc3)cc2)ccn1] | 4-{4-[(3-Pyridin-3-yl- ureido)-methyl]-phenoxy}- pyridine-2-carboxylic acid methylamide | 15000 | | 377.40 |
| 89 | 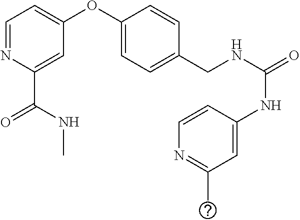 [SMILES: CNC(=O)c1cc(Oc2ccc(CNC(=O)Nc3ccnc(C)c3)cc2)ccn1] | 4-{4-[3-(2-Chloro-pyridin-4- yl)-ureidomethyl]- phenoxy}-pyridine-2- carboxylic acid methylamide | 1000 | 1450 | 411.85 |
| 90 | 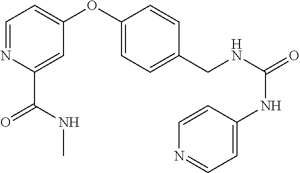 [SMILES: CNC(=O)c1cc(Oc2ccc(CNC(=O)Nc3ccncc3)cc2)ccn1] | 4-{4-[(3-Pyridin-4-yl- ureido)-methyl]-phenoxy}- pyridine-2-carboxylic acid methylamide | 100000 | | 377.40 |
| 91 | 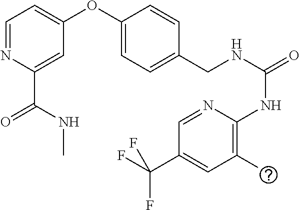 [SMILES: CNC(=O)c1cc(Oc2ccc(CNC(=O)Nc3ncc(C(F)(F)F)cc3C)cc2)ccn1] | 4-{4-[3-(3-Chloro-5- trifluoromethyl-pyridin-2- yl)-ureidomethyl]- phenoxy}-pyridine-2- carboxylic acid methylamide | 6600 | | 479.85 |
| 92 | 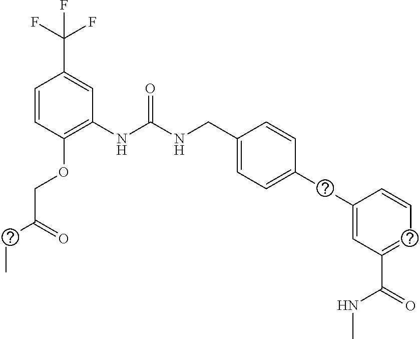 [SMILES: CCC(=O)COc1ccc(C(F)(F)F)cc1NC(=O)NCc1ccc(Cc2cccc(C(=O)NC)c2)cc1] | (2-{3-[4-(2- Methylcarbamoyl-pyridin- 4-yloxy)-benzyl]-ureido}-4- trifluoromethyl-phenoxy)- acetic acid methyl ester | 38 | | 532.47 |
| 93 | 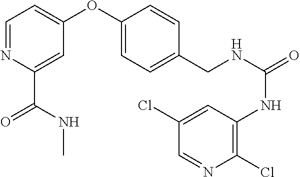 [SMILES: CNC(=O)c1cc(Oc2ccc(CNC(=O)Nc3cc(Cl)cnc3Cl)cc2)ccn1] | 4-{4-[3-(2,5-Dichloro- pyridin-3-yl)-ureidomethyl]- phenoxy}-pyridine-2- carboxylic acid methylamide | 42 | | 446.29 |
| 94 | 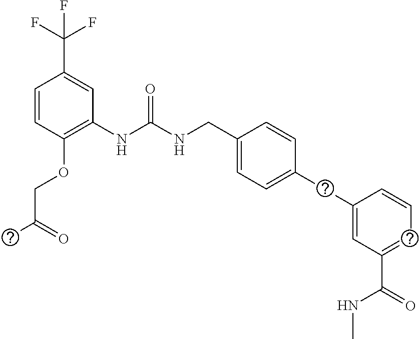 [SMILES: CNC(=O)c1cccc(Cc2ccc(CNC(=O)Nc3cc(C(F)(F)F)ccc3OCC(C)=O)cc2)c1] | (2-{3-[4-(2- Methylcarbamoyl-pyridin- 4-yloxy)-benzyl]-ureido}-4- trifluoromethyl-phenoxy)- acetic acid | 1600 | | 518.45 |
| 95 | 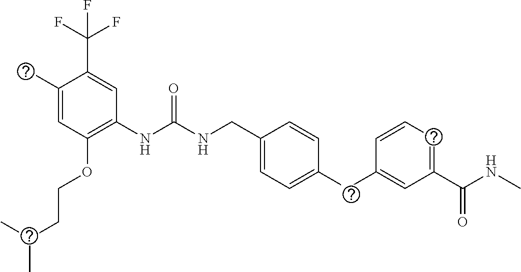 [SMILES: CNC(=O)c1cccc(Cc2ccc(CNC(=O)Nc3cc(C(F)(F)F)c(C)cc3OCCC(C)C)cc2)c1] | 4-(4-{3-[4-Chloro-2-(2- dimethylamino-ethoxy)-5- trifluoromethyl-phenyl]- ureidomethyl}-phenoxy)- pyridine-2-carboxylic acid methylamide | 81 | | 565.98 |
| 96 | 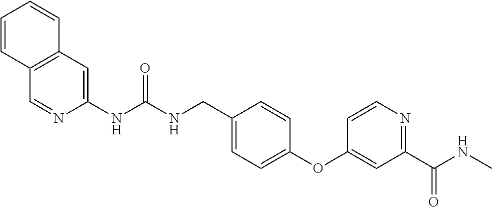 [SMILES: CNC(=O)c1cc(Oc2ccc(CNC(=O)Nc3cc4ccccc4cn3)cc2)ccn1] | 4-{4-[(3-Isoquinolin-3-yl- ureido)-methyl]-phenoxy}- pyridine-2-carboxylic acid methylamide | 170 | | 427.46 |
| 97 | 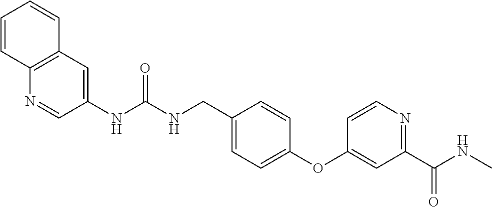 [SMILES: CNC(=O)c1cc(Oc2ccc(CNC(=O)Nc3cnc4ccccc4c3)cc2)ccn1] | 4-{4-[(3-Quinolin-3-yl- ureido)-methyl]-phenoxy}- pyridine-2-carboxylic acid methylamide | 290 | | 427.46 |
| 98 | 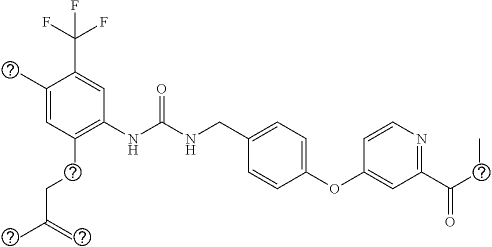 [SMILES: C=C(C)CCc1cc(C)c(C(F)(F)F)cc1NC(=O)NCc1ccc(Oc2ccnc(C(=O)CC)c2)cc1] | (5-Chloro-2-{3-[4-(2- methylcarbamoyl-pyridin- 4-yloxy)-benzyl]-ureido}-4- trifluoromethyl-phenoxy)- acetic acid | 3300 | | 552.89 |
| 99 | 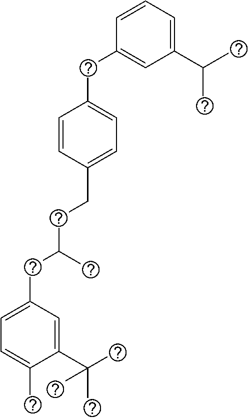 [SMILES: Cc1ccc(CC(C)CCc2ccc(Cc3cccc(C(C)C)c3)cc2)cc1C(C)(C)C] | 4-{4-[3-(4-Chloro-3- trifluoromethyl-phenyl)- ureidomethyl]-phenoxy}- pyridine-2-carboxylic acid | | 2850 | 465.81 |
| 100 | 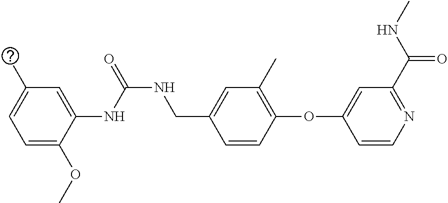 [SMILES: CNC(=O)c1cc(Oc2ccc(CNC(=O)Nc3cc(C)ccc3OC)cc2C)ccn1] | 4-{4-[3-(5-Chloro-2- methoxy-phenyl)- ureidomethyl]-2-methyl- phenoxy}-pyridine-2- carboxylic acid methylamide | 120 | 25 | 454.91 |
| 101 | 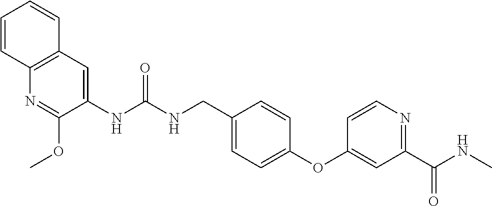 [SMILES: CNC(=O)c1cc(Oc2ccc(CNC(=O)Nc3cc4ccccc4nc3OC)cc2)ccn1] | 4-{4-[3-(2-Methoxy- quinolin-3-yl)- ureidomethyl]-phenoxy}- pyridine-2-carboxylic acid methylamide | 190 | | 457.49 |
| 102 | 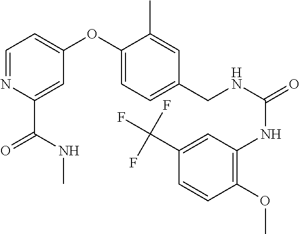 [SMILES: CNC(=O)c1cc(Oc2ccc(CNC(=O)Nc3cc(C(F)(F)F)ccc3OC)cc2C)ccn1] | 4-{4-[3-(2-Methoxy-5- trifluoromethyl-phenyl)- ureidomethyl]-2-methyl- phenoxy}-pyridine-2- carboxylic acid methylamide | 50 | | 488.47 |
| 103 | 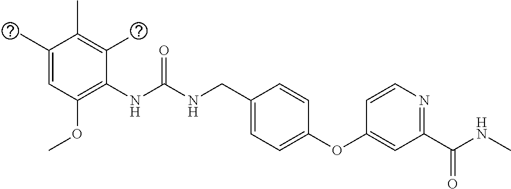 | 4-{4-[3-(2,4-Dichloro-6- methoxy-3-methyl-phenyl)- ureidomethyl]-phenoxy}- pyridine-2-carboxylic acid methylamide | 14000 | 4600 | 489.36 |
| 104 | 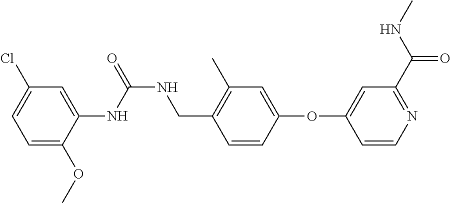 [SMILES: CNC(=O)c1cc(Oc2ccc(CNC(=O)Nc3cc(Cl)ccc3OC)c(C)c2)ccn1] | 4-{4-[3-(5-Chloro-2- methoxy-phenyl)- ureidomethyl]-3-methyl- phenoxy}-pyridine-2- carboxylic acid methylamide | 270 | | 454.91 |
| 105 | 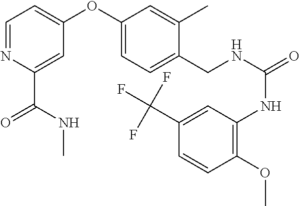 [SMILES: CNC(=O)c1cc(Oc2ccc(CNC(=O)Nc3cc(C(F)(F)F)ccc3OC)c(C)c2)ccn1] | 4-{4-[3-(2-Methoxy-5- trifluoromethyl-phenyl)- ureidomethyl]-3-methyl- phenoxy}-pyridine-2- carboxylic acid methylamide | 180 | | 488.47 |
| 106 | 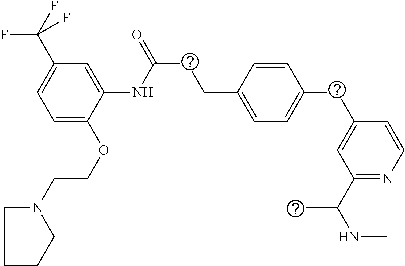 [SMILES: CNC(C)c1cc(Cc2ccc(CCC(=O)Nc3cc(C(F)(F)F)ccc3OCCN3CCCC3)cc2)ccn1] | 4-(4-{3-[2-(2-Pyrrolidin-1- yl-ethoxy)-5- trifluoromethyl-phenyl]- ureidomethyl}-phenoxy)- pyridine-2-carboxylic acid methylamide | 140 | | 557.57 |
| 107 | 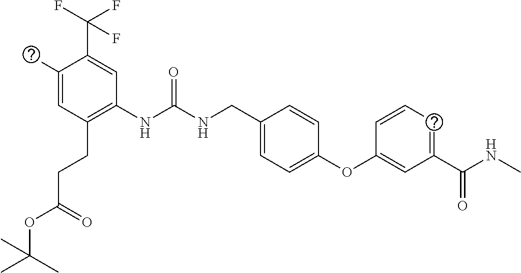 [SMILES: CNC(=O)c1cccc(Oc2ccc(CNC(=O)Nc3cc(C(F)(F)F)c(C)cc3CCC(=O)OC(C)(C)C)cc2)c1] | (5-Chloro-2-{3-[4-(2- methylcarbamoyl-pyridin- 4-yloxy)-benzyl]-ureido}-4- trifluoromethyl-phenoxy)- acetic acid tert-butyl ester | 22 | | 609.00 |
| 108 | 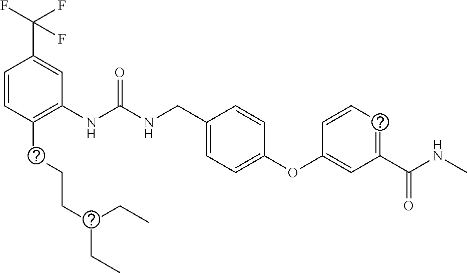 [SMILES: CCC(CC)CCCc1ccc(C(F)(F)F)cc1NC(=O)NCc1ccc(Oc2cccc(C(=O)NC)c2)cc1] | 4-(4-{3-[2-(2-Diethylamino- ethoxy)-5-trifluoromethyl- phenyl]-ureidomethyl}- phenoxy)-pyridine-2- carboxylic acid methylamide | 30 | | 559.59 |
| 109 | 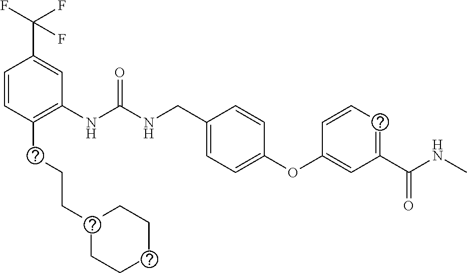 [SMILES: CNC(=O)c1cccc(Oc2ccc(CNC(=O)Nc3cc(C(F)(F)F)ccc3CCCC3CCCCC3)cc2)c1] | 4-(4-{3-[2-(2-Morpholin-4- yl-ethoxy)-5- trifluoromethyl-phenyl]- ureidomethyl}-phenoxy)- pyridine-2-carboxylic acid methylamide | 72 | | 573.57 |
| 110 | 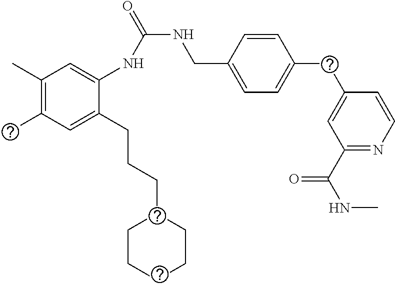 [SMILES: CNC(=O)c1cc(Cc2ccc(CNC(=O)Nc3cc(C)c(C)cc3CCCC3CCCCC3)cc2)ccn1] | 4-(4-{3-[4-Chloro-5-methyl- 2-(2-morpholin-4-yl- ethoxy)-phenyl]- ureidomethyl}-phenoxy)- pyridine-2-carboxylic acid methylamide | 290 | 130 | 554.05 |
| 111 | 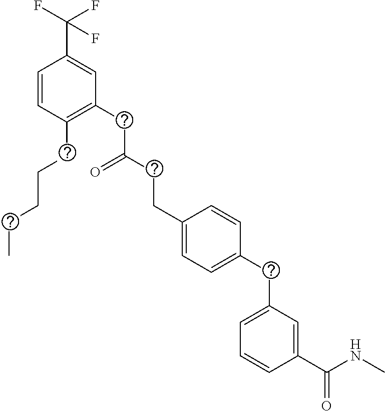 [SMILES: CCCCCc1ccc(C(F)(F)F)cc1CC(=O)CCc1ccc(Cc2cccc(C(=O)NC)c2)cc1] | 4-(4-{3-[2-(2-Methylamino- ethoxy)-5-trifluoromethyl- phenyl]-ureidomethyl}- phenoxy)-pyridine-2- carboxylic acid methylamide | 64 | 24 | 517.51 |
| 112 | 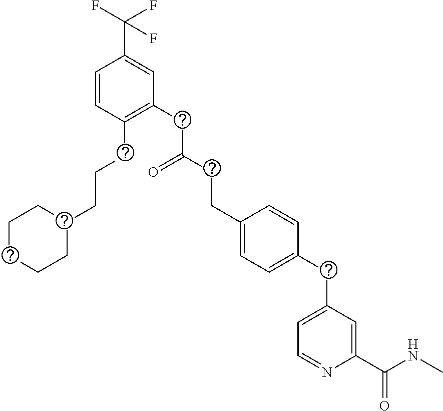 [SMILES: CNC(=O)c1cc(Cc2ccc(CCC(=O)Cc3cc(C(F)(F)F)ccc3CCCC3CCCCC3)cc2)ccn1] | 4-(4-{3-[2-(2-Piperazin-1- yl-ethoxy)-5- trifluoromethyl-phenyl]- ureidomethyl}-phenoxy)- pyridine-2-carboxylic acid methylamide | 130 | 47 | 572.59 |
| 113 | 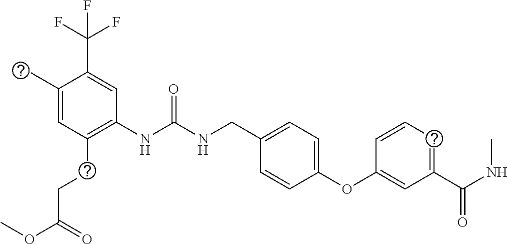 | (5-Chloro-2-{3-[4-(2- methylcarbamoyl-pyridin- 4-yloxy)-benzyl]-ureido}-4- trifluoromethyl-phenoxy)- acetic acid methyl ester | 57 | | 566.92 |
| 114 | 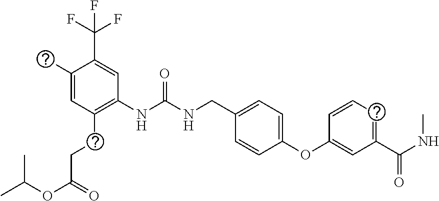 [SMILES: CNC(=O)c1cccc(Oc2ccc(CNC(=O)Nc3cc(C(F)(F)F)c(C)cc3CCC(=O)OC(C)C)cc2)c1] | (5-Chloro-2-{3-[4-(2- methylcarbamoyl-pyridin- 4-yloxy)-benzyl]-ureido}-4- trifluoromethyl-phenoxy)- acetic acid isopropyl ester | 24 | | 594.97 |
| 115 | 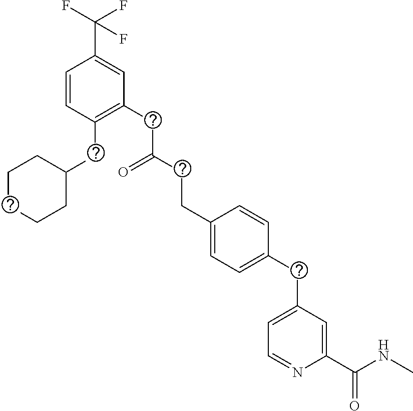 [SMILES: CNC(=O)c1cc(Cc2ccc(CCC(=O)Cc3cc(C(F)(F)F)ccc3CC3CCCCC3)cc2)ccn1] | 4-(4-{3-[2-(Piperidin-4- yloxy)-5-trifluoromethyl- phenyl]-ureidomethyl}- phenoxy)-pyridine-2- carboxylic acid methylamide | 20 | | 543.55 |
| 116 | 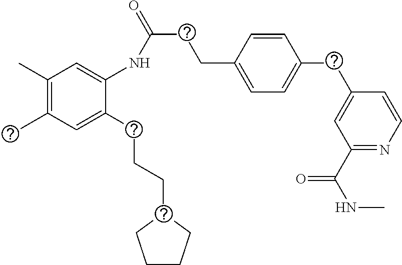 [SMILES: CNC(=O)c1cc(Cc2ccc(CCC(=O)Nc3cc(C)c(C)cc3CCCC3CCCC3)cc2)ccn1] | 4-(4-{3-[4-Chloro-5-methyl- 2-(2-pyrrolidin-1-yl- ethoxy)-phenyl]- ureidomethyl}-phenoxy)- pyridine-2-carboxylic acid methylamide | 410 | | 538.05 |
| 117 | 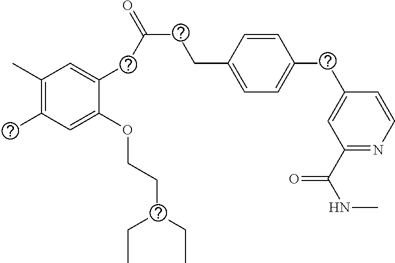 [SMILES: CCC(CC)CCOc1cc(C)c(C)cc1CC(=O)CCc1ccc(Cc2ccnc(C(=O)NC)c2)cc1] | 4-(4-{3-[4-Chloro-2-(2- diethylamino-ethoxy)-5- methyl-phenyl]- ureidomethyl}-phenoxy)- pyridine-2-carboxylic acid methylamide | 425 | | 540.06 |
| 118 | 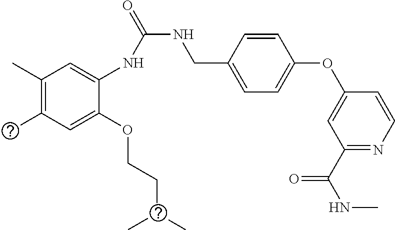 [SMILES: CNC(=O)c1cc(Oc2ccc(CNC(=O)Nc3cc(C)c(C)cc3OCCC(C)C)cc2)ccn1] | 4-(4-{3-[4-Chloro-2-(2- dimethylamino-ethoxy)-5- methyl-phenyl]- ureidomethyl}-phenoxy)- pyridine-2-carboxylic acid methylamide | 200 | 55 | 512.01 |
| 119 | 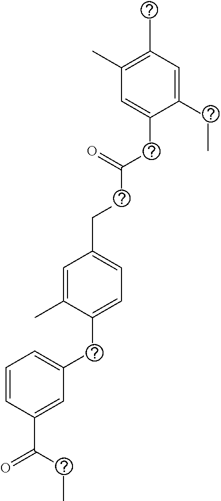 [SMILES: CCC(=O)c1cccc(Cc2ccc(CCC(=O)Cc3cc(C)c(C)cc3CC)cc2C)c1] | 4-{4-[3-(4-Chloro-2- methoxy-5-methyl-phenyl)- ureidomethyl]-2-methyl- phenoxy}-pyridine-2- carboxylic acid methylamide | 210 | | 468.94 |
| 120 | 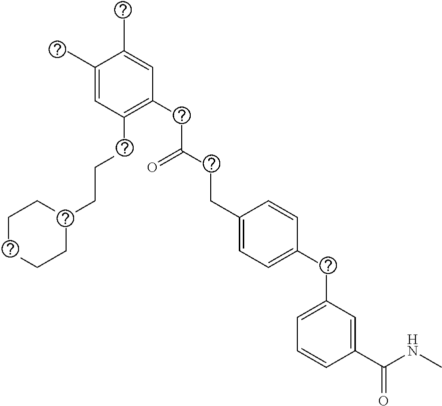 [SMILES: CNC(=O)c1cccc(Cc2ccc(CCC(=O)Cc3cc(C)c(C)cc3CCCC3CCCCC3)cc2)c1] | 4-(4-{3-[4-Chloro-5-methyl- 2-(2-piperazin-1-yl- ethoxy)-phenyl]- ureidomethyl}-phenoxy)- pyridine-2-carboxylic acid methylamide | 176 | | 553.06 |
| 121 | 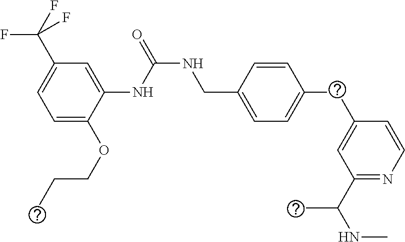 [SMILES: CCCOc1ccc(C(F)(F)F)cc1NC(=O)NCc1ccc(Cc2ccnc(C(C)NC)c2)cc1] | 4-(4-{3-[2-(2-Amino- ethoxy)-5-trifluoromethyl- phenyl]-ureidomethyl}- phenoxy)-pyridine-2- carboxylic acid methylamide | 34 | | 503.48 |
| 122 | 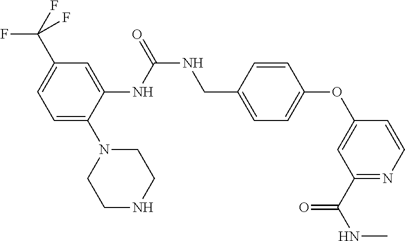 [SMILES: CNC(=O)c1cc(Oc2ccc(CNC(=O)Nc3cc(C(F)(F)F)ccc3N3CCNCC3)cc2)ccn1] | 4-{4-[3-(2-Piperazin-1-yl-5- trifluoromethyl-phenyl)- ureidomethyl]-phenoxy}- pyridine-2-carboxylic acid methylamide | | 135 | 528.53 |
| 123 | 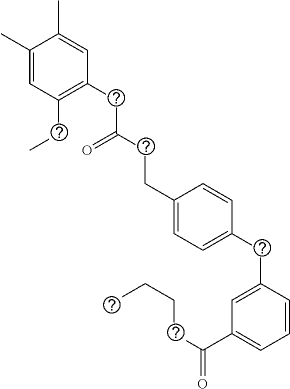 [SMILES: CCCCC(=O)c1cccc(Cc2ccc(CCC(=O)Cc3cc(C)c(C)cc3CC)cc2)c1] | 4-{4-[3-(4-Chloro-2- methoxy-5-methyl-phenyl)- ureidomethyl]-phenoxy}- pyridine-2-carboxylic acid (2-amino-ethyl)-amide | 3100 | | 483.96 |
| 124 | 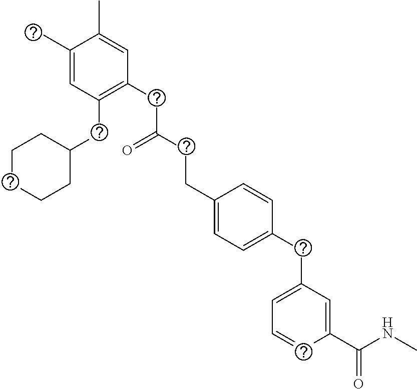 [SMILES: CNC(=O)c1cccc(Cc2ccc(CCC(=O)Cc3cc(C)c(C)cc3CC3CCCCC3)cc2)c1] | 4-(4-{3-[4-Chloro-5-methyl- 2-(piperidin-4-yloxy)- phenyl]-ureidomethyl}- phenoxy)-pyridine-2- carboxylic acid methylamide | 66 | 40 | 524.02 |
| 125 | 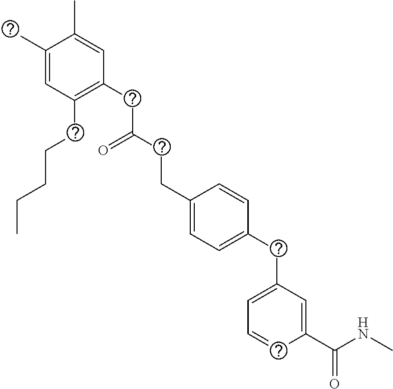 | 4-(4-{3-[4-Chloro-5-methyl- 2-(2-methylamino-ethoxy)- phenyl]-ureidomethyl}- phenoxy)-pyridine-2- carboxylic acid methylamide | 187 | 52 | 497.98 |
| 126 | 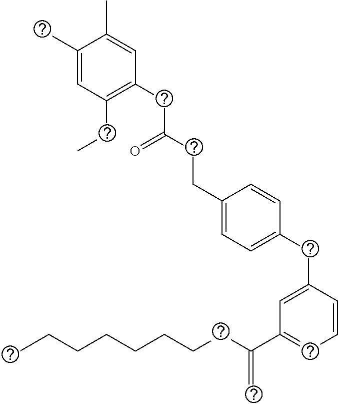 [SMILES: C=C(CCCCCCCC)c1cccc(Cc2ccc(CCC(=O)Cc3cc(C)c(C)cc3CC)cc2)c1] | 4-{4-[3-(4-Chloro-2- methoxy-5-methyl-phenyl)- ureidomethyl]-phenoxy}- pyridine-2-carboxylic acid (6-amino-hexyl)-amide | 150 | | 540.06 |
| 127 | 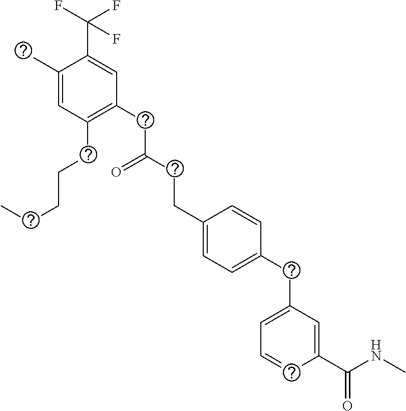 [SMILES: CCCCCc1cc(C)c(C(F)(F)F)cc1CC(=O)CCc1ccc(Cc2cccc(C(=O)NC)c2)cc1] | 4-(4-{3-[4-Chlorc~2-(2- methylamino-ethoxy)-5- trifluoromethyl-phenyl]- ureidomethyl}-phenoxy)- pyridine-2-carboxylic acid methylamide | 47 | 34 | 551.95 |
| 128 | 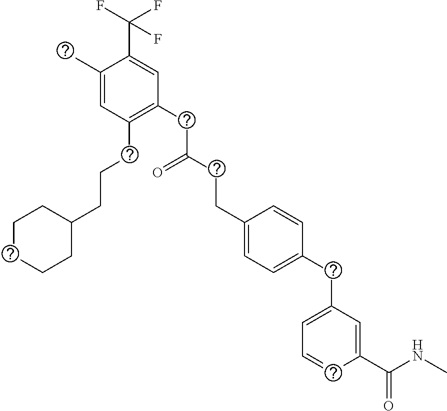 [SMILES: CNC(=O)c1cccc(Cc2ccc(CCC(=O)Cc3cc(C(F)(F)F)c(C)cc3CCCC3CCCCC3)cc2)c1] | 4-(4-{3-[4-Chloro-2-(2- piperazin-1-yl-ethoxy)-5- trifluoromethyl-phenyl]- ureidomethyl}-phenoxy)- pyridine-2-carboxylic acid methylamide | 91 | 63 | 607.03 |
| 129 | 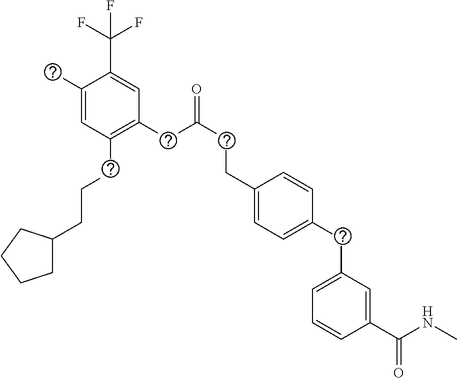 [SMILES: CNC(=O)c1cccc(Cc2ccc(CCC(=O)Cc3cc(C(F)(F)F)c(C)cc3CCCC3CCCC3)cc2)c1] | 4-(4-{3-[4-Chloro-2-(2- pyrrolidin-1-yl-ethoxy)-5- trifluoromethyl-phenyl]- ureidomethyl}-phenoxy)- pyridine-2-carboxylic acid methylamide | 250 | | 592.02 |
| 130 | 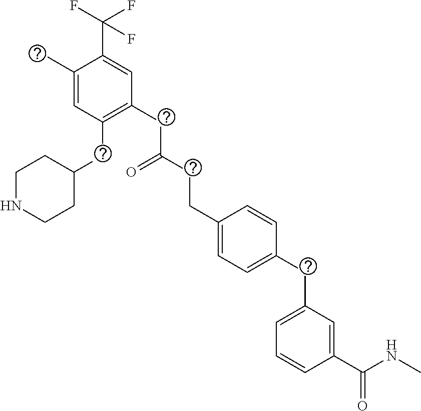 [SMILES: CNC(=O)c1cccc(Cc2ccc(CCC(=O)Cc3cc(C(F)(F)F)c(C)cc3CC3CCNCC3)cc2)c1] | 4-(4-{3-[4-Chloro-2- (piperidin-4-yloxy)-5- trifluoromethyl-phenyl]- ureidomethyl}-phenoxy)- pyridine-2-carboxylic acid methylamide | 34 | 24 | 577.99 |
| 131 | 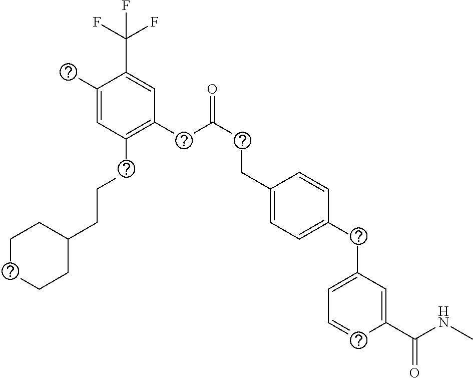 [SMILES: CNC(=O)c1cccc(Cc2ccc(CCC(=O)Cc3cc(C(F)(F)F)c(C)cc3CCCC3CCCCC3)cc2)c1] | 4-(4-{3-[4-Chloro-2-(2- morpholin-4-yl-ethoxy)-5- trifluoromethyl-phenyl]- ureidomethyl}-phenoxy)- pyridine-2-carboxylic acid methylamide | 96 | | 608.02 |
| 132 | 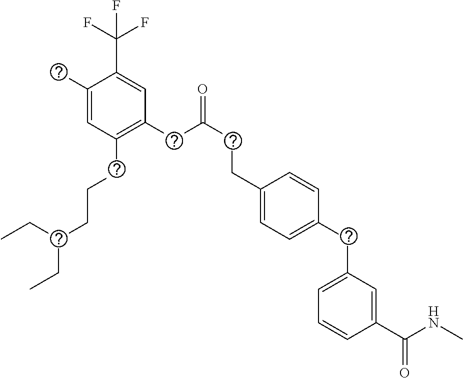 [SMILES: CCC(CC)CCCc1cc(C)c(C(F)(F)F)cc1CC(=O)CCc1ccc(Cc2cccc(C(=O)NC)c2)cc1] | 4-(4-{3-[4-Chloro-2-(2- diethylamino-ethoxy)-5- trifluoromethyl-phenyl]- ureidomethyl}-phenoxy)- pyridine-2-carboxylic acid methylamide | 125 | | 594.03 |
| 133 | 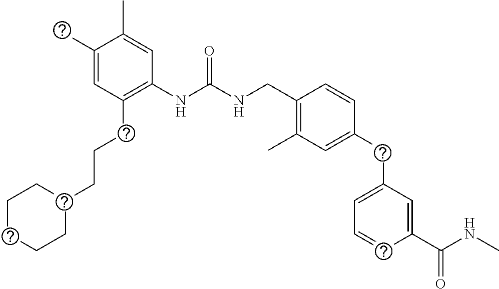 [SMILES: CNC(=O)c1cccc(Cc2ccc(CNC(=O)Nc3cc(C)c(C)cc3CCCC3CCCCC3)c(C)c2)c1] | 4-(4-{3-[4-Chloro-5-methyl- 2-(2-morpholin-4-yl- ethoxy)-phenyl]- ureidomethyl}-3-methyl- phenoxy)-pyridine-2- carboxylic acid methylamide | 2000 | | 568.07 |
| 134 | 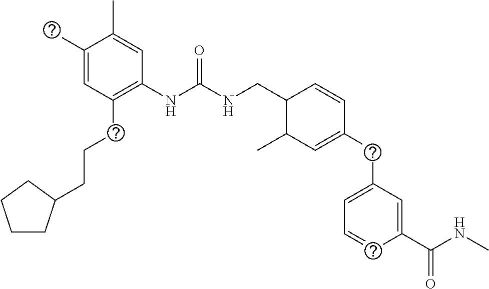 [SMILES: CNC(=O)c1cccc(CC2=CC(C)C(CNC(=O)Nc3cc(C)c(C)cc3CCCC3CCCC3)C=C2)c1] | 4-(4-{3-[4-Chloro-5-methyl- 2-(2-pyrrolidin-1-yl- ethoxy)-phenyl]- ureidomethyl}-3-methyl- phenoxy)-pyridine-2- carboxylic acid methylamide | 1200 | | 552.07 |
| 135 | 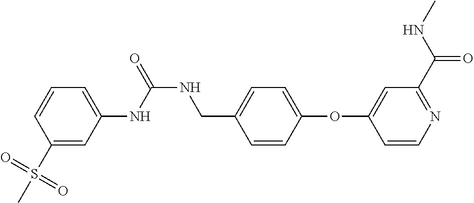 [SMILES: CNC(=O)c1cc(Oc2ccc(CNC(=O)Nc3cccc(S(C)(=O)=O)c3)cc2)ccn1] | 4-{4-[3-(3- Methanesulfonyl-phenyl)- ureidomethyl]-phenoxy}- pyridine-2-carboxylic acid methylamide | | 390 | 454.50 |
| 136 | 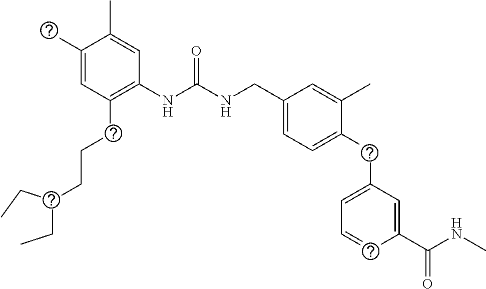 [SMILES: CCC(CC)CCCc1cc(C)c(C)cc1NC(=O)NCc1ccc(Cc2cccc(C(=O)NC)c2)c(C)c1] | 4-(4-{3-[4-Chloro-2-(2- diethylamino-ethoxy)-5- methyl-phenyl]- ureidomethyl}-2-methyl- phenoxy)-pyridine-2- carboxylic acid methylamide | 390 | | 554.09 |
| 137 | 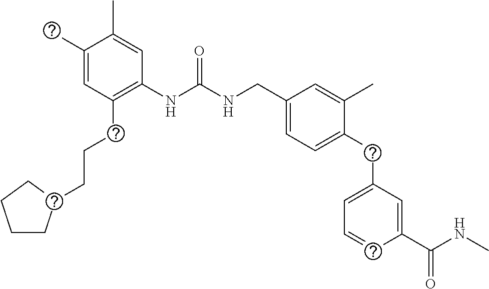 [SMILES: CNC(=O)c1cccc(Cc2ccc(CNC(=O)Nc3cc(C)c(C)cc3CCCC3CCCC3)cc2C)c1] | 4-(4-{3-[4-Chloro-5-methyl- 2-(2-pyrrolidin-1-yl- ethoxy)-phenyl]- ureidomethyl}-2-methyl- phenoxy)-pyridine-2- carboxylic acid methylamide | 140 | | 552.07 |
| 138 | 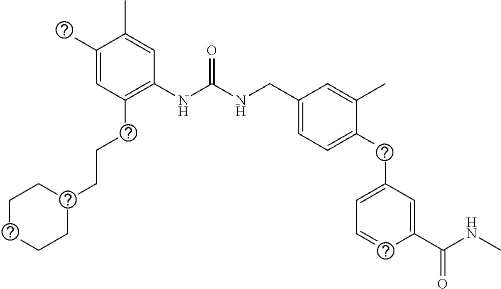 [SMILES: CNC(=O)c1cccc(Cc2ccc(CNC(=O)Nc3cc(C)c(C)cc3CCCC3CCCCC3)cc2C)c1] | 4-(4-{3-[4-Chloro-5-methyl- 2-(2-morpholin-4-yl- ethoxy)-phenyl]- ureidomethyl}-2-methyl- phenoxy)-pyridine-2- carboxylic acid methylamide | 310 | | 568.07 |
| 139 | 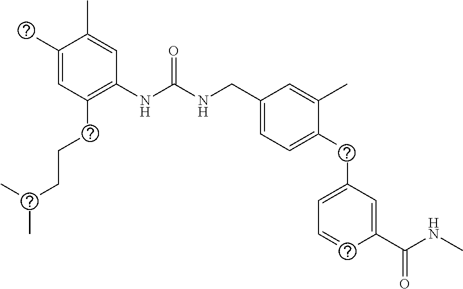 [SMILES: CNC(=O)c1cccc(Cc2ccc(CNC(=O)Nc3cc(C)c(C)cc3CCCC(C)C)cc2C)c1] | 4-(4-{3-[4-Chloro-2-(2- dimethylamino-ethoxy)-5- methyl-phenyl]- ureidomethyl}-2-methyl- phenoxy)-pyridine-2- carboxylic acid methylamide | 26 | 18 | 526.04 |
| 140 | 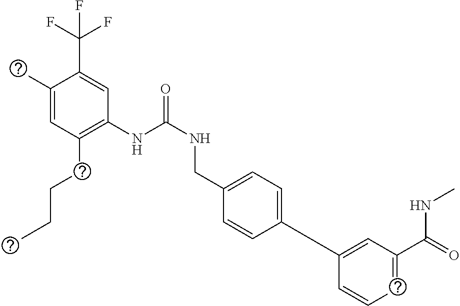 | 4-(4-{3-[2-(2-Amino- ethoxy)-4-chloro-5- trifluoromethyl-phenyl]- ureidomethyl}-phenoxy)- pyridine-2-carboxylic acid methylamide | 121 | | 537.93 |
| 141 | 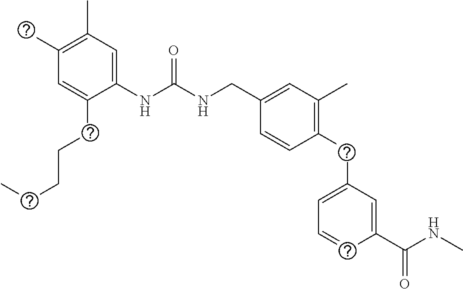 [SMILES: CCCCCc1cc(C)c(C)cc1NC(=O)NCc1ccc(Cc2cccc(C(=O)NC)c2)c(C)c1] | 4-(4-{3-[4-Chloro-5-methyl- 2-(2-methylamino-ethoxy)- phenyl]-ureidomethyl}-2- methyl-phenoxy)-pyridine- 2-carboxylic acid methylamide | 43 | 25 | 512.01 |
| 142 | 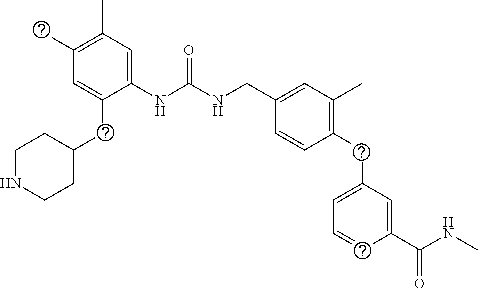 [SMILES: CNC(=O)c1cccc(Cc2ccc(CNC(=O)Nc3cc(C)c(C)cc3CC3CCNCC3)cc2C)c1] | 4-(4-{3-[4-Chloro-5-methyl- 2-(piperidin-4-yloxy)- phenyl]-ureidomethyl}-2- methyl-phenoxy)-pyridine- 2-carboxylic acid methylamide | 36 | 33 | 538.05 |
| 143 | 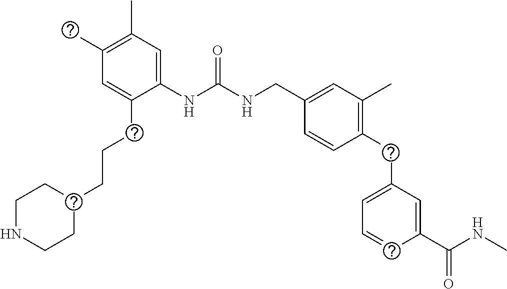 [SMILES: CNC(=O)c1cccc(Cc2ccc(CNC(=O)Nc3cc(C)c(C)cc3CCCC3CCNCC3)cc2C)c1] | 4-(4-{3-[4-Chloro-5-methyl- 2-(2-piperazin-1-yl- ethoxy)-phenyl]- ureidomethyl}-2-methyl- phenoxy)-pyridine-2- carboxylic acid methylamide | 77 | 66 | 567.09 |
| 144 | 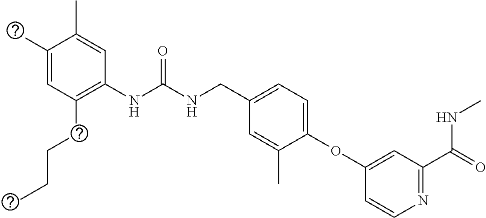 | 4-(4-{3-[2-(2-Amino- ethoxy)-4-chloro-5-methyl- phenyl]-ureidomethyl}-2- methyl-phenoxy)-pyridine- 2-carboxylic acid methylamide | 49 | 53 | 497.98 |
| 145 | 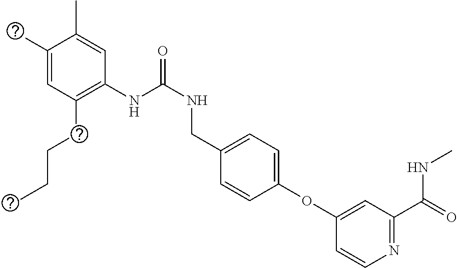 | 4-(4-{3-[2-(2-Amino- ethoxy)-4-chloro-5-methyl- phenyl]-ureidomethyl}- phenoxy)-pyridine-2- carboxylic acid methylamide | 91 | 65 | 483.96 |
| 146 | 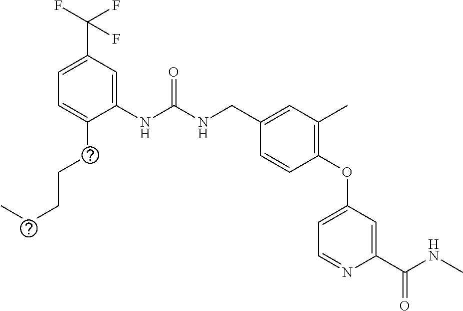 [SMILES: CCCCCc1ccc(C(F)(F)F)cc1NC(=O)NCc1ccc(Oc2ccnc(C(=O)NC)c2)c(C)c1] | 4-(2-Methyl-4-{3-[2-(2- methylamino-ethoxy)-5- trifluoromethyl-phenyl]- ureidomethyl}-phenoxy)- pyridine-2-carboxylic acid methylamide | 33 | 15 | 531.53 |
| 147 | 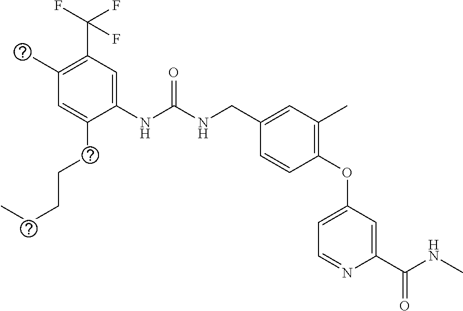 [SMILES: CCCCCc1cc(C)c(C(F)(F)F)cc1NC(=O)NCc1ccc(Oc2ccnc(C(=O)NC)c2)c(C)c1] | 4-(4-{3-[4-Chloro-2-(2- methylamino-ethoxy)-5- trifluoromethyl-phenyl]- ureidomethyl}-2-methyl- phenoxy)-pyridine-2- carboxylic acid methylamide | 41 | | 565.98 |
| 148 | 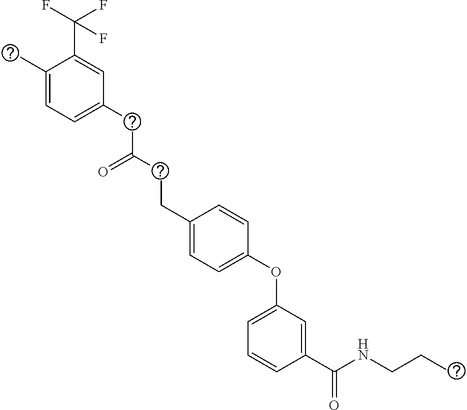 [SMILES: CCCNC(=O)c1cccc(Oc2ccc(CCC(=O)Cc3ccc(C)c(C(F)(F)F)c3)cc2)c1] | 4-{4-[3-(4-Chloro-3- trifluoromethyl-phenyl)- ureidomethyl]-phenoxy}- pyridine-2-carboxylic acid (2-amino-ethyl)-amide | | 785 | 507.90 |
| 149 | 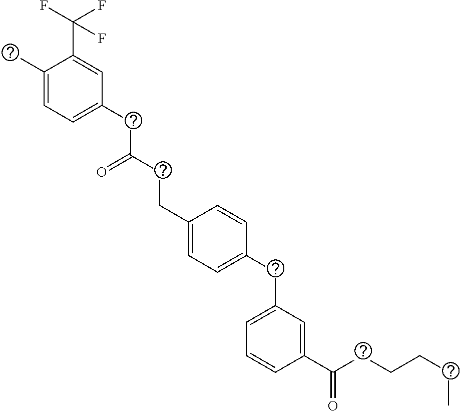 [SMILES: CCCCCC(=O)c1cccc(Cc2ccc(CCC(=O)Cc3ccc(C)c(C(F)(F)F)c3)cc2)c1] | 4-{4-[3-(4-Chloro-3- trifluoromethyl-phenyl)- ureidomethyl]-phenoxy}- pyridine-2-carboxylic acid (2-methylamino-ethyl)- amide | | 925 | 521.93 |
| 150 | 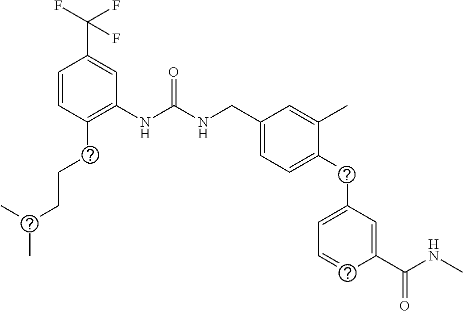 [SMILES: CNC(=O)c1cccc(Cc2ccc(CNC(=O)Nc3cc(C(F)(F)F)ccc3CCCC(C)C)cc2C)c1] | 4-(4-{3-[2-(2- Dimethylamino-ethoxy)-5- trifluoromethyl-phenyl]- ureidomethyl}-2-methyl- phenoxy)-pyridine-2- carboxylic acid methylamide | 42 | 10 | 545.56 |
| 151 | 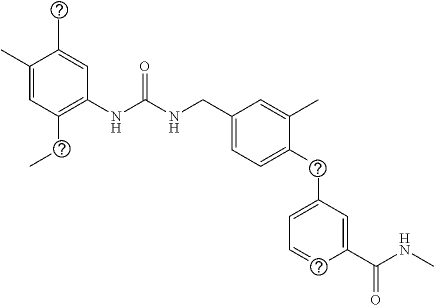 | 4-{4-[3-(5-Chloro-2- methoxy-4-methyl-phenyl)- ureidomethyl]-2-methyl- phenoxy}-pyridine-2- carboxylic acid methylamide | 100 | | 468.94 |
| 152 | 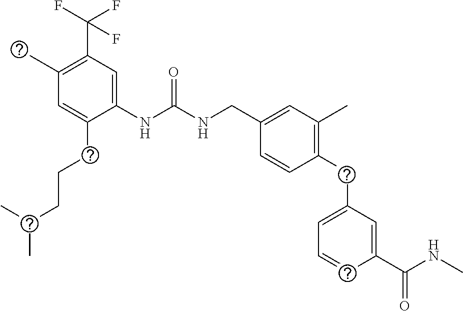 [SMILES: CNC(=O)c1cccc(Cc2ccc(CNC(=O)Nc3cc(C(F)(F)F)c(C)cc3CCCC(C)C)cc2C)c1] | 4-(4-{3-[4-Chloro-2-(2- dimethylamino-ethoxy)-5- trifluoromethyl-phenyl]- ureidomethyl}-2-methyl- phenoxy)-pyridine-2- carboxylic acid methylamide | 25 | | 580.01 |
| 153 | 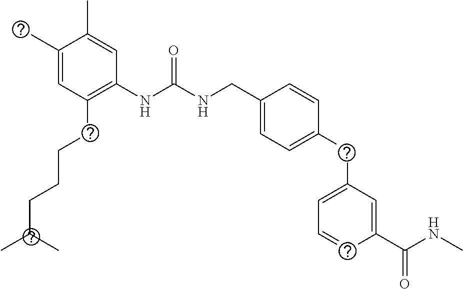 [SMILES: CNC(=O)c1cccc(Cc2ccc(CNC(=O)Nc3cc(C)c(C)cc3CCCCC(C)C)cc2)c1] | 4-(4-{3-[4-Chloro-2-(3- dimethylamino-propoxy)-5- methyl-phenyl]- ureidomethyl}-phenoxy)- pyridine-2-carboxylic acid methylamide | 140 | 55 | 526.04 |
| 154 | 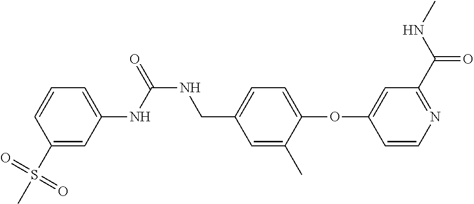 [SMILES: CNC(=O)c1cc(Oc2ccc(CNC(=O)Nc3cccc(S(C)(=O)=O)c3)cc2C)ccn1] | 4-{4-[3-(3- Methanesulfonyl-phenyl)- ureidomethyl]-2-methyl- phenoxy}-pyridine-2- carboxylic acid methylamide | | 1250 | 468.53 |
| 155 | 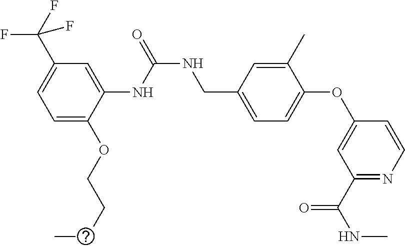 [SMILES: CCCCOc1ccc(C(F)(F)F)cc1NC(=O)NCc1ccc(Oc2ccnc(C(=O)NC)c2)c(C)c1] | 4-(4-{3-[2-(2-Amino- ethoxy)-5-trifluoromethyl- phenyl]-ureidomethyl}-2- methyl-phenoxy)-pyridine- 2-carboxylic acid methylamide | 40 | 18 | 517.51 |
| 156 | 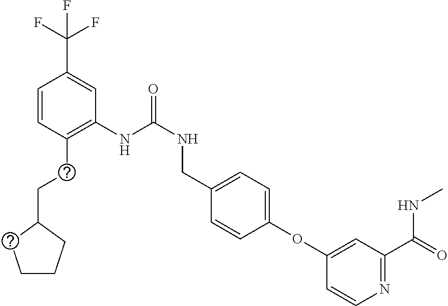 [SMILES: CNC(=O)c1cc(Oc2ccc(CNC(=O)Nc3cc(C(F)(F)F)ccc3CCC3CCCC3)cc2)ccn1] | 4-(4-{3-[2-(Pyrrolidin-2- ylmethoxy)-5- trifluoromethyl-phenyl]- ureidomethyl}-phenoxy)- pyridine-2-carboxylic acid methylamide | 43 | 16 | 543.55 |
| 157 | 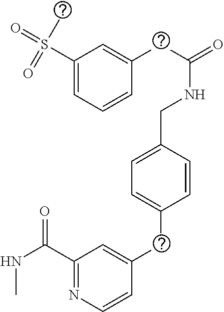 [SMILES: CNC(=O)c1cc(Cc2ccc(CNC(=O)Cc3cccc(S(C)(=O)=O)c3)cc2)ccn1] | 4-{4-[3-(3-Sulfamoyl- phenyl)-ureidomethyl]- phenoxy}-pyridine-2- carboxylic acid methylamide | | 500 | 455.49 |
| 158 | 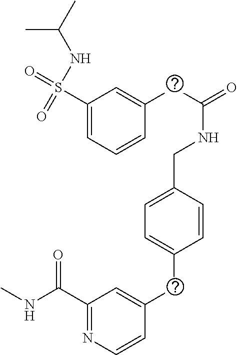 [SMILES: CNC(=O)c1cc(Cc2ccc(CNC(=O)Cc3cccc(S(=O)(=O)NC(C)C)c3)cc2)ccn1] | 4-{4-[3-(3- Isopropylsulfamoyl- phenyl)-ureidomethyl]- phenoxy}-pyridine-2- carboxylic acid methylamide | | 100000 | 497.57 |
| 159 | 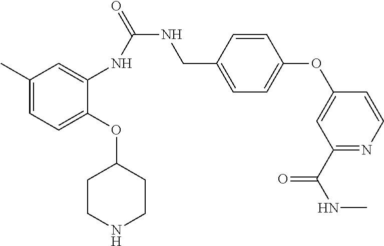 [SMILES: CNC(=O)c1cc(Oc2ccc(CNC(=O)Nc3cc(C)ccc3OC3CCNCC3)cc2)ccn1] | 4-(4-{3-[5-Methyl-2- (piperidin-4-yloxy)-phenyl]- ureidomethyl}-phenoxy)- pyridine-2-carboxylic acid methylamide | 83 | | 489.58 |
| 160 | 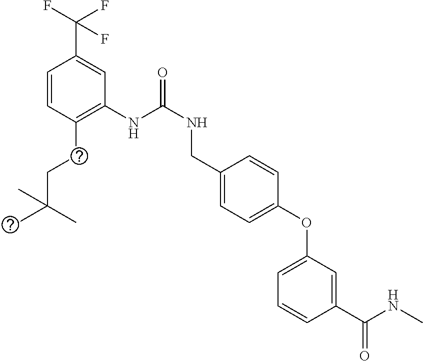 [SMILES: CNC(=O)c1cccc(Oc2ccc(CNC(=O)Nc3cc(C(F)(F)F)ccc3CCC(C)(C)C)cc2)c1] | 4-(4-{3-[2-(2-Amino-2- methyl-propoxy)-5- trifluoromethyl-phenyl]- ureidomethyl}-phenoxy)- pyridine-2-carboxylic acid methylamide | 360 | 95 | 531.53 |
| 161 | 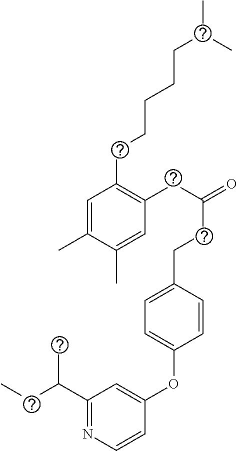 [SMILES: CCC(C)c1cc(Oc2ccc(CCC(=O)Cc3cc(C)c(C)cc3CCCCCC(C)C)cc2)ccn1] | 4-(4-{3-[4-Chloro-2-(4- dimethylamino-butoxy)-5- methyl-phenyl]- ureidomethyl}-phenoxy)- pyridine-2-carboxylic acid methylamide | 160 | | 540.06 |
| 162 | 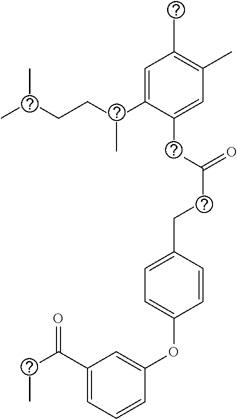 [SMILES: CCC(=O)c1cccc(Oc2ccc(CCC(=O)Cc3cc(C)c(C)cc3C(C)CCC(C)C)cc2)c1] | 4-[4-(3-{4-Chloro-2-[(2- dimethylamino-ethyl)- methyl-amino]-5-methyl- phenyl}-ureidomethyl)- phenoxy]-pyridine-2- carboxylic acid methylamide | | 1600 | 525.05 |
| 163 | 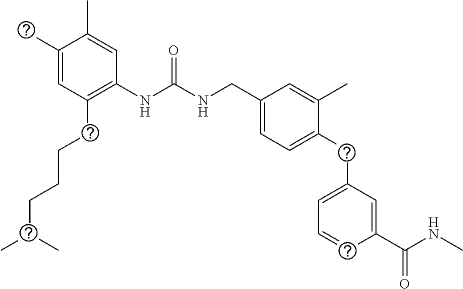 [SMILES: CNC(=O)c1cccc(Cc2ccc(CNC(=O)Nc3cc(C)c(C)cc3CCCCC(C)C)cc2C)c1] | 4-(4-{3-[4-Chloro-2-(3- dimethylamino-propoxy)-5- methyl-phenyl]- ureidomethyl}-2-methyl- phenoxy)-pyridine-2- carboxylic acid methylamide | 40 | 16 | 540.06 |
| 164 | 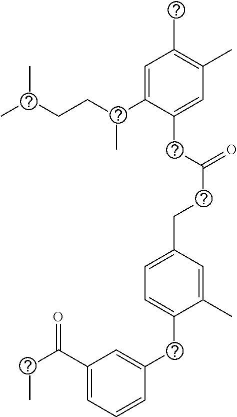 [SMILES: CCC(=O)c1cccc(Cc2ccc(CCC(=O)Cc3cc(C)c(C)cc3C(C)CCC(C)C)cc2C)c1] | 4-[4-(3-{4-Chloro-2-[(2- dimethylamino-ethyl)- methyl-amino]-5-methyl- phenyl}-ureidomethyl)-2- methyl-phenoxy]-pyridine- 2-carboxylic acid methylamide | | 300 | 539.08 |
| 165 | 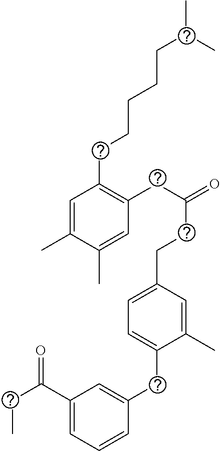 [SMILES: CCC(=O)c1cccc(Cc2ccc(CCC(=O)Cc3cc(C)c(C)cc3CCCCCC(C)C)cc2C)c1] | 4-(4-{3-[4-Chloro-2-(4- dimethylamino-butoxy)-5- methyl-phenyl]- ureidomethyl}-2-methyl- phenoxy)-pyridine-2- carboxylic acid methylamide | 210 | | 554.09 |
| 166 | 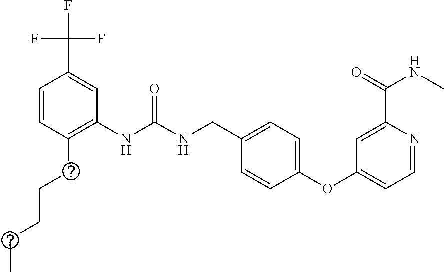 [SMILES: CCCCCc1ccc(C(F)(F)F)cc1NC(=O)NCc1ccc(Oc2ccnc(C(=O)NC)c2)cc1] | 4-(4-{3-[2-(2-Methoxy- ethoxy)-5-trifluoromethyl- phenyl]-ureidomethyl}- phenoxy)-pyridine-2- carboxylic acid methylamide | 140 | | 518.49 |
| 167 | 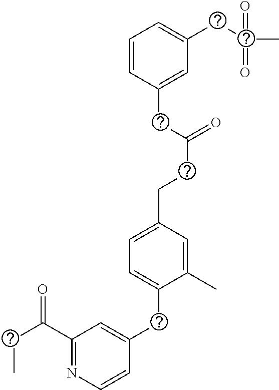 | 4-{4-[3-(3- Methanesulfonylamino- phenyl)-ureidomethyl]-2- methyl-phenoxy}-pyridine- 2-carboxylic acid methylamide | | 1500 | 483.55 |
| 168 | 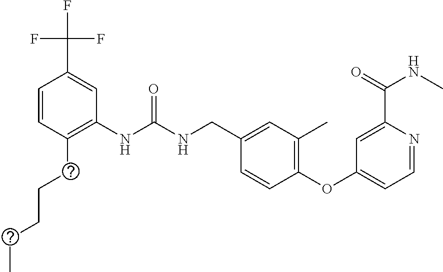 [SMILES: CCCCCc1ccc(C(F)(F)F)cc1NC(=O)NCc1ccc(Oc2ccnc(C(=O)NC)c2)c(C)c1] | 4-(4-{3-[2-(2-Methoxy- ethoxy)-5-trifluoromethyl- phenyl]-ureidomethyl}-2- methyl-phenoxy)-pyridine- 2-carboxylic acid methylamide | 130 | | 532.52 |
| 169 | 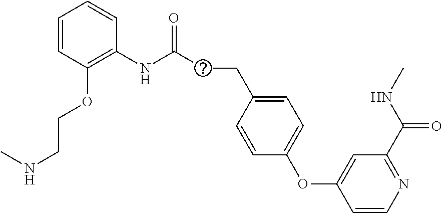 [SMILES: CNCCOc1ccccc1NC(=O)CCc1ccc(Oc2ccnc(C(=O)NC)c2)cc1] | 4-(4-{3-[2-(2-Methylamino- ethoxy)-phenyl]- ureidomethyl}-phenoxy)- pyridine-2-carboxylic acid methylamide | 2800 | 1900 | 449.51 |
| 170 | 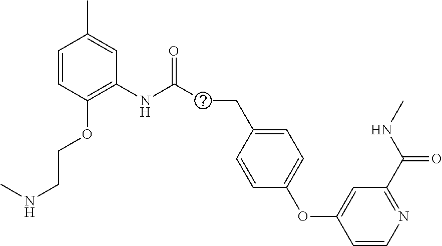 [SMILES: CNCCOc1ccc(C)cc1NC(=O)CCc1ccc(Oc2ccnc(C(=O)NC)c2)cc1] | 4-(4-{3-[5-Methyl-2-(2- methylamino-ethoxy)- phenyl]-ureidomethyl}- phenoxy)-pyridine-2- carboxylic acid methylamide | 310 | 195 | 463.54 |
| 171 | 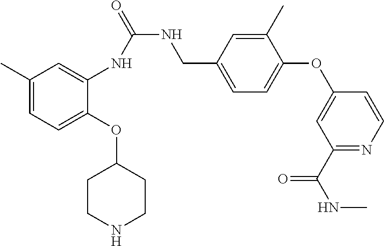 [SMILES: CNC(=O)c1cc(Oc2ccc(CNC(=O)Nc3cc(C)ccc3OC3CCNCC3)cc2C)ccn1] | 4-(2-Methyl-4-{3-[5-methyl- 2-(piperidin-4-yloxy)- phenyl]-ureidomethyl}- phenoxy)-pyridine-2- carboxylic acid methylamide | 47 | | 503.60 |
| 172 | 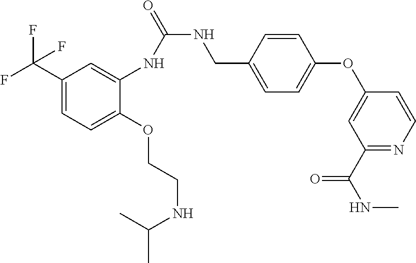 [SMILES: CNC(=O)c1cc(Oc2ccc(CNC(=O)Nc3cc(C(F)(F)F)ccc3OCCNC(C)C)cc2)ccn1] | 4-(4-{3-[2-(2- lsopropylamino-ethoxy)-5- trifluoromethyl-phenyl]- ureidomethyl}-phenoxy)- pyridine-2-carboxylic acid methylamide | 140 | 31 | 545.56 |
| 173 | 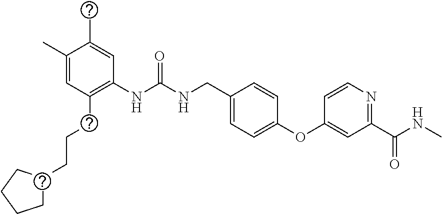 [SMILES: CNC(=O)c1cc(Oc2ccc(CNC(=O)Nc3cc(C)c(C)cc3CCCC3CCCC3)cc2)ccn1] | 4-(4-{3-[5-Chloro-4-methyl- 2-(2-pyrrolidin-1-yl- ethoxy)-phenyl]- ureidomethyl}-phenoxy)- pyridine-2-carboxylic acid methylamide | 260 | | 538.05 |
| 174 | 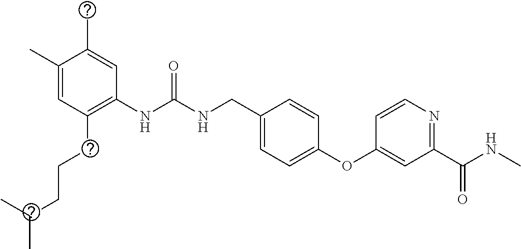 [SMILES: CNC(=O)c1cc(Oc2ccc(CNC(=O)Nc3cc(C)c(C)cc3CCCC(C)C)cc2)ccn1] | 4-(4-{3-[5-Chloro-2-(2- dimethylamino-ethoxy)-4- methyl-phenyl]- ureidomethyl}-phenoxy)- pyridine-2-carboxylic acid methylamide | 140 | 28 | 512.01 |
| 175 | 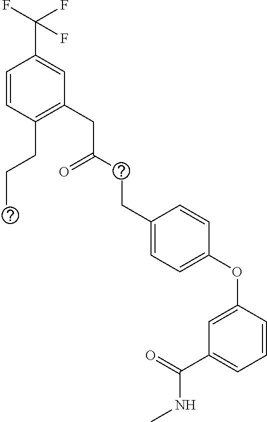 [SMILES: CCCc1ccc(C(F)(F)F)cc1CC(=O)CCc1ccc(Oc2cccc(C(=O)NC)c2)cc1] | 4-(4-{3-[2-(2-Amino-ethyl)- 5-trifluoromethyl-phenyl]- ureidomethyl}-phenoxy)- pyridine-2-carboxylic acid methylamide | | 1300 | 487.48 |
| 176 | 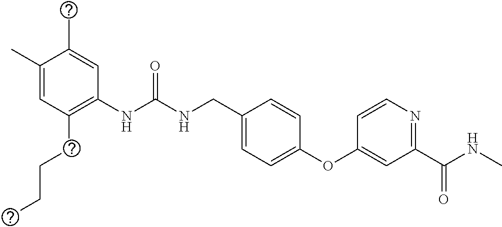 | 4-(4-{3-[2-(2-Amino- ethoxy)-5-chloro-4-methyl- phenyl]-ureidomethyl}- phenoxy)-pyridine-2- carboxylic acid methylamide | 27 | | 483.96 |
| 177 | 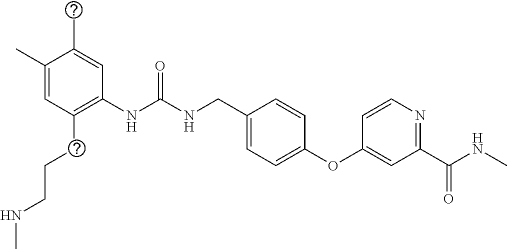 | 4-(4-{3-[5-Chloro-4-methyl- 2-(2-methylamino-ethoxy)- phenyl]-ureidomethyl}- phenoxy)-pyridine-2- carboxylic acid methylamide | 74 | 27 | 497.98 |
| 178 | 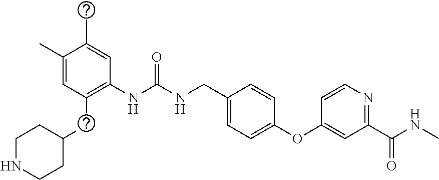 [SMILES: CNC(=O)c1cc(Oc2ccc(CNC(=O)Nc3cc(C)c(C)cc3CC3CCNCC3)cc2)ccn1] | 4-(4-{3-[5-Chloro-4-methyl- 2-(piperidin-4-yloxy)- phenyl]-ureidomethyl}- phenoxy)-pyridine-2- carboxylic acid methylamide | 35 | 25 | 524.02 |
| 179 | 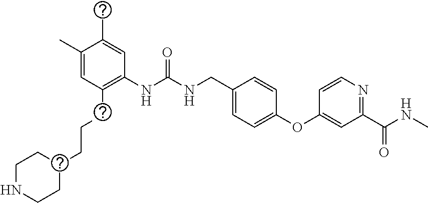 [SMILES: CNC(=O)c1cc(Oc2ccc(CNC(=O)Nc3cc(C)c(C)cc3CCCC3CCNCC3)cc2)ccn1] | 4-(4-{3-[5-Chloro-4-methyl- 2-(2-piperazin-1-yl- ethoxy)-phenyl]- ureidomethyl}-phenoxy)- pyridine-2-carboxylic acid methylamide | 160 | 38 | 553.06 |
| 180 | 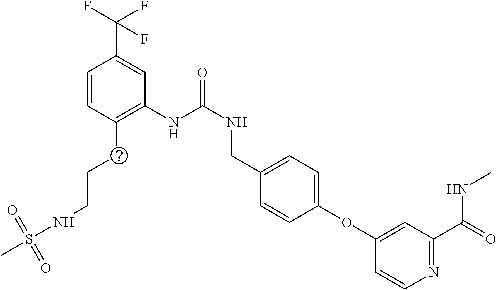 | 4-(4-{3-[2-(2- Methanesulfonylamino- ethoxy)-5-trifluoromethyl- phenyl]-ureidomethyl}- phenoxy)-pyridine-2- carboxylic acid methylamide | 32 | | 581.57 |
| 181 | 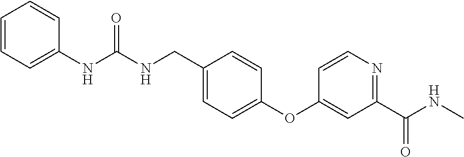 [SMILES: CNC(=O)c1cc(Oc2ccc(CNC(=O)Nc3ccccc3)cc2)ccn1] | 4-{4-[(3-Phenyl-ureido)- methyl]-phenoxy}-pyridine- 2-carboxylic acid methylamide | 4900 | | 376.42 |
| 182 | 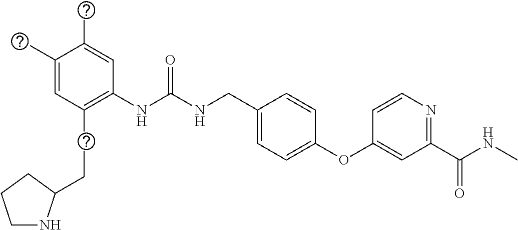 [SMILES: CNC(=O)c1cc(Oc2ccc(CNC(=O)Nc3cc(C)c(C)cc3CCC3CCCN3)cc2)ccn1] | 4-(4-{3-[5-Chloro-4-methyl- 2-(pyrrolidin-2-ylmethoxy)- phenyl]-ureidomethyl}- phenoxy)-pyridine-2- carboxylic acid methylamide | 59 | 30 | 524.02 |
| 183 | 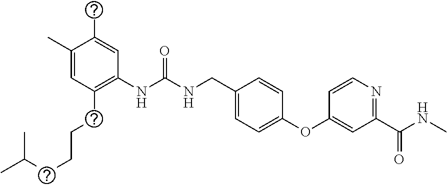 [SMILES: CNC(=O)c1cc(Oc2ccc(CNC(=O)Nc3cc(C)c(C)cc3CCCCC(C)C)cc2)ccn1] | 4-(4-{3-[5-Chloro-2-(2- isopropylamino-ethoxy)-4- methyl-phenyl]- ureidomethyl}-phenoxy)- pyridine-2-carboxylic acid methylamide | 89 | 35 | 526.04 |
| 184 | 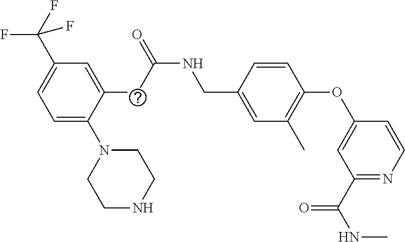 [SMILES: CNC(=O)c1cc(Oc2ccc(CNC(=O)Cc3cc(C(F)(F)F)ccc3N3CCNCC3)cc2C)ccn1] | 4-{2-Methyl-4-[3-(2- piperazin-1-yl-5- trifluoromethyl-phenyl)- ureidomethyl]-phenoxy}- pyridine-2-carboxylic acid methylamide | | 76 | 542.56 |
| 185 | 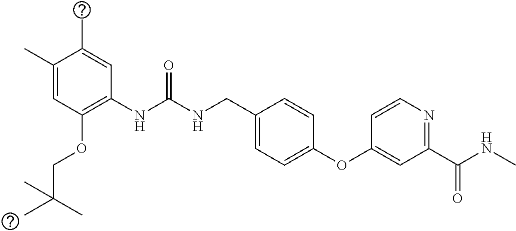 | 4-(4-{3-[2-(2-Amino-2- methyl-propoxy)-5-chloro- 4-methyl-phenyl]- ureidomethyl}-phenoxy)- pyridine-2-carboxylic acid methylamide | 460 | 175 | 512.01 |
| 186 | 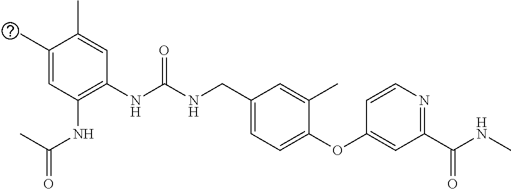 | 4-{4-[3-(2-Acetylamino-4- chloro-5-methyl-phenyl)- ureidomethyl]-2-methyl- phenoxy}-pyridine-2- carboxylic acid methylamide | | 330 | 495.97 |
| 187 | 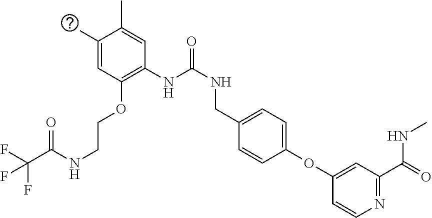 [SMILES: CNC(=O)c1cc(Oc2ccc(CNC(=O)Nc3cc(C)c(C)cc3OCCNC(=O)C(F)(F)F)cc2)ccn1] | 4-[4-(3-{4-Chloro-5-methyl- 2-[2-(2,2,2-trifluoro- acetylamino)-ethoxy]- phenyl}-ureidomethyl)- phenoxy]-pyridine-2- carboxylic acid methylamide | 660 | | 579.96 |
| 188 | 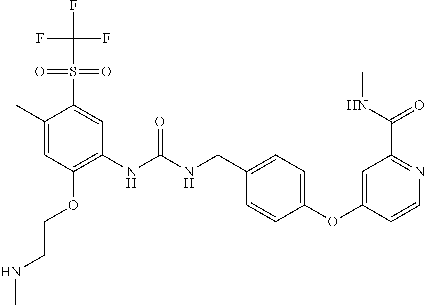 [SMILES: CNCCOc1cc(C)c(S(=O)(=O)C(F)(F)F)cc1NC(=O)NCc1ccc(Oc2ccnc(C(=O)NC)c2)cc1] | 4-(4-{3-[2-(2-Methylamino- ethoxy)-5- trifluoromethanesulfonyl- phenyl]-ureidomethyl}- phenoxy)-pyridine-2- carboxylic acid methylamide | 710 | 320 | 581.57 |
| 189 | 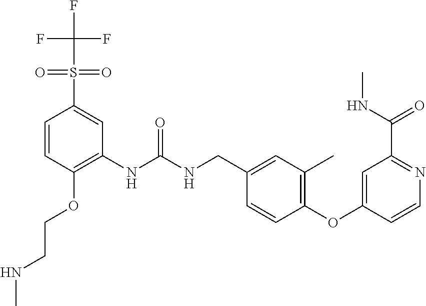 [SMILES: CNCCOc1ccc(S(=O)(=O)C(F)(F)F)cc1NC(=O)NCc1ccc(Oc2ccnc(C(=O)NC)c2)c(C)c1] | 4-(2-Methyl-4-{3-[2-(2- methylamino-ethoxy)-5- trifluoromethanesulfonyl- phenyl]-ureidomethyl}- phenoxy)-pyridine-2- carboxylic acid methylamide | 270 | 115 | 595.60 |
| 190 | 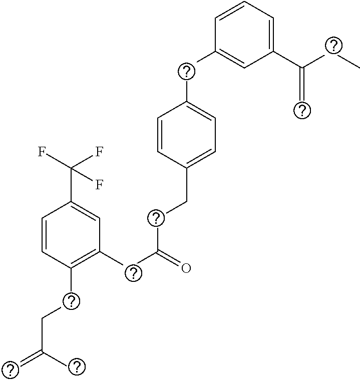 [SMILES: C=C(C)CCc1ccc(C(F)(F)F)cc1CC(=O)CCc1ccc(Cc2cccc(C(=C)CC)c2)cc1] | 4-{4-[3-(2- Carbamoylmethoxy-5- trifluoromethyl-phenyl)- ureidomethyl]-phenoxy}- pyridine-2-carboxylic acid methylamide | 120 | 40 | 517.46 |
| 191 | 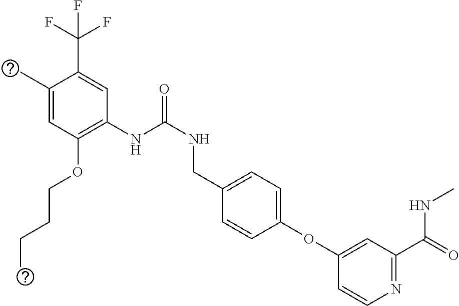 | 4-(4-{3-[2-(3-Amino- propoxy)-4-chloro-5- trifluoromethyl-phenyl]- ureidomethyl}-phenoxy)- pyridine-2-carboxylic acid methylamide | 590 | 106 | 551.95 |
| 192 | 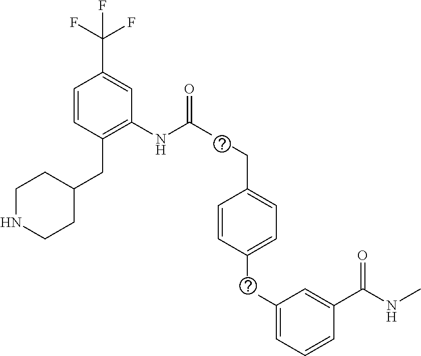 [SMILES: CNC(=O)c1cccc(Cc2ccc(CCC(=O)Nc3cc(C(F)(F)F)ccc3CC3CCNCC3)cc2)c1] | 4-{4-[3-(2-Piperazin-1- ylmethyl-5-trifluoromethyl- phenyl]-ureidomethyl]- phenoxy}-pyridine-2- carboxylic acid methylamide | | 1150 | 542.56 |
| 193 | 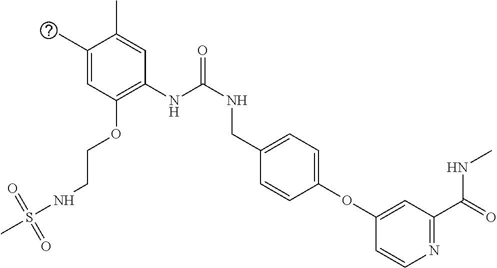 | 4-(4-{3-[4-Chloro-2-(2- methanesulfonylamino- ethoxy)-5-methyl-phenyl]- ureidomethyl}-phenoxy)- pyridine-2-carboxylic acid methylamide | 180 | | 562.04 |
| 194 | 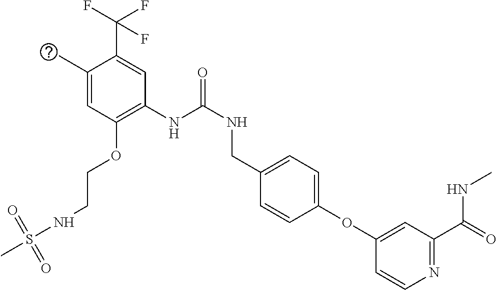 | 4-(4-{3-[4-Chloro-2-(2- methanesulfonylamino- ethoxy)-5-trifluoromethyl- phenyl]-ureidomethyl}- phenoxy)-pyridine-2- carboxylic acid methylamide | 64 | | 616.01 |
| 195 | 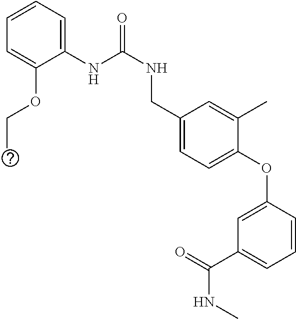 [SMILES: CCOc1ccccc1NC(=O)NCc1ccc(Oc2cccc(C(=O)NC)c2)c(C)c1] | 4-(4-{3-[2-(2-Hydroxy- ethyl)-phenyl]- ureidomethyl}-2-methyl- phenoxy)-pyridine-2- carboxylic acid methylamide | | 15000 | 434.50 |
| 196 | 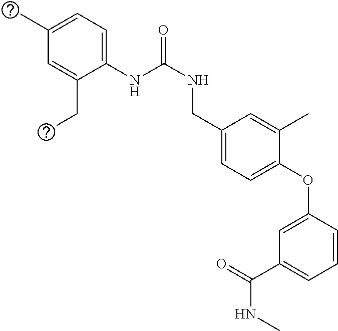 [SMILES: CCc1cc(C)ccc1NC(=O)NCc1ccc(Oc2cccc(C(=O)NC)c2)c(C)c1] | 4-{4-[3-(2-Hydroxymethyl- 4-methyl-phenyl)- ureidomethyl]-2-methyl- phenoxy}-pyridine-2- carboxylic acid methylamide | | 17650 | 434.50 |
| 197 | 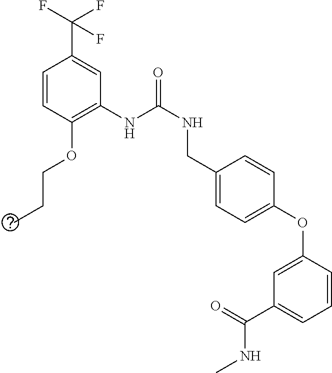 [SMILES: CCCOc1ccc(C(F)(F)F)cc1NC(=O)NCc1ccc(Oc2cccc(C(=O)NC)c2)cc1] | 4-(4-{3-[2-(2-Hydroxy- ethoxy)-5-trifluoromethyl- phenyl]-ureidomethyl}- phenoxy)-pyridine-2- carboxylic acid methylamide | 96 | | 504.46 |
| 198 | 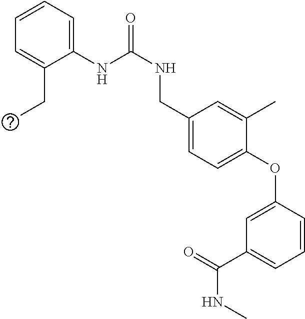 [SMILES: CCc1ccccc1NC(=O)NCc1ccc(Oc2cccc(C(=O)NC)c2)c(C)c1] | 4-{4-[3-(2-Hydroxymethyl- phenyl)-ureidomethyl]-2- methyl-phenoxy]-pyridine- 2-carboxylic acid methylamide | | 7500 | 420.47 |
| 199 | 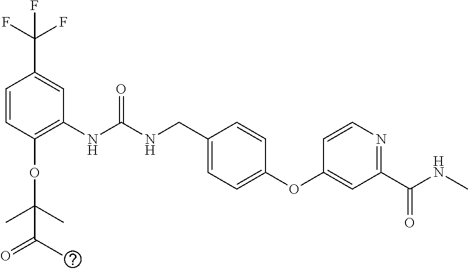 [SMILES: CNC(=O)c1cc(Oc2ccc(CNC(=O)Nc3cc(C(F)(F)F)ccc3OC(C)(C)C(C)=O)cc2)ccn1] | 4-(4-{3-[2-(1-Carbamoyl-1- methyl-ethoxy)-5- trifluoromethyl-phenyl]- ureidomethyl}-phenoxy)- pyridine-2-carboxylic acid methylamide | 100000 | | 545.52 |
| 200 | 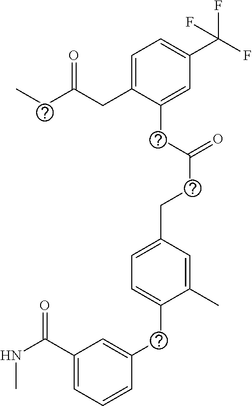 [SMILES: CCC(=O)Cc1ccc(C(F)(F)F)cc1CC(=O)CCc1ccc(Cc2cccc(C(=O)NC)c2)c(C)c1] | 4-{2-Methyl-4-[3-(2- methylcarbamoylmethyl-5- trifluoromethyl-phenyl)- ureidomethyl]-phenoxy}- pyridine-2-carboxylic acid methylamide | | 455 | 529.52 |
| 201 | 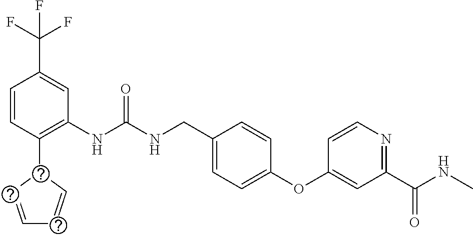 [SMILES: CNC(=O)c1cc(Oc2ccc(CNC(=O)Nc3cc(C(F)(F)F)ccc3C3C=CC=C3)cc2)ccn1] | 4-{4-[3-(2-[1,2,4]Triazol-1- yl-5-trifluoromethyl- phenyl)-ureidomethyl]- phenoxy}-pyridine-2- carboxylic acid methylamide | | 41 | 511.46 |
| 202 | 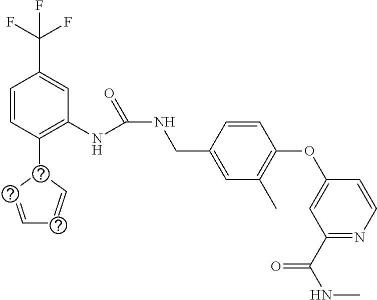 [SMILES: CNC(=O)c1cc(Oc2ccc(CNC(=O)Nc3cc(C(F)(F)F)ccc3C3C=CC=C3)cc2C)ccn1] | 4-{2-Methyl-4-[3-(2- [1,2,4]triazol-1-yl-5- trifluoromethyl-phenyl)- ureidomethyl]-phenoxy}- pyridine-2-carboxylic acid methylamide | | 14 | 525.49 |
| 203 | 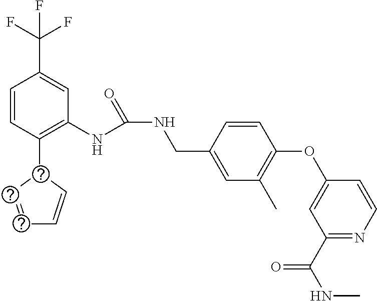 [SMILES: CNC(=O)c1cc(Oc2ccc(CNC(=O)Nc3cc(C(F)(F)F)ccc3C3C=CC=C3)cc2C)ccn1] | 4-{2-Methyl-4-[3-(2- [1,2,3]triazol-1-yl-5- trifluoromethyl-phenyl)- ureidomethyl]-phenoxy}- pyridine-2-carboxylic acid methylamide | | 19 | 525.49 |
| 204 | 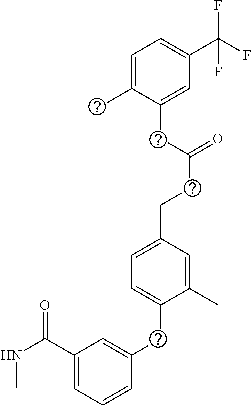 [SMILES: CNC(=O)c1cccc(Cc2ccc(CCC(=O)Cc3cc(C(F)(F)F)ccc3C)cc2C)c1] | 4-{4-[3-(2-Hydroxy-5- trifluoromethyl-phenyl)- ureidomethyl]-2-methyl- phenoxy}-pyridine-2- carboxylic acid methylamide | | 38 | 474.44 |
| 205 | 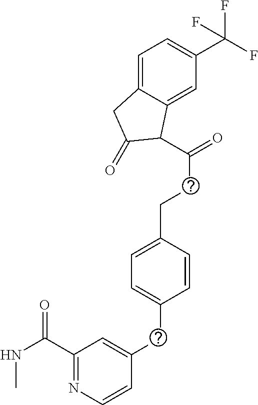 [SMILES: CNC(=O)c1cc(Cc2ccc(CCC(=O)C3C(=O)Cc4ccc(C(F)(F)F)cc43)cc2)ccn1] | 2-Oxo-6-trifluoromethyl- 2,3-dihydro-indole-1- carboxylic acid 4-(2- methylcarbamoyl-pyridin- 4-yloxy)-benzylamide | | 1100 | 484.43 |
| 206 | 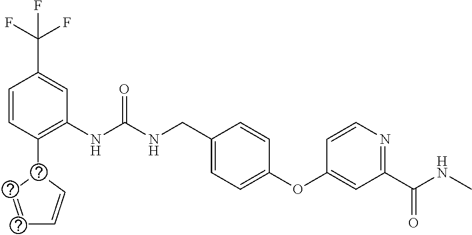 [SMILES: CNC(=O)c1cc(Oc2ccc(CNC(=O)Nc3cc(C(F)(F)F)ccc3C3C=CC=C3)cc2)ccn1] | 4-{4-[3-(2-[1,2,3]Triazol-1- yl-5-trifluoromethyl- phenyl)-ureidomethyl]- phenoxy}-pyridine-2- carboxylic acid methylamide | | 31 | 511.46 |
| 207 | 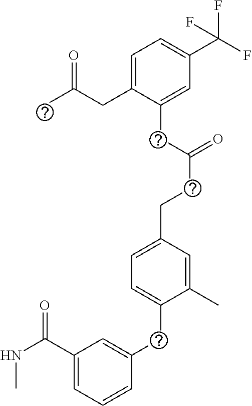 [SMILES: CNC(=O)c1cccc(Cc2ccc(CCC(=O)Cc3cc(C(F)(F)F)ccc3CC(C)=O)cc2C)c1] | 4-{4-[3-(2- Carbamoylmethyl-5- trifluoromethyl-phenyl)- ureidomethyl]-2-methyl- phenoxy}-pyridine-2- carboxylic acid methylamide | | 285 | 515.49 |
| 208 | 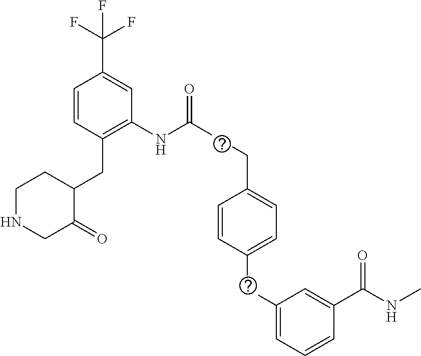 [SMILES: CNC(=O)c1cccc(Cc2ccc(CCC(=O)Nc3cc(C(F)(F)F)ccc3CC3CCNCC3=O)cc2)c1] | 4-(4-{3-[2-(2-Oxo- piperazin-1-ylmethyl)-5- trifluoromethyl-phenyl]- ureidomethyl}-phenoxy)- pyridine-2-carboxylic acid methylamide | | 855 | 556.54 |
| 209 | 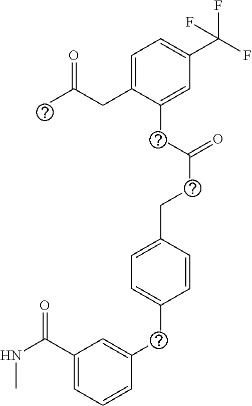 [SMILES: CNC(=O)c1cccc(Cc2ccc(CCC(=O)Cc3cc(C(F)(F)F)ccc3CC(C)=O)cc2)c1] | 4-{4-[3-(2- Carbamoylmethyl-5- trifluoromethyl-phenyl)- ureidomethyl]-phenoxy}- pyridine-2-carboxylic acid methylamide | | 985 | 501.46 |
| 210 | 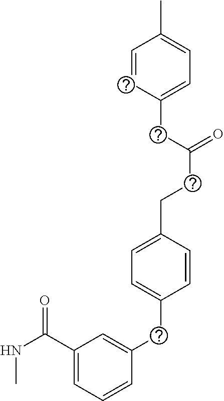 [SMILES: CNC(=O)c1cccc(Cc2ccc(CCC(=O)Cc3ccc(C)cc3)cc2)c1] | 4-{4-[3-(5-Methyl-pyridin-2- yl)-ureidomethyl]- phenoxy}-pyridine-2- carboxylic acid methylamide | 2000 | 1500 | 391.43 |
| 211 | 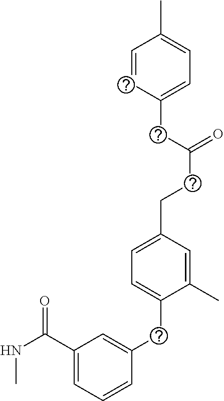 [SMILES: CNC(=O)c1cccc(Cc2ccc(CCC(=O)Cc3ccc(C)cc3)cc2C)c1] | 4-{2-Methyl-4-[3-(5-methyl- pyridin-2-yl)-ureidomethyl]- phenoxy}-pyridine-2- carboxylic acid methylamide | 780 | 630 | 405.46 |
| 212 | 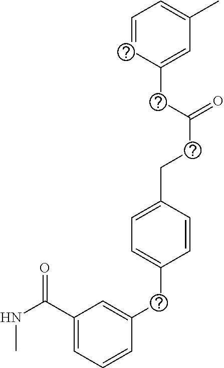 [SMILES: CNC(=O)c1cccc(Cc2ccc(CCC(=O)Cc3cccc(C)c3)cc2)c1] | 4-{4-[3-(4-Methyl-pyridin-2- yl)-ureidomethyl]- phenoxy}-pyridine-2- carboxylic acid methylamide | 570 | 380 | 391.43 |
| 213 | 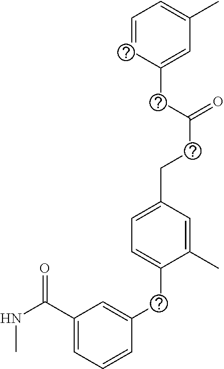 [SMILES: CNC(=O)c1cccc(Cc2ccc(CCC(=O)Cc3cccc(C)c3)cc2C)c1] | 4-{2-Methyl-4-[3-(4-methyl- pyridin-2-yl)-ureidomethyl]- phenoxy}-pyridine-2- carboxylic acid methylamide | 400 | 205 | 405.46 |
| 214 | 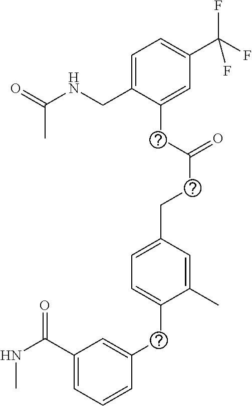 [SMILES: CNC(=O)c1cccc(Cc2ccc(CCC(=O)Cc3cc(C(F)(F)F)ccc3CNC(C)=O)cc2C)c1] | 4-(4-{3-[2-(Acetylamino- methyl)-5-trifluoromethyl- phenyl]-ureidomethyl}-2- methyl-phenoxy)-pyridine- 2-carboxylic acid methylamide | | 45 | 529.52 |
| 215 | 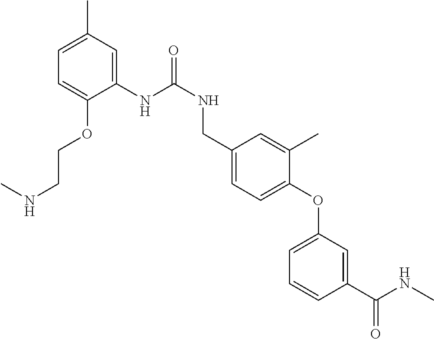 | 4-(2-Methyl-4-{3-[5-methyl- 2-(2-methylamino-ethoxy)- phenyl]-ureidomethyl}- phenoxy)-pyridine-2- carboxylic acid methylamide | 69 | 40 | 477.56 |
| 216 | 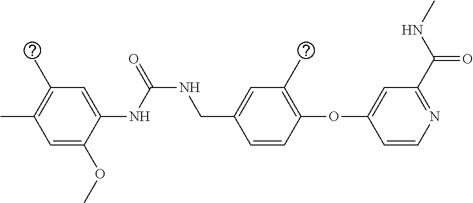 [SMILES: CNC(=O)c1cc(Oc2ccc(CNC(=O)Nc3cc(C)c(C)cc3OC)cc2C)ccn1] | 4-{4-[3-(5-Chloro-2- methoxy-4-methyl-phenyl)- ureidomethyl]-2-fluoro- phenoxy}-pyridine-2- carboxylic acid methylamide | 43 | | 472.90 |
| 217 | 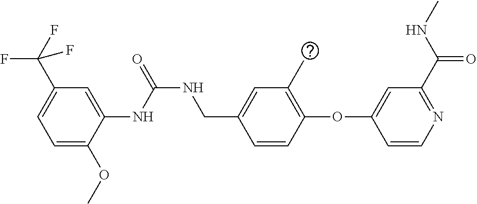 [SMILES: CNC(=O)c1cc(Oc2ccc(CNC(=O)Nc3cc(C(F)(F)F)ccc3OC)cc2C)ccn1] | 4-{2-Fluoro-4-[3-(2- methoxy-5-trifluoromethyl- phenyl)-ureidomethyl]- phenoxy}-pyridine-2- carboxylic acid methylamide | 21 | | 492.43 |
| 218 | 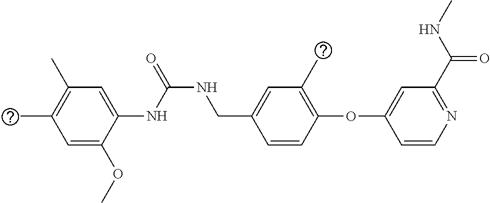 [SMILES: CNC(=O)c1cc(Oc2ccc(CNC(=O)Nc3cc(C)c(C)cc3OC)cc2C)ccn1] | 4-{4-[3-(4-Chloro-2- methoxy-5-methyl-phenyl)- ureidomethyl]-2-fluoro- phenoxy}-pyridine-2- carboxylic acid methylamide | 150 | | 472.90 |
| 219 | 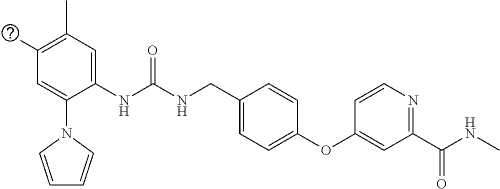 | 4-{4-[3-(4-Chloro-5-methyl- 2-pyrrol-1-yl-phenyl)- ureidomethyl]-phenoxy}- pyridine-2-carboxylic acid methylamide | | 114 | 489.96 |
| 220 | 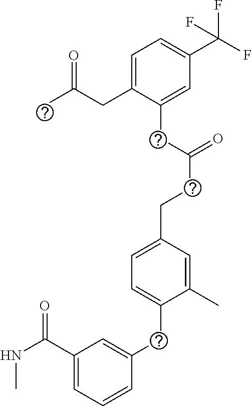 [SMILES: CNC(=O)c1cccc(Cc2ccc(CCC(=O)Cc3cc(C(F)(F)F)ccc3CC(C)=O)cc2C)c1] | (2-{3-[3-Methyl-4-(2- methylcarbamoyl-pyridin- 4-yloxy)-benzyl]-ureido}-4- trifluoromethyl-phenyl)- acetic acid | | 23500 | 516.48 |
| 221 | 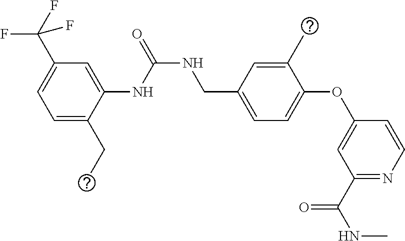 [SMILES: CCc1ccc(C(F)(F)F)cc1NC(=O)NCc1ccc(Oc2ccnc(C(=O)NC)c2)c(C)c1] | 4-{4-[3-(2-Aminomethyl-5- trifluoromethyl-phenyl)- ureidomethyl]-2-methyl- phenoxy}-pyridine-2- carboxylic acid methylamide | | 180 | 487.48 |
| 222 | 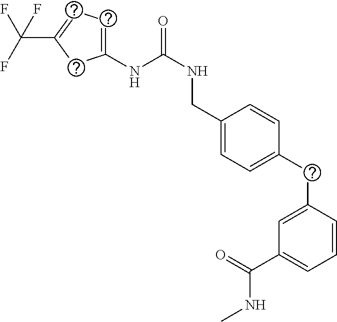 [SMILES: CNC(=O)c1cccc(Cc2ccc(CNC(=O)NC3=CC=C(C(F)(F)F)C3)cc2)c1] | 4-{4-[3-(5-Trifluoromethyl- [1,3,4]thiadiazol-2-yl)- ureidomethyl]-phenoxy}- pyridine-2-carboxylic acid methylamide | | 575 | 452.41 |
| 223 | 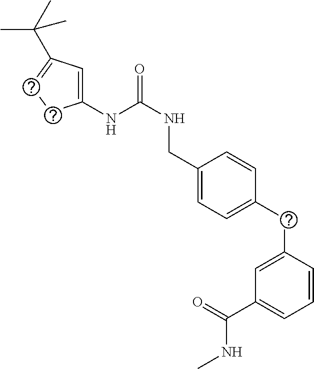 [SMILES: CNC(=O)c1cccc(Cc2ccc(CNC(=O)NC3=CC(C(C)(C)C)=CC3)cc2)c1] | 4-{4-[3-(5-tert-Butyl-2H- pyrazol-3-yl)- ureidomethyl]-phenoxy}- pyridine-2-carboxylic acid methylamide | | 7950 | 422.49 |
| 224 | 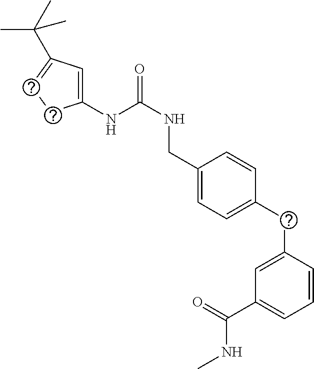 [SMILES: CNC(=O)c1cccc(Cc2ccc(CNC(=O)NC3=CC(C(C)(C)C)=CC3)cc2)c1] | 4-{4-[3-(5-tert-Butyl- isoxazol-3-yl)- ureidomethyl]-phenoxy}- pyridine-2-carboxylic acid methylamide | | 53 | 423.47 |
| 225 | 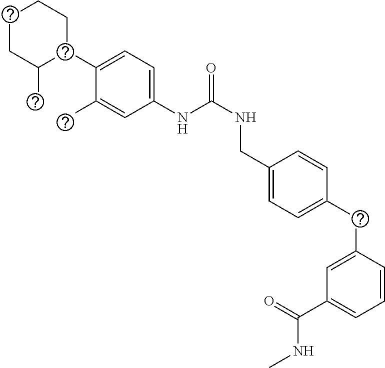 [SMILES: CNC(=O)c1cccc(Cc2ccc(CNC(=O)Nc3ccc(C4CCCCC4C)c(C)c3)cc2)c1] | 4-(4-{3-[3-Chloro-4-(3-oxo- morpholin-4-yl)-phenyl]- ureidomethyl}-phenoxy)- pyridine-2-carboxylic acid methylamide | | 8800 | 509.95 |
| 226 | 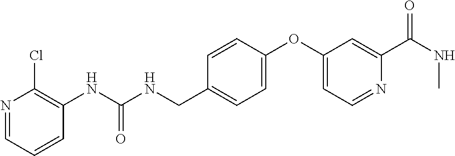 [SMILES: CNC(=O)c1cc(Oc2ccc(CNC(=O)Nc3cccnc3Cl)cc2)ccn1] | 4-{4-[3-(2-Chloro-pyridin-3- yl)-ureidomethyl]- phenoxy}-pyridine-2- carboxylic acid methylamide | 19000 | 11000 | 411.85 |
| 227 | 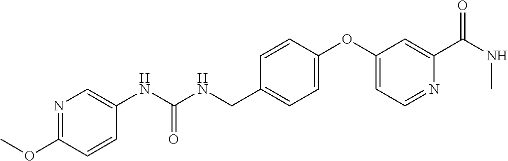 | 4-{4-[3-(6-Methoxy-pyridin- 3-yl)-ureidomethyl]- phenoxy}-pyridine-2- carboxylic acid methylamide | 5500 | 6300 | 407.43 |
| 228 | 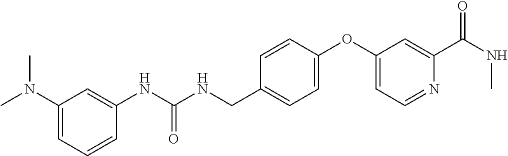 | 4-{4-[3-(3-Dimethylamino- phenyl)-ureidomethyl]- phenoxy}-pyridine-2- carboxylic acid methylamide | | 1500 | 419.48 |
| 229 | 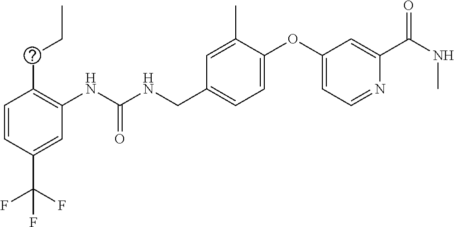 [SMILES: CCCc1ccc(C(F)(F)F)cc1NC(=O)NCc1ccc(Oc2ccnc(C(=O)NC)c2)c(C)c1] | 4-{4-[3-(2-Ethoxy-5- trifluoromethyl-phenyl)- ureidomethyl]-2-methyl- phenoxy}-pyridine-2- carboxylic acid methylamide | 84 | | 502.49 |
| 230 | 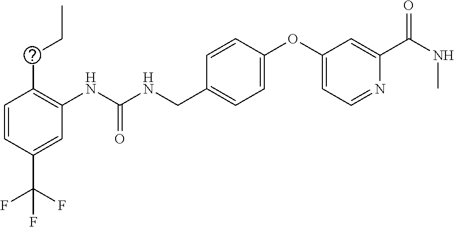 [SMILES: CCCc1ccc(C(F)(F)F)cc1NC(=O)NCc1ccc(Oc2ccnc(C(=O)NC)c2)cc1] | 4-{4-[3-(2-Ethoxy-5- trifluoromethyl-phenyl)- ureidomethyl]-phenoxy}- pyridine-2-carboxylic acid methylamide | 89 | | 488.47 |
| 231 | 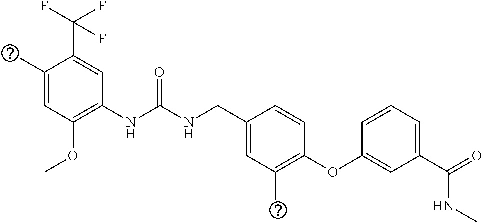 | 4-{4-[3-(4-Chloro-2- methoxy-5-trifluoromethyl- phenyl)-ureidomethyl]-2- fluoro-phenoxy}-pyridine-2- carboxylic acid methylamide | 100 | | 526.87 |
| 232 | 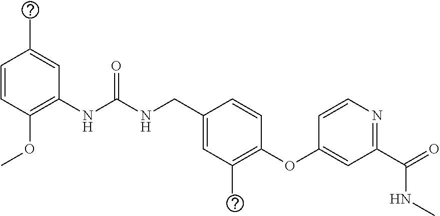 [SMILES: CNC(=O)c1cc(Oc2ccc(CNC(=O)Nc3cc(C)ccc3OC)cc2C)ccn1] | 4-{4-[3-(5-Chloro-2- methoxy-phenyl)- ureidomethyl]-2-fluoro- phenoxy}-pyridine-2- carboxylic acid methylamide | 52 | 19 | 458.88 |
| 233 | 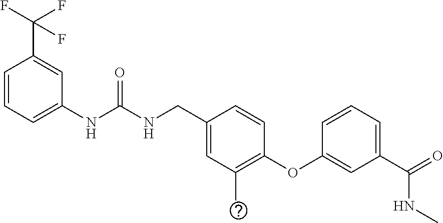 [SMILES: CNC(=O)c1cccc(Oc2ccc(CNC(=O)Nc3cccc(C(F)(F)F)c3)cc2C)c1] | 4-{2-Fluoro-4-[3-(3- trifluoromethyl-phenyl)- ureidomethyl]-phenoxy}- pyridine-2-carboxylic acid methylamide | | 27 | 462.40 |
| 234 | 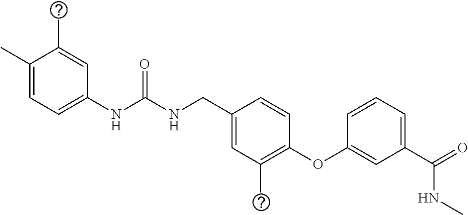 | 4-{4-[3-(3-Chloro-4-methyl- phenyl)-ureidomethyl]-2- fluoro-phenoxy}-pyridine-2- carboxylic acid methylamide | | 72 | 442.88 |
| 235 | 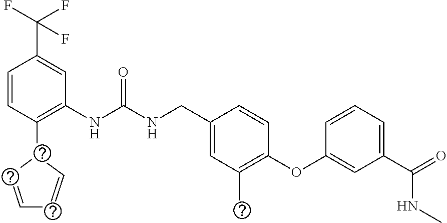 [SMILES: CNC(=O)c1cccc(Oc2ccc(CNC(=O)Nc3cc(C(F)(F)F)ccc3C3C=CC=C3)cc2C)c1] | 4-{2-Fluoro-4-[3-(2- [1,2,4]triazol-1-yl-5- trifluoromethyl-phenyl)- ureidomethyl]-phenoxy}- pyridine-2-carboxylic acid methylamide | | 27 | 529.45 |
| 236 | 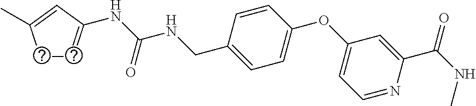 [SMILES: CNC(=O)c1cc(Oc2ccc(CNC(=O)NC3=CCC(C)=C3)cc2)ccn1] | 4-{4-[3-(5-Methyl-isoxazol- 3-yl)-ureidomethyl]- phenoxy}-pyridine-2- carboxylic acid methylamide | | 2050 | 381.39 |
| 237 | 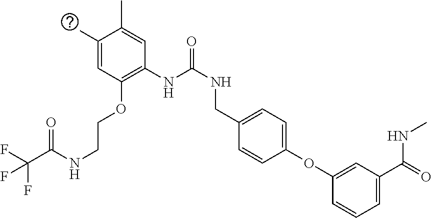 [SMILES: CNC(=O)c1cccc(Oc2ccc(CNC(=O)Nc3cc(C)c(C)cc3OCCNC(=O)C(F)(F)F)cc2)c1] | 4-(4-{3-[2-(2-Acetylamino- ethoxy)-4-chloro-5-methyl- phenyl]-ureidomethyl}- phenoxy)-pyridine-2- carboxylic acid methylamide | 300 | | 525.99 |
| 238 | 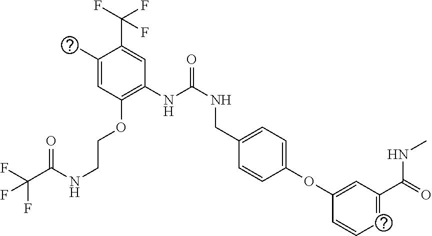 [SMILES: CNC(=O)c1cccc(Oc2ccc(CNC(=O)Nc3cc(C(F)(F)F)c(C)cc3OCCNC(=O)C(F)(F)F)cc2)c1] | 4-(4-{3-[2-(2-Acetylamino- ethoxy)-4-chloro-5- trifluoromethyl-phenyl]- ureidomethyl}-phenoxy)- pyridine-2-carboxylic acid methylamide | 61 | | 579.96 |
| 239 | 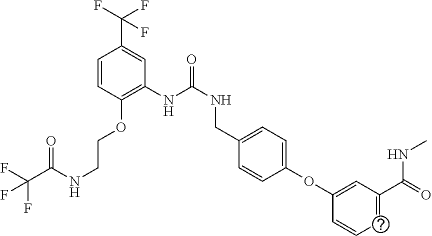 [SMILES: CNC(=O)c1cccc(Oc2ccc(CNC(=O)Nc3cc(C(F)(F)F)ccc3OCCNC(=O)C(F)(F)F)cc2)c1] | 4-(4-{3-[2-(2-Acetylamino- ethoxy)-5-trifluoromethyl- phenyl]-ureidomethyl}- phenoxy)-pyridine-2- carboxylic acid methylamide | 140 | | 545.52 |
| 240 | 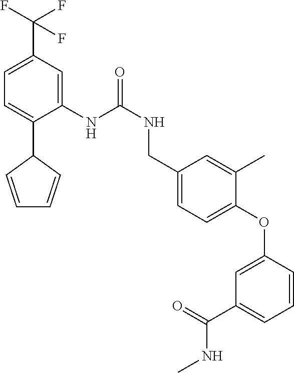 [SMILES: CNC(=O)c1cccc(Oc2ccc(CNC(=O)Nc3cc(C(F)(F)F)ccc3C3C=CC=C3)cc2C)c1] | 4-{4-[3-(2-Imidazol-1-yl-5- trifluoromethyl-phenyl)- ureidomethyl]-2-methyl- phenoxy}-pyridine-2- carboxylic acid methylamide | | 2900 | 524.50 |
| 241 | 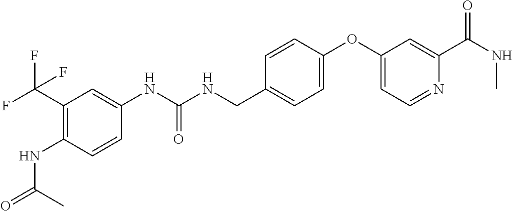 [SMILES: CNC(=O)c1cc(Oc2ccc(CNC(=O)Nc3ccc(NC(C)=O)c(C(F)(F)F)c3)cc2)ccn1] | 4-{4-[3-(4-Acetylamino-3- trifluoromethyl-phenyl)- ureidomethyl]-phenoxy}- pyridine-2-carboxylic acid methylamide | | 710 | 501.46 |
| 242 | 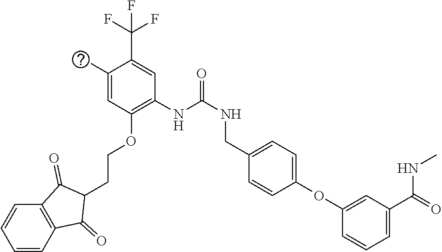 [SMILES: CNC(=O)c1cccc(Oc2ccc(CNC(=O)Nc3cc(C(F)(F)F)c(C)cc3OCCC3C(=O)c4ccccc4C3=O)cc2)c1] | 4-[4-(3-{4-Chloro-2-[2-(1,3- dioxo-1,3-dihydro-isoindol- 2-yl)-ethoxy]-5- trifluoromethyl-phenyl}- ureidomethyl)-phenoxy]- pyridine-2-carboxylic acid methylamide | 440 | | 668.03 |
| 243 | 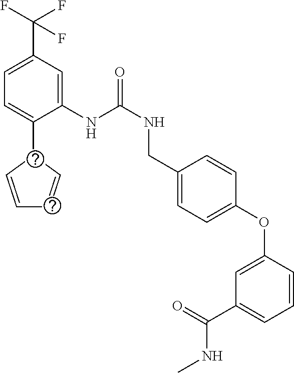 [SMILES: CNC(=O)c1cccc(Oc2ccc(CNC(=O)Nc3cc(C(F)(F)F)ccc3C3C=CC=C3)cc2)c1] | 4-{4-[3-(2-Imidazol-1-yl-5- trifluoromethyl-phenyl)- ureidomethyl]-phenoxy}- pyridine-2-carboxylic acid methylamide | | 9400 | 510.48 |
| 244 | 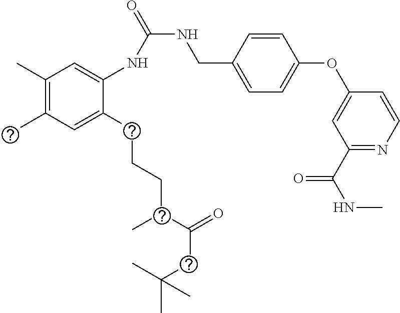 [SMILES: CNC(=O)c1cc(Oc2ccc(CNC(=O)Nc3cc(C)c(C)cc3CCCC(C)C(=O)CC(C)(C)C)cc2)ccn1] | [2-(5-Chloro-4-methyl-2-{3- [4-(2-methylcarbamoyl- pyridin-4-yloxy)-benzyl]- ureido}-phenoxy)-ethyl]- methyl-carbamic acid tert- butyl ester | 620 | | 598.10 |
| 245 | 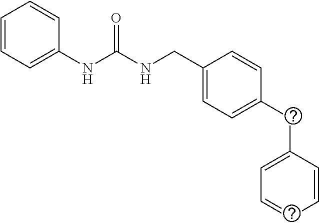 [SMILES: O=C(NCc1ccc(Cc2ccccc2)cc1)Nc1ccccc1] | 1-Phenyl-3-[4-(pyridin-4- yloxy)-benzyl]-urea | 9200 | | 319.36 |
| 246 | 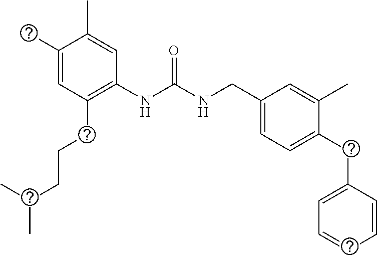 [SMILES: Cc1cc(CCCC(C)C)c(NC(=O)NCc2ccc(Cc3ccccc3)c(C)c2)cc1C] | 1-[4-Chloro-2-(2- dimethylamino-ethoxy)-5- methyl-phenyl]-3-[3- methyl-4-(pyridin-4-yloxy)- benzyl]-urea | 230 | | 468.98 |
| 247 | 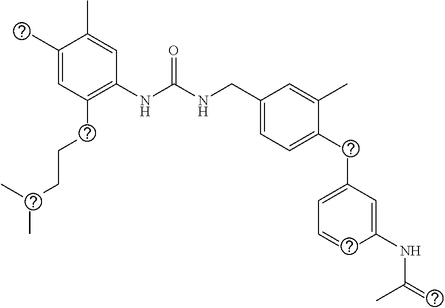 [SMILES: C=C(C)Nc1cccc(Cc2ccc(CNC(=O)Nc3cc(C)c(C)cc3CCCC(C)C)cc2C)c1] | N-[4-(4-{3-[4-Chloro-2-(2- dimethylamino-ethoxy)-5- methyl-phenyl]- ureidomethyl}-2-methyl- phenoxy)-pyridin-2-yl]- acetamide | 6 | | 526.04 |
| 248 | 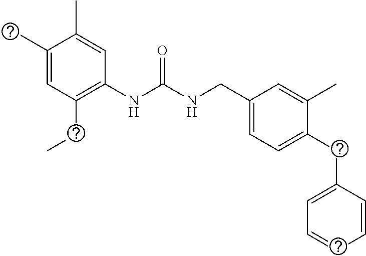 [SMILES: CCc1cc(C)c(C)cc1NC(=O)NCc1ccc(Cc2ccccc2)c(C)c1] | 1-(4-Chloro-2-methoxy-5- methyl-phenyl)-3-[3- methyl-4-(pyridin-4-yloxy)- benzyl]-urea | 770 | | 411.89 |
| 249 | 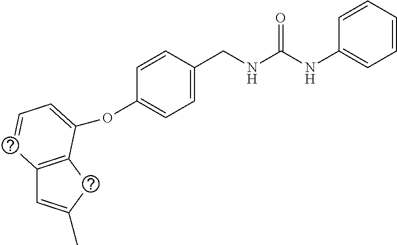 [SMILES: CC1=Cc2cccc(Oc3ccc(CNC(=O)Nc4ccccc4)cc3)c2C1] | 1-[4-(2-Methyl-furo[3,2- b]pyridin-7-yloxy)-benzyl]- 3-phenyl-urea | 650 | | 373.41 |
| 250 | 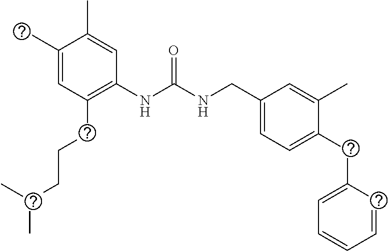 [SMILES: Cc1cc(CCCC(C)C)c(NC(=O)NCc2ccc(Cc3ccccc3)c(C)c2)cc1C] | 1-[4-Chloro-2-(2- dimethylamino-ethoxy)-5- methyl-phenyl]-3-[3- methyl-4-(pyrimidin-4- yloxy)-benzyl]-urea | 1200 | | 469.97 |
| 251 | 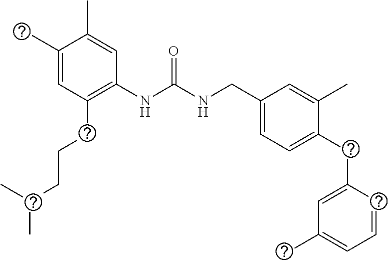 [SMILES: Cc1cccc(Cc2ccc(CNC(=O)Nc3cc(C)c(C)cc3CCCC(C)C)cc2C)c1] | 1-[4-(6-Amino-pyrimidin-4- yloxy)-3-methyl-benzyl]-3- [4-chloro-2-(2- dimethylamino-ethoxy)-5- methyl-phenyl]-urea | 440 | | 484.99 |
| 252 | 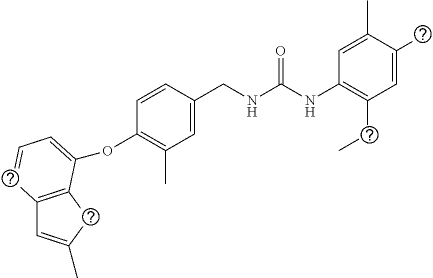 [SMILES: CCc1cc(C)c(C)cc1NC(=O)NCc1ccc(Oc2cccc3c2CC(C)=C3)c(C)c1] | 1-(4-Chloro-2-methoxy-5- methyl-phenyl)-3-[3- methyl-4-(2-methyl- furo[3,2-b]pyridin-7-yloxy)- benzyl]-urea | 750 | | 465.94 |
| 253 | 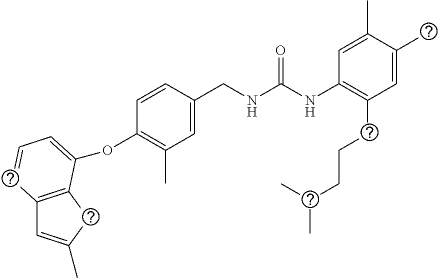 [SMILES: CC1=Cc2cccc(Oc3ccc(CNC(=O)Nc4cc(C)c(C)cc4CCCC(C)C)cc3C)c2C1] | 1-[4-Chloro-2-(2- dimethylamino-ethoxy)-5- methyl-phenyl]-3-[3- methyl-4-(2-methyl- furo[3,2-b]pyridin-7-yloxy)- benzyl]-urea | 70 | | 523.03 |
| 254 | 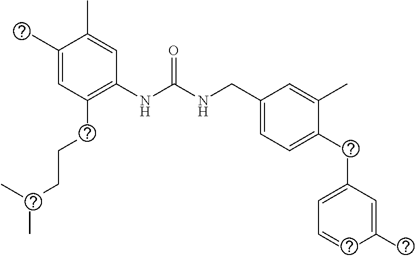 [SMILES: Cc1cccc(Cc2ccc(CNC(=O)Nc3cc(C)c(C)cc3CCCC(C)C)cc2C)c1] | 1-[4-(2-Amino-pyridin-4- yloxy)-3-methyl-benzyl]-3- [4-chloro-2-(2- dimethylamino-ethoxy)-5- methyl-phenyl]-urea | 31 | | 484.00 |
| 255 | 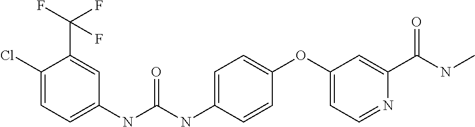 [SMILES: CNC(=O)c1cc(Oc2ccc(NC(=O)Nc3ccc(Cl)c(C(F)(F)F)c3)cc2)ccn1] | 4-[4-[[4-chloro-3- (trifluoromethyl)- phenyl]carbamoylamino] phenoxy]-N-methyl- pyridine-2-carboxamide | 110 | | |

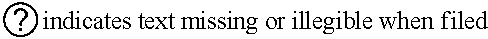

TABLE 3a

| | | | | IC50 | IC50 | stability | ESI |
|---|---|---|---|---|---|---|---|
| | | Compound | IC50 | [p- | [pro- | in | MS |
| | | (chemical | [DDR2] | DDR2] | MMP13] | synovial | Rt/ |
| No. | Compound (structure) | name) | M | M | M | fluid | M + H |
| 1 | 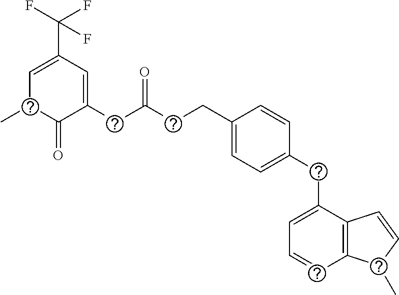 [SMILES: CC1C=C(C(F)(F)F)C=C(CC(=O)CCc2ccc(Cc3cccc4c3C=CC4C)cc2)C1=O] | 1-(1-Methyl-2- oxo-5- trifluoromethyl- 1,2-dihydro- pyridin-3-yl)-3- [4-(1H- pyrrolo[2,3- b]pyridin-4- yloxy)-benzyl]- urea | 4.40E- 07 | | | | 2.115/ 472.2 |
| 2 | 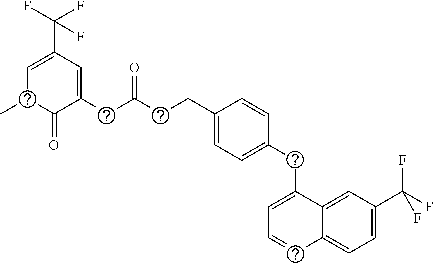 [SMILES: CC1C=C(C(F)(F)F)C=C(CC(=O)CCc2ccc(Cc3cccc4ccc(C(F)(F)F)cc34)cc2)C1=O] | 1-(1-Methyl-2- oxo-5- trifluoromethyl- 1,2-dihydro- pyridin-3-yl)-3- [4-(6- trifluoromethyl- quinolin-4- yloxy)-benzyl]- urea | 4.00E- 07 | 5.60E- 06 | | | 2.442/ 537.2 |
| 3 | 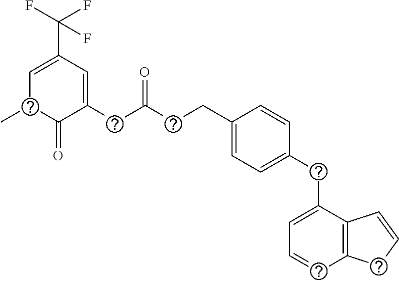 [SMILES: CC1C=C(C(F)(F)F)C=C(CC(=O)CCc2ccc(Cc3cccc4c3C=CC4)cc2)C1=O] | 1-(1-Methyl-2- oxo-5- trifluoromethyl- 1,2-dihydro- pyridin-3-yl)-3- [4-(1H- pyrrolo[2,3- b]pyridin-4- yloxy)-benzyl]- urea | 6.60E- 08 | 2.00E- 08 | | | 1.836/ 458.2 |
| 4 | 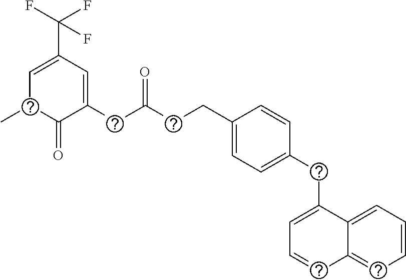 [SMILES: CC1C=C(C(F)(F)F)C=C(CC(=O)CCc2ccc(Cc3cccc4ccccc34)cc2)C1=O] | 1-(1-Methyl-2- oxo-5- trifluoromethyl- 1,2-dihydro- pyridin-3-yl)-3- [4- ([1,8]naphthyri- din-4-yloxy)- benzyl]-urea | 4.80E- 06 | | | | 1.849/ 470.2 |
| 5 | 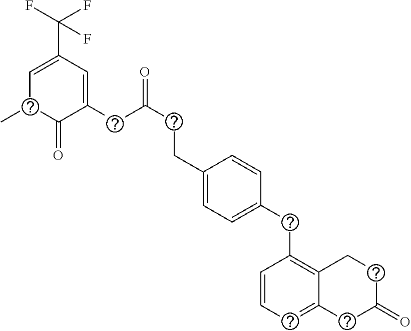 [SMILES: CC1C=C(C(F)(F)F)C=C(CC(=O)CCc2ccc(Cc3cccc4c3CCC(=O)C4)cc2)C1=O] | 1-(1-Methyl-2- oxo-5- trifluoromethyl- 1,2-dihydro- pyridin-3-yl)-3- [4-(2-oxo- 1,2,3,4- tetrahydro- pyrido[2,3- d]pyrimidin-5- yloxy)-benzyl]- urea | 1.50E- 08 | 5.90E- 09 | | | 2.208/ 489.2 |
| 6 | 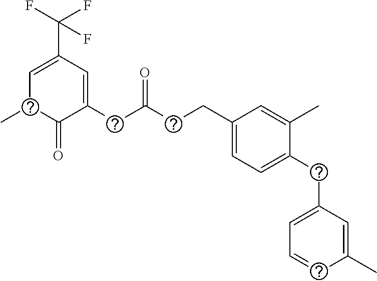 | 1-[3-Methyl-4- (2-methyl- pyridin-4- yloxy)-benzyl]- 3-(1-methyl-2- oxo-5- trifluoromethyl- 1,2-dihydro- pyridin-3-yl)- urea | 1.10E- 07 | 3.00E- 08 | 2.58E-07 | | 1.609/ 447.2 |
| 7 | 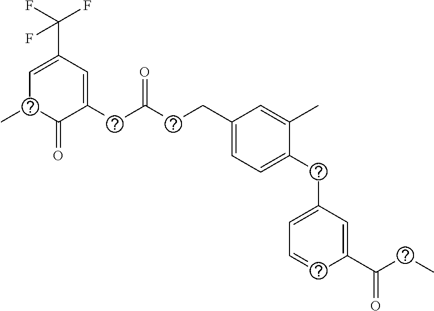 [SMILES: CCC(=O)c1cccc(Cc2ccc(CCC(=O)CC3=CC(C(F)(F)F)=CC(C)C3=O)cc2C)c1] | 4-{2-Methyl-4- [3-(1-methyl- 2-oxo-5- trifluoromethyl- 1,2-dihydro- pyridin-3-yl)- ureidomethyl]- phenoxy}- pyridine-2- carboxylic acid methylamide | 3.70E- 08 | 1.20E- 08 | 3.29E-08 | stable | 2.814/ 490.2 |
| 8 | 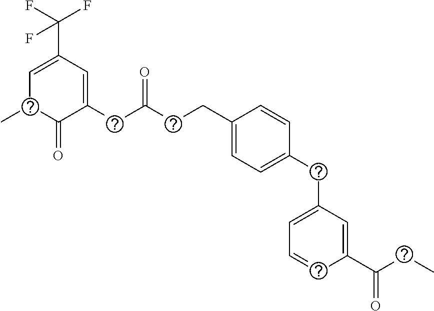 [SMILES: CCC(=O)c1cccc(Cc2ccc(CCC(=O)CC3=CC(C(F)(F)F)=CC(C)C3=O)cc2)c1] | 4-{4-[3-(1- Methyl-2-oxo- 5- trifluoromethyl- 1,2-dihydro- pyridin-3-yl)- ureidomethyl]- phenoxy}- pyridine-2- carboxylic acid methylamide | 9.70E- 08 | 2.80E- 08 | 1.48E-08 | | 2.113/ 476.2 |
| 9 | 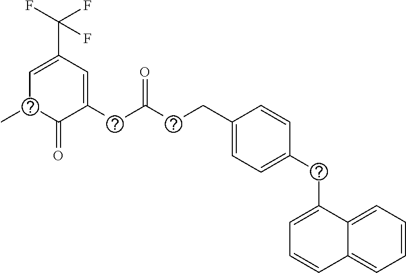 [SMILES: CC1C=C(C(F)(F)F)C=C(CC(=O)CCc2ccc(Cc3cccc4ccccc34)cc2)C1=O] | 1-(1-Methyl-2- oxo-5- trifluoromethyl- 1,2-dihydro- pyridin-3-yl)-3- [4-(quinolin-4- yloxy)-benzyl]- urea | 5.10E- 08 | | | | 1.802/ 469.1 |
| 10 | 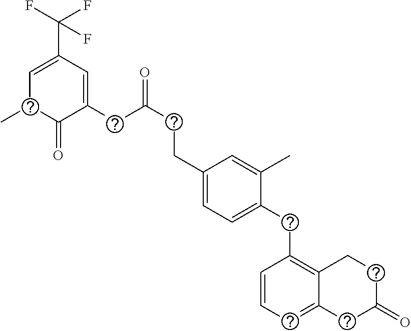 [SMILES: Cc1cc(CCC(=O)CC2=CC(C(F)(F)F)=CC(C)C2=O)ccc1Cc1cccc2c1CCC(=O)C2] | 1-[3-Methyl-4- (2-oxo- 1,2,3,4- tetrahydro- pyrido[2,3- d]pyrimidin-5- yloxy)-benzyl]- 3-(1-methyl-2- oxo-5- trifluoromethyl- 1,2-dihydro- pyridin-3-yl)- urea | | | | | 1.934/ 503.2 |
| 11 | 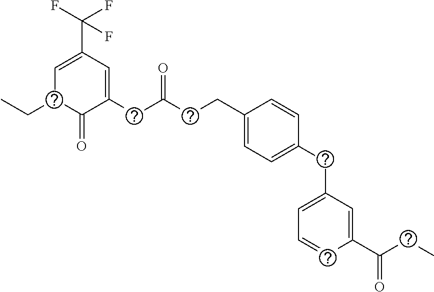 [SMILES: CCC(=O)c1cccc(Cc2ccc(CCC(=O)CC3=CC(C(F)(F)F)=CC(CC)C3=O)cc2)c1] | 4-{4-[3-(1- Ethyl-2-oxo-5- trifluoromethyl- 1,2-dihydro- pyridin-3-yl)- ureidomethyl]- phenoxy}- pyridine-2- carboxylic acid methylamide | 9.10E- 08 | | | | |
| 12 | 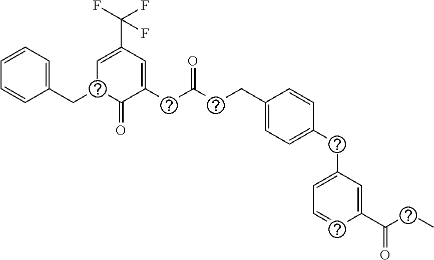 [SMILES: CCC(=O)c1cccc(Cc2ccc(CCC(=O)CC3=CC(C(F)(F)F)=CC(Cc4ccccc4)C3=O)cc2)c1] | 4-{4-[3-(1- Benzyl-2-oxo- 5- trifluoromethyl- 1,2-dihydro- pyridin-3-yl)- ureidomethyl]- phenoxy}- pyridine-2- carboxylic acid methylamide | 2.60E- 07 | | | | |
| 13 | 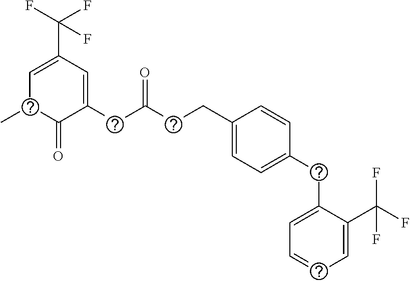 [SMILES: CC1C=C(C(F)(F)F)C=C(CC(=O)CCc2ccc(Cc3ccccc3C(F)(F)F)cc2)C1=O] | 1-(1-Methyl-2- oxo-5- trifluoromethyl- 1,2-dihydro- pyridin-3-yl)-3- [4-(3- trifluoromethyl- pyridin-4- yloxy)-benzyl]- urea | 1.60E- 06 | | | | |
| 14 | 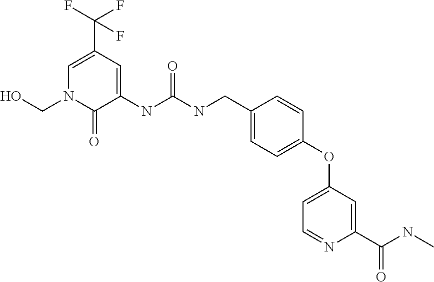 [SMILES: CNC(=O)c1cc(Oc2ccc(CNC(=O)Nc3cc(C(F)(F)F)cn(CO)c3=O)cc2)ccn1] | 4-{4-[3-(1 Hydroxymethl- 2-oxo-5- trifluoromethyl- 1,2-dihydro- pyridin-3-yl)- ureidomethyl]- phenoxy}- pyridine-2- carboxylic acid methylamide | | | | | |
| 15 | 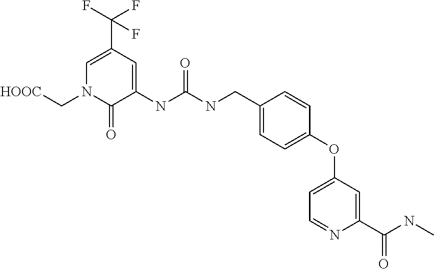 [SMILES: CNC(=O)c1cc(Oc2ccc(CNC(=O)Nc3cc(C(F)(F)F)cn(CC(=O)O)c3=O)cc2)ccn1] | (3-{3-[4-(2- Methylcarbaoyl- pyridin- 4-yloxy)- benzyl]- ureido}-2-oxo- 5-trifluoro- methyl-2H- pyridin-1-yl)- acetic acid | | | | | |
| 16 | 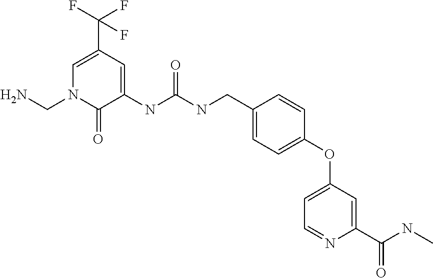 [SMILES: CNC(=O)c1cc(Oc2ccc(CNC(=O)Nc3cc(C(F)(F)F)cn(CN)c3=O)cc2)ccn1] | 4-{4-[3-(1- Aminomethyl- 2-oxo-5- trifluoromethyl- 1,2-dihydro- pyridin-3-yl)- ureidomethyl]- phenoxy}- pyridine- 2-carboxylic acid methylamide | | | | | |
| 17 | 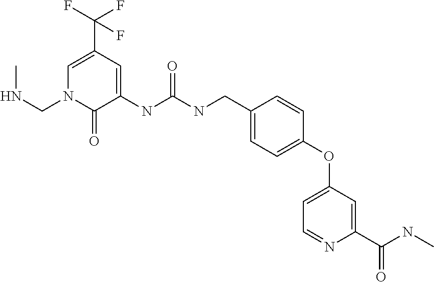 [SMILES: CNCn1cc(C(F)(F)F)cc(NC(=O)NCc2ccc(Oc3ccnc(C(=O)NC)c3)cc2)c1=O] | 4-{4-[3-(1- Methylamino methyl-2-oxo- 5- trifluoromethyl- 1,2-dihydro- pyridin- 3-yl)- ureidomethyl]- phenoxy}- pyridine-2- carboxylic acid methylamide | | | | | |
| 18 | 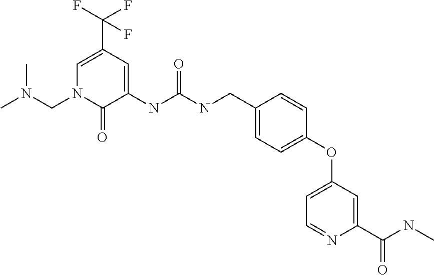 | 4-{4-[3-(1- Dimethylamio methyl-2-oxo- 5- trifluoromethyl- 1,2-dihydro- pyridin-3-yl)- ureidomethyl]- phenoxy}- pyridine-2- carboxylic acid methylamide | | | | | |

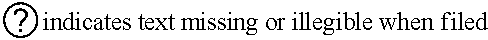

TABLE 3b
NMR data of the compounds of table 3a

| No. of | |
|---|---|
| compound | |
| of table 3a | 1HNMR |
| 1 | 1H NMR (400 MHz, DMSO-d6) ppm = 8.66 (s, 1H), 8.22 (d, J = 2.5, 1H), 8.17 (d, |
| | J = 5.6, 1H), 7.97-7.91 (m, 1H), 7.74 (t, J = 5.8, 1H), 7.43 (d, J = 3.5, 1H), |
| | 7.41-7.35 (m, 2H), 7.22-7.13 (m, 2H), 6.49 (d, J = 5.6, 1H), 6.24 (d, J = 3.5, 1H), 4.34 |
| | (d, J = 5.7, 2H), 3.83 (s, 3H), 3.57 (s, 3H). |
| 2 | 1H NMR (500 MHz, DMSO-d6) ppm = 8.84 (d, J = 5.2, 1H), 8.69 (s, 1H), |
| | 8.66-8.64 (m, 1H), 8.25 (d, J = 8.8, 1H), 8.23 (d, J = 2.5, 1H), 8.10 (dd, J = 8.9, 2.2, 1H), |
| | 7.96-7.93 (m, 1H), 7.79 (t, J = 5.9, 1H), 7.49-7.46 (m, 2H), 7.36-7.32 (m, 2H), |
| | 6.72 (d, J = 5.2, 1H), 4.38 (d, J = 5.9, 2H), 3.57 (s, 3H). |
| 3 | 1H NMR (500 MHz, DMSO-d6) ppm = 12.02 (s, 1H), 8.67 (s, 1H), 8.22 (d, |
| | J = 2.5, 1H), 8.16 (d, J = 5.8, 1H), 7.97-7.93 (m, 1H), 7.75 (t, J = 5.9, 1H), |
| | 7.45-7.38 (m, 3H), 7.23-7.19 (m, 2H), 6.51 (d, J = 5.8, 1H), 6.30-6.27 (m, 1H), 4.35 |
| | (d, J = 5.8, 2H), 3.57 (s, 3H). |
| 4 | 1H NMR (500 MHz, DMSO-d6) ppm = 9.22 (dd, J = 4.3, 1.9, 1H), 8.96 (d, J = 5.7, |
| | 1H), 8.91 (dd, J = 8.4, 1.9, 1H), 8.69 (s, 1H), 8.22 (d, J = 2.5, 1H), 7.98-7.93 (m, |
| | 1H), 7.83 (dd, J = 8.3, 4.3, 1H), 7.80 (t, J = 5.9, 1H), 7.54-7.32 (m, 4H), 6.82 (d, |
| | J = 5.7, 1H), 4.39 (d, J = 5.9, 2H), 3.57 (s, 3H). |
| 5 | 1H NMR (500 MHz, DMSO-d6) ppm = 9.52 (s, 1H), 8.65 (s, 1H), 8.21 (d, J = 2.5, |
| | 1H), 7.96-7.93 (m, 1H), 7.92 (d, J = 5.9, 1H), 7.73 (t, J = 5.9, 1H), 7.40-7.34 |
| | (m, 2H), 7.16-7.11 (m, 2H), 7.00 (s, 1H), 6.18 (d, J = 5.9, 1H), 4.38 (s, 2H), |
| | 4.32 (d, J = 5.8, 2H), 3.56 (s, 3H). |
| 6 | 1H NMR (400 MHz, DMSO) ppm = 8.68 (s, 1H), 8.63 (d, J = 6.7, 1H), 8.21 (d, |
| | J = 2.5, 1H), 8.01-7.92 (m, 1H), 7.81-7.73 (m, 1H), 7.35 (d, J = 2.1, 1H), |
| | 7.32-7.13 (m, 4H), 4.34 (d, J = 5.8, 2H), 3.57 (s, 3H), 2.61 (s, 3H), 2.11 (s, 3H). |
| 7 | 1H NMR (500 MHz, DMSO-d6) ppm = 8.74 (q, J = 4.6, 1H), 8.65 (s, 1H), 8.50 (d, |
| | J = 5.6, 1H), 8.22 (d, J = 2.5, 1H), 7.96-7.92 (m, 1H), 7.76 (t, J = 5.9, 1H), |
| | 7.34-7.31 (m, 1H), 7.29 (d, J = 2.6, 1H), 7.25 (dd, J = 8.2, 2.2, 1H), 7.15-7.11 (m, 1H), |
| | 7.10 (dd, J = 5.6, 2.6, 1H), 4.33 (d, J = 5.8, 2H), 3.57 (s, 3H), 2.79 (d, J = 4.9, 3H), |
| | 2.10 (s, 3H). |
| 8 | 1H NMR (400 MHz, DMSO) ppm = 8.77-8.69 (m, 1H), 8.65 (s, 1H), 8.51 (d, |
| | J = 5.6, 1H), 8.22 (d, J = 2.5, 1H), 7.96-7.92 (m, 1H), 7.76 (t, J = 5.9, 1H), |
| | 7.45-7.40 (m, 2H), 7.38 (d, J = 2.6, 1H), 7.23-7.17 (m, 2H), 7.17-7.13 (m, 1H), 4.35 |
| | (d, J = 5.7, 2H), 3.57 (s, 3H), 2.78 (d, J = 4.8, 3H). |

TABLE 4

| | | | | IC50 | |
|---|---|---|---|---|---|
| | | | IC50 | [p- | |
| | | Compound | [DDR2] | DDR2] | ESI MS Rt/ |
| No. | Compound (structure) | (chemical name) | M | M | M + H |
| 1 | 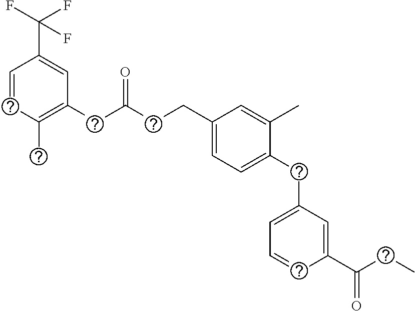 [SMILES: CCC(=O)c1cccc(Cc2ccc(CCC(=O)Cc3cc(C(F)(F)F)ccc3C)cc2C)c1] | (2-Hydroxy-5- trifluoromethyl-pyridin- 3-yl)-carbamic acid 3- methyl-4-(2- methylcarbamoyl- pyridin-4-yloxy)-benzyl ester | 2.90E- 07 | | 1.877/477.2 |
| 2 | 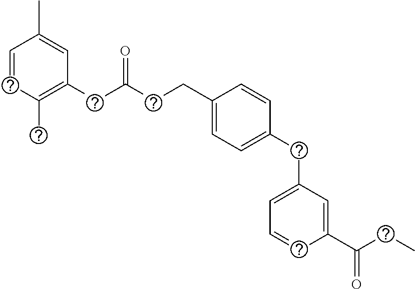 [SMILES: CCC(=O)c1cccc(Cc2ccc(CCC(=O)Cc3cc(C)ccc3C)cc2)c1] | (2-Hydroxy-5-methyl- pyridin-3-yl)-carbamic acid 4-(2- methylcarbamoyl- pyridin-4-yloxy)-benzyl ester | | | 1.915/409.2 |
| 3 | 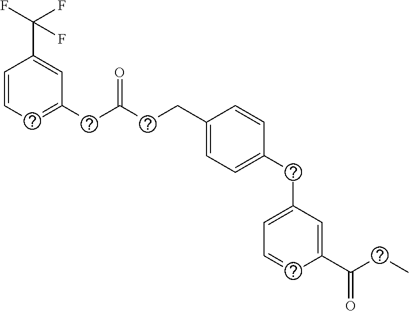 [SMILES: CCC(=O)c1cccc(Cc2ccc(CCC(=O)Cc3cccc(C(F)(F)F)c3)cc2)c1] | (4-Trifluoromethyl- pyridin-2-yl)-carbamic acid 4-(2- methylcarbamoyl- pyridin-4-yloxy)-benzyl ester | 3.30E- 08 | | 1.983/447.1 |
| 4 | 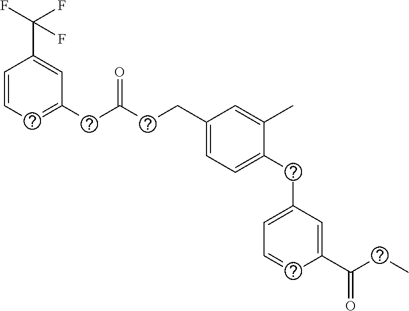 [SMILES: CCC(=O)c1cccc(Cc2ccc(CCC(=O)Cc3cccc(C(F)(F)F)c3)cc2C)c1] | (4-Trifluoromethyl- pyridin-2-yl)-carbamic acid 3-methyl-4-(2- methylcarbamoyl- pyridin-4-yloxy)-benzyl ester | 4.10E- 08 | | 2.470/461.2 |
| 5 | 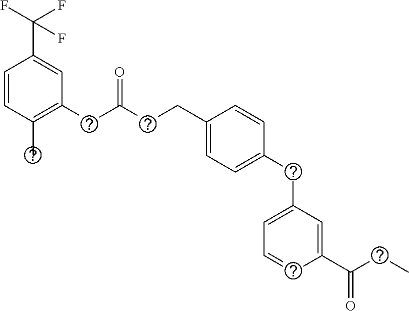 [SMILES: CCC(=O)c1cccc(Cc2ccc(CCC(=O)Cc3cc(C(F)(F)F)ccc3C)cc2)c1] | (2-Hydroxy-5- trifluoromethyl-pyridin- 3-yl)-carbamic acid 4- (2-methylcarbamoyl- pyridin-4-yloxy)-benzyl ester | 6.10E- 07 | | 2.150/463.1 |
| 6 | 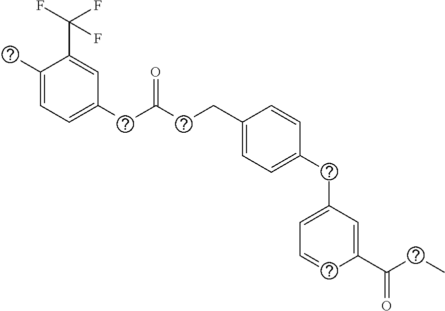 [SMILES: CCC(=O)c1cccc(Cc2ccc(CCC(=O)Cc3ccc(C)c(C(F)(F)F)c3)cc2)c1] | (4-Chloro-3- trifluoromethyl-phenyl)- carbamic acid 4-(2- methylcarbamoyl- pyridin-4-yloxy)-benzyl ester | 4.20E- 07 | 2.70E- 07 | 2.176/480.1 |

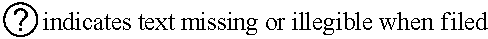

TABLE 4b
NMR data of the compounds of table 4a

| No. of | |
|---|---|
| compound | |
| of table 4a | 1HNMR |
| 1 | 1H NMRT500 MHz, DMSO-d6) ppm = 12.52 (s, 1H), 8.81 (s, 1H), 8.76 (q, |
| | J = 4.5, 1H), 8.51 (d, J = 5.6, 1H), 8.02 (d, J = 2.5, 1H), 7.68-7.64 (m, 1H), 7.53-7.49 |
| | (m, 1H), 7.41 (dd, J = 8.2, 2.1, 1H), 7.32 (d, J = 2.6, 1H), 7.17 (d, J = 8.2, 1H), |
| | 7.11 (dd, J = 5.6, 2.6, 1H), 5.21 (s, 2H), 2.79 (d, J = 4.8, 3H), 2.12 (s, 3H). |
| 2 | 1H NMR (500 MHz, Chloroform-d) ppm = 8.41 (d, J = 5.7, 1H), 8.35-8.30 (m, |
| | 1H), 8.17-8.10 (m, 1H), 7.72 (d, J = 2.5, 1H), 7.61 (s, 1H), 7.51-7.47 (m, 2H), |
| | 7.14-7.09 (m, 2H), 7.01 (dd, J = 5.6, 2.5, 1H), 6.97-6.94 (m, 1H), 5.24 (s, 2H), |
| | 3.01 (d, J = 5.0, 3H), 2.22 (s, 3H). |
| 3 | 1H NMR (500 MHz, Chloroform-d) ppm = 8.70 (s, 1H), 8.42-8.37 (m, 2H), 8.34 |
| | (s, 1H), 8.08-7.99 (m, 1H), 7.71 (d, J = 2.5, 1H), 7.52-7.46 (m, 2H), 7.23-7.19 |
| | (m, 1H), 7.14-7.09 (m, 2H), 6.99 (dd, J = 5.6, 2.6, 1H), 5.27 (s, 2H), 3.00 |
| | (d, J = 5.1, 3H). |
| 4 | 1H NMR (500 MHz, DMSO-d6) ppm = 10.80 (s, 1H), 8.75 (q, J = 4.8, 1H), 8.55 |
| | (d, J = 5.2, 1H), 8.51 (d, J = 5.6, 1H), 8.16 (s, 1H), 7.52-7.49 (m, 1H), 7.44-7.39 |
| | (m, 2H), 7.28 (d, J = 2.6, 1H), 7.21-7.15 (m, 1H), 7.12 (dd, J = 5.6, 2.6, 1H), 5.23 |
| | (s, 2H), 2.78 (d, J = 4.9, 3H), 2.12 (s, 3H). |
| 5 | 1H NMR (400 MHz, DMSO-d6) ppm = 12.50 (s, 1H), 8.82 (s, 1H), 8.78-8.71 |
| | (m, 1H), 8.52 (d, J = 5.6, 1H), 8.01 (d, J = 2.5, 1H), 7.65 (s, 1H), 7.59 (d, J = 8.4, |
| | 2H), 7.40 (d, J = 2.6, 1H), 7.25 (d, J = 8.4, 2H), 7.17 (dd, J = 5.6, 2.7, 1H), 5.24 (s, |
| | 2H), 2.79 (d, J = 4.8, 3H). |
| 6 | 1H NMR (300 MHz DMSO-d6) ppm = 10.34 (s, 1H) 8.81 (q, J = 4.8, 1H), 8.54 |
| | (d, J = 5.6, 1H), 8.05 (d, J = 2.5, 1H), 7.73 (dd, J = 8.9, 2.6, 1H), 7.68-7.63 (m, |
| | 1H), 7.63-7.56 (m, 2H), 7.36 (d, J = 2.5, 1H), 7.32-7.24 (m, 2H), 7.21 (dd, |
| | J = 5.6, 2.6, 1H), 5.23 (s, 2H), 2.79 (d, J = 4.8, 3H). |

TABLE 5a

| | | | | IC50 | |
|---|---|---|---|---|---|
| | | | IC50 | [p- | |
| | | Compound | [DDR2] | DDR2] | ESI MS |
| No. | Compound (structure) | (chemical name) | M | M | Rt/M + H |
| 1 | 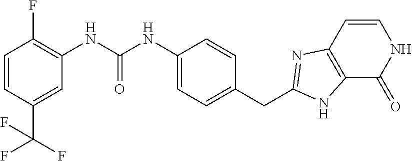 [SMILES: O=C(Nc1ccc(Cc2nc3cc[nH]c(=O)c3[nH]2)cc1)Nc1cc(C(F)(F)F)ccc1F] | 1-(2-Fluoro-5- trifluoromethyl- phenyl)-3-[4-(4-oxo- 4,5-dihydro-3H- imidazo[4,5-c]pyridin- 2-ylmethyl)-phenyl]- urea | 1.10E- 07 | 1.80E- 08 | |
| 2 | 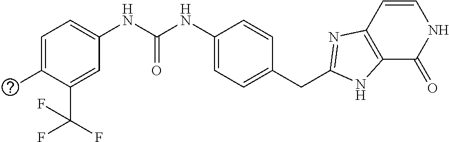 [SMILES: Cc1ccc(NC(=O)Nc2ccc(Cc3nc4cc[nH]c(=O)c4[nH]3)cc2)cc1C(F)(F)F] | 1-(4-Chloro-3- trifluoromethyl- phenyl)-3-[4-(4-oxo- 4,5-dihydro-3H- imidazo[4,5-c]pyridin- 2-ylmethyl)-phenyl]- urea | 2.30E 06 | | |
| 3 | 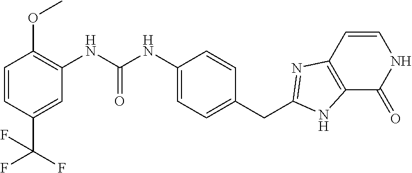 [SMILES: COc1ccc(C(F)(F)F)cc1NC(=O)Nc1ccc(Cc2nc3cc[nH]c(=O)c3[nH]2)cc1] | 1-(2-Methoxy-5- trifluoromethyl- phenyl)-3-[4-(4-oxo- 4,5-dihydro-3H- imidazo[4,5-c]pyridin- 2-ylmethyl)-phenyl]- urea | 6.20E- 08 | 1.10E- 07 | |
| 4 | 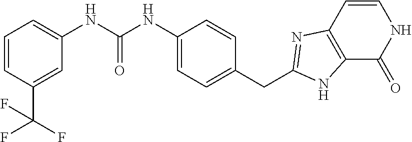 [SMILES: O=C(Nc1ccc(Cc2nc3cc[nH]c(=O)c3[nH]2)cc1)Nc1cccc(C(F)(F)F)c1] | 1-[4-(4-Oxo-4,5- dihydro-3H- imidazo[4,5-c]pyridin- 2-ylmethyl)-phenyl]-3- (3-trifluoromethyl- phenyl)-urea | 2.50E- 08 | 3.20E- 07 | |
| 5 | 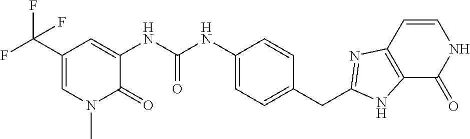 [SMILES: Cn1cc(C(F)(F)F)cc(NC(=O)Nc2ccc(Cc3nc4cc[nH]c(=O)c4[nH]3)cc2)c1=O] | 1-[4-(3-Methyl-4-oxo- 4,5-dihydro-3H- imidazo[4,5-c]pyridin- 2-ylmethyl)-phenyl]-3- (1-methyl-2-oxo-5- trifluoromethyl-1,2- dihydro-pyridin-3-yl)- urea | | | 1.717/ 473.2 |
| 6 | 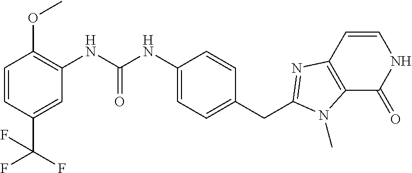 [SMILES: COc1ccc(C(F)(F)F)cc1NC(=O)Nc1ccc(Cc2nc3cc[nH]c(=O)c3n2C)cc1] | 1-(2-Methoxy-5- trifluoromethyl- phenyl)-3-[4-(3- methyl-4-oxo-4,5- dihydro-3H- imidazo[4,5-c]pyridin- 2-ylmethyl)-phenyl]- urea | | | 1.985/ 472.2 |
| 7 | 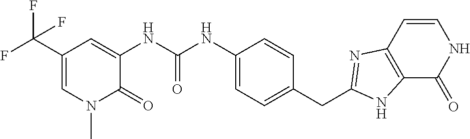 [SMILES: Cn1cc(C(F)(F)F)cc(NC(=O)Nc2ccc(Cc3nc4cc[nH]c(=O)c4[nH]3)cc2)c1=O] | 1-(1-Methyl-2-oxo-5- trifluoromethyl-1,2- dihydro-pyridin-3-yl)- 3-[4-(4-oxo-4,5- dihydro-3H- imidazo[4,5-c]pyridin- 2-ylmethyl)-phenyl]- urea | | | 1.665/ 459.1 |
| 8 | 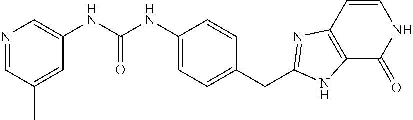 [SMILES: Cc1cncc(NC(=O)Nc2ccc(Cc3nc4cc[nH]c(=O)c4[nH]3)cc2)c1] | 1-(5-Methyl-pyridin-3- yl)-3-[4-(4-oxo-4,5- dihydro-3H- imidazo[4,5-c]pyridin- 2-ylmethyl)-phenyl]- urea | | | 1.108/ 375.1 |

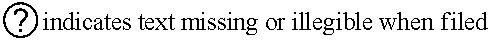

TABLE 5b
NMR data of compounds of table 5a

| No. of | |
|---|---|
| compound | |
| of table 5a | 1HNMR |
| 6 | 1H NMR (500 MHz, DMSO-d6) ppm = 11.16 (s, 1H), 9.41 (s, 1H), 8.66-8.35 |
| | (m, 2H), 7.58-6.82 (m, 7H), 6.49 (s, 1H), 4.15 (s, 2H), 3.98-3.90 (m, 6H). |
| 7 | 1H NMR (400 MHz, DMSO-d6) ppm = 11.16 (s, 1H), 9.61 (s, 1H), 8.79 (s, 1H), |
| | 8.23 (d, J = 2.5, 1H), 7.99 (s, 1H), 7.39 (d, J = 8.3, 2H), 7.23 (d, J = 8.2, 2H), 7.08 |
| | (t, J = 6.1, 1H), 6.46 (d, J = 7.0, 1H), 4.06 (s, 2H), 3.58 (s, 3H). |
| 8 | 1H NMR (400 MHz, DMSO-d6) ppm = 12.78 (s, 1H), 11.08 (s, 1H), 8.93 (s, 1H), |
| | 8.91 (s, 1H), 8.40 (s, 1H), 8.03 (s, 1H), 7.78 (s, 1H), 7.43-7.35 (m, 2H), 7.26-7.17 |
| | (m, 2H), 7.04 (t, J = 5.8, 1H), 6.44 (d, J = 7.0, 1H), 4.03 (s, 2H), 2.27 (s, 3H). |

TABLE 6

| | | Compound | IC50 | | IC50 |
|---|---|---|---|---|---|
| No. | Compound (structure) | (chemical name) | [DDR2] | | [p-DDR2] |
| 1 | 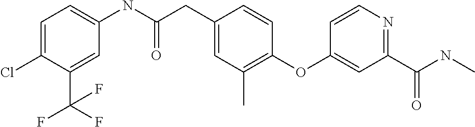 [SMILES: CNC(=O)c1cc(Oc2ccc(CC(=O)Nc3ccc(Cl)c(C(F)(F)F)c3)cc2C)ccn1] | 4-{4-[(4-Chloro-3- trifluoromethyl- phenylcarbamoyl)- methyl]-2-methyl- phenoxy}-pyridine-2- carboxylic acid methylamide | 110 | nM | 44 nM |
| 2 | 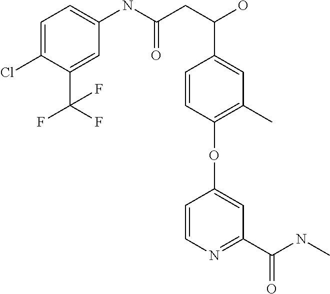 [SMILES: CNC(=O)c1cc(Oc2ccc(C(O)CC(=O)Nc3ccc(Cl)c(C(F)(F)F)c3)cc2C)ccn1] | 4-{4-[2-(4-Chloro-3- trifluoromethyl- phenylcarbamoyl)-1- hydroxy-ethyl]-2- methyl-phenoxy}- pyridine-2-carboxylic acid methylamide | 21 | μM | |
| 3 | 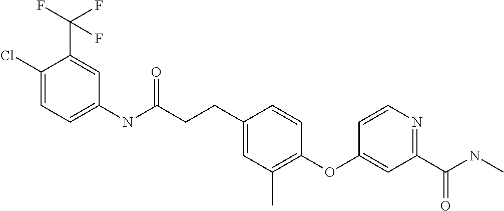 [SMILES: CNC(=O)c1cc(Oc2ccc(CCC(=O)Nc3ccc(Cl)c(C(F)(F)F)c3)cc2C)ccn1] | 4-{4-[2-(4-Chloro-3- trifluoromethyl- phenylcarbamoyl)- ethyl]-2-methyl- phenoxy}-pyridine-2- carboxylic acid methylamide | 1.6 | μM | |
| 4 | 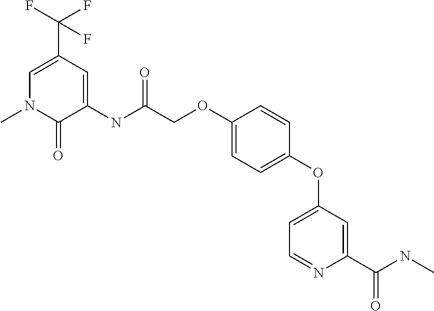 | 4-{4-[(1-Methyl-2-oxo- 5-trifluoromethyl-1,2- dihydro-pyridin-3- ylcarbamoyl)- methoxy]-phenoxy}- pyridine-2-carboxylic acid methylamide | 570 | nM | |
| 5 | 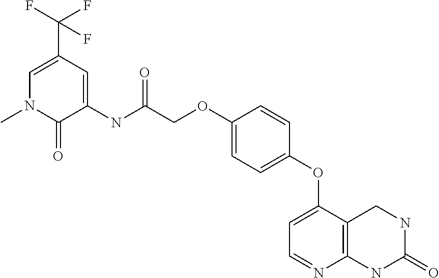 [SMILES: Cn1cc(C(F)(F)F)cc(NC(=O)COc2ccc(Oc3ccnc4c3CNC(=O)N4)cc2)c1=O] | N-(1-Methyl-2-oxo-5- trifluoromethyl- 1,2-dihydro-pyridin-3- yl)-2-[4-(2- oxo-1,2,3,4- tetrahydro-pyrido[2,3- d]pyrimidin-5-yloxy)- phenoxy]-acetamide | | | |
| 6 | 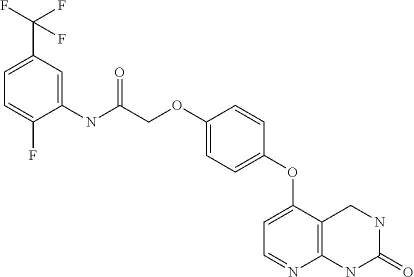 [SMILES: O=C(COc1ccc(Oc2ccnc3c2CNC(=O)N3)cc1)Nc1cc(C(F)(F)F)ccc1F] | N-(2-Fluoro-5- trifluoromethyl- phenyl)-2-[4-(2-oxo- 1,2,3,4-tetrahydro- pyrido[2,3-d]pyrimidin- 5-yloxy)-phenoxy]- acetamide | | | |
| 7 | 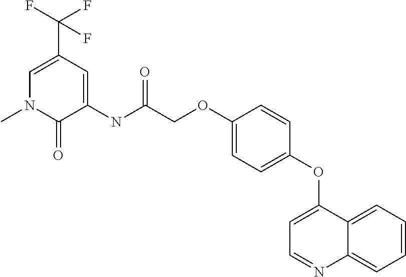 [SMILES: Cn1cc(C(F)(F)F)cc(NC(=O)COc2ccc(Oc3ccnc4ccccc34)cc2)c1=O] | N-(1-Methyl-2-oxo-5- trifluoromethyl- 1,2-dihydro-pyridin-3- yl)-2-[4-(quinolin-4- yloxy)-phenoxy]- acetamide | | | |
| 8 | 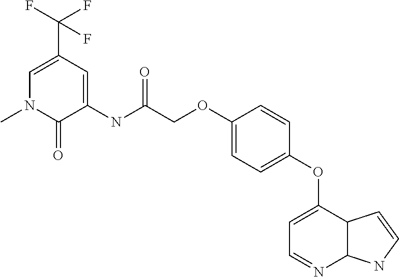 [SMILES: Cn1cc(C(F)(F)F)cc(NC(=O)COc2ccc(OC3=CC=NC4NC=CC34)cc2)c1=O] | 2-[4-(3a,7a-Dihydro- 1H-pyrrolo[2,3- b]pyridin-4-yloxy)- phenoxy]-N-(1- methyl-2-oxo-5- trifluoromethyl-1,2- dihydro-pyridin-3-yl)- acetamide | | | |
| 9 | 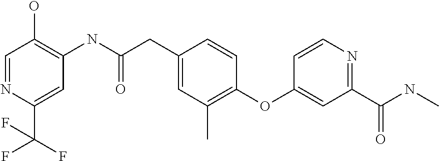 [SMILES: CNC(=O)c1cc(Oc2ccc(CC(=O)Nc3cc(C(F)(F)F)ncc3O)cc2C)ccn1] | 4-{4-[(2-Hydroxy-5- trifluoromethyl- pyridin-3- ylcarbamoyl)-methyl]- 2-methyl-phenoxy}- pyridine-2-carboxylic acid methylamide | | | |
| 10 | 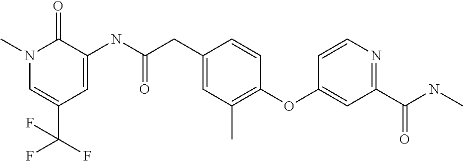 | 4-{2-Methyl-4-[(1- methyl-2-oxo-5- trifluoromethyl-1,2- dihydro-pyridin-3- ylcarbamoyl)-methyl]- phenoxy}-pyridine-2- carboxylic acid methylamide | | | |
| 11 | 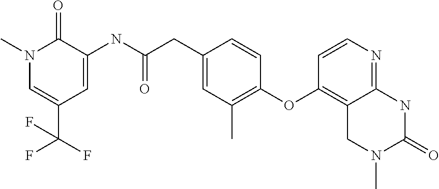 [SMILES: Cc1cc(CC(=O)Nc2cc(C(F)(F)F)cn(C)c2=O)ccc1Oc1ccnc2c1CN(C)C(=O)N2] | 2-[3-Methyl-4-(3- methyl-2-oxo-1,2,3,4- tetrahydro-pyrido[2,3- d]pyrimidin-5-yloxy)- phenyl]-N-(1-methyl- 2-oxo-5- trifluoromethyl-1,2- dihydro-pyridin-3-yl)- acetamide | | | |
| 12 | 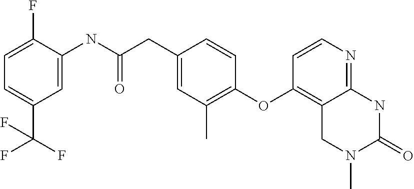 [SMILES: Cc1cc(CC(=O)Nc2cc(C(F)(F)F)ccc2F)ccc1Oc1ccnc2c1CN(C)C(=O)N2] | N-(2-Fluoro-5- trifluoromethyl- phenyl)-2-[3-methyl-4- (3-methyl-2-oxo- 1,2,3,4-tetrahydro- pyrido[2,3-d]pyrimidin- 5-yloxy)-phenyl]- acetamide | | | |

In order to avoid any doubt, in all cases where the chemical name of a compound according to the invention and the depiction of the chemical structure of the compound mistakenly do not agree, the compound according to the invention is defined unambiguously by the depiction of the chemical structure.

### EXAMPLE 2

### Preparation of the Compounds According to the Invention

The compounds according to the invention can be prepared, for example, by methods known to the person skilled in the art by the following synthesis sequences. The examples indicated describe the synthesis, but do not restrict the latter to the examples.

### EXAMPLE 2.1

### Synthesis of 1-(2-Methoxy-5-methyl-pyridin-3-yl)-3-[3-methyl-4-(2-methyl-pyridin-4-yloxy)-benzyl]-urea

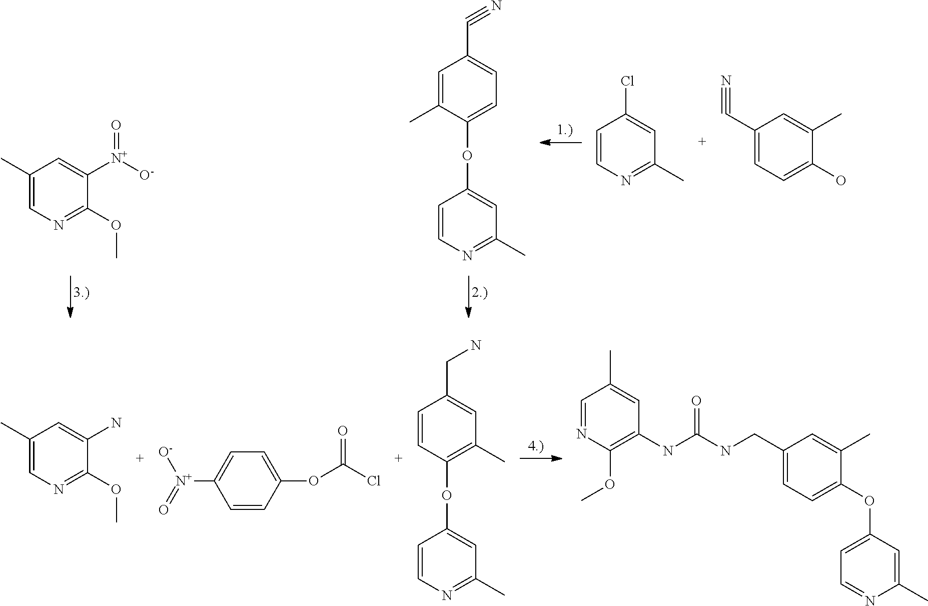

### 1. Synthesis of 3-Methyl-4-(2-methyl-pyridin-4-yloxy)-benzonitrile

4-Chloro-2-methyl-pyridine (1.00 g, 7.84 mmol, 1 eq.) and 4-Hydroxy-3-methyl-benzonitrile (1.57 g, 11.76 mmol, 1.5 eq.) are mixed together and heated for about 16 h to 160° C. Reaction mixture was cooled down to room temperature, EtOAc and 2N NaOH were added, organic phase was separated and washed twice with 2N NaOH and water. The organic phase was separated, washed once with saturated NaCl-solution and dried over Na₂SO₄. After filtration the organic phase was reduced in vacuo. The brown residue (HPLC/MS: Rₜ=1.227 min, M+H 243.1) became crystalline upon standing on air.

### 2) Synthesis of 3-Methyl-4-(2-methyl-pyridin-4-yloxy)-benzylamine

3-Methyl-4-(2-methyl-pyridin-4-yloxy)-benzonitrile (1.20 g, 5.35 mmol, 1 eq.) was dissolved in MeOH/NH₃ (20%, 5 ml), sponge nickel (0.60 g) as catalyst were added and the mixture was stirred under an atmosphere of H₂ (5 bar) at 50° C. for about 16 h. The reaction mixture was reduced in vacuo. The residue (HPLC/MS: Rₜ=0.435 min, M+H 229.1) was used directly in the next reaction without further purification.

### 3) Synthesis of 2-Methoxy-5-methyl-pyridin-3-ylamine

2-Methoxy-5-methyl-3-nitro-pyridine (1.00 g, 5.95 mmol, 1 eq.) was dissolved in THF (10 ml), wet Pd/C (0.50 g) was added. The reaction mixture was stirred at room temperature for about 16 h under an atmosphere of H₂ (400 ml, 17.84 mmol, 3 eq.). The reaction mixture was filtrated and the solvend removed in vacuo. The product (HPLC/MS: Rₜ=1.058 min, M+H 129.3) was obtained as brown crystals, which were used in the next reaction without further purification.

### 4) Synthesis of 1-(2-Methoxy-5-methyl-pyridin-3-yl)-3-[3-methyl-4-(2-methyl-pyridin-4-yloxy)-benzyl]-urea

Synthesis of 2-Methoxy-5-methyl-pyridin-3-ylamine (48.00 mg, 0.35 mmol, 1 eq.) was dissolved in DCM (10 ml), 4-nitro-phenyl-chloro-formiate (78.00 mg, 0.39 mmol, 1.1 eq.) and pyridine (31 ml) were added. The mixture was stirred for 2 h at room temperature. Then were added 3-Methyl-4-(2-methyl-pyridin-4-yloxy)-benzylamine (80.00 mg, 0.35 mmol, 1 eq.) and N-ethyl-diisopropyl-amine (0.06 ml, 0.35 mmol, 1 eq.). The mixture was stirred for about 16 h at room temperature. To the mixture was added DCM. The organic layer was washed once with 1N NaOH and twice with water, it was dried over Na₂SO₄, filtrated and the solvent removed in vacuo. The residue was purified by preparative HPLC.

HPLC (RP-18): Chromolith-prep RP-18e 100-25, Shimadzu LC 8A

Eluent A: H₂O+0.1% TFA

Eluent B: Acetonitrile+0.1% TFA

Gradient: 99:1->1:99 in 15 min.

30 ml/min, Detektion: UV 220 nm

The product (HPLC/MS: Rₜ=1.503 min, M+H 393.2) was obtained as yellow oil.

1H-NMR (DMSO, 500 mHz) σ in ppm=8.66 (d, J=5 Hz, 1H), 8.25 (d, J=5 Hz, 1H), 8.13 (s, 1H), 7.52 (m, 1H), 7.45 (m, 1H), 7.37 (m, 1H), 7.30 (m, 1H), 7.27 (m, 1H), 7.27-7.19 (m, 2H), 4.35 (d, J=5 Hz, 2H), 3.90 (s, 3H), 2.63 (s, 3H), 2.18 (s, 3H), 2.12 (s, 3H)

### Abbreviations:

DCM=dichloromethane
DMA=dimethylacetamide
DMF=dimethylformamide
EA=ethyl acetate
MTBE=methyl tert-butyl ether
PE=petroleum ether
RT=room temperature
TFA=trifluoroacetic acid

### EXAMPLE 2.2

### Synthesis of 4-{2-Methyl-4-[3-(1-methyl-2-oxo-5-trifluoromethyl-1,2-dihydro-pyridin-3-yl)-ureidomethyl]-phenoxy}-pyridine-2-carboxylic acid methyl amide

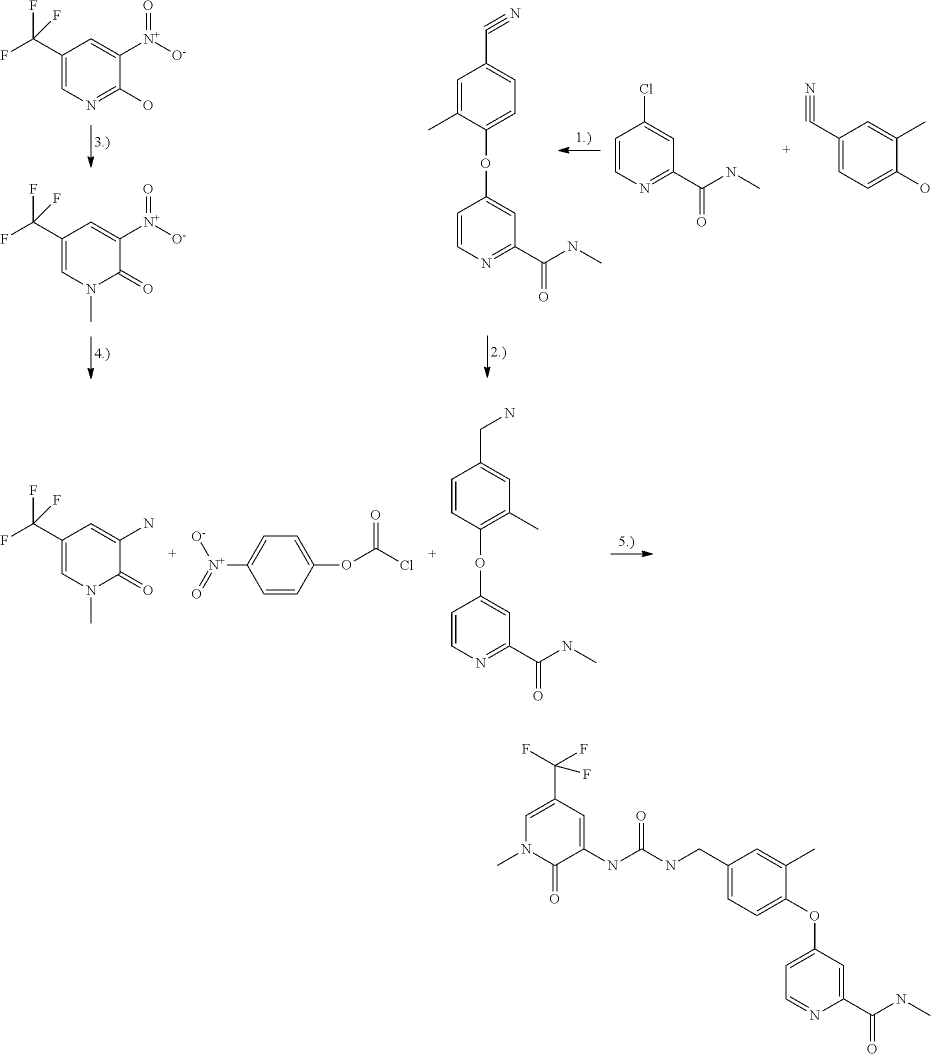

### 1) Synthesis of 4-(4-Cyano-2-methyl-phenoxy)-pyridine-2-carboxylic acid methylamide

A solution of 4-Hydroxy-3-methyl-benzonitrile (0.100 g; 0.717 mmol) in dry DMF (3 mL) was treated with potassium-tert-butyl at (0.088 g; 0.788 mmol). The reaction mixture was stirred at RT for 2 h and 4-Chloro-pyridine-2-carboxylic acid methylamide (0.130 g; 0.717 mmol) and potassium carbonate (0.020 ml; 0.358 mmol) were added. The resulting suspension was then heated to 130° C. for 4 days. For purification the reaction mixture was allowed to cool down to RT and it was washed with 1 N NaOH-solution (5 mL) and water (5 mL). The solid, that precipitated while washing, was filtrated and added into the organic layer. The aqueous phase was extracted with DCM (2×15 mL) and the combined organic layers were evaporated to dryness. The resulting solid was dissolved in DCM (20 mL), dried with Na₂SO₄ and concentrated to afford the crude product. The product was purified with flash column chromatography (Combi Flash RF, Si-60, 24 g-column, gradient PE/EE 95:5 to 50:50 in 12 min then for 7 min isocratic 50:50, flow 35 ml/min, UV 254 nM) resulting in 4-(4-Cyano-2-methyl-phenoxy)-pyridine-2-carboxylic acid methylamide (136,000 mg; 0.443 mmol) as yellow solid.

### 2) Synthesis of 4-(4-Aminomethyl-2-methyl-phenoxy)-pyridine-2-carboxylic acid methylamide

A solution of 4-(4-Cyano-2-methyl-phenoxy)-pyridine-2-carboxylic acid methylamide (0.690 g; 0.836 mmol) in methanol (5 mL) and NH₃ in methanol (20%, 5 mL) was treated with nickel sponge (0.5 g Johnson Matthey, A-7000) and purged with H₂. The reaction mixture was stirred at RT for 17.5 h with a pressure of five bar. The catalyst was filtrated off and the solvent was evaporated. The crude product was then purified by flash column chromatography (Flashmaster, UV 240 nM, 70 g silica gel column, flow 20 ml/min, DCM/MeOH 9:1) yielding 4-(4-Aminomethyl-2-methyl-phenoxy)-pyridine-2-carboxylic acid methylamide (0.182 g; 0.584 mmol) as yellow resin.

### 3) Synthesis of 1-Methyl-3-nitro-5-trifluoromethyl-1H-pyridin-2-one

To a solution of 3-Nitro-5-(trifluoromethyl)pyridin-2-ol (60.0 g; 288.33 mmol) in DMF (500 ml) was added potassium carbonat (120.0 g; 864.99 mmol) and iodomethan (19.7 ml; 317.16 mmol). The resulting suspension was stirred for about 16 h at 80° C. The reaction mixture was diluted with EtOAc and extracted 3× with water, dried over Na₂SO₄, filtrated and the solution evaporated to dryness.

The residue was treated with THF/petroleum ether (PE). The precipitated product was filtered off, rinsed with PE and dried in vacuo to yield a brown solid.

### 4) Synthesis of 3-Amino-1-methyl-5-trifluoromethyl-1H-pyridin-2-one

To a solution of 1-Methyl-3-nitro-5-trifluoromethyl-1H-pyridin-2-one (9.40 g, 42.32 mmol) in THF (100 ml) and MeOH (10 ml) was added 5% Pd/C (54% H₂O, 2 g). The reaction was stirred under an atmosphere of hydrogen at room temperature. After 16 h additional Pd/C (4 g) were added and stirring was continued under hydrogen (1 atm) for 23 hours. The solids were removed via filtration and the filtrate reduced in vacuo to yield 3-Amino-1-methyl-5-trifluoromethyl-1H-pyridin-2-one (7.9 g, 41.1 mmol).

### 5) Synthesis of 4-{2-Methyl-4-[3-(1-methyl-2-oxo-5-trifluoromethyl-1,2-dihydro-pyridin-3-yl)-ureidomethyl]-phenoxy}-pyridine-2-carboxylic acid methyl amide

3-Amino-1-methyl-5-trifluoromethyl-1H-pyridin-2-one (1.64 g, 8.55 mmol) was dissolved in DCM (50 ml), 4-nitro-phenyl-chloro-formiate (1.90 g, 9.437 mmol) and pyridine (0.76 ml) were added. The mixture was stirred for 2 h at room temperature. Then were added 4-(4-Aminomethyl-2-methyl-phenoxy)-pyridine-2-carboxylic acid methylamide (2.32 mg, 8.55 mmol) and N-ethyl-diisopropyl-amine (2.91 ml, 17.10 mmol). The mixture was stirred for about 16 h at room temperature. To the mixture was added DCM. The organic layer was washed once with 1N NaOH and twice with water, it was dried over Na₂SO₄, filtrated and the solvent removed in vacuo. The residual mixture was taken up with MTBE, the resulting white precipitate was filtered off and dried in vacuo.

4-{2-Methyl-4-[3-(1-methyl-2-oxo-5-trifluoromethyl-1,2-dihydro-pyridin-3-yl)-ureidomethyl]-phenoxy}-pyridine-2-carboxylic acid methylamide was obtained as white solid (HPLC/MS: Rₜ=2.184 min, M+H 490.2).

1H NMR (500 MHz, DMSO-d6) ppm=8.74 (q, J=4.6, 1H), 8.65 (s, 1H), 8.50 (d, J=5.6, 1H), 8.22 (d, J=2.5, 1H), 7.96-7.92 (m, 1H), 7.76 (t, J=5.9, 1H), 7.34-7.31 (m, 1H), 7.29 (d, J=2.6, 1H), 7.25 (dd, J=8.2, 2.2, 1H), 7.15-7.11 (m, 1H), 7.10 (dd, J=5.6, 2.6, 1H), 4.33 (d, J=5.8, 2H), 3.57 (s, 3H), 2.79 (d, J=4.9, 3H), 2.10 (s, 3H).

Method Info: HPLC/MS

A: H₂O+0.05% HCOOH|B: MeCN+0.04% HCOOH

T: 30° C.|Flow: 2 ml/min|Column: Chromolith RP-18e 50-4.6 mm|MS: 85-800 amu

1%->100% B: 0->2.8 min|100% B: 2.8->3.3 min

### EXAMPLE 2.3

### Synthesis of (4-trifluoromethyl-pyridin-2-yl)-carbamic acid 3-methyl-4-(2-methylcarbamoyl-pyridin-4-yloxy)-benzyl ester

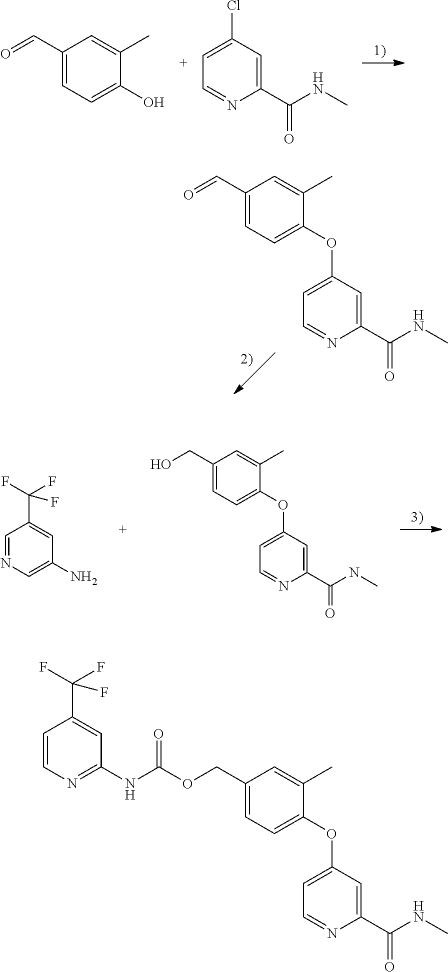

### 1) Synthesis of 4-(4-formyl-2-methyl-phenoxy)-pyridine-2-carboxylic acid methylamide

A solution of 4-hydroxy-3-methylbenzaldehyde (503.7 mg; 3.7 mmol) in dry DMF (7 mL) was treated with potassium-tert-butoxide (456.7 mg; 4.1 mmol). The reaction mixture was stirred at RT for 2 h and then 4-chloro-pyridine-2-carboxylic acid methylamide (672.6 mg; 3.7 mmol) and potassium carbonate (255.7 mg; 1.9 mmol) were added. The resulting suspension was heated to 130° C. for 5 days. For purification the reaction mixture was allowed to cool down to RT and water (50 mL) and DCM (50 mL) were added. The phases were separated and the organic layer was washed with 1 M NaOH-solution (50 mL) brine (20 mL), dried over Na₂SO₄ and the solvent was evaporated. The crude product was purified with flash column chromatography (Combi Flash RF, Si-60, 120 g-column, gradient CH/EE 100:0 to 45:55 in 28 min, flow 85 ml/min, UV 254 nM and 280 nM) obtaining 4-(4-formyl-2-methyl-phenoxy)-pyridine-2-carboxylic acid methylamide (598 mg; 2.21 mmol) as white solid.

### 2) Synthesis of 4-(4-hydroxymethyl-2-methyl-phenoxy)-pyridine-2-carboxylic acid methylamide

4-(4-formyl-2-methyl-phenoxy)-pyridine-2-carboxylic acid methylamide (400 mg; 1.5 mmol) was dissolved in dry THF (6 mL). The mixture was treated with sodium borohydride (61 mg; 1.6 mmol) and stirred for 3 h at 50° C. For purification methanol (15 mL) was added and the mixture was stirred for further 30 min. Then the mixture was evaporated to dryness and water (10 mL) and ethylacetate (30 mL) were added. The phases were separated, the organic layer was washed with brine (10 mL) and dried over Na₂SO₄. Finally the solvent was evaporated yielding in 4-(4-hydroxymethyl-2-methyl-phenoxy)-pyridine-2-carboxylic acid methylamide (370 mg; 1.4 mmol) as white solid.

### 3) Synthesis of (4-trifluoromethyl-pyridin-2-yl)-carbamic acid 3-methyl-4-(2-methylcarbamoyl-pyridin-4-yloxy)-benzyl ester

4-Trifluoromethyl-pyridin-2-ylamine (53.6 mg; 0.33 mmol) was dissolved in DCM (1.1 ml). Additionally, 4-nitrophenylchlorformiate (73.6 mg; 0.37 mmol) and pyridine (0.029 ml; 0.37 mmol) were added and the reaction mixture was stirred for 2 h. Then 4-(4-hydroxymethyl-2-methyl-phenoxy)-pyridine-2-carboxylic acid methylamide (90 mg; 0.33 mmol) dissolved in DMF (1 mL) and N-ethyldiisopropylamina (0.112 mL; 0.66 mmol) were added and the reaction mixture was stirred for further 2 d at RT. For purification the solvent was evaporated and the crude product was directly purified with prep. HPLC (Agilent 1100 Series, SunFire™ Prep C18 OBM™ 5 μm (150-30 mm) column, gradient ACN/H2O 99:1 to 30:70 in 3 min, then 30:70 to 60:40 in 18 min, flow 50 ml/min, UV 220 nM) yielding in (4-trifluoromethyl-pyridin-2-yl)-carbamic acid 3-methyl-4-(2-methylcarbamoyl-pyridin-4-yloxy)-benzyl ester (22.8 mg; 0.05 mmol) as white TFA-salt.

HPLC/MS: (T: 30° C.|Flow: 2 ml/min|Column: Chromolith, RP-18e 50-4.6 mm, 4%->100% B: 0->2.8 min|100% B: 2.8->3.3 min, A: H2O+0.05% HCOOH, B: MeCN+0.04% HCOOH):

Rt=2.470 min, [M+H] 461.2

1H NMR (500 MHz, DMSO-d6) ppm=10.80 (s, 1H), 8.75 (q, J=4.8, 1H), 8.55 (d, J=5.2, 1H), 8.51 (d, J=5.6, 1H), 8.16 (s, 1H), 7.52-7.49 (m, 1H), 7.44-7.39 (m, 2H), 7.28 (d, J=2.6, 1H), 7.21-7.15 (m, 1H), 7.12 (dd, J=5.6, 2.6, 1H), 5.23 (s, 2H), 2.78 (d, J=4.9, 3H), 2.12 (s, 3H).

### EXAMPLE 2.4

### Synthesis of 1-[4-(4-Oxo-4,5-dihydro-3H-imidazo[4,5-c]pyridin-2-ylmethyl)-phenyl]-3-(3-trifluoromethyl-phenyl)-urea and 1-(5-Methyl-pyridin-3-yl)-3-[4-(4-oxo-4,5-dihydro-3H-imidazo[4,5-c]pyridin-2-ylmethyl)-phenyl]-urea

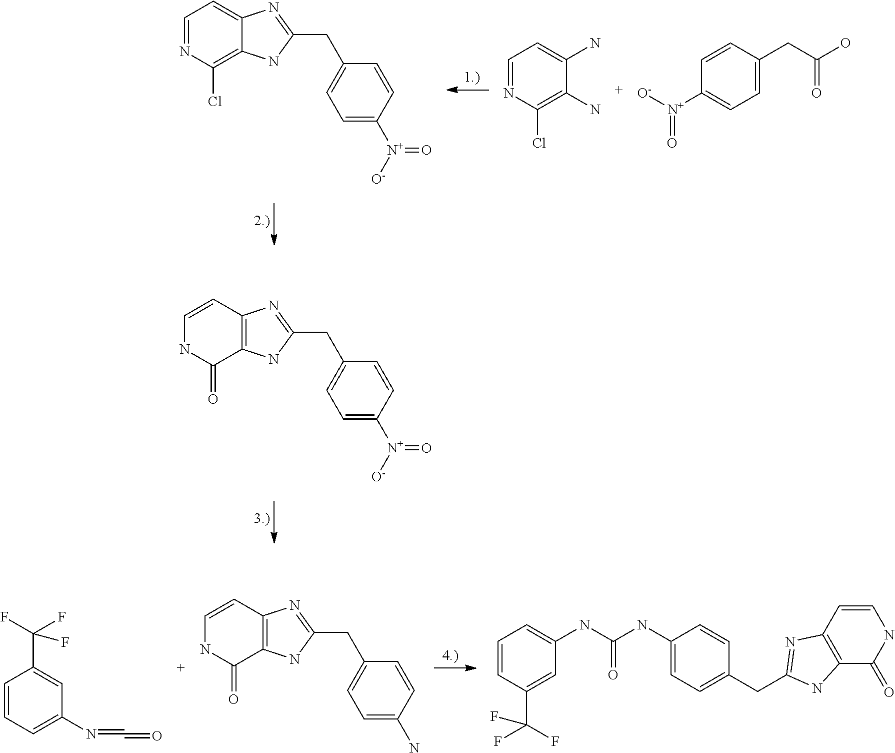

1) Synthesis of 1-[4-(4-Oxo-4,5-dihydro-3H-imidazo[4,5-c]pyridin-2-ylmethyl)-phenyl]-3-(3-trifluoromethyl-phenyl)-urea was prepared as described in WO2006/042599 A1 in four steps.

### 2) Synthesis of 1-(5-Methyl-pyridin-3-yl)-3-[4-(4-oxo-4,5-dihydro-3H-imidazo[4,5-c]pyridin-2-ylmethyl)-phenyl]-urea

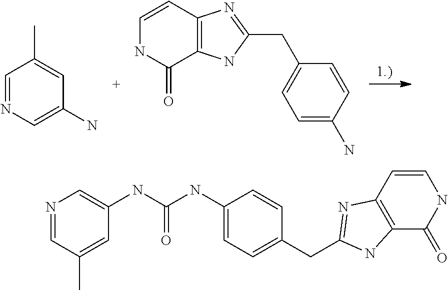

To a solution of 5-Methyl-pyridin-3-ylamine (100.00 mg; 0.925 mmol) in THF was added Triphosgen (109.76 mg; 0.370 mmol) and triethylamine (0.26 ml; 1.849 mmol) at 10° C. The mixture was stirred at r.t. for 2 hours. A solution of 2-(4-Amino-benzyl)-3,5-dihydro-imidazo[4,5-c]pyridin-4-one (177.74 mg; 0.740 mmol) in (5.00 ml) was added and the mixture was stirred at r.t. for about 16 h. Water was added and the mixture was extracted with DCM. The crude product precipitates as a yellow solid. The solid was collected by filtration, washed with water and dried in vacuo. The crude product was purified by flash chromatography on silica gel (Teledyne-Isco Combi Flash RF, Si-60, 4 g, gradient: DCM/MeOH 100:0 to 80:20 in 13 min and 5 min isocratic 80:20, flow-rate: 18 ml/min, UV 254 nm) to afford 1-(5-Methyl-pyridin-3-yl)-3-[4-(4-oxo-4,5-dihydro-3H-imidazo[4,5-c]pyridin-2-ylmethyl)-phenyl]-urea (33.00 mg; 0.085 mmol).

1H NMR (400 MHz, DMSO-d6) ppm=12.78 (s, 1H), 11.08 (s, 1H), 8.93 (s, 1H), 8.91 (s, 1H), 8.40 (s, 1H), 8.03 (s, 1H), 7.78 (s, 1H), 7.43-7.35 (m, 2H), 7.26-7.17 (m, 2H), 7.04 (t, J=5.8, 1H), 6.44 (d, J=7.0, 1H), 4.03 (s, 2H), 2.27 (s, 3H).

HPLC/MS: Rt=1.108 min, [M+H]=375.1

Method Info: HPLC/MS

A: H₂O+0.05% HCOOH|B: MeCN+0.04% HCOOH

T: 30° C.|Flow: 2 ml/min|Column: Chromolith RP-18e 50-4.6 mm|MS: 85-800 amu; gradient 4%->100% B: 0->2.8 min|100% B: 2.8->3.3 min

### EXAMPLE 2.5

### Synthesis of 4-{4-[(4-Chloro-3-trifluoromethyl-phenylcarbamoyl)-methyl]-2-methyl-phenoxy}-pyridine-2-carboxylic acid methylamide

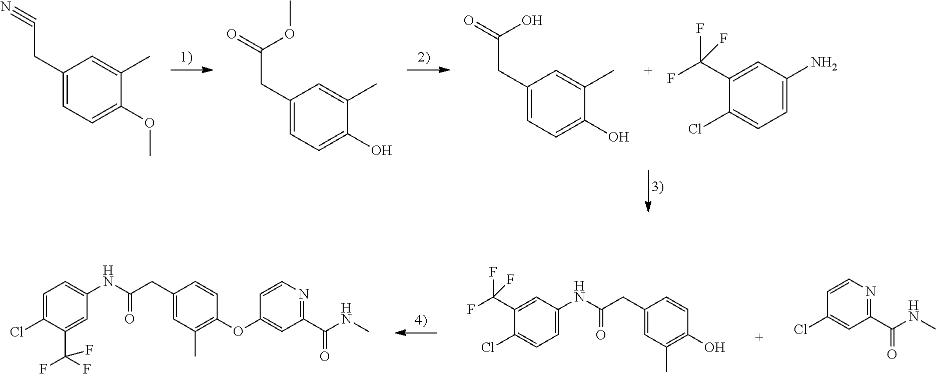

### 1) Synthesis of (4-hydroxy-3-methyl-phenyl)-acetic acid methyl ester

A solution of (4-methoxy-3-methyl-phenyl)-acetonitrile (3.3 g; 20.4 mmol) in DCM (20 mL) was cooled to −78° C. and treated drop wise with boron tribromide (5.8 ml; 61.2 mmol) in DCM (30 mL) over 30 min. The reaction mixture was allowed to warm to RT and was then stirred for 20 h. For purification methanol (50 mL) was added drop wise at 0° C. and the solution was washed with water (2×50 mL). The aqueous phase was back extracted with DCM (5×50 mL) and the combined organic layers were dried over Na₂SO₄. Finally the solvent was evaporated and the crude product was purified with flash column chromatography (Combi Flash RF, Si-60, 120 g-column, gradient CH/EE 100:0 to 75:25 in 29 min then isocratic 75:25 for 13 min, flow 85 ml/min, UV 254 nM and 280 nM) obtaining (4-hydroxy-3-methyl-phenyl)-acetic acid methyl ester (1.8 g; 8 mmol) as colourless oil.

### 2) Synthesis of (4-hydroxy-3-methyl-phenyl)-acetic acid

(4-Hydroxy-3-methyl-phenyl)-acetic acid methyl ester (1.8 g; 8 mmol) was dissolved in a 2 M NaOH-solution (20 mL) and stirred for 1.5 h at RT. The reaction mixture was then adjusted to pH 4 with a 6 M HCl-solution and extracted with DCM (4×70 mL). The combined organic layers were dried over Na₂SO₄ and the solvent was evaporated obtaining (4-hydroxy-3-methyl-phenyl)-acetic acid (1.3 g; 7.9 mmol) as white solid.

### 3) Synthesis of N-(4-chloro-3-trifluoromethyl-phenyl)-2-(4-hydroxy-3-methyl-phenyl)-acetamide

5-amino-2-chlorobenzotrifluoride (455.2 mg; 2.3 mmol) and (4-hydroxy-3-methyl-phenyl)-acetic acid (429.7 mg; 2.3 mmol) were dissolved in dry DMF (9 mL). Additionally HOBT (463.3 mg; 3 mmol), EDCI (490.783 mg; 2.6 mmol) and 4-methylmorpholine (0.27 mL; 2.6 mmol) were added to start the reaction. The reaction mixture was stirred for 24 h at 60° C. Then water (30 mL) and DCM (30 mL) were added and the phases were separated. The organic layer was washed with water (15 mL) and brine (15 mL), dried over Na₂SO₄ and the solvent was evaporated. The crude product was purified with flash column chromatography (Combi Flash RF, Si-60, 40 g-column, gradient CH/EE 100:0 to 50:50 in 16 min then isocratic 50:50 for 8 min, flow 40 ml/min, UV 254 nM and 280 nM) obtaining N-(4-chloro-3-trifluoromethyl-phenyl)-2-(4-hydroxy-3-methyl-phenyl)-acetamide (243 mg; 0.5 mmol) as yellow oil.

### 4) Synthesis of 4-{4-[(4-Chloro-3-trifluoromethyl-phenylcarbamoyl)-methyl]-2-methyl-phenoxy}-pyridine-2-carboxylic acid methylamide

A solution of N-(4-chloro-3-trifluoromethyl-phenyl)-2-(4-hydroxy-3-methyl-phenyl)-acetamide (243 mg; 0.5 mmol) in dry DMF (1.1 mL) was treated with potassium-tert-butoxide (64 mg; 0.6 mmol). The reaction mixture was stirred at RT for 2 h and 4-chloro-pyridine-2-carboxylic acid methylamide (104 mg; 0.6 mmol) and potassium carbonate (35.9 mg; 0.3 mmol) were added. The resulting suspension was heated to 130° C. for 1 day. For purification the reaction mixture was allowed to cool to RT and water (15 mL) and DCM (15 mL) were added. The phases were separated and the organic layer was washed with water (15 mL), brine (15 mL), dried over Na₂SO₄ and finally the solvent was evaporated. The crude product was purified with flash column chromatography (Combi Flash RF, Si-60, 24 g-column, gradient CH/EE 100:0 to 35:65 in 21 min then isocratic 35:65 for 6 min, flow 35 mL/min, UV 254 nM and 280 nM) obtaining 4-{4-[(4-chloro-3-trifluoromethyl-phenylcarbamoyl)-methyl]-2-methyl-phenoxy}-pyridine-2-carboxylic acid methylamide (82.5 mg, 0.2 mmol) as yellow solid.

HPLC/MS (Method Info: A: H2O+0.05% HCOOH|B: MeCN+0.04% HCOOH, T: 30° C.|Flow: 2 ml/min|Column: Chromolith RP-18e 50-4.6 mm|MS: 85-800 amu, 4%->100% B: 0->2.8 min|100% B: 2.8->3.3 min)

Rt=2.526 min [M+H] 478.1

1H NMR (300 MHz, DMSO-d6) ppm=10.63 (s, 1H), 8.82-8.66 (m, 1H), 8.49 (d, J=5.7, 1H), 8.28-8.15 (m, 1H), 7.92-7.81 (m, 1H), 7.65 (d, J=8.8, 1H), 7.41-7.32 (m, 1H), 7.33-7.23 (m, 2H), 7.17-7.04 (m, 2H), 3.72 (s, 2H), 2.78 (d, J=4.8, 3H), 2.09 (s, 3H).

### EXAMPLE 3

### Autophosphorylation Assay for Biochemical Activity Testing of DDR2

The autophosphorylation assay was run in two steps: the enzymatic reaction in which His-tagged DDR2 with ATP as co-substrate phosphorylates itself and the detection reaction where a time resolved FRET between XL665® labelled anti-6His antibody bound to the His-tag of the enzyme and cryptate labelled anti-phospho-Tyrosine-antibody (PT66) bound the phosphorylated Tyrosine residue of DDR2 was analysed. The autophosphorylation activity was detectable directly via the increase in HTRF signal.

The autophosphorylation assay was performed as 1536 well or 384 well HTRF® (Cisbio, Codolet, France) assay format in Greiner low volume medium binding 384-well microtiter plates and was used for high throughput screen. 4 nM His-tagged human recombinant DDR-2 kinase domain (His-TEV-DDR2 467-855 aa) and 150 μM ATP as co-substrate were incubated in a total volume of 6 μl (50 mM HEPES, 10 mM Mg-chloride, 0.01% Brij®-35, 2 mM DTT, 1% DMSO, 1 mM EGTA, 0.1% BSA, pH 7.5) in the absence or presence of the test compound (10 dilution concentrations) for 150 min at 22° C. The reaction was stopped by the addition of 4 μl detection solution (16.5 nM anti-6His antibody-XL665® (Cisbio, Codolet, France) and 2.75 nM Anti-phospho-tyrosine (PT66) labelled with Eu-Cryptate® (PT66-K, Cisbio, Codolet, France) in 50 mM HEPES, 400 mM KF, 0.1% BSA, 20 mM EDTA, pH 7.0). After 1 h incubation at room temperature the HTRF was measured with an Envision multimode reader (Perkin Elmer LAS Germany GmbH) at excitation wavelength 340 nm (laser mode) and emission wavelengths 615 nm and 665 nm. The ratio of the emission signals was determined. The full value used was the inhibitor-free reaction. The pharmacological zero value used was Nilotinib (LC Laboratories, USA) in a final concentration of 4 The inhibitory values (IC50) were determined using either the program Symyx Assay Explorer® or Condosseo® from GeneData (see tables in Example 1).

### EXAMPLE 4

### Phospho-DDR2 Cellular Assay

Assays were performed in a 384 well plate format, using cell line HEK293 transfected with human DDR2 (PLT460F_(DDR2)-P7-1)

### Materials and Methods:

Cells were seeded at a density of 10'000 cells/well in 384well poly-D-lysine coated Black/clear plate (Cellcoat Greiner) and incubated in DMEM medium in the presence of 10% fetal bovine serum at 37° C., 5% CO2 for 48 h. Medium was replaced by serum-free medium and cells were incubated at 37° C., 5% CO2 for 8 h. Compound to be tested in 5% DMSO or 5% DMSO and 50 μg/ml of chicken collagen II were added and cells were incubated at 37° C., 5% CO2 for 16 h

Cells were rinsed with ice-cold PBS, lysed with lysis buffer (M-PER Thermo #78501) for 30 min at room temperature and centrifuged for 1 min at 1000 RPM. In parallel, White High binding 384 well plates (Corning) were coated with mouse anti-human DDR2 capture antibody (R&D Systems kit DuoSet IC Human phospho-DDR2 ELISA), overnight at RT.

An aliquot of cell lysate was transferred to the coated plates and incubated for 2 h at RT. Plates were washed and mouse anti-phospho-tyrosine detection antibody (R&D Systems kit DuoSet IC Human phospho-DDR2 ELISA) conjugated to horse radish peroxidase (HRP) was added for 2 h at RT. Plates were washed and chemiluminescent substrate for HRP (Thermo) was added for 15 min at RT. Luminescence was measured on a luminometer.

Percentage inhibition of Collagen II induced DDR2 phosphorylation was calculated using Inhibitor controls (50 μg/ml collagen II+0.3 μM Dasatinib) and Neutral control (50 μg/ml collagen II+1% DMSO) using Genedata software (see tables in Example 1).

### EXAMPLE 5

### Investigation of the Anti-Hyperalgesic Effect in Animals

In order to induce an inflammation reaction, a carrageenan solution (CAR, 1%, 50 μl) was injected intra-articularly on one side into a rat knee joint. The uninjected side was used for control purposes. Six animals per group were used. The threshold was determined by means of a micrometer screw (medial-lateral on the knee joint), and the thermal hyperalgesia was determined by means of a directed infrared light source by the Hargreaves method (Hargreaves et al., 1988) on the sole of the foot. Since the site of inflammation (knee joint) is different from the site of measurement (paw sole), use is made here of the term secondary thermal hyperalgesia, the mechanism of which is of importance for the discovery of effective analgesics.

Experimental description of thermal hyperalgesia (Hargreaves test): the experimental animal is placed in a plastic chamber on a quartz sheet. Before testing, the experimental animal is firstly given about 5-15 minutes time to familiarise itself with the environment. As soon as the experimental animal no longer moves so frequently after the familiarisation phase (end of the exploration phase), the infrared light source, whose focus is in the plane of the glass bottom, is positioned directly beneath the rear paw to be stimulated. An experiment run is then started by pressing the button: infrared light results in an increase in the skin temperature of the rear paw. The experiment is terminated either by the experimental animal raising the rear paw (as an expression of the pain threshold being reached) or by automatic switching-off of the infrared light source when a prespecified maximum temperature has been reached. Light reflected by the paw is recorded as long as the experimental animal sits still. Withdrawal of the paw interrupts this reflection, after which the infrared light source is switched off and the time from switching on to switching off is recorded. The instrument is calibrated in such a way that the infrared light source increases the skin temperature to about 45 degrees Celsius in 10 s (Hargreaves et al. 1988). An instrument produced by Ugo Basile for this purpose is used for the testing.

CAR was purchased from Sigma-Aldrich. Administration of the specific cathepsin D inhibitor, compound no. 23 (from Example 1, Table 1, (S)-2-[(2S,3S)-2-((3S,4S)-3-amino-4-{(S)-3-methyl-2-[(S)-4-methyl-2-(3-methyl-butyrylamino)pentanoylamino]butyrylamino}-5-phenylpentanoylamino)-3-methylpentanoylamino]-3-methylbutyric acid), was carried out intra-articularly 30 minutes before the CAR. Triamcinolone (TAC) in an amount of 10 μg/joint was used as positive control, and the solvent (vehicle) was used as negative control. The hyperalgesia is quoted as the difference in the withdrawal times between the inflamed and non-inflamed paw.

Result: TAC was capable of reducing the CAR-induced swelling, but the specific DDR2 inhibitor was not. In contrast, the specific DDR2 inhibitor was able to reduce the extent of thermal hyperalgesia as a function of the dose. Assessment: it has been shown that the compounds of the present invention exert an anti-hyperalgesic action. This can be postulated, since the compounds of the present invention exhibited no influence on inflammatory swelling and thus on the hyperalgesia trigger. It can thus be assumed that the compounds of the present invention develop a pain-reducing action in humans.

### EXAMPLE 6

### Stability of the Compounds According to the Invention in Bovine Synovial Fluid

### Extraction of Bovine Synovial Fluid:

In the preparation of bovine explants (for the diffusion chamber or other assays), either cow hoof (metacarpal joints) or cow knee is used. The synovial fluid can be obtained from both joints. To this end, the synovial fluid is carefully removed from the open joint using a 10 ml syringe and a cannula and transferred into prepared 2 ml Eppendorf vessels. The Eppendorf vessels are labelled depending on the animal (cow passport is available). It must be ensured here that blood does not enter the joint gap during preparation of the joints. If this is the case, the synovial fluid will become a reddish colour and must consequently be discarded. The synovial fluid is basically highly viscous and clear to yellowish in colour. The removal together with a macroscopic analysis of the synovial fluid is documented.

### Batch for Stability Testing of Substances in SF:

In order to check the stability of individual compounds, a pool of four different bovine synovial fluids is mixed. To this end, about 1 ml per SF is used. The mixture is prepared directly in a 5 ml glass vessel. The SFs are mixed thoroughly, but carefully. No air bubbles or foam should form. To this end, a vortex unit is used at the lowest speed. The compounds to be tested are tested in an initial concentration (unless required otherwise) of 1 μM. After addition of the substance, the batch is again mixed thoroughly and carefully. For visual monitoring, all SF batches are photographed, and the pictures are filed in the eLabBio file for the corresponding experiment. FIG. 1 shows photodocumentation of this type by way of example. The batches are incubated in the incubator for 48 h at 37° C. and 7.5% CO₂.

### Sampling:

The sampling is carried out after the pre-agreed times (unless required otherwise, see below). 200 μl of the SF are removed from the mixture per time and transferred directly into a 0.5 ml “low-binding” Eppendorf vessel. “Low-binding” Eppendorf vessels are used in order to minimise interaction of the substances with the plastic of the vessels. 200 μl of acetonitrile have already been introduced into the Eppendorf vessel, so that a 1+1 mixture of the SF forms thereafter. This simplifies the subsequent analysis, but precipitation of protein may occur immediately after addition of the SF. This should be noted on the protocol. The 0 h sample is taken immediately after addition of the substance. This corresponds to the 100% value in the stability calculation. Ideally, the concentration employed should be retrieved here. The samples can be frozen at −20° C.
0 h
6 h
24 h
48 h

The negative control used is SF without substance. The positive control used is SF with 1 μM of substance. This corresponds to the 0 h value and thus 100% stability.

The samples are stored in “low-binding” Eppendorf vessels at −20° C. The samples are subsequently measured quantitatively.

### Data Processing:

The concentrations measured (ng/ml) are plotted against the time in a graph (Graph Pad Prism®). The percentage stability of the substance is determined here. The 100% value used is the initial value in the SF at time 0 h. The data are stored in eLabBio under the respective experiment number and reported in the MSR database (as percent stability after the corresponding incubation times).

### Results:

All compounds measured remained stable (see tables in Example 1). Compound stability is defined as >80% compound recovery after 48 h.

### EXAMPLE 7

### Evaluation of the DDR2 Inhibitors on the Production of MMP13 by SW1353 Cells Upon Stimulation with Collagen Type II

### Principle:

The binding of Collagen type II to the DDR2 receptor of the SW1353 cells initiate a signalling cascade resulting in the increase of MMP13 expression. MMP13 is released in the culture medium in its pro-form, the proMMP13 which can be measured with an ELISA.

DDR2 inhibitors are evaluated for their ability to block this signalling cascade and therefore proMMP13 production upon collagen stimulation.

### Material and Methods:

| Name | Supplier | Cat. number |
|---|---|---|
| RPMI 1640 | Gibco | 21765 |
| FCS | Promocell | C37350 |
| L-Glutamin | Gibco | 25030 |
| Natrium | Gibco | 11360 |
| Pyruvate | | |
| HEPES | Gibco | 15630 |
| Eisessig | MERCK | 8.18755.1000 |
| Trypsin/EDTA | Invitrogen | 25300 |
| Collagen Typ II | SIGMA | C9301-5MG |
| DMSO | MERCK | 1.02931.0500 |
| Dasatinib | TRC | D193600 |
| Human Pro- | R&D Systems | DM1300 |
| MMP-13 | | |
| Quantikine | | |
| ELISA Kit | | |

### Cell Culture:

SW1353 cells (ATCC, HTB-94), conserved in liquid nitrogen, are thawed and cultivated at 1.6.10⁶ cells in a T75 in RPMI1640 supplemented with 2 mM Glutamin, 1 mM Na-Pyruvate, 10% FCS, at 37° C., 5% CO₂ for three days. SW1353 cells are then harvested with trypsin/EDTA and resuspended in RPMI1640 supplemented with 2 mM Glutamin, 1 mM Na-Pyruvate, 25 mM HEPES and 0.5% FCS (assay medium) and inoculated in a 96 well plate at 30 000 cells/well in 100 μL of the assay medium and further incubated 24 hours at 37°, 5% CO₂ to enable cell adhesion. For stimulation of the DDR2 receptor, 50 μL of a collagen type II solution 160 μg/mL (final concentration in the well is 40 μg/mL) will be added in each well as well as 50 μL of the different dilutions of the inhibitors (MSCs) from 0.003 μM to 10 μM (final concentration in the plate). Each condition is performed in four-plicates. After three additional days of culture, the supernatant will be harvested for proMMP13 measurement.

The Different Controls Present on Each Plate are Composed of Assay Medium with:
Positive control (with the reference compound): 40 μg/mL Collagen type II, 0.03 μM Dasatinib (reference compound) in 0.1% DMSO
Negative control (no inhibition): 40 μg/mL Collagen type II and 0.1% DMSO
Medium control (no stimulation): 0.005% Acetic acid and 0.1% DMSO
All wells contains 0.1% DMSO and 0.005% acetic acid.

MSCs concentrations used are 10, 3, 1, 0.3, 0.1, 0.03, 0.01 and 0.003 μM

The solutions needed are:
Collagen type II is diluted at 2 mg/mL in 0.25% acetic acid. This stock solution can be stored at 4° C. for a week and is diluted 1/12.5 in the assay medium (to obtain 160 μg/mL in 0.02% acetic acid) before being used in the assay.
MSCs from 0.012 to 40 μM in assay medium with 0.4% DMSO (they are then further diluted ¼ in the assay plate)
Dasatinib 0.12 μM in assay medium with 0.4% DMSO (it is then further diluted ¼ in the assay plate)
Acetic acid 0.02% in assay medium (control).

### ProMMP13 Measurement:

The harvested supernatants are either directly used or stored at −20° C. until use. ProMMP13 is measured with a commercial ELISA kit, according the recommendation of the manufacturer (see Annex). Briefly, 50 μL of each samples are used undiluted and the standard curve is realised in the assay medium. At the end of the assay the ELISA plate is read on a Paradigm MTP-Reader (Beckman Coulter) at 540 (reference wavelength) and 450 nm. All the absorbance values obtained at 450 nm are corrected with the absorbance at 540 nm and a ‘Four Parameter Fit’ is used to establish the standard curve. From the standard curve the concentrations of proMMP13 in all the samples are calculated. All the calculations are realised by the Paradigm software.

### Calculations:

The data reported in the database are the % effect of the compounds at the two highest concentrations (10 and 3 μM) as well as the IC₅₀.

% effect at the two highest concentrations is calculated according to the formula:

$$\% \_effect @ 10 μM = \frac{MMP 13 @ 10 μM - MMP 13 negative\_control}{MMP 13 positive\_control - MMP 13 negative\_control}$$

The IC₅₀s are calculated with the software Graph Pad Prism (see tables in Example 1).

### EXAMPLE 8

### Injection Vials

A solution of 100 g of a compound of the formula I and 5 g of disodium hydrogenphosphate in 3 l of bidistilled water is adjusted to pH 6.5 using 2 N hydrochloric acid, filtered under sterile conditions, transferred into injection vials, lyophilised under sterile conditions and sealed under sterile conditions. Each injection vial contains 5 mg of a compound of the formula I.

### EXAMPLE 9

### Solution

A solution is prepared from 1 g of a compound of the formula I, 9.38 g of NaH₂PO₄2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE 10

### Ointment

500 mg of a compound of the formula I are mixed with 99.5 g of Vaseline under aseptic conditions.

### EXAMPLE 11

### Ampoules

A solution of 1 kg of a compound of the formula I in 60 l of bidistilled water is filtered under sterile conditions, transferred into ampoules, lyophilised under sterile conditions and sealed under sterile conditions. Each ampoule contains 10 mg of a compound of the formula I.

1. Compounds of the formula I, 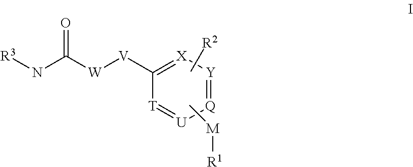
in which
W is O, N, CH₂, CH₂CH₂, CH₂CHOH or —(CH₂)O—,
X, Y, Q, U, T are independently from one another C or N, with the proviso that one or more of X, Y, Q, U and T are carbon atoms and that M is bonded to a carbon atom,
V is a single bond or —CR⁴R⁵—,
M is O or —CR⁴R⁵—,
R¹ is mono- or bicyclic heteroaryl, heterocyclyl or aryl containing 3 to 14 carbon atoms and 1 or 4 heteroatoms, independently selected from N, O and S, which is unsubstituted or mono-, di- or trisubstituted by R⁶,
R² is H, A, CN, OH, OA or Hal,
R³ is mono- or bicyclic heteroaryl, heterocyclyl or aryl containing 3 to 14 carbon atoms and 1 or 4 heteroatoms, independently selected from N, O and S, which is unsubstituted or mono-, di- or trisubstituted by R⁷,
R⁴, R⁵ are independently from one another selected from the group consisting of H and A,
R², R⁶ and R⁷ are independently from one another selected from the group consisting of H, A, ═O, OH, OA, Hal, CH₂Hal, CH(Hal)₂, C(Hal)₃, NO₂, (CH₂)—CN, (CH₂)—NR⁸R⁹, (CH₂)—O(CH₂)ₖNR⁸R⁹, (CH₂)ₙNR⁸(CH₂)ₖNR⁸R⁹, (CH₂)ₙO(CH₂)ₖOR¹⁸, (CH₂)ₙNR⁸(CH₂)ₖOR⁹, (CH₂)ₙCOOR¹⁰, (CH₂)ₙCHOR¹⁰, (CH₂)ₙCHONR⁸R⁹, C(O)NR⁸R⁹, C(O)NHANH₂(CH₂)ₙNR⁸COR¹⁰, (CH₂)ₙNR⁸CONR⁸R⁹, (CH₂)ₙNR⁸SO₂A, (CH₂)ₙSO₂NR⁸R⁹, (CH₂)ₙS(O)ₙR¹⁰, (CH₂)ₙOC(O)R¹⁰, (CH₂)ₙC(O)R¹⁰, (CH₂)ₙSR⁸, CH═N—OA, CH₂CH═N—OA, (CH₂)ₙNHOA, (CH₂)ₙCH═N—R⁸, (CH₂)ₙOC(O)NR⁸R⁹, (CH₂)ₙNR⁸COOR¹⁰, (CH₂)ₙN(R⁸)CH₂CH₂OR¹⁰, (CH₂)ₙN(R⁸)CH₂CH₂OCF₃, (CH₂)ₙN(R⁸)C(R¹⁰)HCOOR⁹, (CH₂)ₙN(R⁸)C(R¹⁰)HCOR⁹, (CH₂)ₙN(R⁸)CH₂CH₂N(R⁹)CH₂COOR⁸, (CH₂)ₙN(R⁸)CH₂CH₂NR⁸R⁹, CH═CHCOOR¹⁰, CH═CHCH₂NR⁸R⁹, CH═CHCH₂NR⁸R⁹, CH═CHCH₂OR¹⁰, (CH₂)ₙN(COOR¹⁰)COOR¹¹, (CH₂)ₙN(CONH₂)COOR¹⁰, (CH₂)ₙN(CONH₂)CONH₂, (CH₂)ₙN(CH₂COOR¹⁰)COOR¹¹, (CH₂)ₙN(CH₂CONH₂)COOR¹⁰, (CH₂)ₙN(CH₂CONH₂)CONH₂, (CH₂)ₙCHR¹⁰COR¹¹, (CH₂)ₙCHR¹⁰COOR¹¹, (CH₂)ₙCHR¹⁰CH₂OR¹¹, (CH₂)ₙOCN and (CH₂)ₙNCO,
R⁸, R⁹ are independently from one another selected from the group consisting of H, A, (CH₂)ₘAr¹ and (CH₂)ₘHet, or in NR⁸R¹⁹R⁸ and R⁹ form, together with the N-atom they are bound to, a 5-, 6- or 7-membered heterocyclus which optionally contains 1 or 2 additional hetero atoms, selected from N, O and S,
R¹⁰, R¹¹ are independently from one another selected from the group consisting of H, Hal, A, (CH₂)ₘAr² and (CH₂)ₘHet,
A is selected from the group consisting of alkyl, alkenyl and cycloalkyl,
Ar¹, Ar² are independently from one another aromatic hydrocarbon residues comprising 5 to 12 and preferably 5 to 10 carbon atoms which are optionally substituted by one or more substituents, selected from a group consisting of A, Hal, NO₂, CN, OR¹², NR¹²R¹³, COOR¹², CONR¹²R¹³, NR¹²COR¹³, NR¹²CONR¹²R¹³, NR¹²SO₂A, COR¹², SO₂R¹²R¹³, S(O)ᵤA and OOCR¹²,
Het is a saturated, unsaturated or aromatic mono- or bicyclic heterocyclic residue containing 3 to 14 carbon atoms and 1 or 4 heteroatoms, independently selected from N, O and S, which is optionally substituted by one or more substituents, selected from a group consisting of A, Hal, NO₂, CN, OR¹², NR¹²R¹³, COOR¹², CONR¹²R¹³, NR¹²COR¹³, NR¹²CONR¹²R¹³, NR¹²SO₂A, COR¹², SO₂R¹²R¹², S(O)ᵤA and OOCR¹²,
R¹², R¹³ are independently from one another selected from the group consisting of H, A, and (CH₂)ₘAr³,
Ar³ is a 5- or 6-membered aromatic hydrocarbon which is optionally substituted by one or more substituents selected from a group consisting of methyl, ethyl, propyl, 2-propyl, tert.-butyl, Hal, CN, OH, NH₂ and CF₃,
k, u, n and m are independently from one another 0, 1, 2, 3, 4, or 5,
Hal is independently selected from one another from the group consisting of F, Cl, Br and I,
and physiologically acceptable salts, solvates and stereoisomers thereof, including mixtures thereof in all ratios.

2. Compounds according to claim 1 in which
W is N,
X, Y, Q, U, T are C,
V is a single bond or —CR⁴R⁵—,
M is O,
R¹ is 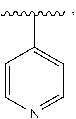 [SMILES: *c1ccncc1]
which is unsubstituted or monosubstituted by R⁶,
R² is H, alkyl with 1 to 5 C-atoms, CN, OH, OA or Hal,
R³ is 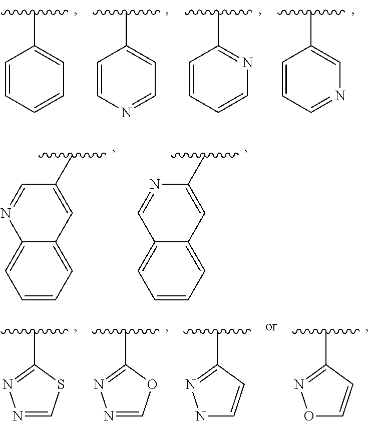
which is unsubstituted or mono-, di- or trisubstituted by R⁷,
R⁴, R⁵ are independently from one another selected from the group consisting of H, alkyl and cycloalkyl,
R⁶ is H, alkyl, C(O)NHA or C(O)NHANH₂,
R⁷ is H, alkyl, cycloalkyl, Hal, CF₃, ═O, CN, SA, C(O)A, COOH, CONH₂, CONHA, CONA₂, CONHANHA, (CH₂)ₙOH, (CH₂)ₙOA, OCH₂C(O)OA, O(CH₂)ₙNH₂, O(CH₂)ₙNHA, O(CH₂)ₙNA₂, O(CH₂)ₙNASO₂A, AOH, OAOH, OAC(O)NH₂, O(CH₂)ₙheterocyclyl, heterocyclyl, SO₂CF₃ or OANAC(O)OA and
n is 0-3
and physiologically acceptable salts, solvates and stereoisomers thereof, including mixtures thereof in all ratios.

3. Compounds according to claim 1 in which
W is N,
X, Y, Q, U, T are C,
V is a single bond or —CR⁴R⁵—,
M is O,
R¹ is 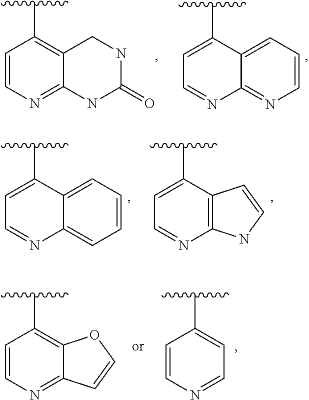
which is unsubstituted or mono-, di- or trisubstituted by R⁶,
R² is H, A, CN, OH, OA or Hal,
R³ is 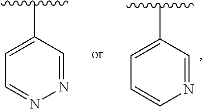 [SMILES: *c1ccnnc1 ; "or" ; *c1cccnc1]
which is unsubstituted or mono-, di- or trisubstituted by R⁷,
R⁴, R⁵ are H,
R⁶ is H, A, ═O, CN, CF₃, Hal, COOH, C(O)NH₂, C(O)NHA, C(O)NA₂, (CH₂)ₙOH, (CH₂)ₙOA, (CH₂)ₙaryl, (CH₂)ₙ heteroaryl or (CH₂)ₙheterocyclyl,
R⁷ is H, A, ═O, CN, (CH₂)ₙOH, (CH₂)ₙOA, CF₃, Hal, COOH, (CH₂)ₙaryl, (CH₂)ₙ heteroaryl or (CH₂)ₙheterocyclyl,
A is alkyl, and
n is 0-3
and physiologically acceptable salts, solvates and stereoisomers thereof, including mixtures thereof in all ratios.

4. Compounds according to claim 1 in which
W is N,
X, Y, Q, U, T are C,
V is a single bond or —CR⁴R⁵—,
M is O,
R¹ and R⁶ together are 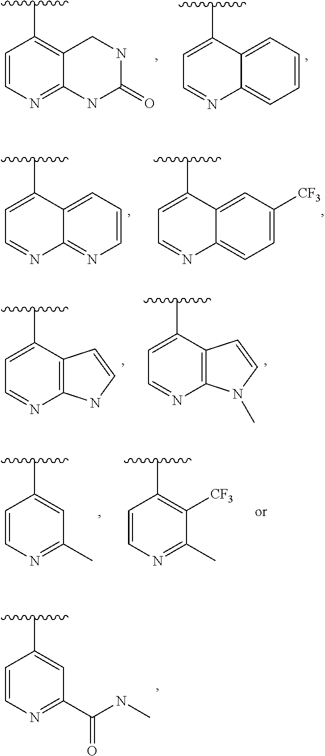
R² is H or alkyl with 1 to 5 C-atoms,
R³ and R⁷ together are 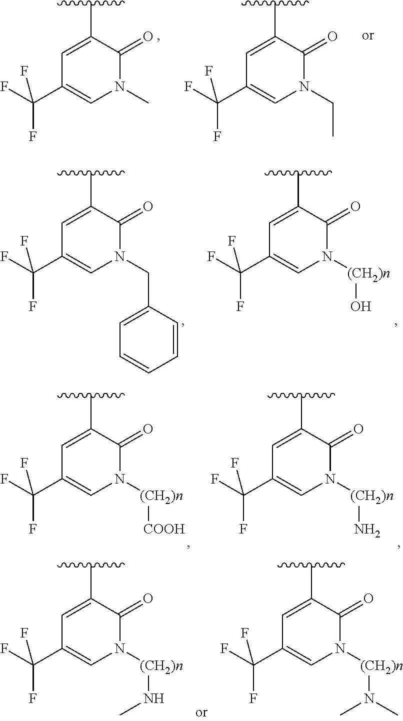
R⁴, R⁵ are H and
n 0-3
and physiologically acceptable salts, solvates and stereoisomers thereof, including mixtures thereof in all ratios.

5. Compounds according to claim 1 in which
W is O,
X, Y, Q, U, T are independently from one another C or N, with the proviso that one or more of X, Y, Q, U and T are carbon atoms and that M is bonded to a carbon atom,
V is a single bond or —CR⁴R⁵—,
M is O,
R¹ is 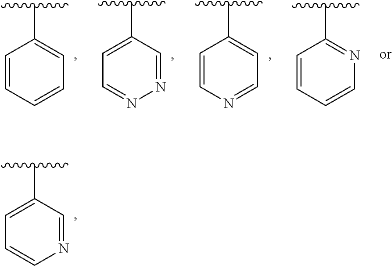
which is unsubstituted or mono-, di- or trisubstituted by R⁶,
R³ is 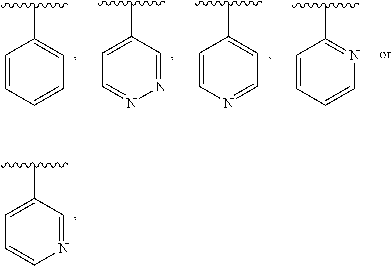
which is unsubstituted or mono-, di- or trisubstituted by R⁷,
R⁴, R⁵ are independently from one another selected from the group consisting of H, alkyl and cycloalkyl, and
R², R⁶ and R⁷ independently from one another have the meanings as disclosed in claim 1
and physiologically acceptable salts, solvates and stereoisomers thereof, including mixtures thereof in all ratios.

6. Compounds according to claim 1 in which
W is O,
X, Y, Q, U, T are C,
V is a single bond or —CR⁴R⁵—,
M is O,
R¹ and R⁶ together are 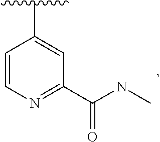 [SMILES: *c1ccnc(C(=O)NC)c1]
R² is H or alkyl with 1 to 5 C-atoms,
R³ is 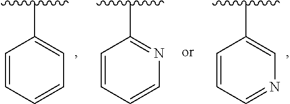
which is unsubstituted or mono-, di- or trisubstituted by R⁷,
R⁴, R⁵ are H,
R⁷ alkyl with 1-5 C-atoms, CN, OH, OA, Hal or CF₃
and physiologically acceptable salts, solvates and stereoisomers thereof, including mixtures thereof in all ratios.

7. Compounds according to claim 1 in which
W N,
X, Y, Q, U, T are C,
V is a single bond,
M is —CR⁴R⁵—,
R¹ is 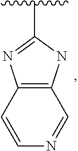 [SMILES: *c1nc2ccncc2[nH]1]
which is unsubstituted or mono-, di- or trisubstituted by R⁶,
R² is H, A, CN, OH, OA or Hal,
R³ is 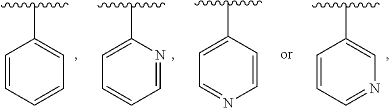
which is unsubstituted or mono-, di- or trisubstituted by R⁷,
R⁴, R⁵ are independently from one another selected from the group consisting of H, alkyl and cycloalkyl, and
R⁶, R⁷ are independently from one another selected from the group consisting of H, alkyl with 1-5 C-atoms, ═O, CN, Hal, CF₃, OH, OA, COOH, C(O)NH₂ and C(O)NHA,
and physiologically acceptable salts, solvates and stereoisomers thereof, including mixtures thereof in all ratios.

8. Compounds according to claim 1 in which
W N,
X, Y, Q, U, T are C,
V is a single bond,
M is —CR⁴R⁵—,
R¹ and R⁶ together are 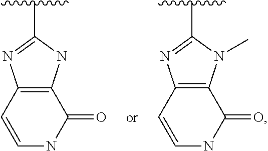 [SMILES: *c1nc2cc[nH]c(=O)c2[nH]1 ; "or" ; *c1nc2cc[nH]c(=O)c2n1C]
R² is H,
R³ is 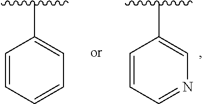 [SMILES: *c1ccccc1 ; *c1cccnc1]
which is unsubstituted or mono-, di- or trisubstituted by R⁷,
R⁴, R⁵ are H,
R⁷ is H, alkyl with 1-5 C-atoms, ═O, CF₃, OH, OA or Hal,
and physiologically acceptable salts, solvates and stereoisomers thereof, including mixtures thereof in all ratios.

9. Compounds according to claim 1 in which
W is CH₂, CH₂CH₂, CH₂CHOH or —(CH₂)O—,
X, Y, Q, U, T are C,
V is a single bond,
M is O,
R¹ is 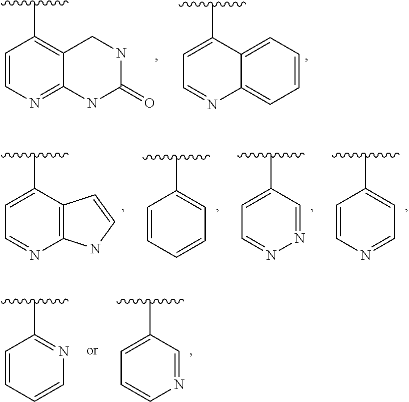
which is unsubstituted or mono-, di- or trisubstituted by R⁶,
R³ is 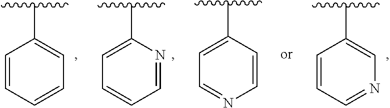
which is unsubstituted or mono-, di- or trisubstituted by R⁷,
R², R⁶ and R⁷ independently from one another have the meanings as disclosed in claim 1
and physiologically acceptable salts, solvates and stereoisomers thereof, including mixtures thereof in all ratios.

10. Compounds according to claim 1 in which
W is CH₂, CH₂CH₂, CH₂CHOH or —(CH₂)O—,
X, Y, Q, U, T are C,
V is a single bond,
M is O,
R¹ is 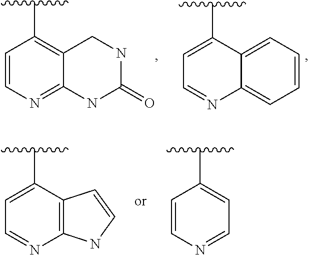
which is unsubstituted or mono-, di- or trisubstituted by R⁶,
R² is H or alkyl with 1-5 C-atoms,
R³ is 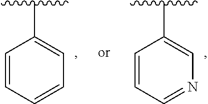 [SMILES: *c1ccccc1 ; *c1cccnc1]
which is unsubstituted or mono-, di- or trisubstituted by R⁷,
R⁶ is H, alkyl, cycloalkyl, ═O, CF₃, CN, (CH₂)ₙOH, (CH₂)ₙOA, Hal, COOH, C(O)NH₂ or C(O)NHA,
R⁷ is H, ═O, A, CN, (CH₂)ₙOH, (CH₂)ₙOA, Hal or CF₃,
A is alkyl, and
n is 0-3
and physiologically acceptable salts, solvates and stereoisomers thereof, including mixtures thereof in all ratios.

11. Compounds according to claim 1
a) 4-{4-[3-(3,5-Dichloro-pyridin-4-yl)-ureidomethyl]-phenoxy}-pyridine-2-carboxylic acid methylamide
b) 4-{4-[3-(2,6-Dichloro-pyridin-4-yl)-ureidomethyl]-phenoxy}-pyridine-2-carboxylic acid methylamide
c) 4-{4-[(3-Pyridin-2-yl-ureido)-methyl]-phenoxy}-pyridine-2-carboxylic acid methylamide
d) 4-{4-[3-(2-Methoxy-pyridin-3-yl)-ureidomethyl]-phenoxy}-pyridine-2-carboxylic acid methylamide
e) 4-{4-[(3-Pyridin-3-yl-ureido)-methyl]-phenoxy}-pyridine-2-carboxylic acid methylamide
f) 4-{4-[3-(2-Chloro-pyridin-4-yl)-ureidomethyl]-phenoxy}-pyridine-2-carboxylic acid methylamide
g) 4-{4-[3-(3-Chloro-5-trifluoromethyl-pyridin-2-yl)-ureidomethyl]-phenoxy}-pyridine-2-carboxylic acid methylamide
h) 4-{4-[3-(2,5-Dichloro-pyridin-3-yl)-ureidomethyl]-phenoxy}-pyridine-2-carboxylic acid methylamide
i) 4-{4-[(3-Isoquinolin-3-yl-ureido)-methyl]-phenoxy}-pyridine-2-carboxylic acid methylamide
j) 4-{4-[(3-Quinolin-3-yl-ureido)-methyl]-phenoxy}-pyridine-2-carboxylic acid methylamide
k) 4-{4-[3-(2-Methoxy-quinolin-3-yl)-ureidomethyl]-phenoxy}-pyridine-2-carboxylic acid methylamide
l) 4-{4-[3-(5-Methyl-pyridin-2-yl)-ureidomethyl]-phenoxy}-pyridine-2-carboxylic acid methylamide
m) 4-{2-Methyl-4-[3-(5-methyl-pyridin-2-yl)-ureidomethyl]-phenoxy}-pyridine-2-carboxylic acid methylamide
n) 4-{4-[3-(4-Methyl-pyridin-2-yl)-ureidomethyl]-phenoxy}-pyridine-2-carboxylic acid methylamide
o) 4-{2-Methyl-4-[3-(4-methyl-pyridin-2-yl)-ureidomethyl]-phenoxy}-pyridine-2-carboxylic acid methylamide
p) 4-{4-[3-(2-Chloro-pyridin-3-yl)-ureidomethyl]-phenoxy}-pyridine-2-carboxylic acid methylamide
q) 4-{4-[3-(6-Methoxy-pyridin-3-yl)-ureidomethyl]-phenoxy}-pyridine-2-carboxylic acid methylamide
r) 1-(2-Methoxy-5-methyl-pyridin-3-yl)-3-[4-(pyridin-4-yloxy)-benzyl]-urea
s) 4-{4-[3-(2-Methoxy-5-methyl-pyridin-3-yl)-ureidomethyl]-phenoxy}-pyridine-2-carboxylic acid methylamide
t) 4-{4-[3-(2-Methoxy-5-methyl-pyridin-3-yl)-ureidomethyl]-2-methyl-phenoxy}-pyridine-2-carboxylic acid methylamide
u) 4-{4-[3-(5-Chloro-2-methoxy-pyridin-3-yl)-ureidomethyl]-phenoxy}-pyridine-2-carboxylic acid methylamide
v) 4-{4-[3-(2-Chloro-5-methyl-pyridin-3-yl)-ureidomethyl]-phenoxy}-pyridine-2-carboxylic acid methylamide
w) 4-{4-[3-(5-Chloro-2-methoxy-pyridin-3-yl)-ureidomethyl]-2-methyl-phenoxy}-pyridine-2-carboxylic acid methylamide
x) 4-{4-[3-(2-Chloro-5-trifluoromethyl-pyridin-3-yl)-ureidomethyl]-phenoxy}-pyridine-2-carboxylic acid methylamide
y) 4-{4-[3-(2-Chloro-5-methyl-pyridin-3-yl)-ureidomethyl]-2-methyl-phenoxy}-pyridine-2-carboxylic acid methylamide
z) 4-{4-[3-(2-Chloro-5-trifluoromethyl-pyridin-3-yl)-ureidomethyl]-2-methyl-phenoxy}-pyridine-2-carboxylic acid methylamide
aa) 1-(5-Chloro-2-methoxy-pyridin-3-yl)-3-[4-(2-methyl-pyridin-4-yloxy)-benzyl]-urea
bb) 1-(5-Chloro-2-methoxy-pyridin-3-yl)-3-[3-methyl-4-(2-methyl-pyridin-4-yloxy)-benzyl]-urea
cc) 1-(2-Chloro-5-trifluoromethyl-pyridin-3-yl)-3-[4-(2-methyl-pyridin-4-yloxy)-benzyl]-urea
dd) 1-(2-Chloro-5-trifluoromethyl-pyridin-3-yl)-3-[3-methyl-4-(2-methyl-pyridin-4-yloxy)-benzyl]-urea
ee) 1-(2-Methoxy-5-methyl-pyridin-3-yl)-3-[3-methyl-4-(2-methyl-pyridin-4-yloxy)-benzyl]-urea
ff) 1-[3-Methyl-4-(2-methyl-pyridin-4-yloxy)-benzyl]-3-quinolin-3-yl-urea
gg) 1-(2-Methoxy-quinolin-3-yl)-3-[3-methyl-4-(2-methyl-pyridin-4-yloxy)-benzyl]-urea
hh) 1-Isoquinolin-3-yl-3-[3-methyl-4-(2-methyl-pyridin-4-yloxy)-benzyl]-urea
and physiologically acceptable salts, solvates and stereoisomers thereof, including mixtures thereof in all ratios.

12. Compounds according to claim 1
1 1-(3-Chloro-phenyl)-3-[4-(pyridin-4-yloxy)-benzyl]-urea
2 1-[4-(Pyridin-4-yloxy)-benzyl]-3-(2,4,5-trichloro-phenyl)-urea
3 4-{4-[3-(3,4-Dichloro-phenyl)-ureidomethyl]-phenoxy}-pyridine-2-carboxylic acid methylamide
4 1-[4-(Pyridin-4-yloxy)-benzyl]-3-(3-trifluoromethyl-phenyl)-urea
5 1-(2,4-Dichloro-phenyl)-3-[4-(pyridin-4-yloxy)-benzyl]-urea
6 1-[4-(Pyridin-4-yloxy)-benzyl]-3-m-tolyl-urea
7 1-(3-Acetyl-phenyl)-3-[4-(pyridin-4-yloxy)-benzyl]-urea
8 4-{4-[3-(2,4-Dichloro-phenyl)-ureidomethyl]-phenoxy}-pyridine-2-carboxylic acid methylamide
9 1-(4-Bromo-phenyl)-3-[4-(pyridin-4-yloxy)-benzyl]-urea
10 1-(2,5-Dichloro-phenyl)-3-[4-(pyridin-4-yloxy)-benzyl]-urea
11 1-(4-Fluoro-phenyl)-3-[4-(pyridin-4-yloxy)-benzyl]-urea
12 4-{4-[3-(4-Fluoro-phenyl)-ureidomethyl]-phenoxy}-pyridine-2-carboxylic acid methylamide
13 1-(2,3-Dichloro-phenyl)-3-[4-(pyridin-4-yloxy)-benzyl]-urea
14 4-{4-[3-(2-Methoxy-phenyl)-ureidomethyl]-phenoxy}-pyridine-2-carboxylic acid methylamide
15 1-(2,5-Dimethoxy-phenyl)-3-[4-(pyridin-4-yloxy)-benzyl]-urea
16 1-(4-Chloro-phenyl)-3-[4-(pyridin-4-yloxy)-benzyl]-urea
17 1-(4-Methoxy-phenyl)-3-[4-(pyridin-4-yloxy)-benzyl]-urea
18 1-[4-(Pyridin-4-yloxy)-benzyl]-3-p-tolyl-urea
19 4-{4-[3-(5-Chloro-2-methoxy-phenyl)-ureidomethyl]-phenoxy}-pyridine-2-carboxylic acid methylamide
20 4-{4-[3-(2,5-Dimethoxy-phenyl)-ureidomethyl]-phenoxy}-pyridine-2-carboxylic acid methylamide
21 1-[4-(Pyridin-4-yloxy)-benzyl]-3-(4-trifluoromethyl-phenyl)-urea
22 1-(3,5-Bis-trifluoromethyl-phenyl)-3-[4-(pyridin-4-yloxy)-benzyl]-urea
23 4-{4-[3-(2,4,5-Trichloro-phenyl)-ureidomethyl]-phenoxy}-pyridine-2-carboxylic acid methylamide
24 1-(2,3-Dimethyl-phenyl)-3-[4-(pyridin-4-yloxy)-benzyl]-urea
25 1-(2,5-Dimethyl-phenyl)-3-[4-(pyridin-4-yloxy)-benzyl]-urea
26 1-[4-(Pyridin-4-yloxy)-benzyl]-3-(2-trifluoromethyl-phenyl)-urea
27 1-(3-Chloro-4-methyl-phenyl)-3-[4-(pyridin-4-yloxy)-benzyl]-urea
28 1-(2-Ethyl-phenyl)-3-[4-(pyridin-4-yloxy)-benzyl]-urea
29 1-[4-(Pyridin-4-yloxy)-benzyl]-3-o-tolyl-urea
30 1-(2,4-Dimethyl-phenyl)-3-[4-(pyridin-4-yloxy)-benzyl]-urea
31 4-{4-[3-(3,5-Dichloro-phenyl)-ureidomethyl]-phenoxy}-pyridine-2-carboxylic acid methylamide
32 1-(5-Chloro-2-methoxy-phenyl)-3-[4-(pyridin-4-yloxy)-benzyl]-urea
33 1-(2-Chloro-phenyl)-3-[4-(pyridin-4-yloxy)-benzyl]-urea
34 1-(3-Methylsulfanyl-phenyl)-3-[4-(pyridin-4-yloxy)-benzyl]-urea
35 1-(4-Bromo-2-chloro-phenyl)-3-[4-(pyridin-4-yloxy)-benzyl]-urea
36 1-(2-Methoxy-phenyl)-3-[4-(pyridin-4-yloxy)-benzyl]-urea
37 1-(2-Chloro-4-trifluoromethyl-phenyl)-3-[4-(pyridin-4-yloxy)-benzyl]-urea
38 1-(3,4-Dichloro-phenyl)-3-[4-(pyridin-4-yloxy)-benzyl]-urea
39 4-{4-[3-(3-Chloro-4-methoxy-phenyl)-ureidomethyl]-phenoxy}-pyridine-2-carboxylic acid methylamide
40 1-(4-Chloro-2-trifluoromethyl-phenyl)-3-[4-(pyridin-4-yloxy)-benzyl]-urea
41 1-(4-Ethoxy-phenyl)-3-[4-(pyridin-4-yloxy)-benzyl]-urea
42 4-{4-[3-(5-Chloro-2-methyl-phenyl)-ureidomethyl]-phenoxy}-pyridine-2-carboxylic acid methylamide
43 1-(2-Chloro-5-trifluoromethyl-phenyl)-3-[4-(pyridin-4-yloxy)-benzyl]-urea
44 1-(3,5-Dimethyl-phenyl)-3-[4-(pyridin-4-yloxy)-benzyl]-urea
45 1-(3-Methoxy-phenyl)-3-[4-(pyridin-4-yloxy)-benzyl]-urea
46 1-(4-Fluoro-3-trifluoromethyl-phenyl)-3-[4-(pyridin-4-yloxy)-benzyl]-urea
47 1-(4-Acetyl-phenyl)-3-[4-(pyridin-4-yloxy)-benzyl]-urea
48 1-(2-Bromo-phenyl)-3-[4-(pyridin-4-yloxy)-benzyl]-urea
49 1-(4-Isopropyl-phenyl)-3-[4-(pyridin-4-yloxy)-benzyl]-urea
50 1-(5-Chloro-2-methyl-phenyl)-3-[4-(pyridin-4-yloxy)-benzyl]-urea
51 1-(4-Methylsulfanyl-phenyl)-3-[4-(pyridin-4-yloxy)-benzyl]-urea
52 1-(4-Ethyl-phenyl)-3-[4-(pyridin-4-yloxy)-benzyl]-urea
53 1-(3-Bromo-phenyl)-3-[4-(pyridin-4-yloxy)-benzyl]-urea
54 1-(4-Chloro-2-methyl-phenyl)-3-[4-(pyridin-4-yloxy)-benzyl]-urea
55 1-[4-(Pyridin-4-yloxy)-benzyl]-3-(4-trifluoromethoxy-phenyl)-urea
56 1-(4-tert-Butyl-phenyl)-3-[4-(pyridin-4-yloxy)-benzyl]-urea
57 1-(3,5-Dichloro-phenyl)-3-[4-(pyridin-4-yloxy)-benzyl]-urea
58 1-(4-Bromo-3-methyl-phenyl)-3-[4-(pyridin-4-yloxy)-benzyl]-urea
59 1-(3,4-Dimethyl-phenyl)-3-[4-(pyridin-4-yloxy)-benzyl]-urea
60 1-(3-Chloro-4-methoxy-phenyl)-3-[4-(pyridin-4-yloxy)-benzyl]-urea
61 1-(3-Ethyl-phenyl)-3-[4-(pyridin-4-yloxy)-benzyl]-urea
62 1-(2-Methoxy-5-trifluoromethyl-phenyl)-3-[4-(pyridin-4-yloxy)-benzyl]-urea
63 4-{4-[3-(2-Methoxy-5-trifluoromethyl-phenyl)-ureidomethyl]-phenoxy}-pyridine-2-carboxylic acid methylamide
64 1-(4-Bromo-3-trifluoromethyl-phenyl)-3-[4-(pyridin-4-yloxy)-benzyl]-urea
65 1-[4-(Pyridin-4-yloxy)-benzyl]-3-(3-trifluoromethoxy-phenyl)-urea
66 4-{4-[3-(5-Chloro-2-methoxy-4-methyl-phenyl)-ureidomethyl]-phenoxy}-pyridine-2-carboxylic acid methylamide
67 4-{4-[3-(4-Chloro-2-methoxy-5-trifluoromethyl-phenyl)-ureidomethyl]-phenoxy}-pyridine-2-carboxylic acid methylamide
68 1-(4-Chloro-3-trifluoromethyl-phenyl)-3-{1-[4-(pyridin-4-yloxy)-phenyl]-cyclopropyl}-urea
69 4-(4-{1-[3-(2-Methoxy-5-trifluoromethyl-phenyl)-ureido]-ethyl}-phenoxy)-pyridine-2-carboxylic acid methylamide
70 4-(4-{1-[3-(2,4,5-Trichloro-phenyl)-ureido]-ethyl}-phenoxy)-pyridine-2-carboxylic acid methylamide
71 4-(4-{1-[3-(3,4-Dichloro-phenyl)-ureido]-ethyl}-phenoxy)-pyridine-2-carboxylic acid methylamide
72 4-(4-{1-[3-(5-Chloro-2-methoxy-phenyl)-ureido]-ethyl}-phenoxy)-pyridine-2-carboxylic acid methylamide
73 4-(4-{1-[3-(3-Chloro-4-methyl-phenyl)-ureido]-ethyl}-phenoxy)-pyridine-2-carboxylic acid methylamide
74 4-{4-[3-(4-Chloro-3-methyl-phenyl)-ureidomethyl]-phenoxy}-pyridine-2-carboxylic acid methylamide
75 4-{4-[3-(2-Methoxy-5-methyl-phenyl)-ureidomethyl]-phenoxy}-pyridine-2-carboxylic acid methylamide
76 4-{4[(3-Benzo[1,2,5]thiadiazol-5-yl-ureido)-methyl]-phenoxy}-pyridine-2-carboxylic acid methylamide
77 4-(4-{3-[2-(2-Dimethylamino-ethoxy)-5-trifluoromethyl-phenyl]-ureidomethyl}-phenoxy)-pyridine-2-carboxylic acid methylamide
78 4-{4-[3-(4-Chloro-2-methoxy-5-methyl-phenyl)-ureidomethyl]-phenoxy}-pyridine-2-carboxylic acid methylamide
79 4-{4-[3-(4-Chloro-2-methoxy-phenyl)-ureidomethyl]-phenoxy}-pyridine-2-carboxylic acid methylamide
80 4-{4-[3-(3,4,5-Trimethoxy-phenyl)-ureidomethyl]-phenoxy}-pyridine-2-carboxylic acid methylamide
81 4-{4-[3-(2,5-Dimethoxy-4-nitro-phenyl)-ureidomethyl]-phenoxy}-pyridine-2-carboxylic acid methylamide
82 3-Methoxy-4-{3-[4-(2-methylcarbamoyl-pyridin-4-yloxy)-benzyl]-ureido}-benzoic acid methyl ester
83 4-{4-[3-(4-Chloro-2,5-dimethoxy-phenyl)-ureidomethyl]-phenoxy}-pyridine-2-carboxylic acid methylamide
84 4-{4-[3-(3,5-Dichloro-pyridin-4-yl)-ureidomethyl]-phenoxy}-pyridine-2-carboxylic acid methylamide
85 4-{4[(3-Pyridin-2-yl-ureido)-methyl]-phenoxy}-pyridine-2-carboxylic acid methylamide
86 4-(4-{3-[2-(3-Dimethylamino-propoxy)-phenyl]-ureidomethyl}-phenoxy)-pyridine-2-carboxylic acid methylamide
87 4-{4-[3-(2-Methoxy-pyridin-3-yl)-ureidomethyl]-phenoxy}-pyridine-2-carboxylic acid methylamide
88 4-{4[(3-Pyridin-3-yl-ureido)-methyl]-phenoxy}-pyridine-2-carboxylic acid methylamide
89 4-{4-[3-(2-Chloro-pyridin-4-yl)-ureidomethyl]-phenoxy}-pyridine-2-carboxylic acid methylamide
90 4-{4[(3-Pyridin-4-yl-ureido)-methyl]-phenoxy}-pyridine-2-carboxylic acid methylamide
91 4-{4-[3-(3-Chloro-5-trifluoromethyl-pyridin-2-yl)-ureidomethyl]-phenoxy}-pyridine-2-carboxylic acid methylamide
92 (2-{3-[4-(2-Methylcarbamoyl-pyridin-4-yloxy)-benzyl]-ureido}-4-trifluoromethyl-phenoxy)-acetic acid methyl ester
93 4-{4-[3-(2,5-Dichloro-pyridin-3-yl)-ureidomethyl]-phenoxy}-pyridine-2-carboxylic acid methylamide
94 (2-{3-[4-(2-Methylcarbamoyl-pyridin-4-yloxy)-benzyl]-ureido}-4-trifluoromethyl-phenoxy)-acetic acid
95 4-(4-{3-[4-Chloro-2-(2-dimethylamino-ethoxy)-5-trifluoromethyl-phenyl]-ureidomethyl}-phenoxy)-pyridine-2-carboxylic acid methylamide
96 4-{4 [(3-Isoquinolin-3-yl-ureido)-methyl]-phenoxy}-pyridine-2-carboxylic acid methylamide
97 4-{4 [(3-Quinolin-3-yl-ureido)-methyl]-phenoxy}-pyridine-2-carboxylic acid methylamide
98 (5-Chloro-2-{3-[4-(2-methylcarbamoyl-pyridin-4-yloxy)-benzyl]-ureido}-4-trifluoromethyl-phenoxy)-acetic acid
99 4-{4-[3-(4-Chloro-3-trifluoromethyl-phenyl)-ureidomethyl]-phenoxy}-pyridine-2-carboxylic acid
100 4-{4-[3-(5-Chloro-2-methoxy-phenyl)-ureidomethyl]-2-methyl-phenoxy}-pyridine-2-carboxylic acid methylamide
101 4-{4-[3-(2-Methoxy-quinolin-3-yl)-ureidomethyl]-phenoxy}-pyridine-2-carboxylic acid methylamide
102 4-{4-[3-(2-Methoxy-5-trifluoromethyl-phenyl)-ureidomethyl]-2-methyl-phenoxy}-pyridine-2-carboxylic acid methylamide
103 4-{4-[3-(2,4-Dichloro-6-methoxy-3-methyl-phenyl)-ureidomethyl]-phenoxy}-pyridine-2-carboxylic acid methylamide
104 4-{4-[3-(5-Chloro-2-methoxy-phenyl)-ureidomethyl]-3-methyl-phenoxy}-pyridine-2-carboxylic acid methylamide
105 4-{4-[3-(2-Methoxy-5-trifluoromethyl-phenyl)-ureidomethyl]-3-methyl-phenoxy}-pyridine-2-carboxylic acid methylamide
106 4-(4-{3-[2-(2-Pyrrolidin-1-yl-ethoxy)-5-trifluoromethyl-phenyl]-ureidomethyl}-phenoxy)-pyridine-2-carboxylic acid methylamide
107 (5-Chloro-2-{3-[4-(2-methylcarbamoyl-pyridin-4-yloxy)-benzyl]-ureido}-4-trifluoromethyl-phenoxy)-acetic acid tert-butyl ester
108 4-(4-{3-[2-(2-Diethylamino-ethoxy)-5-trifluoromethyl-phenyl]-ureidomethyl}-phenoxy)-pyridine-2-carboxylic acid methylamide
109 4-(4-{3-[2-(2-Morpholin-4-yl-ethoxy)-5-trifluoromethyl-phenyl]-ureidomethyl}-phenoxy)-pyridine-2-carboxylic acid methylamide
110 4-(4-{3-[4-Chloro-5-methyl-2-(2-morpholin-4-yl-ethoxy)-phenyl]-ureidomethyl}-phenoxy)-pyridine-2-carboxylic acid methylamide
111 4-(4-{3-[2-(2-Methylamino-ethoxy)-5-trifluoromethyl-phenyl]-ureidomethyl}-phenoxy)-pyridine-2-carboxylic acid methylamide
112 4-(4-{3-[2-(2-Piperazin-1-yl-ethoxy)-5-trifluoromethyl-phenyl]-ureidomethyl}-phenoxy)-pyridine-2-carboxylic acid methylamide
113 (5-Chloro-2-{3-[4-(2-methylcarbamoyl-pyridin-4-yloxy)-benzyl]-ureido}-4-trifluoromethyl-phenoxy)-acetic acid methyl ester
114 (5-Chloro-2-{3-[4-(2-methylcarbamoyl-pyridin-4-yloxy)-benzyl]-ureido}-4-trifluoromethyl-phenoxy)-acetic acid isopropyl ester
115 4-(4-{3-[2-(Piperidin-4-yloxy)-5-trifluoromethyl-phenyl]-ureidomethyl}-phenoxy)-pyridine-2-carboxylic acid methylamide
116 4-(4-{3-[4-Chloro-5-methyl-2-(2-pyrrolidin-1-yl-ethoxy)-phenyl]-ureidomethyl}-phenoxy)-pyridine-2-carboxylic acid methylamide
117 4-(4-{3-[4-Chloro-2-(2-diethylamino-ethoxy)-5-methyl-phenyl]-ureidomethyl}-phenoxy)-pyridine-2-carboxylic acid methylamide
118 4-(4-{3-[4-Chloro-2-(2-dimethylamino-ethoxy)-5-methyl-phenyl]-ureidomethyl}-phenoxy)-pyridine-2-carboxylic acid methylamide
119 4-{4-[3-(4-Chloro-2-methoxy-5-methyl-phenyl)-ureidomethyl]-2-methyl-phenoxy}-pyridine-2-carboxylic acid methylamide
120 4-(4-{3-[4-Chloro-5-methyl-2-(2-piperazin-1-yl-ethoxy)-phenyl]-ureidomethyl}-phenoxy)-pyridine-2-carboxylic acid methylamide
121 4-(4-{3-[2-(2-Amino-ethoxy)-5-trifluoromethyl-phenyl]-ureidomethyl}-phenoxy)-pyridine-2-carboxylic acid methylamide
122 4-{4-[3-(2-Piperazin-1-yl-5-trifluoromethyl-phenyl)-ureidomethyl]-phenoxy}-pyridine-2-carboxylic acid methylamide
123 4-{4-[3-(4-Chloro-2-methoxy-5-methyl-phenyl)-ureidomethyl]-phenoxy}-pyridine-2-carb oxylic acid (2-amino-ethyl)-amide
124 4-(4-{3-[4-Chloro-5-methyl-2-(piperidin-4-yloxy)-phenyl]-ureidomethyl}-phenoxy)-pyridine-2-carboxylic acid methylamide
125 4-(4-{3-[4-Chloro-5-methyl-2-(2-methylamino-ethoxy)-phenyl]-ureidomethyl}-phenoxy)-pyridine-2-carboxylic acid methylamide
126 4-{4-[3-(4-Chloro-2-methoxy-5-methyl-phenyl)-ureidomethyl]-phenoxy}-pyridine-2-carb oxylic acid (6-amino-hexyl)-amide
127 4-(4-{3-[4-Chloro-2-(2-methylamino-ethoxy)-5-trifluoromethyl-phenyl]-ureidomethyl}-phenoxy)-pyridine-2-carboxylic acid methylamide
128 4-(4-{3-[4-Chloro-2-(2-piperazin-1-yl-ethoxy)-5-trifluoromethyl-phenyl]-ureidomethyl}-phenoxy)-pyridine-2-carboxylic acid methylamide
129 4-(4-{3-[4-Chloro-2-(2-pyrrolidin-1-yl-ethoxy)-5-trifluoromethyl-phenyl]-ureidomethyl}-phenoxy)-pyridine-2-carboxylic acid methylamide
130 4-(4-{3-[4-Chloro-2-(piperidin-4-yloxy)-5-trifluoromethyl-phenyl]-ureidomethyl}-phenoxy)-pyridine-2-carboxylic acid methylamide
131 4-(4-{3-[4-Chloro-2-(2-morpholin-4-yl-ethoxy)-5-trifluoromethyl-phenyl]-ureidomethyl}-phenoxy)-pyridine-2-carboxylic acid methylamide
132 4-(4-{3-[4-Chloro-2-(2-diethylamino-ethoxy)-5-trifluoromethyl-phenyl]-ureidomethyl}-phenoxy)-pyridine-2-carboxylic acid methylamide
133 4-(4-{3-[4-Chloro-5-methyl-2-(2-morpholin-4-yl-ethoxy)-phenyl]-ureidomethyl}-3-methyl-phenoxy)-pyridine-2-carboxylic acid methylamide
134 4-(4-{3-[4-Chloro-5-methyl-2-(2-pyrrolidin-1-yl-ethoxy)-phenyl]-ureidomethyl}-3-methyl-phenoxy)-pyridine-2-carboxylic acid methylamide
135 4-{4-[3-(3-Methanesulfonyl-phenyl)-ureidomethyl]-phenoxy}-pyridine-2-carboxylic acid methylamide
136 4-(4-{3-[4-Chloro-2-(2-diethylamino-ethoxy)-5-methyl-phenyl]-ureidomethyl}-2-methyl-phenoxy)-pyridine-2-carboxylic acid methylamide
137 4-(4-{3-[4-Chloro-5-methyl-2-(2-pyrrolidin-1-yl-ethoxy)-phenyl]-ureidomethyl}-2-methyl-phenoxy)-pyridine-2-carboxylic acid methylamide
138 4-(4-{3-[4-Chloro-5-methyl-2-(2-morpholin-4-yl-ethoxy)-phenyl]-ureidomethyl}-2-methyl-phenoxy)-pyridine-2-carboxylic acid methylamide
139 4-(4-{3-[4-Chloro-2-(2-dimethylamino-ethoxy)-5-methyl-phenyl]-ureidomethyl}-2-methyl-phenoxy)-pyridine-2-carboxylic acid methylamide
140 4-(4-{3-[2-(2-Amino-ethoxy)-4-chloro-5-trifluoromethyl-phenyl]-ureidomethyl}-phenoxy)-pyridine-2-carboxylic acid methylamide
141 4-(4-{3-[4-Chloro-5-methyl-2-(2-methylamino-ethoxy)-phenyl]-ureidomethyl}-2-methyl-phenoxy)-pyridine-2-carboxylic acid methylamide
142 4-(4-{3-[4-Chloro-5-methyl-2-(piperidin-4-yloxy)-phenyl]-ureidomethyl}-2-methyl-phenoxy)-pyridine-2-carboxylic acid methylamide
143 4-(4-{3-[4-Chloro-5-methyl-2-(2-piperazin-1-yl-ethoxy)-phenyl]-ureidomethyl}-2-methyl-phenoxy)-pyridine-2-carboxylic acid methylamide
144 4-(4-{3-[2-(2-Amino-ethoxy)-4-chloro-5-methyl-phenyl]-ureidomethyl}-2-methyl-phenoxy)-pyridine-2-carboxylic acid methylamide
145 4-(4-{3-[2-(2-Amino-ethoxy)-4-chloro-5-methyl-phenyl]-ureidomethyl}-phenoxy)-pyridine-2-carboxylic acid methylamide
146 4-(2-Methyl-4-{3-[2-(2-methylamino-ethoxy)-5-trifluoromethyl-phenyl]-ureidomethyl}-phenoxy)-pyridine-2-carboxylic acid methylamide
147 4-(4-{3-[4-Chloro-2-(2-methylamino-ethoxy)-5-trifluoromethyl-phenyl]-ureidomethyl}-2-methyl-phenoxy)-pyridine-2-carboxylic acid methylamide
148 4-{4-[3-(4-Chloro-3-trifluoromethyl-phenyl)-ureidomethyl]-phenoxy}-pyridine-2-carboxylic acid (2-amino-ethyl)-amide
149 4-{4-[3-(4-Chloro-3-trifluoromethyl-phenyl)-ureidomethyl]-phenoxy}-pyridine-2-carboxylic acid (2-methylamino-ethyl)-amide
150 4-(4-{3-[2-(2-Dimethylamino-ethoxy)-5-trifluoromethyl-phenyl]-ureidomethyl}-2-methyl-phenoxy)-pyridine-2-carboxylic acid methylamide
151 4-{4-[3-(5-Chloro-2-methoxy-4-methyl-phenyl)-ureidomethyl]-2-methyl-phenoxy}-pyridine-2-carboxylic acid methylamide
152 4-(4-{3-[4-Chloro-2-(2-dimethylamino-ethoxy)-5-trifluoromethyl-phenyl]-ureidomethyl}-2-methyl-phenoxy)-pyridine-2-carboxylic acid methylamide
153 4-(4-{3-[4-Chloro-2-(3-dimethylamino-propoxy)-5-methyl-phenyl]-ureidomethyl}-phenoxy)-pyridine-2-carboxylic acid methylamide
154 4-{4-[3-(3-Methanesulfonyl-phenyl)-ureidomethyl]-2-methyl-phenoxy}-pyridine-2-carboxylic acid methylamide
155 4-(4-{3-[2-(2-Amino-ethoxy)-5-trifluoromethyl-phenyl]-ureidomethyl}-2-methyl-phenoxy)-pyridine-2-carboxylic acid methylamide
156 4-(4-{3-[2-(Pyrrolidin-2-ylmethoxy)-5-trifluoromethyl-phenyl]-ureidomethyl}-phenoxy)-pyridine-2-carboxylic acid methylamide
157 4-{4-[3-(3-Sulfamoyl-phenyl)-ureidomethyl]-phenoxy}-pyridine-2-carboxylic acid methylamide
158 4-{4-[3-(3-Isopropylsulfamoyl-phenyl)-ureidomethyl]-phenoxy}-pyridine-2-carboxylic acid methylamide
159 4-(4-{3-[5-Methyl-2-(piperidin-4-yloxy)-phenyl]-ureidomethyl}-phenoxy)-pyridine-2-carboxylic acid methylamide
160 4-(4-{3-[2-(2-Amino-2-methyl-propoxy)-5-trifluoromethyl-phenyl]-ureidomethyl}-phenoxy)-pyridine-2-carboxylic acid methylamide
161 4-(4-{3-[4-Chloro-2-(4-dimethylamino-butoxy)-5-methyl-phenyl]-ureidomethyl}-phenoxy)-pyridine-2-carboxylic acid methylamide
162 4-[4-(3-{4-Chloro-2-[(2-dimethylamino-ethyl)-methyl-amino]-5-methyl-phenyl}-ureidomethyl)-phenoxy]-pyridine-2-carboxylic acid methylamide
163 4-(4-{3-[4-Chloro-2-(3-dimethylamino-propoxy)-5-methyl-phenyl]-ureidomethyl}-2-methyl-phenoxy)-pyridine-2-carboxylic acid methylamide
164 4-[4-(3-{4-Chloro-2-[(2-dimethylamino-ethyl)-methyl-amino]-5-methyl-phenyl}-ureidomethyl)-2-methyl-phenoxy]-pyridine-2-carboxylic acid methylamide
165 4-(4-{3-[4-Chloro-2-(4-dimethylamino-butoxy)-5-methyl-phenyl]-ureidomethyl}-2-methyl-phenoxy)-pyridine-2-carboxylic acid methylamide
166 4-(4-{3-[2-(2-Methoxy-ethoxy)-5-trifluoromethyl-phenyl]-ureidomethyl}-phenoxy)-pyridine-2-carboxylic acid methylamide
167 4-{4-[3-(3-Methanesulfonylamino-phenyl)-ureidomethyl]-2-methyl-phenoxy}-pyridine-2-carboxylic acid methylamide
168 4-(4-{3-[2-(2-Methoxy-ethoxy)-5-trifluoromethyl-phenyl]-ureidomethyl}-2-methyl-phenoxy)-pyridine-2-carboxylic acid methylamide
169 4-(4-{3-[2-(2-Methylamino-ethoxy)-phenyl]-ureidomethyl}-phenoxy)-pyridine-2-carboxylic acid methylamide
170 4-(4-{3-[5-Methyl-2-(2-methylamino-ethoxy)-phenyl]-ureidomethyl}-phenoxy)-pyridine-2-carboxylic acid methylamide
171 4-(2-Methyl-4-{3-[5-methyl-2-(piperidin-4-yloxy)-phenyl]-ureidomethyl}-phenoxy)-pyridine-2-carboxylic acid methylamide
172 4-(4-{3-[2-(2-Isopropylamino-ethoxy)-5-trifluoromethyl-phenyl]-ureidomethyl}-phenoxy)-pyridine-2-carboxylic acid methylamide
173 4-(4-{3-[5-Chloro-4-methyl-2-(2-pyrrolidin-1-yl-ethoxy)-phenyl]-ureidomethyl}-phenoxy)-pyridine-2-carboxylic acid methylamide
174 4-(4-{3-[5-Chloro-2-(2-dimethylamino-ethoxy)-4-methyl-phenyl]-ureidomethyl}-phenoxy)-pyridine-2-carboxylic acid methylamide
175 4-(4-{3-[2-(2-Amino-ethyl)-5-trifluoromethyl-phenyl]-ureidomethyl}-phenoxy)-pyridine-2-carboxylic acid methylamide
176 4-(4-{3-[2-(2-Amino-ethoxy)-5-chloro-4-methyl-phenyl]-ureidomethyl}-phenoxy)-pyridine-2-carboxylic acid methylamide
177 4-(4-{3-[5-Chloro-4-methyl-2-(2-methylamino-ethoxy)-phenyl]-ureidomethyl}-phenoxy)-pyridine-2-carboxylic acid methylamide
178 4-(4-{3-[5-Chloro-4-methyl-2-(piperidin-4-yloxy)-phenyl]-ureidomethyl}-phenoxy)-pyridine-2-carboxylic acid methylamide
179 4-(4-{3-[5-Chloro-4-methyl-2-(2-piperazin-1-yl-ethoxy)-phenyl]-ureidomethyl}-phenoxy)-pyridine-2-carboxylic acid methylamide
180 4-(4-{3-[2-(2-Methanesulfonylamino-ethoxy)-5-trifluoromethyl-phenyl]-ureidomethyl}-phenoxy)-pyridine-2-carboxylic acid methylamide
181 4-{4[(3-Phenyl-ureido)-methyl]-phenoxy}-pyridine-2-carboxylic acid methylamide
182 4-(4-{3-[5-Chloro-4-methyl-2-(pyrrolidin-2-ylmethoxy)-phenyl]-ureidomethyl}-phenoxy)-pyridine-2-carboxylic acid methylamide
183 4-(4-{3-[5-Chloro-2-(2-isopropylamino-ethoxy)-4-methyl-phenyl]-ureidomethyl}-phenoxy)-pyridine-2-carboxylic acid methylamide
184 4-{2-Methyl-4-[3-(2-piperazin-1-yl-5-trifluoromethyl-phenyl)-ureidomethyl]-phenoxy}-pyridine-2-carboxylic acid methylamide
185 4-(4-{3-[2-(2-Amino-2-methyl-propoxy)-5-chloro-4-methyl-phenyl]-ureidomethyl}-phenoxy)-pyridine-2-carboxylic acid methylamide
186 4-{4-[3-(2-Acetylamino-4-chloro-5-methyl-phenyl)-ureidomethyl]-2-methyl-phenoxy}-pyridine-2-carboxylic acid methylamide
187 4-[4-(3-{4-Chloro-5-methyl-2-[2-(2,2,2-trifluoro-acetylamino)-ethoxy]-phenyl}-ureidomethyl)-phenoxy]-pyridine-2-carboxylic acid methylamide
188 4-(4-{3-[2-(2-Methylamino-ethoxy)-5-trifluoromethanesulfonyl-phenyl]-ureidomethyl}-phenoxy)-pyridine-2-carboxylic acid methylamide
189 4-(2-Methyl-4-{3-[2-(2-methylamino-ethoxy)-5-trifluoromethanesulfonyl-phenyl]-ureidomethyl}-phenoxy)-pyridine-2-carboxylic acid methylamide
190 4-{4-[3-(2-Carbamoylmethoxy-5-trifluoromethyl-phenyl)-ureidomethyl]-phenoxy}-pyridine-2-carboxylic acid methylamide
191 4-(4-{3-[2-(3-Amino-propoxy)-4-chloro-5-trifluoromethyl-phenyl]-ureidomethyl}-phenoxy)-pyridine-2-carboxylic acid methylamide
192 4-{4-[3-(2-Piperazin-1-ylmethyl-5-trifluoromethyl-phenyl)-ureidomethyl]-phenoxy}-pyridine-2-carboxylic acid methylamide
193 4-(4-{3-[4-Chloro-2-(2-methanesulfonylamino-ethoxy)-5-methyl-phenyl]-ureidomethyl}-phenoxy)-pyridine-2-carboxylic acid methylamide
194 4-(4-{3-[4-Chloro-2-(2-methanesulfonylamino-ethoxy)-5-trifluoromethyl-phenyl]-ureidomethyl}-phenoxy)-pyridine-2-carboxylic acid methylamide
195 4-(4-{3-[2-(2-Hydroxy-ethyl)-phenyl]-ureidomethyl}-2-methyl-phenoxy)-pyridine-2-carboxylic acid methylamide
196 4-{4-[3-(2-Hydroxymethyl-4-methyl-phenyl)-ureidomethyl]-2-methyl-phenoxy}-pyridine-2-carboxylic acid methylamide
197 4-(4-{3-[2-(2-Hydroxy-ethoxy)-5-trifluoromethyl-phenyl]-ureidomethyl}-phenoxy)-pyridine-2-carboxylic acid methylamide
198 4-{4-[3-(2-Hydroxymethyl-phenyl)-ureidomethyl]-2-methyl-phenoxy}-pyridine-2-carboxylic acid methylamide
199 4-(4-{3-[2-(1-Carbamoyl-1-methyl-ethoxy)-5-trifluoromethyl-phenyl]-ureidomethyl}-phenoxy)-pyridine-2-carboxylic acid methylamide
200 4-{2-Methyl-4-[3-(2-methylcarbamoylmethyl-5-trifluoromethyl-phenyl)-ureidomethyl]-phenoxy}-pyridine-2-carboxylic acid methylamide
201 4-{4-[3-(2-[1,2,4]Triazol-1-yl-5-trifluoromethyl-phenyl)-ureidomethyl]-phenoxy}-pyridine-2-carboxylic acid methylamide
202 4-{2-Methyl-4-[3-(2-[1,2,4]triazol-1-yl-5-trifluoromethyl-phenyl)-ureidomethyl]-phenoxy}-pyridine-2-carboxylic acid methylamide
203 4-{2-Methyl-4-[3-(2-[1,2,3]triazol-1-yl-5-trifluoromethyl-phenyl)-ureidomethyl]-phenoxy}-pyridine-2-carboxylic acid methylamide
204 4-{4-[3-(2-Hydroxy-5-trifluoromethyl-phenyl)-ureidomethyl]-2-methyl-phenoxy}-pyridine-2-carboxylic acid methylamide
205 2-Oxo-6-trifluoromethyl-2,3-dihydro-indole-1-carboxylic acid 4-(2-methylcarbamoyl-pyridin-4-yloxy)-benzylamide
206 4-{4-[3-(2-[1,2,3]Triazol-1-yl-5-trifluoromethyl-phenyl)-ureidomethyl]-phenoxy}-pyridine-2-carboxylic acid methylamide
207 4-{4-[3-(2-Carbamoylmethyl-5-trifluoromethyl-phenyl)-ureidomethyl]-2-methyl-phenoxy}-pyridine-2-carboxylic acid methylamide
208 4-(4-{3-[2-(2-Oxo-piperazin-1-ylmethyl)-5-trifluoromethyl-phenyl]-ureidomethyl}-phenoxy)-pyridine-2-carboxylic acid methylamide
209 4-{4-[3-(2-Carbamoylmethyl-5-trifluoromethyl-phenyl)-ureidomethyl]-phenoxy}-pyridine-2-carboxylic acid methylamide
210 4-{4-[3-(5-Methyl-pyridin-2-yl)-ureidomethyl]-phenoxy}-pyridine-2-carboxylic acid methylamide
211 4-{2-Methyl-4-[3-(5-methyl-pyridin-2-yl)-ureidomethyl]-phenoxy}-pyridine-2-carboxylic acid methylamide
212 4-{4-[3-(4-Methyl-pyridin-2-yl)-ureidomethyl]-phenoxy}-pyridine-2-carboxylic acid methylamide
213 4-{2-Methyl-4-[3-(4-methyl-pyridin-2-yl)-ureidomethyl]-phenoxy}-pyridine-2-carboxylic acid methylamide
214 4-(4-{3-[2-(Acetylamino-methyl)-5-trifluoromethyl-phenyl]-ureidomethyl}-2-methyl-phenoxy)-pyridine-2-carboxylic acid methylamide
215 4-(2-Methyl-4-{3-[5-methyl-2-(2-methylamino-ethoxy)-phenyl]-ureidomethyl}-phenoxy)-pyridine-2-carboxylic acid methylamide
216 4-{4-[3-(5-Chloro-2-methoxy-4-methyl-phenyl)-ureidomethyl]-2-fluoro-phenoxy}-pyridine-2-carboxylic acid methylamide
217 4-{2-Fluoro-4-[3-(2-methoxy-5-trifluoromethyl-phenyl)-ureidomethyl]-phenoxy}-pyridine-2-carboxylic acid methylamide
218 4-{4-[3-(4-Chloro-2-methoxy-5-methyl-phenyl)-ureidomethyl]-2-fluoro-phenoxy}-pyridine-2-carboxylic acid methylamide
219 4-{4-[3-(4-Chloro-5-methyl-2-pyrrol-1-yl-phenyl)-ureidomethyl]-phenoxy}-pyridine-2-carboxylic acid methylamide
220 (2-{3-[3-Methyl-4-(2-methylcarbamoyl-pyridin-4-yloxy)-benzyl]-ureido}-4-trifluoromethyl-phenyl)-acetic acid
221 4-{4-[3-(2-Aminomethyl-5-trifluoromethyl-phenyl)-ureidomethyl]-2-methyl-phenoxy}-pyridine-2-carboxylic acid methylamide
222 4-{4-[3-(5-Trifluoromethyl-[1,3,4]thiadiazol-2-yl)-ureidomethyl]-phenoxy}-pyridine-2-carboxylic acid methylamide
223 4-{4-[3-(5-tert-Butyl-2H-pyrazol-3-yl)-ureidomethyl]-phenoxy}-pyridine-2-carboxylic acid methylamide
224 4-{4-[3-(5-tert-Butyl-isoxazol-3-yl)-ureidomethyl]-phenoxy}-pyridine-2-carboxylic acid methylamide
225 4-(4-{3-[3-Chloro-4-(3-oxo-morpholin-4-yl)-phenyl]-ureidomethyl}-phenoxy)-pyridine-2-carboxylic acid methylamide
226 4-{4-[3-(2-Chloro-pyridin-3-yl)-ureidomethyl]-phenoxy}-pyridine-2-carboxylic acid methylamide
227 4-{4-[3-(6-Methoxy-pyridin-3-yl)-ureidomethyl]-phenoxy}-pyridine-2-carboxylic acid methylamide
228 4-{4-[3-(3-Dimethylamino-phenyl)-ureidomethyl]-phenoxy}-pyridine-2-carboxylic acid methylamide
229 4-{4-[3-(2-Ethoxy-5-trifluoromethyl-phenyl)-ureidomethyl]-2-methyl-phenoxy}-pyridine-2-carboxylic acid methylamide
230 4-{4-[3-(2-Ethoxy-5-trifluoromethyl-phenyl)-ureidomethyl]-phenoxy}-pyridine-2-carboxylic acid methylamide
231 4-{4-[3-(4-Chloro-2-methoxy-5-trifluoromethyl-phenyl)-ureidomethyl]-2-fluoro-phenoxy}-pyridine-2-carboxylic acid methylamide
232 4-{4-[3-(5-Chloro-2-methoxy-phenyl)-ureidomethyl]-2-fluoro-phenoxy}-pyridine-2-carboxylic acid methylamide
233 4-{2-Fluoro-4-[3-(3-trifluoromethyl-phenyl)-ureidomethyl]-phenoxy}-pyridine-2-carboxylic acid methylamide
234 4-{4-[3-(3-Chloro-4-methyl-phenyl)-ureidomethyl]-2-fluoro-phenoxy}-pyridine-2-carboxylic acid methylamide
235 4-{2-Fluoro-4-[3-(2-[1,2,4]triazol-1-yl-5-trifluoromethyl-phenyl)-ureidomethyl]-phenoxy}-pyridine-2-carboxylic acid methylamide
236 4-{4-[3-(5-Methyl-isoxazol-3-yl)-ureidomethyl]-phenoxy}-pyridine-2-carboxylic acid methylamide
237 4-(4-{3-[2-(2-Acetylamino-ethoxy)-4-chloro-5-methyl-phenyl]-ureidomethyl}-phenoxy)-pyridine-2-carboxylic acid methylamide
238 4-(4-{3-[2-(2-Acetylamino-ethoxy)-4-chloro-5-trifluoromethyl-phenyl]-ureidomethyl}-phenoxy)-pyridine-2-carboxylic acid methylamide
239 4-(4-{3-[2-(2-Acetylamino-ethoxy)-5-trifluoromethyl-phenyl]-ureidomethyl}-phenoxy)-pyridine-2-carboxylic acid methylamide
240 4-{4-[3-(2-Imidazol-1-yl-5-trifluoromethyl-phenyl)-ureidomethyl]-2-methyl-phenoxy}-pyridine-2-carboxylic acid methylamide
241 4-{4-[3-(4-Acetylamino-3-trifluoromethyl-phenyl)-ureidomethyl]-phenoxy}-pyridine-2-carboxylic acid methylamide
242 4-[4-(3-{4-Chloro-2-[2-(1,3-dioxo-1,3-dihydro-isoindol-2-yl)-ethoxy]-5-trifluoromethyl-phenyl}-ureidomethyl)-phenoxy]-pyridine-2-carboxylic acid methylamide
243 4-{4-[3-(2-Imidazol-1-yl-5-trifluoromethyl-phenyl)-ureidomethyl]-phenoxy}-pyridine-2-carboxylic acid methylamide
244 [2-(5-Chloro-4-methyl-2-{3-[4-(2-methylcarbamoyl-pyridin-4-yloxy)-benzyl]-ureido}-phenoxy)-ethyl]-methyl-carbamic acid tert-butyl ester
245 1-Phenyl-3-[4-(pyridin-4-yloxy)-benzyl]-urea
246 1-[4-Chloro-2-(2-dimethylamino-ethoxy)-5-methyl-phenyl]-3-[3-methyl-4-(pyridin-4-yloxy)-benzyl]-urea
247 N-[4-(4-{3-[4-Chloro-2-(2-dimethylamino-ethoxy)-5-methyl-phenyl]-ureidomethyl}-2-methyl-phenoxy)-pyridin-2-yl]-acetamide
248 1-(4-Chloro-2-methoxy-5-methyl-phenyl)-3-[3-methyl-4-(pyridin-4-yloxy)-benzyl]-urea
249 1-[4-(2-Methyl-furo[3,2-b]pyridin-7-yloxy)-benzyl]-3-phenyl-urea
250 1-[4-Chloro-2-(2-dimethylamino-ethoxy)-5-methyl-phenyl]-3-[3-methyl-4-(pyrimidin-4-yloxy)-benzyl]-urea
251 1-[4-(6-Amino-pyrimidin-4-yloxy)-3-methyl-benzyl]-3-[4-chloro-2-(2-dimethylamino-ethoxy)-5-methyl-phenyl]-urea
252 1-(4-Chloro-2-methoxy-5-methyl-phenyl)-3-[3-methyl-4-(2-methyl-furo[3,2-b]pyridin-7-yloxy)-benzyl]-urea
253 1-[4-Chloro-2-(2-dimethylamino-ethoxy)-5-methyl-phenyl]-3-[3-methyl-4-(2-methyl-furo[3,2-b]pyridin-7-yloxy)-benzyl]-urea
254 1-[4-(2-Amino-pyridin-4-yloxy)-3-methyl-benzyl]-3-[4-chloro-2-(2-dimethylamino-ethoxy)-5-methyl-phenyl]-urea
255 4-[4-[[4-chloro-3-(trifluoromethyl)-phenyl]carbamoylamino]phenoxy]-N-methyl-pyridine-2-carboxamide
and physiologically acceptable salts, solvates and stereoisomers thereof, including mixtures thereof in all ratios.

13. Compounds according to claim 1
a) 1-(1-Methyl-2-oxo-5-trifluoromethyl-1,2-dihydro-pyridin-3-yl)-3-[4-(1H-pyrrolo[2,3-b]pyridin-4-yloxy)-benzyl]-urea
b) 1-(1-Methyl-2-oxo-5-trifluoromethyl-1,2-dihydro-pyridin-3-yl)-3-[4-(6-trifluoromethyl-quinolin-4-yloxy)-benzyl]-urea
c) 1-(1-Methyl-2-oxo-5-trifluoromethyl-1,2-dihydro-pyridin-3-yl)-3-[4-(1H-pyrrolo[2,3-b]pyridin-4-yloxy)-benzyl]-urea
d) 1-(1-Methyl-2-oxo-5-trifluoromethyl-1,2-dihydro-pyridin-3-yl)-3-[4-([1,8]naphthyridin-4-yloxy)-benzyl]-urea
e) 1-(1-Methyl-2-oxo-5-trifluoromethyl-1,2-dihydro-pyridin-3-yl)-3-[4-(2-oxo-1,2,3,4-tetrahydro-pyrido[2,3-d]pyrimidin-5-yloxy)-benzyl]-urea
f) 1-[3-Methyl-4-(2-methyl-pyridin-4-yloxy)-benzyl]-3-(1-methyl-2-oxo-5-trifluoromethyl-1,2-dihydro-pyridin-3-yl)-urea
g) 4-{2-Methyl-4-[3-(1-methyl-2-oxo-5-trifluoromethyl-1,2-dihydro-pyridin-3-yl)-ureidomethyl]-phenoxy}-pyridine-2-carboxylic acid methylamide
h) 4-{4-[3-(1-Methyl-2-oxo-5-trifluoromethyl-1,2-dihydro-pyridin-3-yl)-ureidomethyl]-phenoxy}-pyridine-2-carboxylic acid methylamide
i) 1-(1-Methyl-2-oxo-5-trifluoromethyl-1,2-dihydro-pyridin-3-yl)-3-[4-(quinolin-4-yloxy)-benzyl]-urea
j) 1-[3-Methyl-4-(2-oxo-1,2,3,4-tetrahydro-pyrido[2,3-d]pyrimidin-5-yloxy)-benzyl]-3-(1-methyl-2-oxo-5-trifluoromethyl-1,2-dihydro-pyridin-3-yl)-urea
k) 4-{4-[3-(1-Ethyl-2-oxo-5-trifluoromethyl-1,2-dihydro-pyridin-3-yl)-ureidomethyl]-phenoxy}-pyridine-2-carboxylic acid methylamide
l) 4-{4-[3-(1-Benzyl-2-oxo-5-trifluoromethyl-1,2-dihydro-pyridin-3-yl)-ureidomethyl]-phenoxy}-pyridine-2-carboxylic acid methylamide
m) 1-(1-Methyl-2-oxo-5-trifluoromethyl-1,2-dihydro-pyridin-3-yl)-3-[4-(3-trifluoromethyl-pyridin-4-yloxy)-benzyl]-urea
n) 4-{4-[3-(1-Hydroxymethyl-2-oxo-5-trifluoromethyl-1,2-dihydro-pyridin-3-yl)-ureidomethyl]-phenoxy}-pyridine-2-carboxylic acid methylamide
o) (3-{3-[4-(2-Methylcarbamoyl-pyridin-4-yloxy)-benzyl]-ureido}-2-oxo-5-trifluoro-methyl-2H-pyridin-1-yl)-acetic acid
p) 4-{4-[3-(1-Aminomethyl-2-oxo-5-trifluoromethyl-1,2-dihydro-pyridin-3-yl)-ureidomethyl]-phenoxy}-pyridine-2-carboxylic acid methylamide
q) 4-{4-[3-(1-Methylaminomethyl-2-oxo-5-trifluoromethyl-1,2-dihydro-pyridin-3-yl)-ureidomethyl]-phenoxy}-pyridine-2-carboxylic acid methylamide
r) 4-{4-[3-(1-Dimethylaminomethyl-2-oxo-5-trifluoromethyl-1,2-dihydro-pyridin-3-yl)-ureidomethyl]-phenoxy}-pyridine-2-carboxylic acid methylamide
and physiologically acceptable salts, solvates and stereoisomers thereof, including mixtures thereof in all ratios.

14. Compounds according to claim 1
a) (2-Hydroxy-5-trifluoromethyl-pyridin-3-yl)-carbamic acid 3-methyl-4-(2-methylcarbamoyl-pyridin-4-yloxy)-benzyl ester
b) (2-Hydroxy-5-methyl-pyridin-3-yl)-carbamic acid 4-(2-methylcarbamoyl-pyridin-4-yloxy)-benzyl ester
c) (4-Trifluoromethyl-pyridin-2-yl)-carbamic acid 4-(2-methylcarbamoyl-pyridin-4-yloxy)-benzyl ester
d) (4-Trifluoromethyl-pyridin-2-yl)-carbamic acid 3-methyl-4-(2-methylcarbamoyl-pyridin-4-yloxy)-benzyl ester
e) (2-Hydroxy-5-trifluoromethyl-pyridin-3-yl)-carbamic acid 4-(2-methylcarbamoyl-pyridin-4-yloxy)-benzyl ester
f) (4-Chloro-3-trifluoromethyl-phenyl)-carbamic acid 4-(2-methylcarbamoyl-pyridin-4
and physiologically acceptable salts, solvates and stereoisomers thereof, including mixtures thereof in all ratios.

15. Compounds according to claim 1
a) 1-[4-(4-Oxo-4,5-dihydro-3H-imidazo[4,5-c]pyridin-2-ylmethyl)-phenyl]-3-(3-trifluoromethyl-phenyl)-urea
b) 1-[4-(3-Methyl-4-oxo-4,5-dihydro-3H-imidazo[4,5-c]pyridin-2-ylmethyl)-phenyl]-3-(1-methyl-2-oxo-5-trifluoromethyl-1,2-dihydro-pyridin-3-yl)-urea
c) 1-(2-Methoxy-5-trifluoromethyl-phenyl)-3-[4-(3-methyl-4-oxo-4,5-dihydro-3H-imidazo[4,5-c]pyridin-2-ylmethyl)-phenyl]-urea
d) 1-(1-Methyl-2-oxo-5-trifluoromethyl-1,2-dihydro-pyridin-3-yl)-3-[4-(4-oxo-4,5-dihydro-3H-imidazo[4,5-c]pyridin-2-ylmethyl)-phenyl]-urea
e) 1-(5-Methyl-pyridin-3-yl)-3-[4-(4-oxo-4,5-dihydro-3H-imidazo[4,5-c]pyridin-2-ylmethyl)-phenyl]-urea
and physiologically acceptable salts, solvates and stereoisomers thereof, including mixtures thereof in all ratios.

16. Compounds according to claim 1
a) 4-{4-[(4-Chloro-3-trifluoromethyl-phenylcarbamoyl)-methyl]-2-methyl-phenoxy}-pyridine-2-carboxylic acid methylamide
b) 4-{4-[2-(4-Chloro-3-trifluoromethyl-phenylcarbamoyl)-1-hydroxy-ethyl]-2-methyl-phenoxy}-pyridine-2-carboxylic acid methylamide
c) 4-{4-[2-(4-Chloro-3-trifluoromethyl-phenylcarbamoyl)-ethyl]-2-methyl-phenoxy}-pyridine-2-carboxylic acid methylamide
d) 4-{4-[(1-Methyl-2-oxo-5-trifluoromethyl-1,2-dihydro-pyridin-3-ylcarbamoyl)-methoxy]-phenoxy}-pyridine-2-carboxylic acid methylamide
e) N-(1-Methyl-2-oxo-5-trifluoromethyl-1,2-dihydro-pyridin-3-yl)-2-[4-(2-oxo-1,2,3,4-tetrahydro-pyrido[2,3-d]pyrimidin-5-yloxy)-phenoxy]-acetamide
f) N-(2-Fluoro-5-trifluoromethyl-phenyl)-2-[4-(2-oxo-1,2,3,4-tetrahydro-pyrido[2,3-d]pyrimidin-5-yloxy)-phenoxy]-acetamide
g) N-(1-Methyl-2-oxo-5-trifluoromethyl-1,2-dihydro-pyridin-3-yl)-2-[4-(quinolin-4-yloxy)-phenoxy]-acetamide
h) 2-[4-(3a,7a-Dihydro-1H-pyrrolo[2,3-b]pyridin-4-yloxy)-phenoxy]-N-(1-methyl-2-oxo-5-trifluoromethyl-1,2-dihydro-pyridin-3-yl)-acetamide
i) 4-{4-[(2-Hydroxy-5-trifluoromethyl-pyridin-3-ylcarbamoyl)-methyl]-2-methyl-phenoxy}-pyridine-2-carboxylic acid methylamide
j) 4-{2-Methyl-4-[(1-methyl-2-oxo-5-trifluoromethyl-1,2-dihydro-pyridin-3-ylcarbamoyl)-methyl]-phenoxy}-pyridine-2-carboxylic acid methylamide
k) 2-[3-Methyl-4-(3-methyl-2-oxo-1,2,3,4-tetrahydro-pyrido[2,3-d]pyrimidin-5-yloxy)-phenyl]-N-(1-methyl-2-oxo-5-trifluoromethyl-1,2-dihydro-pyridin-3-yl)-acetamide
l) N-(2-Fluoro-5-trifluoromethyl-phenyl)-2-[3-methyl-4-(3-methyl-2-oxo-1,2,3,4-tetrahydro-pyrido[2,3-d]pyrimidin-5-yloxy)-phenyl]-acetamide
and physiologically acceptable salts, derivatives, solvates, prodrugs and stereoisomers thereof, including mixtures thereof in all ratios.

17. Compounds according to claim 1 and physiologically acceptable salts, solvates and stereoisomers thereof, including mixtures thereof in all ratios, as DDR2 inhibitors.

18. Pharmaceutical composition comprising at least one compound according to claim 1 and/or physiologically acceptable salts, solvates and stereoisomers thereof, including mixtures thereof in all ratios.

19. Pharmaceutical composition according to claim 18 comprising further excipients and/or adjuvants.

20. Pharmaceutical composition comprising at least one compound according to claim 1 and/or physiologically acceptable salts, solvates and stereoisomers thereof, including mixtures thereof in all ratios, and at least one further medicament active ingredient.

21. Process for the preparation of a pharmaceutical composition, characterised in that a compound according to claim 1 and/or one of its physiologically acceptable salts, solvates and stereoisomers, including mixtures thereof in all ratios, is brought into a suitable dosage form together with a solid, liquid or semi-liquid excipient or adjuvant.

22. Medicament comprising at least one compound according to claim 1 and/or one of its physiologically acceptable salts, solvates and stereoisomers, including mixtures thereof in all ratios, for use in the treatment and/or prophylaxis of physiological and/or pathophysiological states.

23. Medicament comprising at least one compound according to claim 1 and/or one of its physiologically acceptable salts, derivatives, solvates and stereoisomers, including mixtures thereof in all ratios, for use in the treatment and/or prophylaxis of physiological and/or pathophysiological states, selected from the group consisting of osteoarthritis, hepatocirrhosis, traumatic cartilage injuries, pain, allodynia or hyperalgesia.

24. A method comprising administering a pharmaceutical composition according to claim 18 by intra-articular administration for the treatment and/or prophylaxis of physiological and/or pathophysiological states selected from the group consisting of osteoarthritis, traumatic cartilage injuries, pain, allodynia or hyperalgesia.

25. Set (kit) consisting of separate packs of
a) an effective amount of a compound according to claim 1 and/or physiologically acceptable salts, derivatives, solvates and stereoisomers thereof, including mixtures thereof in all ratios, and
b) an effective amount of a further medicament active ingredient.
